SEQUENCE LISTING (3 sequences)

SEQ ID NO: 1 - DNA, 21322 bp, synthetic construct
  source 1..21322: mol_type = other DNA
ttttgtttattggggcaaaactctgtatagttttggagcctgatgtggaagtttctctgtagagcaggctagccccaaactatagaggtcttcctgcctctgacttctgagtgctgggattaaaggtgtgggccactaaagactaaagaaatagaagcaaaccacaactaagttgtgaagtagaagagtataattgctgatataaaggatttgccaattcttaggttttgtgagtccccctaaatagaattgagctgagggagtactgcctataaaataacccaaagataagtagtatgctaactaggcctcttacagtttagcatgctaagaaacacatttgttctccagggttgtccacacatttggctccaaaccatgacaaaggagaaacccctggttttaaaacagcctccaggccagaacatggcttttacaagctgctaagggagcattactggatcacaatgagctttagtgcatggtgtgctataaaggtccagtttccttctagggaaaatcatagtcatgccaaatgggcaagatgagaccaggtgactaatatttgaaaagggaaacactgctgttggacttagcaagcaaaactttctgtttaggtcctcaatcatggagaagtaacatagctccttcccaaataaaggaacatgcttggaagagccagtaacagcctggggcctagccttgggagaatcccatcagtccctggggtatggagaatcaagatgttttggagagaatgtttgttctggagccagggcctttgtggaacagttgcttcagatcctgagaacacaactcttcccaccacatggagctgtgattattctgtcccagggccactatgggattagacagtaatgacagtaatgttgttgggataccctgtgttccccttgtgttgcattgttggagtaatcttttcctgagtttgtcccattgtatgataatttctgctgacttcctagaagtctcctatttccctccatttccccatgaaattggtataacagaagctattcctcttaagaagcttagttagaacaagacatagctcaagcaagacctccctaccccagttctatctctttttaaaatgccttacctgtatttcttatcttctggcaactccgcctctggacctgtcagtaaattatggtgtgctgtttcgtgctactgtgtactatgaaatacttctggtaatatcttttcagtcgaagtctttcatttgtctacctagcctaacttgcattttcctgtcactatttaaattctttaaagtgtaagctggagtcggaccctaaaggaactgaaatatgataacatatgtggaaagcaaaatctgttttaggagccaggtggtggtggtgcataactttaatcccagcaatgcagagtcaaaggtagggtgatctatgtgattttgaggccagcgtgatctacactgctagttccaggacaagtagtgctcatacacagagaaaccaatatctaagaaaataaacaaaaaatctgttttagcctctctcctctttgagaattagccagagatggatgcatgttatcattcaagagatgtttctttattagtctcttaagtcagtggaattatttttcactggaaaggcatattctagaacagctgcaattccagccttttagaacaaggtttcagagacaagctatctcatgttctccagggcaaagaccttgctgtcttctgtacaaaaccttcaaaaatcttaaatgaatgtacaacctcagagatgtaaatttgtccatgagagttacatagccaactatccaatgaattcacagaaaattattccaatggaggagcatggcttctaccttcaggccaaaataggaggaataaattaaaatgcaggaggattcagggaaggaaccaggtgctggagtcatctttatgaaaagggacctgattgtggtagtgggaattattttgtaattccccaacatgttttaattcataacatgtaaataaacaccgaccagattgtgtgggtgtacagtacggaaggttatcttgaaccaatgaatattgaattttcaattagtcaactatcctgtgcagatatgttacgtcagctcagaggtttgtgaatcaggccattgactgtaactttgtgtcagattacaaatctcagtagctaacatatgcctatggatgacatcaatctatctcttgttaatgactatagtaaaattttgtttgtaatctaacaaataaggcttccctgaatatcagtgtgcagagctaagtcactagttagctacacatctggttaaaacacactaagtcactagttaagcacaaacctaatctactagttagccatagaggcagagaggtcctgcttaggatatgtcttattataagtaagcatcttaagaggaatagcttcttttttactatttcccgggggaaatggagggaaaccagagacttctaggaagtaagcagaaattgtcataaaatggaacaaactgaggagaaggttactcaaacaatgcagcacacagggaatacagggtatcccaagaacattactgatgaagctcacagaggccttcggtccaataaccacagctccatgtggtgaggagagttgtgttttaagaatgtgaaccaatgaccccacaaaggtgctggatccatacaattatattctctctaaaacatctgcctagttttagtggatgagctctattttattctccatacctcagggcctaatgtaaaattcctagggctaggcaccactggctctgccaagcatgttcctgggtttgagaaggagctatgttccttctccatgagagaggacgcaaacagaaagtcttcctttctcagaccaaccccaaacacaacagtgttttccttttcaaatacctgtcacctggtttcatcttgcccatctggcagaactgtgattttctctagaaggaaactgcaactttatggcacaccataacctaatgatcattgtaaatattattctttagtaatctcttaagtctgtggagatatttctcactggaaaggaatattctagaacagctgcacttccatccattgagaaaaagttatctgagacaaactctttcatgttgacacaattgtcttgatcccagtctgttgctttccagggaaatgacctcactgtcctcatgattacacctccttcaattaagttactatgtgaccacagagttttgaaatcatgcattcaagtcatgcagatgactgtcagttgcagtcacaaaaaaaggatttgcgtgtggagcatggtttcagccttcatatcagagtagaaagaatagtttattgagttcactgcaagggggaaacagggtaggaaatcaactacttaagtcaccatagtgaaacagtacctgattgtggtaatgtgatagtttgaatgtaatttcatagggaggggcactattagaaggtgtgactttgttgcagcaagtttagaatttttggaagaagtgtgtcatcactggtgataggctttggggttttccatccttcacatcacactccccactatggtgacaattgactgaacctctgaatctgtaagtcagccagtccaattaagtgttttatttataagagttatggtagtcatgaaggttattcatagcaataagaatcctatataagacaggcagattcagtgatttttttctttaaattctcaggatttattaattactgtaatgtgaataaagatagacctgatttgtgggtttacattttgtgaaggttatattgatctcagacctttccacaggatatgtagttgagagttgctttgaacaaagctaatcctgaatctgctaatcattactgggaagacactgctgcaattctttcaagagtgggatttgactggtgattgcgaaggaaatagagaccttttaattagtgttttccaatccctttggcttgtatcagggactgcaaataaaagagatatatgaaagaattgagttcatttaccaattttaaagctggtataggcacattgcaaaaaggggtgggagaaggaaggaacatgggcaaatacctgctggtaggcagtggaaattagcagtatggagtcttttcatcaattttagaagatcaataaaatgcccgagtggccatttcttggggtccacagacaagggtaatcaataatcttgaaaaaaaggcctgatgttatcctgataccaagttgttgtagttatgctagaaaatatgtagatgatatatagaaagtcatcataaattgctgtgaccctgcctaacgagcctgttattttactgggttaagatgtatagcctgagaaaagatgtgagttatacttggtgttctctcatccagatacctagtctctggagcagatagccataaagtctgtttcaacttttcaaaagaattgtcaagattataactgcatcatatctatagtatccatagtctctttcatccctctaatccataccatgatgcctcactatcagttacactgtaacaatactgtgtgacgtatgaatgtttgaatgtgacctgtgtttggctgtgggacttcccagaattaatcaaggcattggctagcctggatagatactgagtgtgtaatagtatgtgcacaaaacttaccctgtgtcttggaagagaacaggaagtgctctggatgactgggagaaagtctcctaaaacacaaactttccagactagggcactgtctgtggggatggaagggcaaagctaggaatgcctatgctgtctgagttatttggtctttccataccgtggaggggagctgctgatgctctatggtccaactgtgtgtccctagctatttaggcactctgtgaaggtttagcactcaggaggaccagttgaaggtagctagcatgttatctccaagactctctgctggctctcttgtaggagctataacggagacgacagccacaacatggtgagtgtggaggcttcagaacaggaacagcaggtctgcctggctgtcattgctgttggaaatgagagggtctgtgagccatgtactgacctgggcagtggaccttatcctttgatcctccatgaatgtaatttccctggggaaagagagggaggtgttttagccctggctgtgggaagttgatatgcttgtaatttcagtgcccagtgtgcatcacgggattgttttgggtgtgtgctggaacatatattgaaaaactgaaaatgagaaaagatcccttttttctgactggcgggtctcctagaggttctgattggctagtgaggcctggtgacaggaccaggtaagcagaagagacatagaacaatggcttaatcttgagggtgtggccccaggagcacagaaatccagaaaccaggaggtgagtcttaaggtgggggcttttaatgcttctgagggtgggcctgccaatgtcgggcttactaagggcagagagaactgaggtgatttcagggtagggcccaccatggtaccttgaggatggggtttggaatggggctaccaagtattctaactactttgcatagatatagaagcctgtaaggcttatacccaaaagacccagactccctcggtagaggggtgtccatttaagtctggcttcttcctccctttgacaagtaacaatgtggggagggtgtaagtcaggacttggtgggttcatacacagcaggaggtatgtgtggagaagtattcctttagctgccattctgtgtacgttaggcagctgttccttgaggaggtggagaaggcaagttgggtgaagcttctgccttatcagggagtttcaggtagagaaaggctgatgccagtccagatgttgattagcagcaccattccaaaccccaccctccaggtgccaagggcaggcaaacccctggattcttgtcagttcacacccagcctttaaaaagtccaccattttccaccagagaaggagaaagtgtcaacatctcctcaataaattgggagaatggggaggggagagtgaggtaggaaaaagagaagaggaggagaggagaggagaggagaggaggacatgagggagcaggaagattgagttgggtgaagaaaaggggggagcaagaaaagatataccacaatagagggagccattataggtttatagagaaatctggcagtatggaactgtccagatatccacaaggatgaccccaactaacaatctaggcaataatggagagcccttcaaggcccttcccctataatgagactgatgactaccttaaatgccaccctagagccttcatcttgtagctcatagaagcagaagcagacacccacagctaagcacagagctctactcctggaatccagttgtagtgagggaagattgatgagcaaagtggtcaagaacagtctggagaaacccacagatacagctgacctaaacaagtgagagcgaatggacctaggctgatatctggttagccagcataggactgatccagaacccctgaatgtgggtgagcattaggaggccacagcaatctatggggcctctggtagtgcaacaatatttatccctaatataccaatggattttttgaagcccattccccatatagggatactatctcaggctagaagcatgggggcaggcctgagccctacccaaaataaaatcacagatttggtgatccctctaggaaggcctcaccctcactgtggagtagatgagggatggtatttggggggtttgtggggagcatggcagaatgagaagatgagggaactgggattgatatataaaataacaaacattgtttataatttaaatacaatttataaaatacaaaaagaataggaaagacagggtggcaaataccttagctaactattgctggtgccatagccatgaaatactggagctgcagcgaagtttatgtacattacccagaatcagtcacagtgtccctttcttctgtggtcctcccaccatacaccattgaccattttcatcttggatgaatttgaaagcctgaagttcctgtctccacattctaagtaccatgattacaggcgtctgccaacacgcctgggttaggtggtttcagcttcacttagtgaggcattagaaggagtaacatgaggagtttcacttgactatattcaaactgtgtttacctctgtgagcctgggttttaaccatctgcagaataaggcagaactcttcccacgtcctttgatgagttataacagtctcagtgataaagaaaaaaagtatttataataattggaagacagtgggctttgagtcaggaaggctgcatttgatgggtgccttcttctgttactgaagttaggatcagcacctgtatcctggtttccctaaccagccaggctcagtcactcattctcaggaagcaaagtagatgaagcaacttatctagtggcatgcccaactatgatgcaggtactctggagtcaagtttgacagatctctctgatgtcctaggataccctagtctatactgtgagttctaggccagacagggagccacagtaatactagtcttgaaaagaagagaaaaataatacaaatggaaaaataacagaaataataacaatttaaaacttatttattatgtttgactaatttgggacaataaagaaaatgtattgcaaataatacatccctaggggtcctgacagactgagatagaaagaaaatatatgcaaaactaaataatctaatgaagccaaataatgaattttccccatctctgaaacactcccacattctcagtttctgtaattacatcacacacacacacacacacacacacacacacacacacacacacacacaccagaattttgttttgtttttaatgaatcttattggcatgatctataaagcatctctggattatctatccccttggaaggcaggagtacttcaagttgagctgtcccgtgggaaaataatggggacctgaaaattcatggaaattgaaataataattacttatatgtatatctatagcataggaagctaggttgtatgataatgttatggaccatgtggtgaacaatgctgcttgacaaagttccagcattttaacactgcagcctgccaaacttctccaaaacatgctgaggcaggtagagctctatgagctcctggtcagactggcagaatgacttccaggccatccaaagccacagagagaagcactgtatcaaaaaagttgatgacatccaccttcatctgcttggtagcaatgcacatgtttaaccccagcactcaggaggcagagacagtcatattttctgagtttgaggccagccaggttcacagaatgagctccagggctacacagataaactctgtcttgacaacacaaaaatgaggcacaacaaaacaaaacacatcaaaaaagaataaaatccgcctttagtatggatcacaccatcttatggcagcctgcatatatgaagggtatttaaggagcaagctctcactctttggatgcttaccctagctcttgctaccaagttcagttctttactagcatttcagcctatgcatttggaattttggtgtgtaatgaaaccagttgagacatccaggctgatgaactaaacaactactggacttgaattttcattggtcaacagccattgttggacaagttgggcctgagcctgtcagacattctaatgaatctactttctacatacatggagagatcattctaccagtcctgttcatgtggagaaccttgaataatgaagttagttatttttaatttttgttttatttaaactgttcactgaagaagttgcactggcaacaattgtgtaaggagccttgtaaccaggcaggcaaagcctggctggagagagattaagacaaaggttacaacccagcaggcaaaggctggctgaaaaaagattgaaagaaagggagtataggcccactacccaaccattgtagataagaatgagaatgtgtgacaaagtgtgattagcaagatttggcaaattatttaaggaccagctcagaacaggtaactttagagtgtaccatgactctatgtcctggagctctcctacggtaacctgaaataaccagtccatcaagctgagtgccacccgttctcaacccgattccatgacactgggtcctcagcagactagacagcctgtatcaagaggaacacaagtctggagccctaatctgcagggctgctgcagaagaggtaccccctgctgtgagcctgttgtctgtggttcttaggattgtttgcgtatgcttgtgtgttactttgacataagtgcgaagtaaaacttatatactcaaaaccaactcactgagtttgtcattcttcccaggtcaaaacctggtaagggatcgagacaaatagtgtttataaacacaatcttcctagctcaaacactctaaatattcaacaatctataaagactactcttttattattatgcatttgccaagagtttgctctgaattccaggaataaaactcctctagcaatggagcaattacccatctttctaatgggagaaagacctattatcttgtttgtgatctggtttctacttttctttcctaattgtctcaagttcctgtgacacccaggtaaaaagtaccttcaattcacctgaccacagtgtaaagccacataacaaacatctccttttgtattcttaaatctctctttagtagtaaatattctgggaccctaccagttttttgagaaagaatcattccatacacatagtaagtgtagtggattttacaagatagctagccacattgtgtccaggaggaagaaggggcaatgcctacggagtgtaaaatgatagcagtgtctgcttttgagccatgggcaattgctgtagaaatgcatatctccgctgattagtacaggtctttttctggggtggaaagtagggtcaggaaccattgtactgtgtctttaagcgaactgtaaccttatgggaggtactcttgtcactcaaacattactagccaatcaggcccctaggggaactgccagatccaagggcaggccccttttggccactggcttcaggcttggctttgaagagaaactggaaccagtggttttgtgtcctatgctttgagatgggtcaaacaagaaaactacaacatggtgagtgtggggctgctgtcctcagatggggagtggcagaggatgaggtgctctaggcagtgtggtgggaactccctcggtctgggtgggaagctgcaaccacatgccctgtcttgtggggtcccatgcagtccttgaaatgcccttgacccagaaaggcaggcttctgaataactttgctttgtcggatgagtctgttctgagcattgggaacacggagcagtgtgtagaatcatcttggtagtccacttcaacaaccttttggtgcacccctggaaagcaggttgccaaacctgaagacacctagtttctttgttaacttcttgcatttaatctttaggtgtaatatctgtttattgctaattttgtggaagctataaaaggcatctcatttgttgggtagcacaccaataataagctgtgatgttgaagagcataatttgtaatacaaagcatttaccaaggcttaggagatagaaagtctcccaaagcagaaattacctgagggaggactgcctctaaaacaaccaaagataagtatatgctaacaaggcctgttaaaaattaggatgctaggatataaatttcttcaccaggatttgctaaacatttggctcccaatcattacaaatgagcttcccctgatttacaaccagccactaggcccatctccatatcaaaggacaggcatgtgacaagctgctcaaggtggcattacagggtcacagcaaccttatggcctgttgtgtcaaaaagttccagttttcttcttgagaaaagcagagttctgccaggtgggctatagaacaccaggagacaactatttgaaatgtaaaataatgccttaaggacttccctgatcaattaaggctttcatttaggccctctgtcaggaagggggaagagtcttgtgatagctagtaacagcctggggcctaggatttggcagctttccctcagggcgtgatatattaacaataaaacagaaagcctcctctaaaaccaggaatttgcttgcaagagctgatagtggtctggaagaaagcctttgtggagaggttgcttcagatctgtacacaggggtgagtaatatcgaaacttacatttcagcactttttattgaaagactccccccccatctcagaccaggaccgcagccaccccaatcaccatgaggaggcagaaacttgatgcaaacagcaagaggctttaatgaaaggtgaatgtttgtacaaaggggcctcctcagcacgcaggctagagggcatcccccgtcccccaggcacaggtgtttttaaaggcaaaacccacaagtttagggaggggcctcgactctgtccattgagtacctgatcagagtcatgggttcctaggaggccctgtgtcttgagtggagggctgggaatcagatggcctctggacccaccagttgtaaaacccacagacctcaggatgtgctaactaatcttacctgagcaaatggtaactatctctttgtttcatagggacccttaattgttaattattgacacattggccagtggggcaatctgtttccttcatgagccccttgggtgagggtggccatctgggaattcttggtacccagttatcttatggtcttgtaagggagtaattgctggcgtccggtaaattccaggcactaactcatagtagttgccttggttagtttctgggctatatttctttgctaatttctaagtttttcattataaaaattccaggtttggagtagaagcccgggccagtgacacccatgctgtgtgtttaagcacaccctaacccttaaggaagtgctcttgttgctcaagcctcactagccaatcagggacaccaggccaacatgtcagtcaaaagtcttgcaggatgcttcctggagccatgctgcaagcttggttttatagcagaaaaggtgccagtggatttacttctgtgctgtgagtaatactgtagaggattgtgggggtagcatggatgagtgcagaagccacagcatggtgattgaggggatgctatcccaagatggggagtacagaatggggaggcatgggagattgtgtggcaggccatccaggaagctgggtgggaacctgctgccacattgtgttctgtgaggtcccatttgatccgcaaaacttcccagactcatgtgctggcctatgaatacttcactctgtgggctgcatccgtggaaagcattttcatcagggatctttgtgtagaaacatctttgtagtcaataccagtgtgctttgggacatatggaaggcaacttgtcaaacttggagaggctttgtttctcccatattttctcaaatttctgcacttcatatttgacatttaggtttataatgcatttggagttaattttgtgtatggtataaatggcattctttttctgcattttatatttacttatttgtggattctgagacaagatttcactgtatagctttggagcctgacctgaaacttgctctgtagagaaggttagccacaaacatagagaccagcctgactctgcctctcaagtgctgggattaaacgtgtgtgccactaaatacttctggagggaagggtgctataatacacatttattctccaagattgtccagactcatggctccaaaccatgaaaaaggagaaacccctgatttaaaaatggcccctaggtccattcacatatcaaagaacagggcatttgcaagcttcacaagggggcattactggctcactatgaacactaggacatggtgtaccaaaaacgtctagttttcttataaggaaaatcatagttctgccagatgggcaagatgagatgaggtgatgactgtttaaaaatgaaaacagtgctgtgttgtgggttgagctgagcaagcaaaacttactctttaggtcctcactcatggagaaggaacatgtctctttctcaaacccaggaacatgcttggcagaggtagtaatagcctggggcctagacctaggagtttttcatcaggccctgaatcatcaagaataaaatatatgtccaccattaaaactaggaagatgttttgaagagaatgtaatggtctggatccagggcctttgtggagacattctgagaatataactcttcccaccacatggagctgtggttattggttccaaggccactgtgagcttcatcagtaatgttcttgggatatcctgtgttccccttgtactgcattgtttgagtagccttctcctgagtttgtcacatgggatgataatttctgctgacttcctagaagtctctgcttcccctccattttccctggaagtagtataaaagaagctattcctcttaagcttacttagaatcagacgtagatttagcaagacttccttgccccagcttttatcttttctttgttttgctcttcctgtctttctcatcttctgtcacctctacctcaggacttgtcggtaaagtatgatgttctgtctcattttacccatgtacggtgaaataattttggtaatatcttttctgttgaagtctttcatttgcctaactggcccaacttgcagtttcctgtcattatttttcaaagtgtaaatttttcaaataaatctgaccctaaataaattaaagatgacagcatatatttaatgcaaagtctgttttaggagccagactgaactggtgcacaccttcaatccctgcctttgggattgctgtgaggttgaggccagcctggtctccatagcaagttccaggacaacttctgctgttgcatagataaaccctgtcttaaaaaaacaaaaaaaaatctgttttagcttctcttctctttgagaattagctagaggtggaagtatggtatcattacagagaatttccaacttttcatctcttaaatatgtagagtttcttctcattggaaagagatattaattctagaacacctgctcttccagtcttttcaaaaaaggtttctgaggcaaactctctcttgttgacacatgtggtcttgatcccactctgttgatttccagggaaatgacctcgatgttctcttgattatgccttctcaaattaggtgactttcatgtattttactgtccattgcagtcacaagaatcggattttcatgtggatcatggcttctgccttcagacaagagtatcaagaagagttaatttagttcaatgcaaggggaaaacagtaggaaatcaattgcttgattcacaatcatgaaaaaggaactgattgtggcaatgtgatagtttgcatgtaattggcacaataatatagggagtatcactaatagaaatcatggaacttagctgaggaaggactgcttctaaaacacccaaagctaaatttatgctaaccaatgttagaaattagtatgtttctgggtgttggtgatgcacacctttaatcccagcactcaggaggtggatctgtgaaactgaggccagcatcctctacagagcaagtgtaggtataggctccaaagctgcagagagatgttctgaaaaaaaaaagaaattagggtaataggatacacatgtgttcactaggatggtctagattgtagctctaaacaattacaaatgaaccatccataatttttctttttttccctttttttccttccccgtgtgacccacttcagtgccaacagcctgcaggcagaggccaggcaacggttaggagccagtttgctctaggatgggacactatgaggaaccatccataatttaaaaccagcctttggcccatccctatatcaaagcacaggcctgtaacaagctgctcaaggaggcattacagggtcaaaagcaaccttaaggcattctgtgccataaagctccagttttcttcttgagaaaagcagagttctgccaggtgggcaataggacaccaggctacaactgtttgaaaattaaagcactgctgtgttaaggatttgcctggtcattcaaggctgtcatttaggcctcaagctggagggggaacattcttgggatagctagtgacatcctggggcctacgccttgggagcttcccctaagcacctggtgtattgacaacgaaacatagagccttgtctaaaatcagtaaatacctagaggagatggttatgttctggaacaaagcctttctggagagtaggcttcatatttggagaacaaacttcttcccactacctggagctattgttattggtcacaaggccactatgggcttggtcagtaatgtttgtgggaaaccgtctgttccccttgtgctgcattgattgagtagtcttctgagtttgtctcattgtatgaaaatttctgttcacttcctagaagtctgctattcccctgcgtttccacctggatgtagtaaaaaagaagctattcctcttaaaaagcttacttagaataagacatagatttagcaaggctttccttccccagcttttttttctttattctatactgcctttcctgtttttctcatcttctggcacctctacctcaggaactgtcagtaaagtatgacctgctgtttcatgttactcttgtactataaaataattatggtaatatcttatgtattgaagttttcatttgcttaactggcccaacttgtagtgttctgttactatttaaaattttcaaagtgcaagctgtagtctgaacctaaaagaacttaaagatgacagcacatgtggaatgcaaatcctgttttaggagctaggcagaggtggtgcatgcctttaatcccagcactcaggagtcaaacgctggcagattgctgttagtttttggacagcctgatctacagagatagttccagtaaaaccaggactttgcacagagaaacactatctcaaaaaaacaaaaaatatattttacgctctctcctctttgagcattagccagagatggatgcatgctatcaatacacagattattcttcattaatctcttaagtctgtggagttatttctcatgggaaaaggcatattctacaacagctgcccttctatccttttagaaaatgatttctaagaaaagtcatgctgacacaggtggccttgatcccaggttgttgctttccagggcaatgacctcgccttcctcttgattaccccttcttaaattaagtgactgtgtgacctcagagttttaaaatcattcatttgggtcatgtagttgactgtcctttgcagtcacaagaaaagtatttgcatatggagaaaggtttctggcttccgaccagagtagaaagaatagattattgagtgtgaatgtaacatatgtatggctgaaggacttccaagaacttagcaaagggatggcttccctagataatgtgtgtgtagtggtatgggcaccaaaataccctgttccttggaagaacaggaagtgctctgggagactgggaggatgtctactaagacacaaactttccagacttgggccctgtatattgggatggaaggacagagcaaggaatgcctatggcgtctgatttatttgatctctacatactcaagaggggagctgctgatgttctgtggttcaactgtgtggacccatttaaggaggcactgtgggaaggcttaggactcaggaggacaagttagagttatctaacctttttccttccaggattctctgctggatatttcgtaggagatgtaatgaagaccacagtcacagcaaggtaagtgtggaggcttcagagcaggagcagcagttctgtctagctgtcattgctgctgggattgggagggactatgagccatgcactgacctgggtagtgatccctgtcatctgagcctccctgagtttaggtttcctgggaagagagagagaggtatgcaaccctgggtgtgatacttataatatcagtgtacagagtgtattacaggttgctttgaatgtggggtggaaccatacattataaaactgaaattgaggcttctggaatttatgaaaactgcattccactttcaggtttgtcttacatgtgttacacagtttagaaactggtagaaggaagttatggggttcatccttcagcaatacataagaatccttttaactttctgctaccaccagctacaatgatgggatttaggaaagttcagttctagagaacaagaggaatttctgatctgcagaagtttctcagttgttcatggtgttggctgtgtggatagggcagattccacattttgtgtcatgatacatgtggcaagaagaagatgttgaattattcttctacattattttctttgtgggcagaaatggagcttggtttgccacaaaccacaaggggacatgagagtttcagagagatagcaagagtttacccacatttgtagacagagctctagatcatagggaatctctctttggaaaaatattgaggactggagggttagaagcagaagagtttaggggtccttttgagttaattttgtggtagttgtgaagtctgtttatttagtttctaaacacatacaattaagggtaacttggtaacattggacatgatctcagtttacctggttttcctttggtcttttgatgaaaagttgattgtattttcctgccattgttcctgtctctttctctccatcaactttattctttcccaaatattgtacaccttgattactgtagatttgtttatgtatagaagtcactttgtgttaatgtgtatactatttttgtaatcctcttgagaattctgtttccaattttcatatagctttggaatcagatattctgtcataagttgttgagaattttgtttggattcagttcatgtggcaaattttgcttacatggtatcttaatattggggtatcaagttcctaaaattaatcatttacttcttcattaatgtagactcttttcttcttgttgtttttaatatgtgaaagacttttactgtttcaactaaaaaggactatgggtgtctcaggacacattcttcttggggatgacctcagttcagatcctgccacccaaatctggaggctcatacctacctgcttctcaagatctggtatatctgacaccctcttctctggtatgtcatttgtacaaacatggaaaacctctctcccaaaccaactctagataccaaaaaaagtgggataaacttcaaaattttccatagactactcagggagaaaaccacactgggagagaatcagggtcttggtactcatgtagggatggattcgtcatgacagacagacacaaacacaggagaagtttgatgctgctacaaaaatctttttccttagtgacagtagtaggaggtgtggctttgctgtgatggactatcctttgttagaggaagtatgtccctgtgggttcaggcattgaggtttcttatactggtgatgctgcccagtgtgtcaatcaaattcctgctgcctctgagtcaagatttcaggactctaaacttcatcaccacatctgcctgcatagtgccatgacccgtgacaagatgatgattaactgaaactctgaaattttaagggagatgtctaaatgaaatttttgtaagagctgccttagttcatagtgtctcttcacgggggaaaaaaaaactaacaaagacagacattggtaccaggatctggggtattttttgataggctggccctatcatacaatttctctgatgtgttctcagtagtgttgctctgacagcttgtctctccctttatgtctattcattgggatttttttttagtagctctttttttaaatttatttattagttcaagttagggaacaagcttttttcacatgtaaatcccttctcccttctccctctcccacccctccccccatcactcctcccccatcattgagatttctttctcaaggttgtatgctatttaaagggccaagaattggtgcatgtgaacctcctattgagtttccttctcaagtgactgggtgaatacgttataatgtgcatgtcatgaaataagtgtggcaaacaccagcattttatgactatattgtcaaagaagcatacacttgcaacgtatcacacttcctgtctcacacatgtctgggtcattttaggatgcagtgacctatgatgatgtgcacatcaacttcactcaggaagagtgggatttgctggatccttctcagaaaagtctccacaaagatgtgatgctggagacctacaggaacctcattactataggtaagagtgaatttcccttcatgatttaaaataagggaaagactttttcttggttattgagattcttctgtaatttcaattgagaaaatggataattatgtgaacaagtcatatttctaaagttcattgaagatattacataattttatacctttccattaatatatcatatatttcctggtattgtattttaggctacaggtgggaagaccataatattgaagaacattctcaaagttctagaacaaatagaaggcaattttcatttaaatgttcatacaaatttgcctctgaagaaattttaatgtgtcctggaaattttaaagaaaaacaacagtttaaataagcacaattaaagtgtattgatggttcttggctgtgtagtggtgatgcaccactttaattccatcacttggaaggcagaggcagccagatctctgtgagttcaagaccagcctgttgtacagagcatgttccaggacagcctctaaagccaaagagaaaccctgtctcaaccctccctaaaaagtatattgatgagtcttattttctcaaaaacccatgtattccaatatcatgtagctattcagtgtttgcaaggcatgtcttctgaaaaagaagacaaggaaacaataccttaacagatacctccatttgcaatataaccctatgagagctatgctgtagaaatgtcaatccacttagtttcattccattcagatattttaagtggtatacatgtggattagatgatacatatatgtccaagaccttgtaataaaagtatctatagtgaagctgtcattcctcatttatttgtgactttgctaagtatattgaaagcatcagtttatgagagtcaagaatattgtcgtggagaaactttaatccctataccacacatgtgtgaggaaaagaaagtaaaactgtgtgaatgcaatatggaaaacttttattagttattcttcctttaatacaaatatcatcaacaactgcatggtggtggtgcacacctttatctcagcacttgataggtagaggcaggcagatcttgtaagtaggctagcctggtctaaaggttccaggataggatccataactacacagagaaacctatctcaaaaaaacaagaaaaaaaaggaatataatatgtcactgtaaatacaagccgtgtgagcatagaatatggaaagaggcaatgtacatctctgacactctcactacaattagaagatataaacaatatttagcagtctccactttgagtagatttgctgaatgtgatacaagtttacaattaattgcttttccaacttcattgaaaagaagtaaaaaaaaaactcacactgcagaaaagccctataagttttagaatgtaggactactttcagttgtcctgatacattctgagactgtagtatgactcataatatggaaaaccttttgaatgcagtaagtatagctaagctctgagttcctccagttctcttcaaatatgtgagaaatctcacattgaaaacatgacatgtggccctttccaccgactcatcacagaccagatccctccaggattgtccctgcttgaaacagacacacccaggcaaggaggagctccctgaatctgggtacaggccactcttccttccgttcccggcgaccgatccaacaggtgagtgaggagactacgaggagaggcaagaccatccagagctttggacattgaatttctggcccacagacactacagcgtaaaaagaggagatagagagaccccacctgcactcactggaaaaagatatgggaagaagacagaataagaactcgctcaacaacagaaag

SEQ ID NO: 2 - DNA, 23480 bp, synthetic construct
  source 1..23480: mol_type = other DNA
gaagggagtgtttcaaggctgtggaatattgaacaattaatacgaggatagttaataaaagatatttaatgatgcggctgtaattattacaaagccatctgcctggtggtgatattataaggcctcgttgagcatttatattaacataaaactgaatgttaagttggttctgttaaacaatctatgtaaatgactgagaaagaaccatggtctccctagtctcgcagatggtataaaggacttctaattcattgtagaaggagaggcgtgggcttcagggtaacagttaattatccctgagctggatgggaagattacagagatattaccaatgatggtaggatttccgttttactgacaacactctagctctaaaaacaataaagtgggaattaaacatcacaacacactaagcatttttattttttagtgcttctttgtatacttggcgagcttacataaagctcctcaaacttgggtcatattttaacggcattgtttgcaaaaagcaaaagttatataagctctgatgagaaccataagcatcaattcggagctaacacacagtattagctggtggcgcggaggacctttggagttaaagaaacagtggaagctatgtgggaaggctgtgttctaaacaaacaacccacaaccacatagtacaggggacagcagaaaatcaagctggtggccccagtctggacggcaaccctctgaaagtgccttttggtcttcatagatcaccagctctccatccactcttttctctgacttgcctaggtttgtaggatgttgttttttttttttttttctgtgaagtgagttatgagtcattcagaagagcacaggcctctgtacattttttaccatgtgtcatcaaatgtcacacagctcaacagcaggaattttttttctcataatgataaatgcaaagtctgggttagtttgggggtttcagatttttgtttctgctaatcctgagaaaacattagtgaacagagagagagagagagagagagagagagagagagagagagagagcatcttccatgcacggagaccatcttgctgctaaactatttcccatcttccagattaatagacttttcagtattatttgaaaataattttacaatgcatatttccaaaatagaagtatctgaataaaataaatgaagagaattatgaatgtcgtctttttttttttttttactttctacaaaatgaaaacacagtcttatgtctgggcaataactacattcactttactctcacacctcttgacttaccaagtaatagtaacagtaatagtaatagtaagcctaacaataatttaaaatcaagacacttacttagtgctaactgtgtatcaaaaactactctaaatatttccccaacttaactcatttagcacccagctctgtaagctaaccttactattttttcttggaaaagataagggagatagaagatggagagggagacaaagacagagacacagagaatctcctcagctgtagggaccaggatactaacctagcagttttcttacagaacacaattgatcaaagacaacaggtgtcttttattatcagctttaaattctaaagaagaacacgtccaatcttcttaagcaaaggtcagatggtggctcccttttcagtcccagctctgggagttcgagacagcggaatccttgtagattactagctaggtggtctagccaagatggcagatgcctggtttcatgcaagatctttcctcaaaaaaacaggaagaagagcaattgaagaaattatgctgatgtaaacctctgcctccaacacacaaatgattccctatacaagtacacatgtgaaaacatccccgacacactagtttaaaaaaatagtgggaggcccctggcagtgggaggagaacttaacccagttgcatgaacaggtgggatacctagctcagccttgatcctgcctcaacttggtcttccagactttgttgattccctaagggaggccttacccccctctgaggagtggatggagagtgtgggaagggggtgaggtgggggcgggagaaggggagggagagggaactggggctgctatgtaaaatgaattttttttaaataaaaaaacagaaactcatctaacatggatgtcttaccagttgccctcttctatccactcctttggctactagatacttggttactagatccaggttagtcagcatgagatgaagctgggatctttagccactgtgaaaggatgtggtctctcagggaaactgatggcacaagtgtgcaaaacttctgtggtttcaggacatggctattaaggtgtgatttaaaagatggcttttcactcacatttggcatgatatctttatcaccaatgtgatatgtgtgtgttggttggttggtttgttcccagacagggtctccccatgtatctctgactgtcctggaactcactatgtaaatgaggctggcctcaaactcacaaatccacttgcctctgcctcccaagtggtagtattaccacgcctagctaactttatcactgatatcaatggtgagcaaagtatccagtaattctaattctgcatgaagaggaagacagttttttgtttttgtttttttttttttttgcaaatgtactagtttacctattgaacaaacacttatggagccatggtcctgctataactagagttaaaatatgaataaagcagacagccccatcttaaaggaattcatcaactattggaggcatgactatttaggcctttagtcatacaacatatatttcaaaaagataattccactaccaaaatttgaagttttctcagaaaagtaactactaatactaatttaaaagccaaatggtgttaaactacaaaatgtaggaatatgagaaatgcatgtaaattagtcaataaatgagcaataccttgagtcttatcattgttcttctggccccaataacaaacaggtggagataaaaataatagaaaagcacaggagagggtgttaactctgcctggggaagacacttttagaaagaataagaagctaagtatttggcaggaagacaaattgacaaagagtaatttcaagcaagtgacacatattcagctcgtgttacctattatcactataattaaaggaagtgtccttatagtaggttttgaaagataaaatgagactagggcaaaagaagggtcttggaagcagagaaaattgttgtgcaaaagaggggtaccgtgcttattgtgccaccacaaaatgagcaatggtccaggaccacagcaatgttgtgtgtgagaagctgatgcagttatacccagtccaagagggctttcatatcagacttacaagtgtatattgtgccctactgcctgcaagagctcaataacatggtttacttgtttttgttgttgttgttgttgttgttgtttgagataccattgatcaccttttaaactggtcaccagtgtggcaacagacagaacgttttaatggacagcgaggggcagaaaaatgtttgaaatacacaagaacatgataaagtttttcatgcggagggctttcaatgtttcccaggctgatctcatatccctgggttaaagggactctcttgcctcagactcccttttagctaggaagtttgtaccttagagaaaaatcaaacacaatagaacttttctctttccttaaaacatcacttataatagcaaaaaggagaaaggcattatccaaacatccaacttccagggtagaattatatgaacagcactatttgatgaaggcatgaatagaatattatgcaccaaagctaatgagtatgagtgtgattatgtaagcgggattgaaactataaattggcattattaaatataatccatctaatgttgtgactcgttagggaacattcctgcttgtggatgcacacaacacaaatatagctctacccttatctgttccagcaacttcgacttaagcccaagaattgggtaggggtagtcattgcatactcctagctaccaactctcacaaataagactaaggttcttcacagttttcaaatgaaggaaggaaatgagaccaagtactggccatgaagaggagtcttcaggggagatagaagtggggtgggtgtgatagcccaagctttgcagcaggaatgagtcagtcttggggtttcagggaaatgggacagtacctttgcagctcctgttgaaacattttacttcccctcctacctcctttcacaaatagctttaaattgtttttaagggagggatggaaaatattatcttccatcatacctttttgctgtgactgaaagttgcaatgaaagaccttactgaagagagacatgatactgtaacacagacccaaaatgggtcaaagagaagagaaggaactgcagagttcttggtgctaacagtgacatatgtatcacacactcttttcccaaggctcagggatcattatggaaatgggggagcagattatgatagccagaggaggtgagggactacacagaaacagtgatttcaagacaaagcagggtgaccacacagggtgattcacagcagttgagacaatatgttcactggagctaaacaaaagctcacagttgagacaatatgcacaaagtctcagcatgaagatgggaggtgggcagggagtcagcatcctaggtgagggtctattagcagctcatagctgctaggagagggagagtctcttttctctaagggtggcccctagtgagtagactgtgcttccttcctggaagtctatgtatctaagagtatgtgggcatcatcaaaggtgcttgatagtttaaaagggacacagggtttgagggggtggataagagtggagtttggagggcatggatatcctcaaaatatatgttataacattctgaaagaactaataaaaaatgagggaaaataatataaatgaaataccctcttcaatatttgcagcattccattcagttcaggttcagcaatccccttgtgcttgctttccgcagtatgttctgaaatttgttacacgttgactacagagggtataaactgtaaggaaatactttttgcaatcatgaatatttacagtctaaattgcaagaaggaaagccccttgagtggctagctgtaaactggtcatctgcatcacctttcccaagcctcaggtaaccttacaaggaagaagggggagagattggaagagccagggggtgggggtctttgtttcaaaaaggcacctgaataggacagggccactgtatttgtgaactcactgaagctgagattgtatatgtcctgcatgagattggacctctcggtatttcatcataggaagggaaaggactcatggggctccacctatcccttgagaattattgacaattaatagcatctgtggaggagtattgtttcactttcttcagtgactgacagccactgacaaattgcatatgcttctgtaaatgacctctgacccatgcacattcgagtcactatagttaaactcagtggataacacatacacacaaacatacatacaggatgtcggaaaggttctcactggggaacagaaggatttaagtgggaggagaagaggggataagtgagggtaatgagggtgaaatgtttaaaaatcattttaaacaaatatgaaatagcgaaagaatacaaatgaatttaaaataccgtgtttacactcaaaagtgctgttcttaaatgatatacaataagatatgtgtttcatgcatgccatgattatcatctcaaagccggaaaaccaaacaaatattgcataacaagagggataatgggtacaagtatttgcattttcactgtgtcttataaagataacagattttggggaagaaaagatcaaagaaaagaatggttggacatggaatgaggaaatccatcaggaaggagttacagctattaggaagtctgctgatagtgcagtctaggtacctgcaaggtttaagaactcatagagtatagatctttggaaatatgcccatcttcaacaggcaataaaaaggagttcagaaaaacgtatgtaggtgtaatactttggagcattcctttagttgcttcgatcctgaaccatagacagcttcctgtttcacacacttggaaatccctactgggagaccaaaaccaggggtaaccaaaacccctcttccaaagatattaaagataccatgagcaatgcccaggaatttcaacgcaaccatcccagaccatcaaatcaccaggtgccttaactactactgaaggtgcctgaaaaagtacagtagggacacccaattatatcactttcccatggaaccatgacccagtaattggttgttctgttaacaatcacataacaatgtgatatgtcagggcgtttcactacctagtaatagaaatacatatagtctttatcattcaggcatgaattaaaatggaggcatggctgggaggtggtggcacatacctttaatcccagcactcaggtcgcagaggcaggcagatctctgtgagtttgacatcaatctggtctacagagcgagtgccaggataggcttcaaagctacacagagaaaccctgtcttgaaataaccaagaaaggaagaaagaaagaaagaaagaaagaaagaaggaaggaaggaaggaaggaaggaaggaaggaaggaaggaaagaaagaaagaaagaaaggaaggaaggtaggaaggaaggaagaaaggaaggaagaaaggaagaaaggaggcatgtgtttacattattcagtattgagttatatttattaactactgcaagtcttagatgtgatagaactccatagaactccacccttcctaatgttttactagctttacctgtctactctcatgcctcttctctgtgcgtttgaggaaacataacccttagtatttccttctattcctttcaggccaaacagagtcagagtcaagattaaggcaccttctcttccacccctgcccatctctcttcgtcctcctcccattgtaccaggatgctcacacttagggcgtgtaaatgaaggatatgaagaaagacaagccaggctgcagtttttgtcctttctaatgacaccaagaagaaaagccctagctacttcttcacttacaattcttaccatttctctccatttaaacatatattttcctgtcctattgtgtttgatttctgtccatgtatttatcacatttactaactataaagcctcacgtctcacactcaacctaagcttgctgagtagagaatatgactacaatttacatagagcctggtggtgcatagtagacagccaagaagtagctagagaaagtggccacagactcttttcatgtcagggaattcttcttgccattctgggcatggaatacatggttccattttacattcagttagaatgttaactttggcataaatcaaagaaacaaaaatcagatctccacagatttcctgagtgttaaagacaactgtggaagaatggctgagcagatttatccatgtttcttaatgtctcaccagatccacctttttaactataaagggatagtttaatcccactgacgggaccaaaatattgaagactcaatgaaaacttatttcaactgttatgactagtgcctccttgctatggtttcagtaagtgcttactgacttggctcccagatcttcttgtaaacaactaccacatatcacttgatgcgtttatcagtcatctacttgggagtataaaccacttcaaacattggcatttagcaagcagttaagtggttcatgattcttcataccaagaaattggggtattctcagcttagaggtacttttgctggtcttgcttgaagtcatttttgtgcctagaagctccacaaggggctgatggtccaagcttgcctctcctgctcatgtttcagacaaacaattccgctgttgatgagctactcttggaccttcagtgcggatgtgctgtctcttctccatcttacttctacttttccatgaaaccaagcttctttatgggagggtagaaccattaccagagaaaaaccaagacgagcctaaaatctcccccgcctgagttttgcaagttgtaagatgtaactgctgatacatttagttgttcacagaaatcttaggatctgcctaaagtacaatgagggaaaatagtcataactggatattaaaaaaataaaacagtcagataaagggctgtgtgatggttcagtgggtaaaagtacaacacaagtgtgtgaacttgacttaggatattcagaacctaccgaaaagccaggcatgaacagctataatccctttggtccttccaggtgagaggaagtcgtgaggggcaaatcctcgaagcttcaatgccagccagcctacagttcacagcagaaaaacaacagaggcactgtctcaagcaatgtgagaagatgatggcatgaatatgtgcttgttctctggcccctacatgcatgtgtggcatacatatactagggttaacatatgtactacacacttgtacaatgtgcacacatacagaaacctacagacacataagtgaaatgatcgcattgcaaatatgtgtgccaggataggagaaactcttgaggccatccttacaaactacatgctaaagttggtttgttgttctctgacaaaggacactagatgcctcagactggtttgtctagccagtcaatggtgaacaaaagacgatctcggtccctgtttgaatttcacagaatccagtcttcatacagacttattctgtgactgcataaagatttctctgacagtgtctattttaactattcatttctttcttcccagcaatatatagagcatagacttcagagccaataaaaaaaggtgcattttaattatttcctaaagcattatctctccaaagagagccccagtgtgaaggcatttttctatactccatttatttttgttaggatgttgaaacttccagttaaattttacagtatagcatccttcaatgtgaccacatctacaggactgacgtgagagttaaatttggcaaggcagcgccgtaagaattgtggttagctccagggagctttcagcctgaagagcggattgatttacacatggcgataagtaccacagaaagaatgtgcaacttctttgcttcaccttgctccgtgatctatgagtccctactgactccaatgcctttttttaattttttaaggaactatgtgtatttgtgtttgtgtgcggagtaggtgtgcatagagtatacatgccaggaattgacactgggtatcttttccagctactcaccatcttagtttcttttagtatgatattctttgagaattttgcacatgcatacaatatattttgatcaaatgcactctccactccttcccttaactcctcagcatctatgtcttctctcatcttcccaactctgtgctttgtgtctttttttttgtttgttttttgacacccaccaaattcagtttttgctgccttaatgtttttgctacccacagcacggtcaacataccaaggactagacacctaaagaaaactccacgttagtcttttgaagcagggtctctcccttaacctggattagctagactggctggctagtgagctctaagggtctgatggaactcaagccactctacctgcctttgacagtgtactagggatcgggccttaggcccccatacttgggaggtgggattttccaactgagccatctgctcaagcctcactccattgtcctaaagaggcagcttatagtttcaacctgaattgctgcaaagccagtctttaaacatattttctgctattactacttagagggccatctatgcacacataaatatatgagtagcataagagagaaatgaacaatggacacatggggaagggcctaggcctggtccaggatgatgtggtagactttggggagccccttttgagggccttaccctgcctggggagtggaggggggatggctagaggcaggtgggatgttgggggaggggagggagaaggagaagggattgacatgtgaagcaagcttgttcctaatttgaactaataaaataaaaaaataaataaacatagaaaaaaagagagagaaatggacaaagtccatgggaaactagattgtgtgtgtgtgtgtgtgtgtgtgtgtgtgtgtgtgtgtgtgtgtgtgtgtgtgttaagccttatctccaagtgtctaccttttacttacataacaggctaatgagagacagtggacctcaaatgtagcaatcccaaagcacagaattttgtacttttaaacaaagggaactcttcctgcttgtacagagctgcccctacagtaagcatatcaccacttgtcagtttcattgctctgctattcaaatagcacaaagaggaagcagaaatatttgttgccagctagcgagctccaatgctcatctctccatctgttggacagctggacaaggcaacagctgcccattaaagcttctttgcctggcatgattaggtctattggcacccaaagcctgtgccagctggctcctgctcttagtgaatagaccatatttgaacagagtaagtctccagttgctacagaacacagtcagactcctcacctcccttcaagaggcaagacttaacctcataacaagaaaaatgaaccagactcattgctgaggcaagtttactttggagggctaattttcaaacaggacaggacacaagtcctgcattttccatctctcagctgtgaaatgaaagttccttcatctacagtggaaacagcacccgccttggtgatgtttcttccaattggatcaatgtcagtgaaagggccctgtgtgttttggactttccttctgtgtgaaccaattctaaagccacagaagccctaactcgggagcaatctcaagaaaggaagtctctggggtcatttgtcgtcaatccttaattacccgaaagggcttgaatgtgctactttgcatgactctcgtgagagtacatgcaatggaagttttaaacattgcccccaaaataaaattagatcaaaattcatagcaactttccatcaccctaagagttgagcagtaggaataggccatcaagttatatacactcacctgacagccccctaggaagactgtaagaaccaacctctatataatccacagagagtgccttctctttccattctttcttgcattcaccaatcgggccactccccattccaaatggattgtgcagtgatgcaagtcactaataaatatccttaccttgaagaaacaacacacatcatgaggcagtacgtgaaaagaaacggagacaacgtgttgtagaaattagcaggactgagaggcaatgaaaatctgcatgtattcatcgtgtgataagtaagtgttaagtcaccggggaagcgtcctgtgtgttttcatcaccaagctaaacccctttgaagccaaggaaataacttcaccacatccatctcatgagcaattcttttgcaacgcagtacgtttggactgaattagtctcctagggctgctacacagccgatggtttaagaatagacttgctttctctcatagtatgtgtgttcagcatctaagatgtaggggtcagcagcgtagacttttcctgggactgtgtagcaccatcagttccacacttctccctgaaattcttctgttttggctagcattaagcccccttgcctgtgaaagtttgatctctgcctccatctccctttgagcattcaaatatgctcatttcttcacctgaatttccttgtgttccattatggcctgtcctaatgacctcatcctaaacaacatcatgggtcataactatttccaataagtcacactctgagatactgaaagtgagaactttaatatatgagttttatgtgaacatagtttaactcataatgcggccttgactggctaatcggactccctgtgtttctccataagcatacctgaaaatatcatcttcaaacctgtgagttacaagtaaatggatgaagcatgctctgtacctcctatacttaaatcctatttgcaatttcagcattccttccaaccccacgattacagtaatcaggtacaagactttggattcacaaaggtaggataaatatttcagcattctctctgaatttgtcaaatgcaaatggactagacattgttgatgtacttattgcctgtatatgttgcattatacatattctatatagttttttcatatattagttatagccttctttttgtattagacaagaaggggaattgtggtgatgtattatttatgatttattaaagctttatggcttaattaaagggaaaacaaatgaacacggaggacactagattaataaacgattgcttaaagaatttgactctcgccaggtgttggtggcacatgcctttaatcccagcacttgggaggcagaggcaggcagatctctgtgagtttgaggccagtctggtctccagagcgagtgccagggtaggttccaaagatacacagagaaacccggtcaaaaaaagccaaaaaaaaaaaaaagaaaagaatttgactctccttgtttgttagtaaactgttaatgccttctgtgtaactaccaaaagcaaaatattgaaaacagacacttaaaaattctatgaatcacttatgatgtgccagagactatacaaggttatttctctgagtgtgtgagaaacatgatacagcagcaaaagtataggcattcaccctcggcgcacagaggagatagttagacttcaggtaatgtgtggactatcttacaggttgaaaagtatctagaggtttgggggaaaatggggacaaaaaaacctcagcttatgccaaagtctcatgtagcaatcctggtctgtatttactctatgttttcttcatgatgcaattaaaataaagcctcactggcccagggtgaaagtggagtgtggtaaggatgtttacataagcagccatgaataaaaccaaagacttttaaagagaaaaggaagtggggaggaagaataagagactcagttcaaagaagggaaaatactactttatgttcctcccaaggaggtggtaagaaggtgggtggtttacaacttgctctcttcaagttaaatatggctgttggcaccaccaagcaatacatgccattcagaaaatcttgtttcctagaaaggcactgggccctctcttctgtgggggtgtgagatagagcatcagatgtgagggtttgtctacctagcattcattcaatggtcttcaccactgtctctagaaaagaaattatggtaggtgagttcattgaattaaagaggaaatggagttgtacttggtctgtgacaccacaatagtgctgaatagtctttgctattctgtggcttgatgtcagaaaactatctgtgaacctgttgaccaaagcaagaaggcataagatgttcaatatgctgcctttcctggcacatacactgacattaactcttcaaatatcttcccatttattcccaacttgatgctgaagacgtctactctttcaccaaagttccattgtggaggattccagaatatcctgaccatgtttctctatatccgtcatccacacatttgtctgacagtctccaaatgtgtgtcactatggatgcgtgtggtatatttactcagtatggtaaagttatttgatatgaattatctaaccatcagaaatatagagttgcgaagtacctagggaaaatgatgtttcttattatataagtttaaagcatttaaacagcagttttatgtacaactcatttgttcaataaaaatttatattgtgctaatggggatagaaaataaatagtgagatattatataagtgctagacacttatagctcctataaatatacattaaagaaagtgtgtataaatgaaagtccccatctggcctgttagaagtcctttgaggaaacataacttaagtcaagacatgacagataagcaaatgttagcctgcaagggagatggctgatgagagtcattatccagcaatacataggagaacaaagacccagatgaatgagaaggcacagcgtgtaatatggttggcgttatagaatgaaatagaatcagaaaacttgactgaatccatttcaaagccggtatatatccttagaatgagagaacacaaagtaaaggagagtcacaggtcagaaagatcacttttgctagaatagggagaacaggggcaggaacagatggttgaaatcttgttataattctctggagattccacagaatcatgacacaaaattatgactgcaaagctttaaatatgtaaccagagttagaaaataaatgatgtcacgatgatatttgattcttcagttggtatgcaattgaggatgaaggcgatatccaagatgactgtttgggtaggcagagatgccagttgttgagataatcattggagatgtggctgataactgggggaagagggaatgtttggcaacattagttttgagctacttatgaaacaaccagacaccagtgactggatatcaggcagggatctagaatctgtagacctgatttaatttggaaatatcagcatgaaattcatggtctttgaggtgttaatgaatcccccaatacataaaaaattatcaaattagaagcctacagacaagataaccaagaaatccctgaggaaacttgaagtgtagaggtctagatgtcaagtagggtggttgggaaaggctgtgtccaaaggcacagttagcaaaaggaaattattgcattaggaaattcaaagggttgcctcaaggaggcagagaccaagagcaagagtggtagaaagttgaatgatgttgctaagtccacagattgtatggaattgatggattgggtgtcactgaagcccttctggccagcgactgctaagaattagcatgcagagaaaaccagaacagagtgggaggtgagcaagtgggtcatacccagaaatctctgaaagcgtggtaagaaaaggggaaaagcagaaagcagccagagtggaataacatgctccagacatcacagaagaaacttttctccactgtcctctgatcccaattaccacccccaccaaaagcccaccagagtatcatagtagacagtctgataacttcaaacgttctgaaattgtgacagaaagcttaaagactaaagttagtatggtacagtctctgctaagctcaagaaatacccacaggaaatggaagaactttttgactggaggcctaatcagagtaaaggccaaggtcttgaaaaaccaaatatgtgatatccttgtgagcagaatgactaagtgagcaaggggggccacagagctcctgctaaggggctgtactcttactggtcaatgaaaccaccaatgcagacacatctcattagaaatgattaatttagaaagcagaatggtccaacagttagaagcccctagaaggtctactgtaacagtcagtattgaaatgacctttctcaccatgtactgctatgaaattagtgttagacctcaaggaaatgatttaaatccaaacagagtaaagacgaaagtgccttgtaccttcagtttctcaaaaaaaatgtattagcttgattgcttatcaataatcattgggttactggttttcaaccctgacactgggaaagtttgagaatttttctaggttgattattttctcatgggcctaatattaagtgataccccaaacccataccaataacccagtttgtgccacaccacccttggtaacagaatatctacaccctccactaaaattaatagggcctgcccatttgcttttaaatttgttggttgggcttgcttaatgtttaaaattaataatgatagtcacaactgagactgatttagtggttaatatttgtcaaacactagataagactaagctcaaaataatctccattccatagaaaaaattaagatgggcaatgtgttgattgagattgtgtaactgtaagtgcttcatgagcatgctaatcaaaattaatgatgtttactggtattttatatggaatgcaatccaagcaaatactgggtattgggtaagaaggttccattcctaacaaagatgcagtgacaggagggcagatcaaagatcccatcagaaggtagaggaagctctctgatactcaacagtgtccctgtttgaaagaagcaaaaggagtgaggaaagtgaaatgtagatggtttactccaaaaatgttcttttcaaggaattctgtaggaatcctaagcatcgagaggagtgtcatgggccagaaaggtgcttcttacgtagaagatcccggacaaattgttaaatacgtaaaggccatctctgtccccaggaattgtctaacatatgaaaaattaacacattgctgcaacagaaatacatgatcattttgaagaacacattctaatattatttttattaaaatctcatgtattactgtgcatggaaacaatgtatatgctgatgaatgcatagaatcatgagttctctggagcagggaaggacagaaggaagaaggaaaaaaataaaacaacagcatgtaatccctgggctgttctgaagagccctttatgtttgaattgagatttttatttgatctgggaattcactgcttcacaaagtacaaaaatgacaaatctaactgactctttaattttcaaagttaggaaattggagatcaagaagttaagtctcttgacagggttccagggctggttagaagccaaagagacgtgaggggaagcagtctgcttcctgtgtgatagcccttccaccaggcttcctctcttccttcttcacttgaatcaatagtcccttgatggaatgtgaaaaccactgacccttgttcccgcacgtgagggtggcatttctgtatgtcttatagaataatttggctcttgaacccttaacagaactggactggctttcactaagagatctgtgggacaggacagttttcctattccccggaagaatactctgtatttcatcaaagctgccatcataataaagagagcagatgtgtttcaaatcacccattaaagttggcacagaagtacatatggatagcattatgcagaatgagcaggttgtatttatggatatatatgtatatctacacacacacacacacacacacacacacacacacacacacacacacacacatatgcatgggaaggaggccatggatttgaaagagagaaaggaaggggtatcagggagggtttggaggggtgaaagtgaagggggaaatgatgtgattatattgtaaccctaaaaatataagaatgattgggaaaaattattacttctctaagtacaataatccatgataacaacaatcaaataaaataagagtcatgtgtgtagtcacaatcaaaatgaaaacccagcacacatatgcaaatatacttaggaaggagtctatgaaaaggaacatacaacttcaggcgtcacagtcaacatgagagaataatattatctacctaaaaaacagaacggtatgaatttttttttattttatcctgtcattttgtccgtagatgatcatcagtgaacatggacgagttttacacctaattatattatttgatactcatattattatatgtaaatacaaactaaactattgtgattggcatgtaaaatgttctcagaagcccatgcttttaaccaattcatcttcagctataagtaagaccttttcaggagctggagagatggctcacaggttaaaagccctggctgctcttccagaggacttgagttcaattcccagaaggtacgatctgggggaagaagcagggaaggcattgggtttataacttatttcaccttctggatctgtctcttacttgctgatccaatttaatgtaaacaatgttctgtagccaccgttatgagctgctcctactataatgtctatgcatcatggacagtatctcctcaaactaaataatatataatgtgtatatattatataatattgacaaatataatataatacattatatgtattataaagactatctttccttccttcagttgcatttttgggtattttattattgcaatgagtaaattgacttatatatgagtcaatatatgaatgattatgaataattacttatgaacatgagttccagttagagttctttttacaagaccaactaaaagccaaagcatattgtgagatttttgtcggctgtcttttgtaaactatcttgaaagctagactcaaaggcgttagcacccatctgctctgaattctccattacactgacctggcattctttcctataaaaggtcaaatagtaactgtattatattgattctctgacacatatcatttctatcaagagtcaactcttgatacatgacgtgtcagcaaacaggcatggctgtctttcaaaaaatctaaatgtttttatatagtctctgagaatatcatatattttggtcatatccattgccctcttgcaactcattgtatattatgctttgtgttactatgcaataaacattgtgttctttctcggtcttaatgaataagcaagctatgaaatccatagtagactatgactgctactgcataaggggcctgcctggaagtgtggtggttatactcaatatcactccattgaagaaaactcactctccttcttccaggttctgtcaattgtcaatagctagaacctgaggctagaaagggcctgggcctgagggacaaccaactactattatcaagcaaccttttgtaagccaatcattttaatttcatcataattttaacaacactatttcttccgcaaagcaggatgcatttgacttttattccataatataaaggccctttcctacagggtcacagactctttcatatgcagtcaagaaggtttcttacccataacaacagagatagctctctggaggatagaatagcatgctcttctgatcatgaatccaatgggttgtaatgggaagtactaagtgccataaatttgagttgatcaaatattatggatacttggctctcaaaaactcagagaggtttacaaacacagcagggaaatgataaccttttgaaaatgtatccccaagctccacagtttcacatttacggctcaatatttgataatttgatatagtttttaaaggtttttttcctctttcttttaatttatgaagtatctttcatgtagacacttatggagtgtgtcttcccaaaactcatatgtatttcaaatttatttaaattcagaatgctttaagattatttatagcatagcattgtatgctcagcctctgaatagataagattgatggcttcccatcaaatcactcagcaaatacctattaagcaacaatgaatgctgggcacatttctaagaataggtctacacaacactgaacaaaggtgacgttcttgctctccaaaagcctacattctaggttcctcattatattgttaaacatagataaagacatcgatgaatcagcttattgtatatattgcaagtgacggaggtgcaaagcaatggtggcttgatagcctggaatatgaatgacattaggagccagtcatggaatattctgaagaaacctcatccaagtccagaggtatgtctaatgcaaaggccagacgtaggaaggaaatggaaatgttaaagaaaaaacaatgaaagttactcatggccaaggtacaggtacagcactaaggtgaattagagaacagagccttatacaccatggttgatggctttgatgctgctcatattctatggacaggataaatatacatacacacacacacacacacacacacacacacacacacacacacacacacatatatatatatatatatatcaggcaaagtcaactttatatttgtacaaatgttacctcttcataaaaaatagaaaataaacagtagggagtctagtcacattttgttaaggaaatccaaataaagatgaagatgatacagaaaatcatgaaacttggggtcaatgtgaattcagctacatccatctggaagatgcagttgctaatgtatccattttactgcatgaagggaataggggcttctgtgaggaactcctgaatgctaccatgaggaattgtgtagctattgtcatttacaaaagaaattatcgccgggcattggtggtgcacgcctttaatgccagcactcgggaggcagaggcagaggcaggcagatctctgtgagttcgaggccagcctggtctccagagcgagtgccaggataggctccaaagctacacagagaaacgctgtctcgaaaaacagaagaagaaggagacgaagaagaagacaaagaagaagacgaagaagacgaagaagacgaagaagaagaagaagaagaagaagaagaagaagaagaagaagaagaagaagaagaagaagaagaagaagaagaagaagaagaagaagaagaaatcttctgtaacggctgttcacagtaccaccacaccaatcagactacttctttcattgtttcctgtaatcttaccacaccgtgcctctcattcttccctatgaaacaaggacataagacccgtcttagtccaccgaccaatgagatattctatctgtgctgtttcatcttcattgttaatctgataggattacaaatcaccaaggaaacacactcttgtgtgtatctatgaaatgttttaataaagggttacttgaataaaagataaatccttagtgtggtggcaccgttctgcatactgggaccctacagtggataaggaaataggcctaagatccagccaggcaatgtctcagcagaacttgtgaacagtgttcacatgggactgggtttgtaatgcaagattcaggatccagaaatgcttgtgcctggttactgagagctactaagactgtgttagggatagactgtctgcaaggaagatccccaatgccattgtgaattgctgtaagagtgaatcttaaattataaaatataacacaagatattagagatatcaaaacgatgggttatctgcgtattaaagctgcaaacaaggaatagactggtctaaaagagatgctgtatctgcagcagatagtagagctgaagataaggaggctactactactctatcccctcaaatcccaaataattccatcacaaatcccatatgacagaatttggtatttgccctgctagatttcagtcttgtttggttcagtatagccttgttattctcccgtgactacctttgggatggaaagttcactctatgacactgtgtattttgttattttatggggtctcacaatttagagactcttttcatctcagaagaaacttttgacttttgcatagtggtagccctgttaaagaccatgaggactatttgagacacagttagtgaattgttcataatgaaataaaaagacattatgtgactgaaaagtgatgtcttgtgtcgggttaacagggaaagaatttgtgatccttcctcttgattgtcaacttgagaggactaagagtctccctggaagcatctatctgtctctgaggagatttgtagaaatatttatttggagatggaaggcatgccttgactgtggcaccactgctccttgggaaggggtccaaactgaagaatacagaagcagccatctctaggcctcctggtactacgggcacactgtgactagccaccatggctttcccagaacaatggactacaccctctcaagctataaacttcttaagctctttcttgtcagctattgttcacagcaatgaggaaaaaaatacttaaaacagagtattacccatctgcatagtgccagaatttgttacaaattactatgtggtccttggaactatctgaacttgatttttaatacaagtcattaaacacaaattatgaataaattaggcatgctataattctttatcttcatatttcaaatgcccatgtaaggtatatatttatagttctacatcatatgtcatacaagtaggccacctgaattaataattacttggttatttctcctcatgaaaactcacattttatcatttatacatgcacttacttttcctcaaaaatggtcttttttttttcaagaaagaaatatctgttgcaaataaaggtaattacttttaaaaaagaaaccatcctgaccacctttgatcatcatgaagcctgttggaaagataaccacttacattattctctttcttatggatatatgttaaaccaagcccaggtagaagattctggcttcaaagattccactacatctttggctttaaaacagaggacataaagttgcaaagggtggaaaatgagaagacggtctaggagaggtcggtgaatataatagaaatccactgtgtgcatgtatcaaattctcaaaaacaatgaaataatacatacatctgtgtatctgttattcgaggacctcaaattacttccttttgcaggagctgagcaggaaatgacagccaaagcatccatgaggggagttccagtcagtggactcaatgagtcagtgctgtagttaggaatttagtcctgtggtagagtgttgcccagtctagtatgcatgcaagcagggcccctgagagggacaaaggaagggagaaaatcagggaagaagggagggagggaggaaggagtgatggataaaatagagagggcaagaagaggaagagtaggaaaggaaaaaagagagttattattggtgagtatagaatttggagaaactctaacctcattaatcagttatgggggggttgtataattagaaaaaattctctcttacacatgaggttcaaaaagaaaaacaggataaagtttcacatttgggttgacacaaattagaaagaggtacaatatattgaggatttgttttattgtattttaaatactacgtatggagggagggaggcttatgaattcatgtctctaaagaatccaggagacggcatctgatcccctggaactagagttacaggcagttgtgaggcacccaatatggtgctggggccttctgctagaataatacatactcttaatcgtcgagctacgtgtcttgccctagccacaatttctgggtgcaagaatctaggaaagaatcttaggggaaagcaagtatgggcagaataggtgcccttgtcaggcagagatctgagcaaataagagggttgctgtggctgtaatctgtggtgtatacagaggcagtgcggcccctggatctcagtgttaggcagtggtggctgaggactatcagctggaggccctacgatatctggctaccggctcacagagagaactttgtagtg

SEQ ID NO: 3 - DNA, 18346 bp, synthetic construct
  source 1..18346: mol_type = other DNA
tatcccaggctcaatctccttatatagctgaagatggccttgaacccctgatcctgctgcttcagcctcccaagtactaggatgacaggcatgggctgccagtacccaacttcagccagcccttgctcacaagtctgtctctgttcatgctattgaaaccctgtcaccagaccataaggcaatctatcggaatgaatttgaactttgtgtcagaaagaactggcctggctgggtggtggtggcacatacctttaataccacacttgggaggcaaaggcaggcagatttctgtgatctctgtgagttcaaggccagcctggtctacaaagtgaggtccaggacaggctccaaagctacacagagaaaccctgtataaaaaaaaagaagaaagaaagaaagaaagaaagaaagaaagaaagaaagaaagaaagaaaaaactggcctggaaaatgaagagatggctccacagttaagagcacatactgctctttcagaggacctgtgttcagttcccagcacccaagtggtagctgacaaccatccacaacttcagtttcaggagatgtggtgccctcttctgagctctgcgggcacccggcacacatatggtgcacagacataccacacagacaaaacactcacacacataaaataaaattcaaaaatccaaaaacaaggagggaaaggaagggctatcccgaatcctgctactaattccaccaagtttcttcatcagaataaagagcatccctagccccagttcacagcctcttggtgtgctttagatgctccattcagctcaggaggctgagtgcttggattacagtcaagaaccactgcactagccaggtttttggtttgtttttaatagggatatggaaaactttcaaacactggacatgcataatggcacatgcctgcagtcccaggacttagagggcagaggcaggaggattatcataaattagaggctaactggtctacacagcgagtttcaggttagctgaggctgtatattaagatctggattcaaaaagactcaaaccagggggccatggctccgttggtggaatgcttgtctagcatgcttgatcttcagcgctgcacaaaccaagtgtggaagcacacacctgtaatcccaaatcttggctaggaggctagagggttgggagttcaagaccaacccttgctacatgagacccttcctcagctgaaaacaaactgaaagttatccagggttgagaattcctttgagggccttaaggactttttcagggagacactgtggtagctattgtacaaccgtatccatttctcaaactcaacaaattgggttggagataaggttctgtggttaagagcactggctgaacttccagagggcccgggttctattcccagtactcacatgacaacttatgagaattgattccctccttccattaagttggctccagggattgaactcaggttgtccggcttggctgcaagcaccttgaacacgtaagccatctcgccagcccttggggttttctctgttgtttcccagtgaagacagtggcaccagcttgagctggacagacggaacactttcatgatgaaacaggataaagcctggtggtacaggctttaaggtacaaaaattgaagctgggcctggtgtctcatgtctggatgtcagcacctagaacactgaagcacaatggtcttgagttggagaccagcctagactaggtgacaaggccctacttaagaagagaaggggtagggctcgagagatggctcagaggttaagagcattgactgctcttccagaggtcctgagttcaatcccagcaaccacatggtggctcacaaccatcccttatgagatctggtgccctcttctgatgtacagatatacatggaggcagaatgttgtatacataataaataaataaaatttaaaaaaaaagaagagagggggtaaatgggagggacagaaacaaattaacatggattttccaacccctagagaggtttggaatgaatcaattgtcatttctccgaagaagggaagtagagagaaccagatgttgagtgaaggcctactgtgcaccagatcccttttcagagctgcagaactaaggagagctgattgtgcgttctctctgcacgcttgtatgactcctactatgtgccgcacacaggcggtgtgtcctatgaaacacgcagcaaataaaacgctgactcccgtcggtgcactgtttgcatcacaacgcccttctctgggaaacgcatcatgtagccactttttgacatttcttcctttggaggcttagaatatgtgttggtgggaatcccagaaacggccaagagggctgggttggcatcccagaggggtaacagcagagggcccctcacctgttctccaaccaactcctgagcagtacctgagtctggtcctgtctgcttcccggtggcaccagggacacgggatcttgtctgattaactgcctaattccaagaacaacacggacccaaggcacagtgaaacttctgtggggactctgggccgggaaaatacctcaggcctcttgggagacaccctgtggtggcactgcccaagccaccctgtccctggcaggcagcagaacaaggatacccttgggttctgaactcacctgccagcctttccttatttctcttgcggttccccaaaccactgaaatatctttttgagaagccgatgggtgtggccttggaaggaggcagtgtaaacagaggccccacaaaggaactccgaaaattcagaggaggtttctgaatttcgaacagcctgagaaggccgagctaaacaggagaccccaggtacttccactatggttttccacaatgagcggccgcctgagaaagcataggaactcagatctgtgggaatctcatttccatgacagacaaggagagatgtcttcccacaaaactgcagagctgggcgggtgtgcgcgccataagcatatacgcatgcgcgtgcagctgcggaaaaagtcatgcgttacagttgtcagaagcaagctggagcgtttgggttggcatagctatctctgtctgtccgtctgtcttttgagatgaggtcttaccaaatagcattgactgtcctagaatgtgatatgtacatcagactggtcttgaacttttggaaatcctcctgccccagccttcccagtattggaattatagtcttgtgctttttttcccctctgttttgtttttttaattatatttacttacttttcttgttttgttttttttttattttgtttgtttcattttgttttgtttagagatggggtctctctctctctctacacagccctggctgtcctggaactcagtagatcaggcttgaactcaaagagatccacctgcctctgcctctcaagtgctgtgattaaaggtatgtgacaccatgcccagctttttttctacatatttatttgtttgtttacttatgtattatgtgtatgtatgtcatgcttgcatgtatgtctgtgcaccttgtgaatgcctggtgtcctcagaggctagaagagggagacagatcccctgatcccctgtaactggagttatagactatcgtgagctgctgtgtgtgtgtgtgtgtgtgtgtgtgtgtgtagcatgccaggacacagagagagcaaccgaaatcaaggaataccttcaggagttgcttctgtcttgtgggtcctgtgctcaaactcaggtcatcaggctgggcagcaagaagtttcaaacccagagccatcttgacagcccattgtctttgaaatatcaataagttaatttagtgggtgtttgtgtgagtgaatgggaacccaggtatcacaggttctcaaaccagcgcatgaacacagaggccagaaagggccctgacatcctcctctgtcatttcccattcattcatttgaggcaaggactcttcctgaccctggagcccatgttttctcaggtaaagccagaagccagggagcccctgtgttgctcctgtgttcatcctcctgggagctgaggtcgcagatgtgggatgctcggcttattcttgttctatggccgtgcgtgtcagaacactggtgctcactctcagtggctgagccatctccccggatcctttccttgttagttgatttttgttttcatttgtttattgtttgtttgtttttaaagacagagtctcatgttatagtcctgaactccctatttagtccagggcagctgattagcagctgccctcctgcctcagtgtcttgagttctaggatcgcaggcacacgacagtattcaaaagactctcccacactttcaaattctcaccacacaaccgtgatgccagaggaactgtgttcggcaaaagcccaaagttgactaagcatgaaagctggccgtagttcgttttctaaccagcagtgctacctcacgcggctattttatctccccatgactccacttcctcatctgtaagtgggaacaataattcttcccagtaatcgtatgggaagaaagacacagaacagtgtctggcctgtggtgttgcatcagtacacagtagcatccggtgtggctaagcttgaacaatgagacaatggaagtgctgagttagccccacccccaccctcgagtctgcatgatctgggccaggactagaaccctggtctctagactcctgctccataaggtcttctacagcgttacccatctaagaatagtttttttttgttgtttgtttgtcttttaaaaaaactaggtctcagtctacagccagggctggcctgggattccctatgtaccctaggctggcttccaactgtgtgttgtcttcctgcctcagcttcccaggtgctggaattacaggcaggcatcagcacattctccatgtacccaggctgacctcgaactcagagacctacctgcttctgcctccccaatgctgggattaaaggcgtgtgccaccactgactgagatttacttatttttaatttatgcatatgagtgctttgcctgaatgtatgactatgcaccacccaaccacctggtgccctcagagaccagaagaggtaacaaatccgctggacctgtagttacagatggttttgagccaccatgtgcatgctggaaaccaaacccaggtcctctgcaaaagcagccagtgctcttagcttctgaaccaatctctccagcccagaatagtagttttaaataataaactctgccattctgcctttccagacagggtttggatatggggtccagggtagccttaaactttcagagatcttgtctctttttttattttgcacttttcttctctttttctttaaagtaaaatttatttatcttgttttcaaagcagtataaagaagaagaagagagggagggagggaggggaagaaatctgtctgtcgactccagtgttgagcagtgcaaggctcacggcctgccactgtggattcatttgtgccctgcccccaacaacacacacaaattcacatgccaaagtctcaaccctcagtgcatcacaaaaccaagtcctctgtccacagctttgcagaaaaactgcgaaacaggccctagtaaaaagaaatgggtgtgtggggtgtgtgtgtgagagagagagacagagagggaggaagggagggagggaaggagggaggaaaggagggcttctgtctgcctggccagtagcaagcctacaatttacagagaagactgaacaagggccaggagcgtgcatatgtagattagcagggagataatcttgcccctcagggcatttagcttctgccttggcaggtctcacccttaaccccacccagctatgggattttctacaaacaatttacttccttagggttcccactacctttactacagtgtgatgatatctggaggcaagaccattaggagtcactggctttaggtggggtccaggttagggtttcatgatggcacccgtgtcttcttaagagtggacgccaaacgctgggatgcagctcagctgggagactgcttgcctagcagacacaaagccctgggtctgttctccagcactgcgtgcatgggctgtggtggcccacggtgcttctgtggacagagctgtgcaccacaccctccacaaattcatatattgaagcccagcactcaagacgtgcaggcaggaggatgaggagttccaggccaggctggactacttgagaccctgtctcaaaaacaagtaacaacaacaaaggaatatgaaaggaaaatgcactacgtgtatgtttgaaaaatggcacaatgaaacacattagtatgcataacatttcccagtaggttgcaaaaaaaacccaaaaaaccaaaaaaacaaggaagagatgtgtatggctttttatcgatttatttttaactatatgtgtgtctcttgggggtggggggaggtacacttgtgagtgcagatggcttcagagtccatccagaaggcattgggtcccctggagctggagttctaagtgttgcgagccaccaacatgggtgggaactgaacttagtgatgtgtgatcttcacggctaggctgtctctccagtcccaacatatagcctttaatttttctttttattatttttatttagtgcgtacgtgtgtgtgtgtgtgtgtgtgtgtgtgtgtgtacatgagtgcaggtgtctgtgaaggagatggccatcagctgcccaggagcagaagttacatgatgctgggaactgaactcagaccctcagaaaaacagcttgtgctattaagcagttggccatcattctagctccaagaatagttttattttgttttgtctaaaccagtatagtttcttttacatgataatgacaactgatagccagaattttctgaaagacttaaagttgtgtcattttttaacaggccagaaactctacactgacagtactggttcattgtcttcacttttgttttgttttggtcgcattttgtttgtttgtttggttggttggttggctggttggttggtttggtttttgagataacatcatgtagcctaggctggccgtaaacttgctatatagtctaggctgccatgcttgcctcaaacatgtgacaatcctcttgcctcccaggtgctgggattccaggcatgaactacacataaccacatcaccttcctctccatccctctgtcccttgtgaggtcatctggtccacctgtggctgactctatagggacttaggtgactatgccactttctccatggagaactcagcagcctcccttggggccccaccctgctgagttcctctgtagctggtgatgccttaggctcacacagtcccttggtctatttcctagactgttaggtttggcagcgtcacagttcctcctttaactgttctttcccaagtgaaaatagctggcacagtgacctcaccggagccaggctggcatctctcggccagcgcctccacccacggtttgttttccatcattcgtgtttgcagtgttactcaatttccctgcactctaccacacagggcgaagctctccaaagttgtcaagattttgtgttaaccatggacaaggcctcatttggggggtcttgagggcatttcaggcgaactgtccataacaaactgggaagggcatgatgctcttaagaactatggctccagccagtccctgtgtccctcccatgtactctctgtcctctaaacacctgccatctccaagttgcctccttatctttatcccagaaatccattacttagtttatatattgtcacctccttcccttcctccccctcctccaggataagcaataatttattctggagccaaatgtgagcaaccgtggaccaagaacaaacacattttcaggatacactaaatgccctctttctttctctccctggtaaggtttcatgtggctcagactgggcttcaacttgctatacagtgaggatgaccttgaactcatcatcatcctgcacccccccccccagtgctggatcacaggcatgcaccactacatccagtctgtgcagtgctgaggaccaaactcaagctccccgtttgtcaagcaagcactccaccgactgagatacatctccaggtatttgtttgtttatgtatgtgcattggtgttttgcctgcatgtatgtctgtatgaaggtgtcagattccccagaactggagatacaatcaattgtaagtggccatgtgggtgctgggaattgaacctgggtcctctggaagagtaggaggtgttctgaattgctgagccatctctctggcccatattttttaaattattgttgtttgttaggttgatccaggttctgtaacccaggctggctggagccaatggcaatcctcctaccccaaccttccaaggtctaggattacagcatgcacctgcaggctgcttatctagattcactatctgagtcaactttcccaggcacaggaaacggaagccaggtgtggtgatctcagcactcaggagactgaggcgggagagctcctcaagctcaacgcttggctacatagtacattttaggccagccttggttacagagcaaaatcctgcctcaaaaagaagaggaggaggaggaacaggaagtagtcaagaataagaaatggctggaggatgaagacaggatatagccagggtttgtttcgacaaggacttgtttgggttttgatttattaacctatatctttgccgaatggaaattaaacctaggacttcgcactggcagtagccaaattaccctatcactgagatccacccatatagcctcagggcaggattcatgagacagtctgttaagtggcttaactcctgaactctgaaggggaagagttgagacagatgtctccctcccctcccctctcctcccctttccctttccttctggccatacaaagattgctgtgcccaaagcttcccaaactccagagatcatggaaagtctgtgtacctgccactcttcagagggaaagtttcagatttaccagcgattcagaaagacggcttcacaagtgtgtactgcaagataagcctgcgtatatcaaggcacttttcaaaagtatataaacagagctaatttattagcataggagagagagagagagagagagagagagagagagagagagagagagagagagagaggcctcttcttctgttccatctgttcccagagcatgctgaactggcgtggaaaggaaagatcttttgactactggcttttatcctctggccccctccagtcactaccagggagtgatgtgatgtttaccctcaggcatttggccaatcagaaaggcccagtaagtgatcagcttttgcttaatggggccactgatattccccgtcactcctgtatgtcagctgtttgacttgttccctcagctatatggctctcaagatgaatggagttttgttgatgtttgttcatttggggagagggggttggggttttggggggtttgattttgtgtttgttttgagacaaggtctcactttgcagccctggctgacctggcactcactgtgtataccaggcgggtcttgaactcacagacatccacctgctgcttctgccttccaaatgctaggattacaagtgtgggacaccatgcccagctgtctttatatttaccctacctactcacttactttgagacaggttctcacatagtctaagctgactttcaactctcaacagagccaaagatggctttgaacttctgattctcctgtttgtacctcagcagtgctggaattgtgggcatgtcccaccatggtggctcatcaccatgctcagtttatacagtgttggggacggaactcagggtatagtgcatgctggcaagcaagcactcttcttagctgcagctccaggcccaagggaaatagttttaattacacacccacccacatacgatttaggcaacactcagctttgccaactggttcattcatttagtgaacagccaccttcttcccagaggccaggctctggagtggagggtgtaagcatatcccctgtgaaaggttcaggctttaatgctgatcggctctacctctttaagagtcagaccccaccccctgacaacagtcagggcatgcacaggaaaatgtgctacgtcatcaccacctcacctgacatgcattatggccagcgtggggactctgggcgtgcgtggaacctcagtgcccatgcgcagtcttccctttaaaagttccaacttccctgcttcgctctttctgcttcttctaccctctctctctactcactccttctctgctctcacctacgtgctctctatggtggtcagccattacccctgttcctcttctctcttggtctccctactctgcccggcctataataaactagtcaatatgtctcatcttgcgccctcctctttttctacaaaaacataacaccctgtccatcagccctcaccatctactggaggagaaagatgtcaaagcacatgattagaacacagtcagtcaacgaagaggagcggatagcctgggaattcaaaagcaagatcatgtgtgccgccctcctcatagcgcagggtaggagcagactcggccggacaacggacatcatgggatggcttaccgatccccgcctcacatgctgtgtctcaaggcagcactggcaacaagctgggttaattttcttttccattgtcctccctggttgaggtgcccttccctccttcacactcgttttctgggcttccaattagctgataacctgatgactatgtagcccaggcttgccttaaactctgggctgaccatcctgctgcctcggcttcccaggtgctaggattatatgcatgagctaccacaccaggctcagcatgagcaccttaaaacaggacccttaggcatgtttaggtcaggcatctggggacatcgaggagctggagaaacatcggtaatcaaattcctccagaggcaggttatcatttgactagcttttctcgtaaacttcccaggctcacaaatcccttgctgttcctcaagtgtgtagccagcctttgcaatgcaaagtaacctcattctgcaggagaagcagagaccacgcttgcacgcaatttgactatggtgacagctatgaagtggtggcagacagagagaagttagtgtttagtttgagaagggtgtcaggttagggtttgttgagtaaatgcataaaggaaggaaaggctacttaggtagagggagtgttgtcatttctaccttaaatcccacccctcaccccacccccaccccccaccatcttcacctcccacactgcactataaggagggctgtacacacatctcacaccaccagacggaggtctgaggatatagctcagtggtcccccacgttcctggtatgcacaaggccctgggttctgccccagcaacatacataaagcaataattataacaaacatccagaaggagtgggccagcaatgggcttaataaggtaaataaaatggcccaaggtaaaaacctagccagagagccggccaggataacccaagtctcactgaccagagactgggtctatcttcatacccagggtgaaggctgatgcgtgcacctctctaagaccttctaggaattcagtctaggaattcagtcagatctagacagaagaaagtcagaagcataaccaggactcggtggctcacatatgtaatcctaacatagaggaggtagcagcagaaggatcaggagttcaagaccatccttggctacatatgaagctagcctgagttacatgtctcaaaaaaactcaagcacattaaggaggagactcattccataaagtccttgcctcacaagcttgatgacctgagttgggattttcagagtccataggtgtagtgggaacatttaacttttttttgtttttaaatttctacaagttaacacaatgtattagatatgtaacagctacactggtttgactacagaaaataataaaagtgttctcttaacaatgaaggaaaatatctgtaacctggatatatatggtggcacacatctgtcatcccagcactcaggagacagaggcaggaagatgtcaagttcaaggtccgcctgggctgcttaatgatttttgaggccagcctagactatgcagcaaggcttagtctcaaaaataaaatagagctgagtatacagtgcctaccatccttcaggggttgtttagagaggacagaagtggtacaaagatgacctctaagaccagtatagtaaagggggcctctctccagggaacctggtccctatctctcagtcaaatcccacatagtatcagatgttcccagacaaaggtctgtgggatcccaaaggcatcattggccatcctggccactttactcagtgcctgaggtctccaccaggctcttgctgtgcctcagttccagcagccacctttgttgtctgtgatggtttgaatgagaatggtccccataggctcttacggttgaatacttgatccccagttgttggactgtttgggaaggattagaaggtatggtataccaaggttttttctttggggcctccaaccacctcccaaatcatgacacggagatttctcagtagaccaggctagcctcaaactcacagagacccacctgcctctgcctccccagtgctgggattaaaggcaagtgccaccaccgcccggctacaaggcgtattagttatgaatgctcagtctagcttaggctcgtttctggctagctctttcaacttagattaaactgtttctccttaactaccttttgtctcagggctttttacatttctttccttctgtatatcttcctttctctgcttctggggtctgactggctggtggctggctggcttcttgtttcaggcttgcccctctctttctctctcattctctcctcctcctcctcctcctcgctcttctctcctatttattctctctgtctggcagccccacctatccctctcctgcctagctattggccgttcagttctttattagaccaatcaggtgccttaggcacacaaggcgaaacaaatgcatcacatcattaaataattaaacaaagcattaaacaaatgtaacacatctttgcccagttaaactaatattccacaatagtatggccctgttgaaaaaggtgccttactagaagtgggtgttgaggtctcaaaaggctcctgtcaaccccagtatgctctctctttcctgtttgagatgtgaactctaaggcattcaggcagccaggccttcaccccaccgtaatggactctaaccctctgaaagggtaagcccaagtaaacatatatgtgaggctgtgctggctggtcttgacgcactaactggagttatctggaaggaaaggagtgaatttcaatagagataatgcctcaatggttatgtagatccagctatatataaccacatggtggctctcggccatctgtaatggaatcagactcctggtatgcatgaagacagagcactcacatacataaaatacatgaataaatacatcttaaaaaaacatttatactagataatttttaaggctggagagatggctcagcctttaagagcactgtctgctcttacagaggaccagggttcaatttccagcactcacatggcagctcacaactgtctgtaacgccagttctagggtatctgacatcctcactcaagacacacatacaagtaagacaccaatgcacataaaataaaaataaatttttgtttgttttgtttttcaagacagggtttctctgggtaacagaaaaataaaataaaataaaataaatgtaagatccaactgtaaagcattttcttaatgattcatggggggagggttgagcccattgagggtggttccatccctgggctggtggccctgggttctatcagaaacacactgaggccaggcagtggtgggcatgcttttaatctcagcagaggcaggtggcaggaggcagagacaggtggatctctgtgagtttgaggccagcctggtctacaaagctgcacagagaaaccctgtctcgaaaagcgggggaaaaaaagaaggaaggaaggaaggaaggaaggaaggaaggaaggaaggcagactgagcaagccagggggagcaagccagaaagcatgcaggaccctccatggcctctgcatcaactcctgccctgattgagttcctgccctgacttccttcaatgatgaacagtgctgtggaagtgtaaactgaaaaaatcctatccttcttaacttgctttttgtttggtcgtggtgtttcatcccagcaatagaaaccttaagaagtgccttgatcatgatgttttagcacaacaatcaaaatgtaactaaaaccccttcctcagaacccctcccccacacggcaacctccagaagcctttgtctatataacggggtcttcagcacaaacttgtgaccactgaggatgtttaggaaacacatcctggaaagctagcgacatccccaacacagcggccagtcacgtgtaactgttattttacatggcaattaaatgagaaaatgcaaataaacgctcccaggctgacccaaaggagaacaaataaagcggccatgagaaggtggggtgtgaccccacaaacaacctggaggtttggagtgagggaaaaaatcacctcaaagacaccggtggactttgtgtggtttccccaggtgcactggccacttggctgagccattatcaatcacacagatccattatcagccctgcaagagtgggcaggagaaaggcaacctgttcttccctcctaagccacctgtgctggtcactcctcctatatatatctgtctctatcaaacaacacatattaaactcaggcctaaacaaacaagtaaaaaaggttaatttcctggcaggaaattcctgtgtaatcagtcagcagctccggtcccagcacagtcgctacccagcactgtgacctttggcaggcccacccctctccagacccattagtcaccccaaccctgcaggctgcggcctctctcctggtctctgcatctctggtttcccttccctagtagagacccgcagagtggagatccttctaaaagacctgctctgtagctcagagttagggcatgcgcaaagccccaggatcagtctccagtgctgtcggtacttgaaggagggatctggtcggtactgcctggtttaaatctataatctctgggttaatggggtaccaagagaacacacacttgtcatgcacgaaaccccgggctcaatctccagtgccaccaaaacaaacaaacaaacaaacaaaagccttatatttaaaatctgtaacctctagaaaaacaataataaaatctgtatcctttactcctggatggaatttgtaatgattaatattgccaacgtggcagaatcttgaatcacctggaaggcaagcctctgagttcccctgaaacagattatgtagatcaagttaattgagtgggaagtagcattgctgggctacataaaaggaagaaagcaaactgaacaggagatcccgcactgcttccggctgtgaccaggagcctgcctccaggacctcccccaccatgatggactgacgggattcttgaactgtcggcccatataacccttctcccttcagtggctttatttatttgtttggaaagtaggttttcaagctgggcatggtggtacaaacctttaatcccagtactcaggaggcagaagcaagtggatctctgtgagttcaaagccagcctggtctacagatcgagttcctaagacaagcagctctgctaagagaccctttgtcttcatcgctgttccactcactgctgtaaagagacaccatgaccaaggcaacattttaaagagaaatcatttaattggggcttgcttacagtttcacaggtttagaggcagggaaggggggaggaagggctagggagaggagaaacactgactggctggtggggaagccccccccccaccatccagtgatgcactttgtccaacaaggctacacctccgaatccttctaatcacgtcaatctccacaccctggagactaagcattcaaacatatcagcctatgggggccactcttattcaagccaccataacctgtctcagataaaaagaagtttttagttgaaatagaattacatcactttcctccctacagcccctctccttactcactccgtagctggggatgactttgaaccgaccctgcttcctctaccttctccgtactgagattgggggcatgcaccactatgatctaagtctctctacaggttccctctcttgtttcatgtccctgtctagtttcatgtcaccgagccccatttacccaggttattctgcattgtgccaagctcaacaatcaaaactaaacatcacaactatacagtaagattccaaacagaacaaataaacaaatacgataaaaacaaatagagccaactctagaaaaaaaagtcaggtctgatgataactcaggcttgagaaagtgcttatcagatgaacacaacgggagacgtggcttctaagcagggagcttacctccatgctgtaaaagtcatgactactttgttaatcacctcccacatgaaaggccttatctttggaggaaataaaaaggagttctttctgtttgtctttctttgaggcaggatcttactatataccatggctgtcctggagctcgttctgtagaacaggctggcctcaaactcacaggatctgcctgcctctgcctcccaagtgtaataggggatgcccttctgtatatatgtttctcttgttggttgatgaataaaacactgtttggccaatgaggcaggaagattctgggaaaggtaggcagaggaagacagcaaaacagtcgccatgtggagaccaggaggataggatgcaccaggagttctccagtaagataaggccatgtagaaatacatagacttatagaaatggattaataattaagacagagctagccaataagaagtcctagccatcagccaacagttttataattaatacagcatcagtgtttttatttggggctcaaggacagcaggacctggcaagacaagaaaaccgccggcaacacaagtgctagcattaaaggcaggctccaccatgcctggtttgcctcagcctctggagtgttaggataaaaggatgagcactttgcccaacttgggatgcttttgtttattggtttgtttggagtcagggtctcatagaaccaggctgacctttaacttgctatgtgctgaagaagcctttaagcttgtgctctttctatctcaactacccagagtggtacaattacagttgtgaaccatgcagccagtttcatgtgtgctgggaaataacctcggagcttcctacaagctaggtaggcactctaccaaccgagagctacatacccagcccccagcattctttcttattcttagccttatactgaatgggctggcctccttagatcctgacattgatgatagtacctggaaggtggtagctgctgcatgaatgaatgaatgaatgaatgaatgaatgaatgaatgaatgaatagctcacagtaactaagagaatccagtgccagcagtgagcagaggactttacttagagtgtctcattgactgctctaattgccctactagatatggcattctttctagttgaagccattctccagttcatgcagaacagtgtgatgtttctatgtcggcttagcggctgcacatgaatctactgatattttaattatcgtctgtggccccaaggctctgcagtcagcaccctgccagccactcagtcctcaatcttacggggcttcaggtcccacagccccaattctgctctggggcagagatacgtaggtttaaattaagtctttatccttctatttaccaacaaagactcagaagtcagatgtggggttgaaaacctgctagatcagagaagtcgagaagcaatcagctttcatcccccaaaagagacatgatgggggaggtccttctgcatatatgattgtcttattggttgataaataaagcactattggccaataggggagcaagttaggcaggactaagagagaaggagaattctgggaaatgtagtaagagaagtcttgtgatgcaggcaggaagtgacatagcaggcagactcagaatataagcagggacaagcaggaaatcgctgtcttcctctttctcctgctctcttctctgtggagccaccatgtgatcc

(19) United States
(12) Patent Application Publication
(10) Pub. No.: US 2026/0275373 A1
(43) Pub. Date: Sep. 17, 2026

(54) PRODUCTION CELL LINES WITH TARGETED INTEGRATION SITES

(71) Applicant: Seagen Inc., Bothell, WA (US)

(72) Inventors: Sofie Alice O'BRIEN, Bothell, WA (US); Neha Amish PATEL, Bothell, WA (US); Howard Robert Grahame CLARKE, Bothell, WA (US)

(21) Appl. No.: 19/471,423

(22) PCT Filed: Apr. 2, 2024
(86) PCT No.: PCT/US2024/022638

§ 371 Date: Oct. 1, 2025

Related U.S. Application Data
(60) Provisional application No. 63/493,964, filed on Apr. 3, 2023.

(51) Int. Cl. C12N 15/85; C12N 5/071; C12N 9/06; C12N 15/90; C12P 21/00

(52) U.S. Cl. CPC C12N 15/85; C12N 5/0682; C12N 9/003; C12N 15/907; C12P 21/00; C12Y 105/01003; C12N 2510/02; C12N 2510/04

(57) ABSTRACT

The present disclosure relates to Chinese hamster ovary (CHO) cells with a targeted integration, at particular genomic sites, of an expression construct (e.g., for expressing one or more heterologous polypeptides) or a landing pad sequence for mediating recombinase-mediated cassette exchange (RMCE) to introduce an expression construct (e.g., for expressing one or more heterologous polypeptides), as well as methods, polynucleotides, and vectors related thereto.

### CROSS REFERENCE TO RELATED APPLICATIONS

This application claims the priority benefit of U.S. Provisional Application Ser. No. 63/493,964, filed Apr. 3, 2023, which is incorporated herein by reference in its entirety.

### SUBMISSION OF AN ELECTRONIC SEQUENCE LISTING

The content of the electronic sequence listing (761682010240seqlist.xml; Size: 66,761 bytes; and Date of Creation: Apr. 2, 2024) is herein incorporated by reference in its entirety.

### FIELD

The present disclosure relates to Chinese hamster ovary (CHO) cells with a targeted integration, at particular genomic sites, of an expression construct (e.g., for expressing one or more heterologous polypeptides) or a landing pad sequence for mediating recombinase-mediated cassette exchange (RMCE) to introduce an expression construct (e.g., for expressing one or more heterologous polypeptides), as well as methods, polynucleotides, and vectors related thereto.

### BACKGROUND

Chinese hamster ovary (CHO) cells have commonly been used for many years as mammalian production cell lines for expressing genes of interest. Particular CHO cell lines such as the DG44 line that is deficient in dihydrofolate reductase have become a dominant mammalian host for recombinant protein manufacturing due to, e.g., the availability of a well-characterized genetic selection and amplification system.

Production cell lines (e.g., CHO cell lines) used for production of recombinant proteins (e.g., antibodies or other biologics) are typically generated by randomly integrating varying copies of construct(s) of interest into a cell line by stable transfection, followed by screening for high-producing cell clones. However, the particular transgene integration site(s) in biologics-producing cell lines can influence productivity and stability.

Therefore, a need exists for production cell lines that allow for reliable integration of transgene(s) of interest and high, stable levels of production. An ideal production cell line would be characterized by high levels of production of the correctly assembled product that are stable over time, as well as integration into a well-characterized, single site with open chromatin that does not interfere with endogenous gene activity.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

The present disclosure relates, inter alia, to CHO cells (e.g., isolated CHO cell lines) that allow for targeted integration of expression construct(s) encoding heterologous or recombinant polypeptide product(s) at genomic loci characterized to provide stable, high levels of protein production. The present disclosure contemplates CHO cells with expression construct(s) integrated at one of these genomic sites as well as CHO cells with a landing pad sequence integrated at one of these sites, which could be used to integrate an expression construct at one of the sites via recombinase-mediated cassette exchange (RMCE).

In some aspects, provided herein are CHO cells (e.g., isolated CHO cells) comprising an expression construct that comprises one or more open-reading frames (ORFs) encoding one or more heterologous polypeptide(s), wherein the expression construct is integrated in the CHO cell genome at an integration site within about 20 kilobases (kb) of a sequence having at least 97% sequence identity to a sequence selected from the group consisting of SEQ ID Nos:1-3. In some aspects, provided herein are CHO cells (e.g., isolated CHO cells) comprising an expression construct that comprises one or more open-reading frames (ORFs) encoding one or more heterologous polypeptide(s), wherein the expression construct is integrated in the CHO cell genome at an integration site within about 20 kilobases (kb) of a sequence having at least 97% sequence identity to a sequence within chromosomal coordinates NC_048602.1:16770620-16791941, within chromosomal coordinates NC_048603.1:25162471-25185950, or within chromosomal coordinates NC_048601.1:13898813-13917158 according to Chinese Hamster reference genome CriGri-PICRH-1.0. In some embodiments, the integration site is within a sequence between 50 and 1000 base pairs in length from a sequence having at least 97% or at least 99% sequence identity to a sequence selected from the group consisting of SEQ ID Nos:1-3. In some embodiments, the integration site is within a sequence between 50 and 1000 base pairs in length from a sequence selected from the group consisting of SEQ ID Nos:1-3.

In other aspects, provided herein are CHO cells (e.g., isolated CHO cells) comprising a landing pad sequence for mediating recombinase-mediated cassette exchange (RMCE), wherein the landing pad sequence is integrated in the CHO cell genome at an integration site within about 20 kilobases (kb) of a sequence having at least 97% sequence identity to a sequence selected from the group consisting of SEQ ID Nos:1-3. In other aspects, provided herein are CHO cells (e.g., isolated CHO cells) comprising a landing pad sequence for mediating recombinase-mediated cassette exchange (RMCE), wherein the landing pad sequence is integrated in the CHO cell genome at an integration site within about 20 kilobases (kb) of a sequence having at least 97% sequence identity to a sequence within chromosomal coordinates NC_048602.1:16770620-16791941, within chromosomal coordinates NC_048603.1:25162471-25185950, or within chromosomal coordinates NC_048601.1:13898813-13917158 according to Chinese Hamster reference genome CriGri-PICRH-1.0. In some embodiments, the integration site is within a sequence between 50 and 1000 base pairs in length from a sequence having at least 97% or at least 99% sequence identity to a sequence selected from the group consisting of SEQ ID Nos:1-3. In some embodiments, the integration site is within a sequence between 50 and 1000 base pairs in length from a sequence selected from the group consisting of SEQ ID Nos:1-3.

In some embodiments, the landing pad sequence is heterologous to a Chinese hamster genome. In some embodiments, the landing pad sequence comprises a first and a second target sequence recognized by a site-specific DNA recombinase, wherein the first and second target sequences are heterologous to a Chinese hamster genome. In some embodiments, the landing pad sequence further comprises a sequence encoding a selectable marker.

In some embodiments according to any of the embodiments described herein, the integration site is within about 10 kb of a sequence having at least 97% sequence identity or at least 99% sequence identity to a sequence selected from the group consisting of SEQ ID Nos:1-3. In some embodiments, the integration site is within about 5 kb of a sequence having at least 97% sequence identity or at least 99% sequence identity to a sequence selected from the group consisting of SEQ ID Nos:1-3. In some embodiments, the integration site is within a sequence having at least 97% or at least 99% sequence identity to SEQ ID NO:2. In some embodiments, the integration site is within the sequence of SEQ ID NO:2. In some embodiments, the integration site is within a sequence having at least 97% or at least 99% sequence identity to SEQ ID NO:3. In some embodiments, the integration site is within the sequence of SEQ ID NO:3. In some embodiments, the integration site is within a sequence having at least 97% or at least 99% sequence identity to SEQ ID NO:1. In some embodiments, the integration site is within the sequence of SEQ ID NO:1. In some embodiments, the integration site is within about 10 kb of a sequence having at least 97% sequence identity or at least 99% sequence identity to a sequence within chromosomal coordinates NC_048602.1:16770620-16791941, within chromosomal coordinates NC_048603.1:25162471-25185950, or within chromosomal coordinates NC_048601.1:13898813-13917158 according to Chinese Hamster reference genome CriGri-PICRH-1.0. In some embodiments, the integration site is within about 5 kb of a sequence having at least 97% sequence identity or at least 99% sequence identity to a sequence within chromosomal coordinates NC_048602.1:16770620-16791941, within chromosomal coordinates NC_048603.1:25162471-25185950, or within chromosomal coordinates NC_048601.1:13898813-13917158 according to Chinese Hamster reference genome CriGri-PICRH-1.0. In some embodiments, the integration site is within a sequence having at least 97% or at least 99% a sequence identity to a sequence within chromosomal coordinates NC_048603.1:25162471-25185950 according to Chinese Hamster reference genome CriGri-PICRH-1.0. In some embodiments, the integration site is within chromosomal coordinates NC_048603.1:25162471-25185950 according to Chinese Hamster reference genome CriGri-PICRH-1.0. In some embodiments, the integration site is within a sequence having at least 97% or at least 99% a sequence identity to a sequence within chromosomal coordinates NC_048602.1:16770620-16791941 according to Chinese Hamster reference genome CriGri-PICRH-1.0. In some embodiments, the integration site is within chromosomal coordinates NC_048602.1:16770620-16791941 according to Chinese Hamster reference genome CriGri-PICRH-1.0. In some embodiments, the integration site is within a sequence having at least 97% or at least 99% a sequence identity to a sequence within chromosomal coordinates NC_048601.1:13898813-13917158 according to Chinese Hamster reference genome CriGri-PICRH-1.0. In some embodiments, the integration site is within chromosomal coordinates NC_048601.1:13898813-13917158 according to Chinese Hamster reference genome CriGri-PICRH-1.0.

In some embodiments according to any of the embodiments described herein, the integration site is the only genomic site at which the expression construct or landing pad sequence is integrated in the CHO cell genome. In some embodiments, the cell lacks dihydrofolate reductase (DHFR) activity. In some embodiments, the cell lacks glutamine synthetase (GS) activity. In some embodiments, the cell comprises loss-of-function mutations or deletions in both copies of a DHFR gene. In some embodiments, the cell comprises loss-of-function mutations or deletions in both copies of a GS gene. In some embodiments, the CHO cell is a DG44, DXB11, CHOK1, or CHOK1SV CHO cell. In some embodiments, the integration site is an intergenic site not within an intron or exon. In some embodiments, the integration site is located in open chromatin in the CHO cell genome. In some embodiments, the integration site is within about 5 kb or less of a peak based on Assay for Transposase Accessible Sequencing (ATACseq) analysis.

In some embodiments according to any of the embodiments described herein, the expression construct comprises one or more ORFs encoding a polypeptide (e.g., a therapeutic polypeptide), such as an antibody, enzyme, or fusion protein, e.g., a therapeutic antibody, enzyme, or fusion protein. In some embodiments, the expression construct comprises a first ORF encoding an antibody light chain or antigen-binding fragment thereof and a second ORF encoding an antibody heavy chain or antigen-binding fragment thereof. In some embodiments, the expression construct comprises an ORF encoding a single chain antibody or antigen-binding fragment thereof. In some embodiments, the expression construct further comprises a promoter operably linked to the one or more ORFs. In some embodiments, the expression construct further comprises a sequence encoding a selectable marker.

In other aspects, provided herein are methods of producing one or more heterologous polypeptide(s), the method comprising culturing the CHO cell according to any one of the above embodiments (comprising an expression construct) under conditions suitable for production of the heterologous polypeptide(s). In some embodiments, the methods further comprise recovering the heterologous polypeptide(s) from the CHO cell. In some embodiments, the CHO cell is cultured for at least about 40 days, and wherein an amount of the heterologous polypeptide(s) produced by the CHO cell on Day 40 of the about 40 days is at least about 85% of an amount of the heterologous polypeptide(s) produced by the CHO cell on Day 1 of the about 40 days.

In other aspects, provided herein are methods of generating a cell line that expresses one or more heterologous polypeptide(s), the method comprising introducing a polynucleotide comprising an expression construct that comprises one or more open-reading frames (ORFs) encoding the one or more heterologous polypeptide(s) into the CHO cell according to any one of the above embodiments (comprising a landing pad sequence) under conditions suitable for RMCE between the landing pad sequence of the CHO cell and the expression construct. In some embodiments, the methods further comprise selecting for cell(s) that integrated the expression construct at the integration site.

In other aspects, provided herein are polynucleotides comprising an expression construct that comprises one or more open-reading frames (ORFs) encoding one or more polypeptide(s); and a first homology arm and a second homology arm, wherein the first and second homology arms flank the expression construct, wherein the first homology arm comprises a first sequence of about 50 to about 1000 nucleotides in length from SEQ ID NO:1, and wherein the second homology arm comprises a second sequence of about 50 to about 1000 nucleotides in length from SEQ ID NO:1, wherein the second sequence is 3′ relative to the first sequence within SEQ ID NO:1. In other aspects, provided herein are polynucleotides comprising an expression construct that comprises one or more open-reading frames (ORFs) encoding one or more polypeptide(s); and a first homology arm and a second homology arm, wherein the first and second homology arms flank the expression construct, wherein the first homology arm comprises a first sequence of about 50 to about 1000 nucleotides in length from SEQ ID NO:2, and wherein the second homology arm comprises a second sequence of about 50 to about 1000 nucleotides in length from SEQ ID NO:2, wherein the second sequence is 3′ relative to the first sequence within SEQ ID NO:2. In other aspects, provided herein are polynucleotides comprising an expression construct that comprises one or more open-reading frames (ORFs) encoding one or more polypeptide(s); and a first homology arm and a second homology arm, wherein the first and second homology arms flank the expression construct, wherein the first homology arm comprises a first sequence of about 50 to about 1000 nucleotides in length from SEQ ID NO:3, and wherein the second homology arm comprises a second sequence of about 50 to about 1000 nucleotides in length from SEQ ID NO:3, wherein the second sequence is 3′ relative to the first sequence within SEQ ID NO:3.

In other aspects, provided herein are vectors comprising the polynucleotide according to any one of the above embodiments. In some embodiments, the vectors further comprise a sequence encoding a selectable marker, wherein the selectable marker sequence is not flanked by the first and second homology arms.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A-2B show interpretation of genome and vector strand information from the SAM Filtering Pipeline (SFP program) integration site output. FIG. 1A shows the integration site for PCL6-C126 1a, in which both the reads for the genome and for the vector are on the negative strand. A browser snapshot is shown (top) in which sequencing depth is indicated by the distribution in grey and individual sequencing reads are indicated by arrows below the distribution. Nucleotides that do not match the reference genome are shown by vertical lines. Also shown is an example read for the PCL6-C126 1a integration site (bottom) showing the direction of the sequence in relation to the integration junction. Arrows indicate directionality of DNA, running from the 5′ tail to the arrowhead at the 3′ end. The wider arrow indicates the sequencing read, with genomic sequence shown to the left and vector sequence shown to the right. The integration junction is indicated by the black vertical line between the genome and the vector sequence. Narrower arrows indicate the origin of the sequence within the sequencing read, and the directionality of the original sequence in relation to the integration junction site. Numbers below the infographic indicate chromosomal coordinates for the read (left) or coordinates for the read within the vector sequence (right). FIG. 1B shows the integration site for PCL3-C31, in which both the reads for the genome and for the vector are on the positive strand. A browser snapshot is shown (top) in which sequencing depth is indicated by the distribution in grey and individual sequencing reads are indicated by arrows below the distribution. Nucleotides that do not match the reference genome are shown by vertical lines. Also shown is an example read for the PCL3-C31 integration site (bottom) showing the direction of the sequence in relation to the integration junction. Arrows indicate directionality of DNA, running from the 5′ tail to the arrowhead at the 3′ end. The wider arrow indicates the sequencing read, with genomic sequence shown to the left and vector sequence shown to the right. The integration junction is indicated by the black vertical line between the genome and the vector sequence. Narrower arrows indicate the origin of the sequence within the sequencing read, and the directionality of the original sequence in relation to the integration junction site. Numbers below the infographic indicate chromosomal coordinates for the read (left) or coordinates for the read within the vector sequence (right). FIG. 2A shows the integration site for PCL2-C10, in which the reads for the genome are on the negative strand and the reads for the vector are on the positive strand. A browser snapshot is shown (top) in which sequencing depth is indicated by the distribution in grey and individual sequencing reads are indicated by arrows below the distribution. Nucleotides that do not match the reference genome are shown by vertical lines. Also shown is an example read for the PCL2-C10 integration site (bottom) showing the direction of the sequence in relation to the integration junction. Arrows indicate directionality of DNA, running from the 5′ tail to the arrowhead at the 3′ end. The wider arrow indicates the sequencing read, with genomic sequence shown to the left and vector sequence shown to the right. The integration junction is indicated by the black vertical line between the genome and the vector sequence. Narrower arrows indicate the origin of the sequence within the sequencing read, and the directionality of the original sequence in relation to the integration junction site. Numbers below the infographic indicate chromosomal coordinates for the read (left) or coordinates for the read within the vector sequence (right). FIG. 2B shows the integration site for PCL2-C31 site 2, in which the reads for the genome are on the positive strand and the reads for the vector are on the negative strand. A browser snapshot is shown (top) in which sequencing depth is indicated by the distribution in grey and individual sequencing reads are indicated by arrows below the distribution. Nucleotides that do not match the reference genome are shown by vertical lines. Also shown is an example read for the PCL2-C31 site 2 integration site (bottom) showing the direction of the sequence in relation to the integration junction. Arrows indicate directionality of DNA, running from the 5′ tail to the arrowhead at the 3′ end. The wider arrow indicates the sequencing read, with genomic sequence shown to the left and vector sequence shown to the right. The integration junction is indicated by the black vertical line between the genome and the vector sequence. Narrower arrows indicate the origin of the sequence within the sequencing read, and the directionality of the original sequence in relation to the integration junction site. Numbers below the infographic indicate chromosomal coordinates for the read (left) or coordinates for the read within the vector sequence (right).

FIG. 3 shows a histogram of the number of integration sites identified per clone. The Production Cell Line (PCL) from which each clone was generated is indicated. The x-axis indicates the number of integration sites identified per clone, and the y-axis indicates the number of clones with the given number of integration sites per clone. These data are also depicted in tabular format in Table 3.

FIGS. 4A-4C show evidence of sister clones. FIG. 4A shows a browser snapshot of the integration site for PCL3 clones 19 and 22, located on chromosome 4 at the coordinates NC_048597.1:153138726-153139331. Tracks shown from top to bottom: PCL3-C19 Targeted Seq reads, PCL3-C22 Targeted Seq reads, DG44 ATACseq reads, DG44 ATACseq significant called peaks, DG44 ATACseq Genome States (E0: background, E1: nucleosome, E2: open), PCL3-C19 RNAseq reads from SET1, DG44 RNAseq reads, and genome annotation. FIG. 4B shows a browser snapshot of part of the integration site for PCL4 clones 100, 126, 133, and 186, located on chromosome X at the coordinates NC_048604.1:110256587-110300274. Tracks shown from top to bottom: PCL4-C100 Targeted Seq reads, PCL4-C126 Targeted Seq reads, PCL4-C133 Targeted Seq reads, PCL4-C186 Targeted Seq reads, DG44 ATACseq reads, DG44 ATACseq significant called peaks, DG44 ATACseq Genome States (E0: background, E1: nucleosome, E2: open), PCL4-C100 RNAseq reads from SET1, PCL4-C126 RNAseq reads from SET1, PCL4-C133 RNAseq reads from SET1, PCL4-C186 RNAseq reads from SET1, DG44 RNAseq reads, and genome annotation. FIG. 4C shows a browser snapshot of the integration site for PCL7 clone a1-a11 and clone a2-b5, located on chromosome 7 at the coordinates NC_048600.1:132545701-132545705. Tracks shown from top to bottom: PCL7-a1-a11 Targeted Seq reads, PCL7-a2-b5 Targeted Seq reads, DG44 ATACseq reads, DG44 ATACseq significant called peaks, DG44 ATACseq Genome States (E0: background, E1: nucleosome, E2: open), PCL3-C11 RNAseq reads from SET1, PCL2-C76 RNAseq reads from SET1, PCL6-C67 RNAseq reads from SET1, PCL4-C100 RNAseq reads from SET1, DG44 RNAseq reads, and genome annotation.

FIGS. 5-6B show analysis of the integration sites across normal Chinese hamster chromosomes. FIG. 5 shows a map of integration sites onto normal Chinese hamster chromosomes. Assembled chromosomes from the CriGri-PICRH-1.0 genome are shown along with 4 unplaced scaffolds that contained integration sites. The chromosomes are represented to scale based on size, with tick markers placed every 100 million bases (100 Mb). Integration sites are indicated by production cell line, with a reference legend shown in the bottom middle, and the integration sites are labeled with the clone number. FIG. 6A shows a histogram of the number of integration sites on each chromosome. Bars are split by production cell line, with a legend in the upper middle. The x-axis indicates the normal Chinese hamster chromosome, with “NA” representing unplaced scaffolds. The y-axis indicates the number of integration sites found on sequence corresponding to each normal Chinese hamster chromosome. FIG. 6B shows a histogram of the number of integration sites per Mb (million bases) for each chromosome. Unplaced scaffolds were not included. The x-axis indicates the chromosomes. The y-axis indicates the number of integration sites per Mb of the given chromosome.

FIGS. 7A-7B show categorization of the gene features of integration sites. FIG. 7A shows a key for differentiating gene features. From top to bottom, rows indicate the following: RNAseq reads, the full DNA sequence, gene annotation presence or absence, and gene annotation structure. In the gene annotation structure, a vertical bar indicates an exon, and an absence of vertical bars indicates an intron. Non-expressed gene intron sites are within introns of genes that did not have RNAseq reads. Non-expressed gene exon sites are within exons of genes that did not have RNAseq reads. Intergenic sites are between genes. Expressed gene intron sites are within introns of genes that did have RNAseq reads. Expressed gene exon sites are within exons of genes that did have RNAseq reads. FIG. 7B shows a pie chart of the gene features of the 106 integration sites. Genome states shown starting from the right and moving clockwise: intergenic (57 sites, 54%), non-expressed exon (1 site, 1%), expressed exon (4 sites, 4%), non-expressed intron (11 sites, 10%), and expressed intron (33 sites, 31%).

FIGS. 8A-8B show exemplary high- and low-expression regions surrounding integration sites measured in RPKM (reads per kilobase per million reads). FIG. 8A shows a genome browser snapshot of an exemplary high expression integration region, with 44.3 RPKM. Tracks shown from top to bottom: PCL3-C46 Targeted Seq reads, PCL3-C46 RNAseq reads from SET1 replicate 1, PCL3-C46 RNAseq reads from SET1 replicate 2, PCL3-C46 RNAseq reads from SET3, DG44 replicate 1 RNAseq reads, DG44 replicate 2 RNAseq reads, and genome annotation. The box denotes the integration region for which RPKM was calculated. FIG. 8B shows a genome browser snapshot of an exemplary low expression integration region, with 0 RPKM. Tracks shown from top to bottom: PCL6-C67 Targeted Seq reads, PCL3-C2 RNAseq reads from SET, PCL3-C31 RNAseq reads from SET1, DG44 replicate 1 RNAseq reads, DG44 replicate 2 RNAseq reads, and genome annotation. The box denotes the integration region for which RPKM was calculated.

FIGS. 9A-9C show analysis of gene expression of genes containing integration sites. FIG. 9A shows a histogram of expression of genes containing integration sites in the cell lines containing those sites. Bars indicate production cell line, with a legend in the top right. Gene expression is quantified as transcripts per million transcripts (TPM). Only integration sites within genes from cell lines with RNAseq data are represented. The x-axis indicates gene TPM in PCLs with integration sites. The y-axis indicates the number of integration sites within a gene with the given TPM. FIG. 9B shows a histogram of expression of genes containing integration sites in DG44 cells that do not contain those integration sites. Bars indicate production cell line, with a legend in the top right. Gene expression is quantified as transcripts per million transcripts (TPM). All integration sites within genes are represented. The x-axis indicates gene TPM in DG44 cells. The y-axis indicates the number of integration sites within a gene with the given TPM in DG44 cells. FIG. 9C shows a scatterplot of the expression of genes containing integration sites in cell lines containing the integration sites on the x-axis versus expression in DG44 of genes containing integration sites (y-axis). Points indicate production cell line, and a legend is provided at the top left. Gene expression is quantified as transcripts per million transcripts (TPM). Only integration sites within genes from cell lines with RNAseq data are represented. The equation for linear regression along with R value, R² value, and p-value for Pearson correlation are shown on the graph.

FIGS. 10A-10D show categorization of genome states of integration sites. FIG. 10A shows a graphical depiction of genome states predicted from ATACseq. The horizontal line represents a DNA chromosome. The grey circles represent nucleosomes. The oval represents the transposase used in ATACseq. Regions that are open and accessible to the transposase are designated E2. Regions containing nucleosomes flanking open regions are designated E1. Regions of inaccessible DNA are the background closed state and are designated E0. Adapted from FIG. 1 of Tarbell and Liu (Tarbell and Liu, 2019, Nucleic Acids Research. 47(16): e91). FIG. 10B shows a pie chart of the distribution of genome states among the 106 analyzed integration sites. Genome states shown starting from the right and moving clockwise: E1—nucleosome regions (52 sites, 49%), E2—open regions (8 sites, 8%), and E0—background regions (46 sites, 43%). FIG. 10C shows a genome browser snapshot with exemplary genome states as determined by ATACseq. Tracks shown from top to bottom: ATACseq read distribution, ATACseq significant called peaks, genome states as determined from ATACseq data, RNAseq read distribution, and genome annotation. FIG. 10D shows a genome browser snapshot of the endogenous Eeflal (CHEF1) region of the genome, which is very accessible and highly transcribed. Tracks shown from top to bottom: ATACseq read distribution, ATACseq significant called peaks, genome states as determined from ATACseq data, RNAseq read distribution, and genome annotation.

FIGS. 11A-11C show analysis of copy number at integration sites based on DG44 Whole Genome Sequencing (WGS) read depth. FIG. 11A shows a genome browser snapshot with exemplary copy number variations. Tracks shown from top to bottom: WGS read pileup, estimated copy number, and mappability metric. The left box indicates a region with increased reads, indicating an increased local copy number. The middle box indicates a baseline region with diploid copy number. The right box indicates a region with a low mappability score, in which increased reads do not conclusively indicate increased local copy number. FIG. 11B shows a pie chart of the distribution of the 106 integration sites based on whether the genome region was diploid, gained copies, or lost copies. Copy number states shown starting from the right and moving clockwise: diploid (68 sites, 64%), copy loss (5 sites, 5%), and copy gain (33 sites, 31%). FIG. 11C shows a histogram of the estimated genome copy number of integration regions. Bars indicate production cell line, with a legend in the top right. The x-axis indicates estimated genome copy number of an integration region, and the y-axis indicates the number of integration sites with the corresponding genome copy number.

FIGS. 12A-12B show exemplar integration site regions. FIG. 12A shows a genome browser snapshot with an approximately 100 kb integration region, defined as −50 kb from the left side of the integration site to +50 kb from the right side of the integration site. Tracks shown from top to bottom: PCL3-C31 Targeted Seq reads, DG44 ATACseq reads, DG44 ATACseq significant called peaks, DG44 ATACseq Genome States (E0: background, E1: nucleosome, E2: open), PCL3-C31 RNAseq reads from SET1 replicate 1, PCL3-C31 RNAseq reads from SET1 replicate 2, DG44 RNAseq reads, and genome annotation. FIG. 12B shows a genome browser snapshot with an approximately 118 kb integration region, defined as −50 kb from the left side of the integration site to +50 kb from the right side of the integration site. Tracks shown from top to bottom: PCL6-C126 Targeted Seq reads, DG44 ATACseq reads, DG44 ATACseq significant called peaks, DG44 ATACseq Genome States (E0: background, E1: nucleosome, E2: open), PCL6-C126 RNAseq reads from SET1, DG44 RNAseq reads, and genome annotation.

FIGS. 13A-13D show analysis of expression for integration sites. FIG. 13A shows a histogram of expression level in the region ±50 kb around each integration site in cell lines containing those integration sites. Bars indicate production cell line, with a legend in the top right. Only integration regions from cell lines with RNAseq data are represented. The x-axis indicates expression levels in RPKM (reads per kilobase per million reads) for the region around integration sites in cell lines containing the integration sites at SET1. The y-axis indicates the number of integration sites with the corresponding expression level. FIG. 13B shows a histogram of expression level in the region ±50 kb around each integration site in DG44 cells that do not contain the integration site. Bars indicate production cell line, with a legend in the top right. The x-axis indicates expression levels in RPKM (reads per kilobase per million reads) for the region around integration sites DG44 cells. The y-axis indicates the number of integration sites with the corresponding expression level. FIG. 13C shows a scatterplot of expression level in the region ±50 kb around each integration site in cell lines containing the integration sites at SET1 (x-axis) versus in DG44 (y-axis). Points indicate production cell line, and a legend is provided at the top left. Expression is quantified as reads per kilobase per million reads (RPKM). Only integration regions from cell lines with RNAseq data are represented. The equation for linear regression along with R value, R² value, and p-value for Pearson correlation are shown on the graph. FIG. 13D shows a scatterplot of expression level in the region ±50 kb around each integration site in cell lines containing the integration sites at SET1 (x-axis) versus the average expression for all other clones of the same production cell line that did not contain the integration sites (y-axis). Points indicate production cell line, and a legend is provided at the top left. Expression is quantified as reads per kilobase per million reads (RPKM). Only integration regions from cell lines with RNAseq data are represented. The equation for linear regression along with R value, R² value, and p-value for Pearson correlation are shown on the graph.

FIGS. 14A-14D show analysis of chromatin state and distance to features of interest for integration sites. FIG. 14A shows a histogram of ATACseq read depth for integration sites. Bars indicate production cell line. A legend is shown in the top right. The x-axis indicates ATACseq read depth in RPKM (reads per kilobase per million reads) for the region ±50 kb around an integration site in DG44 cells. The y-axis indicates the number of integration sites that have the corresponding ATACseq read depth. FIG. 14B shows a histogram of the percent of the regions surrounding integration sites covered by ATACseq significant called peaks, for all integration sites. Bars indicate production cell line. A legend is shown in the top right. The x-axis indicates the percentage of the region ±50 kb around an integration site that is covered by significant called ATACseq peaks. The y-axis indicates the number of integration sites covered by the corresponding percentage. FIG. 14C shows a histogram of the distance from integration sites to the nearest expressed gene. Bars indicate production cell line. A legend is shown in the top right. The x-axis indicates the distance in kilobases from an integration site to the nearest expressed gene in DG44. The y-axis indicates the number of integration sites at the corresponding distance. FIG. 14D shows a histogram of the distance from integration sites to the nearest ATACseq significant called peak. Bars indicate production cell line. A legend is shown in the top right. The x-axis indicates the distance in kilobases from an integration site to the nearest significant called ATACseq peak. The y-axis indicates the number of integration sites at the corresponding distance.

FIGS. 15A-15B show mapping of integration sites from single integration site production cell lines. FIG. 15A shows a map of single integration sites onto normal Chinese hamster chromosomes. Assembled chromosomes from the CriGri-PICRH-1.0 genome are shown to scale, with tick markers placed every 100 million bases (100 Mb). Integration sites indicate production cell line, with a reference legend shown on the right, and the integration sites are labeled with the clone number. FIG. 15B shows a histogram of single integration sites by chromosome. Bars indicate production cell line, with a legend provided in the top right. The x-axis indicates normal Chinese hamster chromosomes by number, with “NA” representing unplaced scaffolds. The y-axis indicates the number of single integration sites found on the corresponding chromosome.

FIGS. 16A-16G show analyses of integration sites from single integration site production cell lines. FIG. 16A shows a pie chart of the gene features of the 13 single integration sites. Genome states shown starting from the right and moving clockwise: intergenic (5 sites, 38%), expressed exon (2 sites, 15%), non-expressed intron (3 sites, 23%), and expressed intron (3 sites, 23%). FIG. 16B shows a pie chart of the distribution of genome states among the 13 analyzed single integration sites. Genome states shown starting from the right and moving clockwise: E0—background regions (5 sites, 38%), E2—open regions (3 sites, 23%), and E1—nucleosome regions (5 sites, 38%). FIG. 16C shows a pie chart of the distribution of the 13 single integration sites based on whether the genome region was diploid or gained copies. Copy number states shown starting from the right and moving clockwise: diploid (8 sites, 62%), and copy gain (5 sites, 38%). FIG. 16D shows a histogram of the estimated genome copy number of single integration regions. Bars indicate production cell line, with a legend in the top right. The x-axis indicates estimated genome copy number of a single integration region, and the y-axis indicates the number of single integration sites with the corresponding genome copy number. FIG. 16E shows a histogram of the distance from single integration sites to the nearest ATACseq significant called peak. Bars indicate production cell line. A legend is shown in the top right. The x-axis indicates the distance in kilobases from a single integration site to the nearest significant called ATACseq peak. The y-axis indicates the number of single integration sites at the corresponding distance. FIG. 16F shows a histogram of the percent of the regions surrounding single integration sites covered by ATACseq significant called peaks. Bars indicate production cell line. A legend is shown in the top right. The x-axis indicates the percentage of the region ±50 kb around a single integration site that is covered by significant called ATACseq peaks. The y-axis indicates the number of single integration sites covered by the corresponding percentage. FIG. 16G shows a scatterplot of the percent of the region f50 kb around a single integration site covered by a significant called ATACseq peak (x-axis) versus IgG heavy chain expression measured in transcripts per million (TPM) (y-axis). Points indicate clones, and a legend is provided on the right. The equation for linear regression along with R value, R² value, and p-value for Pearson correlation are shown on the graph.

FIG. 17 shows a summary chart of features of the identified single integration sites. Columns indicate clones containing single integration sites. Rows, from top to bottom, indicate: chromosome, whether the integration site is within a gene in either an intron or exon, the genome state as determined by ATACseq, the distance to the nearest ATACseq significant called peak, the percentage of the integration region covered by ATACseq significant called peaks, the presence or absence of expressed genes within the integration region, the copy number at the integration site, the titer of antibody produced in the clone at Stability Evaluation Timepoint 1 (SET1), and the percent change in titer of antibody produced from SET1 to SET3 (time between Stability Evaluation Timepoints was approximately 40 days). Light cells indicate favorable conditions; blank cells indicate neutral conditions; darker cells indicate non-favorable conditions.

FIGS. 18A-18B show the design for targeted integration. FIG. 18A shows a targeted integration vector including an expression vector with homology arms specific to each target location. The circular black line indicates the circularized expression plasmid. The box labeled “LHA” indicates the left homology arm. The grey boxes and black arrows indicate the expression construct. The box labeled “RHA” indicates the right homology arm. The box labeled “LS” indicates an optional linearization site. The arrow labeled “GFP” indicates the green fluorescence protein marker gene. FIG. 18B shows schematics for using GFP outside of homology arms for screening purposes. In the top-scenario, the absence of GFP indicates correct integration of the cassette. In the middle scenario, the presence of GFP flanking either the left or right homology arms indicates incomplete integration of the cassette. In the bottom scenario, the presence of GFP flanking the left homology arm is used to diagnose off-site integration of the cassette.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%” When “about” is applied to the first value of a set of values, it applies to all values in that set. Thus, “about 7, 9, or 11 mg/kg” is equivalent to “about 7, about 9, or about 11 mg/kg.”

As used herein, a “heterologous” polypeptide may refer to a polypeptide not normally produced by a host cell as it exists in nature, e.g., a Chinese hamster cell. For example, a heterologous polypeptide may be encoded by a gene or open-reading frame not naturally present in a Chinese hamster cell genome. In some embodiments, a heterologous polypeptide is a recombinant polypeptide. In some embodiments, a heterologous polypeptide is a polypeptide normally produced by a different type of cell (e.g., a mammalian cell other than a Chinese hamster cell such as a human or mouse cell), including but not limited to human, humanized, murine, or chimeric polypeptides.

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)₂, and Fv).

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6.

The L chain from any vertebrate species can be assigned to one of two clearly distinct types, called kappa and lambda, based on the amino acid sequences of their constant domains.

Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in the present disclosure. Common allotypic variants in human populations are those designated by the letters a, f, n, z.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

An “antibody fragment” comprises a portion of an intact antibody, the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

### II. Cells and Integration Sites

In certain aspects, the present disclosure provides CHO cells (e.g., isolated CHO cells) comprising an expression construct that comprises one or more open-reading frames (ORFs) encoding one or more heterologous polypeptide(s), wherein the expression construct is integrated in the CHO cell genome at an integration site of the present disclosure.

In other aspects, the present disclosure provides CHO cells (e.g., isolated CHO cells) comprising a landing pad sequence for mediating targeted integration, such as RMCE, wherein the landing pad sequence is integrated in the CHO cell genome at an integration site of the present disclosure.

Sequences and chromosomal coordinates for the integration sites of the present disclosure are provided herein. In some embodiments, the integration sites of the present disclosure include the sites PCL2-C122, PCL3-C31, and PCL6-C126 as described herein. Descriptions, chromosomal coordinates, and sequences for the integration sites of the present disclosure are provided in Table 1.

TABLE 1
Targeted integration sites.

| | SEQ | | | | | |
|---|---|---|---|---|---|---|
| | ID | | Region | Region | Region | |
| Name | No | Scaffold | Start | End | Size (bp) | Description |
| PCL2- | 1 | NC_048602.1 | 16770620 | 16791941 | 21321 | Open/nucleosome |
| C122 | | | | | | region encompassing |
| | | | | | | the integration site |
| | | | | | | (bounded by E0 |
| | | | | | | closed region). |
| PCL3- | 2 | NC_048603.1 | 25162471 | 25185950 | 23479 | Region between and |
| C31 | | | | | | including the 2 |
| | | | | | | ATACseq peaks on |
| | | | | | | either side of the |
| | | | | | | integration site. |
| PCL6- | 3 | NC_048601.1 | 13898813 | 13917158 | 18345 | Region between the |
| C126 | | | | | | two ends of the |
| | | | | | | integrated plasmid. |

A variety of Chinese hamster genomes are known in the art and publicly available. In some embodiments, chromosomal coordinates provided for an integration site of the present disclosure refer to coordinates according to the Chinese Hamster Genome Chromosome Assembly 2020 (CriGri-PICRH-1.0; RefSeq assembly accession number GCF_003668045.3; GenBank assembly accession number GCA_003668045.2). Other available Chinese Hamster genome assemblies are described in Table 2.

TABLE 2
Chinese hamster genome assemblies.

| Genome | Accession # | Description |
|---|---|---|
| CriGri-PICRH- | GCF_003668045.3 | Chinese Hamster Genome Chromosome |
| 1.0 | | Assembly 2020 |
| CriGri-1.0 | GCF_000223135.1 | CHO K1 Genome BGI 2011 |
| C_griseus_v1.0 | GCF_000419365.1 | Chinese Hamster Genome BGI 2013 |
| Cgr1.0 | GCA_000448345.1 | Chinese Hamster Genome CeBiTec 2013 |
| CHOZN v2.4 | GCA_026545065.1 | CHOZN GS Genome 2022 |
| CHOKIS HZDv1 | GCA_900186095.1 | Horizon Discovery CHO K1 GS Genome 2017 |

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to a sequence selected from the group consisting of SEQ ID Nos:1-3, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to a sequence selected from the group consisting of SEQ ID Nos:1-3, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence selected from the group consisting of SEQ ID Nos:1-3, e.g., in a CHO cell genome. In some embodiments, an integration site of the present disclosure is integrated within a sequence having at least 97% sequence identity to SEQ ID NO:1, 2, or 3, e.g., in a CHO cell genome. In some embodiments, an integration site of the present disclosure is integrated within a sequence having at least 99% sequence identity to SEQ ID NO:1, 2, or 3, e.g., in a CHO cell genome. In some embodiments, an integration site of the present disclosure is integrated within SEQ ID NO:1, 2, or 3, e.g., in a CHO cell genome. In some embodiments, the sequence having at least 97% or at least 99% sequence identity to SEQ ID NO:1, 2, or 3, e.g., in a CHO cell genome, is between 50 and 1000 base pairs in length. In some embodiments, the integration site is within a sequence between 50 and 1000 base pairs in length from a sequence having at least 97% or at least 99% sequence identity to a sequence selected from the group consisting of SEQ ID Nos:1-3. In some embodiments, the integration site is within a sequence between 50 and 1000 base pairs in length from a sequence selected from the group consisting of SEQ ID Nos:1-3.

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to SEQ ID NO:1, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to SEQ ID NO:1, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about lkb of SEQ ID NO:1, e.g., in a CHO cell genome. In some embodiments, an integration site of the present disclosure is integrated within SEQ ID NO:1, e.g., in a CHO cell genome.

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to a region spanning chromosomal coordinates NC_048602.1:16770620-16791941 according to Chinese Hamster reference genome CriGri-PICRH-1.0, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to a region spanning chromosomal coordinates NC_048602.1:16770620-16791941 according to Chinese Hamster reference genome CriGri-PICRH-1.0, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence spanning chromosomal coordinates NC_048602.1:16770620-16791941 according to Chinese Hamster reference genome CriGri-PICRH-1.0, e.g., in a CHO cell genome. In some embodiments, an integration site of the present disclosure is integrated within chromosomal coordinates NC_048602.1:16770620-16791941 according to Chinese Hamster reference genome CriGri-PICRH-1.0, e.g., in a CHO cell genome.

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to SEQ ID NO:2, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to SEQ ID NO:2, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about lkb of SEQ ID NO:2, e.g., in a CHO cell genome. In some embodiments, an integration site of the present disclosure is integrated within SEQ ID NO:2, e.g., in a CHO cell genome.

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to a region spanning chromosomal coordinates NC_048603.1:25162471-25185950 according to Chinese Hamster reference genome CriGri-PICRH-1.0, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to a region spanning chromosomal coordinates NC_048603.1:25162471-25185950 according to Chinese Hamster reference genome CriGri-PICRH-1.0, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence spanning chromosomal coordinates NC_048603.1:25162471-25185950 according to Chinese Hamster reference genome CriGri-PICRH-1.0, e.g., in a CHO cell genome. In some embodiments, an integration site of the present disclosure is integrated within chromosomal coordinates NC_048603.1:25162471-25185950 according to Chinese Hamster reference genome CriGri-PICRH-1.0, e.g., in a CHO cell genome. Coordinates homologous to NC_048603.1:25162471-25185950 (according to Chinese Hamster reference genome CriGri-PICRH-1.0) that could alternatively be used include KE382060.1:6158430-6181940 according to reference genome C_griseus_v1.0, KE686185.1:5,025,523-5,049,002 according to reference genome Cgr1.0, LT883755.1:67,489-44,001 according to reference genome CHOK1S_HZDv1, or any of the coordinates shown in Table 2B.

TABLE 2B
Alternate chromosomal coordinates for PCL3-C31.

| Genome | Position |
|---|---|
| CriGri-1.0 | NW_003630189.1:813-1; |
| | NW_003704848.1:1-240; |
| | NW_003717482.1:1-209; |
| | NW_003633658.1:1-1341; |
| | NW_003623233.1:3993-1; |
| | NW_003623970.1:1-3308; |
| | NW_003627463.1:2050-1; |
| | NW_003693508.1:292-1; |
| | NW_003622781.1:1-4662; |
| | NW_003625172.1:1-2710; |
| | NW_003684776.1:1-356; |
| | NW_003629753.1:1689-1: |
| | NW_003626639:1-1233 |
| CHOZN v2.4 | JAPKFS010010040:2111-1; |
| | JAPKFS010290701:588-1; |
| | JAPKFS010005844:1-4082; |
| | JAPKFS010005844:4183-7529; |
| | JAPKFS010004010:1-2460; |
| | JAPKFS010004010:2561-7344; |
| | JAPKFS010009612:1-5781; |
| | JAPKFS010272209:1-453 |

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to SEQ ID NO:3, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to SEQ ID NO:3, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of SEQ ID NO:3, e.g., in a CHO cell genome. In some embodiments, an integration site of the present disclosure is integrated within SEQ ID NO:3, e.g., in a CHO cell genome.

In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 97% sequence identity to a region spanning chromosomal coordinates NC_048601.1:13898813-13917158 according to Chinese Hamster reference genome CriGri-PICRH-1.0, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence having at least 99% sequence identity to a region spanning chromosomal coordinates NC_048601.1:13898813-13917158 according to Chinese Hamster reference genome CriGri-PICRH-1.0, e.g., in a CHO cell genome. In some embodiments, an integration site of the present disclosure is integrated within about 20 kb, within about 15 kb, within about 10 kb, within about 9 kb, within about 8 kb, within about 7 kb, within about 6 kb, within about 5 kb, within about 4 kb, within about 3 kb, within about 2 kb, or within about 1 kb of a sequence spanning chromosomal coordinates NC_048601.1:13898813-13917158 according to Chinese Hamster reference genome CriGri-PICRH-1.0, e.g., in a CHO cell genome. In some embodiments, an integration site of the present disclosure is integrated within chromosomal coordinates NC_048601.1:13898813-13917158 according to Chinese Hamster reference genome CriGri-PICRH-1.0, e.g., in a CHO cell genome. Coordinates homologous to NC_048601.1:13898813-13917158 (according to Chinese Hamster reference genome CriGri-PICRH-1.0) that could alternatively be used include NW_003613840.1:1,670,335-1,651,868 according to reference genome CriGri-1.0, KE380049.1:1,586,202-1,604,684 according to reference genome C_griseus_v1.0, KE682606.1:1,236,056-1,217,629 according to Cgr1.0, JAPKFS010001178:56,051,208-56,028,018 according to CHOZN v2.4, and LT883702.1:11,813,986-11,832,387 according to CHOK1S_HZDv1.

In some embodiments, an integration site of the present disclosure is the only genomic site at which an expression construct or landing pad sequence of the present disclosure is integrated in the CHO cell genome. In some embodiments, an integration site of the present disclosure is in a diploid region of the genome. In some embodiments, an integration site of the present disclosure is in a copy gain region of the genome, e.g., is present in greater than 2 copies in the genome. In some embodiments, e.g., for integration site PCL6-C126, the integration site of the present disclosure has a copy number of 2 in the Chinese hamster cell genome. In some embodiments, e.g., for integration site PCL3-C31, the integration site of the present disclosure has a copy number of 6 in the Chinese hamster cell genome. In some embodiments, e.g., for integration site PCL2-C122, the integration site of the present disclosure has a copy number of 53 in the Chinese hamster cell genome.

In some embodiments, an integration site of the present disclosure is an intergenic site not within an intron or exon, e.g., of a native Chinese hamster gene. Any of the Chinese hamster genome assemblies described herein can be used to identify intergenic sites based on standard genome annotation.

In some embodiments, an integration site of the present disclosure is located in open chromatin in the CHO cell genome. For example, the integration site can be within about 5 kb or less of a peak and/or in E2 state according to Assay for Transposase Accessible Sequencing (ATACseq) analysis. ATACseq refers to an assay that uses transposase-mediated fragmentation followed by sequencing in order to determine chromatin accessibility across the genome. Exemplary ATACseq assays are known in the art and described herein.

In some embodiments, the CHO cell lacks dihydrofolate reductase (DHFR) activity. For example, in some embodiments, the CHO cell comprises loss-of-function mutations or deletions in both copies of a DHFR gene. DHFR catalyzes the conversion of folate to tetrahydrofolate in the de novo synthesis pathway for purines and pyrimidines. Cells lacking DHFR activity are useful for recombinant protein expression and cell culturing because polynucleotides/vectors encoding a DHFR polypeptide can be introduced into DHFR-deficient cells and used as a marker to select for clones that have DHFR activity and thus carry the polynucleotide or vector. Methotrexate, an inhibitor of DHFR, can also be used to select for cells with a certain level of DHFR expression or activity. Chinese hamster DHFR genes are known in the art; see. e.g., NCBI Gene ID No. 100689028, NM_001244016, and NP_001230945. DHFR polypeptides suitable for CHO cell expression and selection are known in the art.

Another system that can be used alternatively or in addition to DHFR selection is glutamine synthetase (GS). In some embodiments, the CHO cell lacks glutamine synthetase (GS) activity. For example, in some embodiments, the CHO cell comprises loss-of-function mutations or deletions in both copies of a GS gene. GS catalyzes the production of glutamine from glutamate and ammonia. Cells lacking GS activity are useful for recombinant protein expression and cell culturing because polynucleotides/vectors encoding a GS polypeptide can be introduced into GS-deficient cells and used as a marker to select for clones that have GS activity and thus carry the polynucleotide or vector. Methionine sulfoxamine, an inhibitor of GS, can also be used to select for cells with a certain level of GS expression or activity. Chinese hamster GS genes are known in the art; see, e.g., NCBI Gene ID No. 100764163, NM_001416242, and NP_001403171. GS polypeptides suitable for CHO cell expression and selection are known in the art.

A variety of CHO cells and cell lines are known in the art and contemplated for use in the present disclosure. In some embodiments, the CHO cell is a DG44 CHO cell or cell line, including the original DG44 cell line (see, e.g., Urlaub, G. et al. (1983) Cell 33(2):405-412) and cell lines descending or derived therefrom. In some embodiments, the CHO cell is a CHOK1 CHO cell or cell line, including the original CHOK1 cell line (see, e.g., Kao, F. T. and Puck, T. T. (1968) Proc Natl Acad Sci 60(4):1275-1281) and cell lines descending or derived therefrom. In some embodiments, the CHO cell is a CHOK1SV CHO cell or cell line, including the original CHOK1SV cell line (see, e.g., de la Cruz Edmonds, M. et al. (2006) Molecular Biotechnology 34:179-190) and cell lines descending or derived therefrom. In some embodiments, the CHO cell is a DXB11 CHO cell or cell line, including the original DXB11 cell line (see, e.g., Urlaub, G. and Chasin, L. A. (1980) Proc Natl Acad Sci 77(7):4216-4220) and cell lines descending or derived therefrom. CHO cell lines are widely available; see, e.g., ATCC cell line CCL-61, Gibco™ CHO DG44 cells (cGMP banked) (MermoFisher Scientific Cat. No. A1100001), ATCC cell line CRL 9096 and ECACC Cat. No. 94060607, Cellosaurus Accession No. CVCL_1977, etc. As used herein, references to a particular CHO cell line are meant to encompass the original cell line and any subsequent cell lines descending or derived therefrom.

### Heterologous Polypeptides

In some embodiments, a CHO cell of the present disclosure comprises an expression construct integrated into the genome at an integration site of the present disclosure. In some embodiments, the expression construct comprises one or more ORFs encoding one or more heterologous polypeptide(s).

A variety of heterologous polypeptides are contemplated for use herein. CHO cells are widely used in the art for production of heterologous (e.g., recombinant) polypeptides.

In some embodiments, the expression construct comprises an ORF encoding a polypeptide (e.g., a recombinant, heterologous, or therapeutic polypeptide). In some embodiments, the expression construct comprises an ORF encoding a fusion protein (e.g., a recombinant, heterologous, or therapeutic fusion protein). In some embodiments, the expression construct comprises an ORF encoding an enzyme (e.g., a recombinant, heterologous, or therapeutic enzyme). In some embodiments, the expression construct comprises an ORF encoding an antibody (e.g., a recombinant, heterologous, or therapeutic antibody). In some embodiments, the expression construct comprises an ORF encoding a biologic drug (e.g., a recombinant, heterologous, or therapeutic biologic). In some embodiments, the expression construct comprises an ORF encoding a cytokine or chemokine (e.g., a recombinant, heterologous, or therapeutic cytokine or chemokine). In some embodiments, the expression construct comprises an ORF encoding a growth factor or hormone (e.g., a recombinant, heterologous, or therapeutic growth factor or hormone). In some embodiments, the expression construct comprises an ORF encoding a vaccine peptide or subunit (e.g., a recombinant, heterologous, or therapeutic vaccine peptide or subunit). In some embodiments, the expression construct comprises an ORF encoding a blood factor (e.g., a recombinant, heterologous, or therapeutic blood factor). In some embodiments, the expression construct comprises an ORF encoding a neurotoxin (e.g., a recombinant, heterologous, or therapeutic neurotoxin).

In some embodiments, the expression construct comprises an ORF encoding an antibody or antigen-binding fragment thereof. In some embodiments, the expression construct comprises an ORF encoding an antibody light chain or antigen-binding fragment thereof. In some embodiments, the expression construct comprises an ORF encoding an antibody heavy chain or antigen-binding fragment thereof. In some embodiments, the expression construct comprises a first ORF encoding an antibody light chain and a second ORF encoding an antibody heavy chain or antigen-binding fragment thereof. In some embodiments, the expression construct comprises an ORF encoding a single chain antibody or antigen-binding fragment thereof, including, without limitation, a scFv, single chain antibody, nanobody, camelid antibody, or VHH antibody.

In some embodiments, the expression construct further comprises a promoter operably linked to the one or more ORFs. In some embodiments, the promoter drives gene expression in a CHO cell. In some embodiments, the promoter is an inducible or constitutive promoter. In some embodiments, in the case of multiple ORFs, each ORF can be operably linked to its own promoter, or multiple ORFs can be operably linked to the same promoter. A variety of promoters suitable for use in CHO cells are known in the art. DNA regions are generally considered to be operably linked when they are functionally related to each other. For example, a promoter can be operably linked to a coding sequence if the promoter is capable of participating in the transcription of the sequence. Similarly, a ribosome-binding site can be operably linked to a coding sequence if it is positioned so as to permit translation.

In some embodiments, the expression construct further comprises a 5′ enhancer, 5′ intron, translational initiation region (TIR), splice spacer, Kozak sequence, internal ribosome entry sequence (IRES), 3′ UTR, and/or polyadenylation signal, e.g., in operable linkage with an ORF.

In some embodiments, the expression construct further comprises a sequence encoding a selectable marker. A variety of selectable markers suitable for CHO cells are known in the art, including antibiotics, auxotrophic markers, visual markers (e.g., fluorescent or bioluminescent proteins, or enzymes that catalyze chemical reactions resulting in visible products), GS, and DHFR as described herein. See also U.S. Pat. No. 11,268,109.

### Landing Pad Sequences and RMCE

In some embodiments, a CHO cell of the present disclosure comprises a landing pad sequence integrated into its genome at an integration site of the present disclosure. In some embodiments, the landing pad sequence mediates recombinase-mediated cassette exchange (RMCE). As is known in the art, RMCE refers to the precise replacement of a target cassette integrated in the genome (e.g., a landing pad) with a donor cassette (e.g., comprising a sequence of interest, such as one or more ORFs encoding one or more heterologous polypeptide(s)) using a recombinase, e.g., a site-specific recombinase (SSR). The molecular compositions typically provided in order to perform this process include 1) a genomic target cassette flanked both 5′ and 3′ by recognition target sites specific to a particular recombinase (e.g., a landing pad sequence), 2) a donor cassette flanked by matching recognition target sites, and 3) the site-specific recombinase. SSRs enable precise cleavage of DNA and recombination at recognition sites, resulting in precise exchange of DNA between recognition sequences of the donor cassette and the genomic target cassette. In some embodiments, the SSR is a site-specific DNA recombinase.

SSRs can be used to perform targeted DNA rearrangements such as deletions, inversions, integrations, and translocations when two recombinase recognition sites are placed strategically in the genome of an organism. Different outcomes of a site-specific recombination system, such as but not limited to Cre-loxP, depend on the position and orientation of the recombinase recognition sites, for example the two loxP sites. If the loxP sequences have the same orientation, the recombination results in the excision of the DNA fragment flanked by the two loxP sequences. If the orientation of loxP elements is in opposite, the result of the reaction is the inversion of the DNA segment flanked by the two loxP sites. Recombination between the two loxP sites located on different DNA molecules produces strand exchange or translocation. Variants or mutants of recombinase recognition sites, for example lox sites, may also be employed (Araki et al. (2002) Nucleic Acids Research 30:19, e103). Several site-specific recombination systems are known in the art, including, without limitation, FLP/FRT (see, e.g., O'Gorman et al. (1991) Science 251: 1351-1355), Cre/loxP (see, e.g., Sauer and Henderson (1988) Proc. Natl. Acad. Sci. 85: 5166-5170), phi C31-att (see, e.g., Groth et al. (2000) Proc. Natl. Acad. Sci. 97: 5995-6000), R recombinase/Rs recombination sites (see, e.g., Onounchi et al. (1991) Nucleic Acid Res. 19: 6373-6378), Dre recombinase/rox sites (U.S. Pat. No. 7,422,889), and Gin recombinase/gix sites (see, e.g., Maeser et al. (1991) Mol. Gen. Genet. 230: 170-176). See also Tian, X. and Zhou, B. (2021) J Biol Chem 296:100509.

In some embodiments, the landing pad sequence is heterologous to a Chinese hamster genome. In some embodiments, the landing pad sequence comprises a first and a second target sequence recognized by a SSR (e.g., recognition target sites specific to a particular recombinase). In some embodiments, the first and second target sequences are heterologous to a Chinese hamster genome. Target sequences suitable for a number of RMCE strategies and specific for a variety of SSRs are known in the art; exemplary and non-limiting descriptions can be found in the references cited supra. The person of ordinary skill in the art may select a particular target sequence or pair of target sequences using knowledge common in the art.

In some embodiments, the landing pad sequence further comprises a sequence encoding a selectable marker of the present disclosure. A variety of selectable markers suitable for CHO cells are known in the art, including antibiotics, auxotrophic markers, visual markers (e.g., fluorescent or bioluminescent proteins, or enzymes that catalyze chemical reactions resulting in visible products), GS, and DHFR as described herein.

Methods suitable for integrating a landing pad sequence of the present disclosure into the genome of a CHO cell at an integration site of the present disclosure are known in the art. Various methods for editing a host cell genome at a specific target location are known in the art. Genetic editing techniques such as are described below can be used to stably integrate a nucleic acid sequence into a eukaryotic cell in which the nucleic acid sequence is a foreign sequence to the host cell genome.

Homologous recombination can be used to insert a nucleic acid molecule into a target locus. A construct is designed with the desired insertion sequence (e.g., a landing pad sequence of the present disclosure) flanked by sequence homologous to the genomic sequence flanking the desired genomic insertion site (e.g., homology arms). This construct is provided to the host cell by transfection or a similar method. Cells frequently repair double-strand breaks by non-homologous end-joining (NHEJ). However, if homologous sequence in the form of a donor template is available in the cell, homology directed repair or homology directed recombination (HDR) may occur instead. Donor template DNA molecules include DNA molecules comprising, from 5′ to 3′, a first homology arm, a replacement DNA sequence, and a second homology arm, wherein the homology arms containing sequences that are partially or completely homologous to genomic DNA sequences flanking the targeted locus and wherein the replacement DNA can comprise an insertion, deletion, or substitution of 1 or more DNA base pairs relative to the targeted locus. In certain embodiments, a donor DNA template homology arm can be about 20, 50, 100, 200, 400, or 600 to about 800, or 1000 base pairs in length. In certain embodiments, a donor template DNA molecule can be delivered to a eukaryotic cell (e.g., a CHO cell) in a circular (e.g., a plasmid or a viral vector including a geminivirus vector) or a linear DNA molecule. Donor DNA templates can be synthesized either chemically or enzymatically (e.g., in a polymerase chain reaction (PCR)).

Use of donor templates other than double-stranded DNA are also contemplated. Donor templates can be precursors to double-stranded DNA, single stranded RNA templates for reverse transcriptase, single-stranded DNA, single- or double-stranded RNA, or a DNA/RNA hybrid.

Homologous recombination in eukaryotic cells can be facilitated by introducing a break in the chromosomal DNA at the desired integration site. This may be accomplished by targeting a zinc finger nuclease (ZFN), transcription activator-like effector nuclease (TALEN), or site-specific nuclease or RNA-guided nuclease, such as a Cas nuclease, to the specific integration locus. Gene targeting vectors are also employed to facilitate homologous recombination.

Zinc finger nucleases (ZFNs) have a modular structure and contain individual zinc finger domains which recognize a particular 3-nucleotide sequence in the target sequence. Typically, the engineered zinc finger DNA binding domain has a novel binding specificity, compared to a naturally-occurring zinc finger protein. Engineering methods include but are not limited to rational design and various types of selection. Rational design includes, for example, the use of databases of triplet or quadruplet nucleotide sequences and individual zinc finger amino acid sequences, in which each triplet or quadruplet nucleotide sequence is associated with one or more amino acid sequences of zinc fingers which bind the particular triplet or quadruplet sequence. See. e.g., U.S. Pat. Nos. 6,453,242 and 6,534,261. Exemplary selection methods (e.g., phage display and yeast two-hybrid systems) are well known and described in the literature. In addition, enhancement of binding specificity for zinc finger binding domains has been described, e.g., in U.S. Pat. No. 6,794,136. Individual zinc finger domains may be linked together using any suitable linker sequences. Examples of linker sequences are publicly known, see. e.g., U.S. Pat. Nos. 6,479,626; 6,903,185; and 7,153,949. The nucleic acid cleavage domain is non-specific and is typically a restriction endonuclease, such as FokI. This endonuclease must dimerize to cleave DNA. Thus, cleavage by FokI as part of a ZFN requires two adjacent and independent binding events, which must occur in both the correct orientation and with appropriate spacing to permit dimer formation. The requirement for two DNA binding events enables more specific targeting of long and potentially unique recognition sites. FokI variants with enhanced activities have been described; see, e.g., Guo et al. (2010) J. Mol. Biol., 400:96-107.

Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare sequence-specific endonucleases referred to as TAL-effector nucleases or TALENs. As in the case of ZFNs, a restriction endonuclease, such as FokI, can be conveniently used in a fusion protein with the TAL in order to recognize and cleave DNA at a target sequence within the locus of the invention (Boch et al. (2009) Science 326: 1509-1512).

RNA-guided endonucleases such as those in a CRISPR (clustered regularly interspaced short palindromic repeats)/Cas (CRISPR-associated) system can also be used. Site-specificity of Cas endonuclease is conferred by association with a guide RNA that is complementary to a target DNA sequence. Various RNA-guided Cas nucleases are known in the art, including but not limited to Cas9, Cas12a (Cpf1), Cas12e (CasX), Cas12d (CasY), C2c1, C2c2, C2c3 (see WO2018176009), Cas12h, Cas12i (see Yan et al. (2019) Science 363(6422): 88-91) and Cas12j (Pausch et al. (2020) Science, 369(6501): 333-337), homologs thereof, or modified versions thereof). CRISPR/Cas systems are part of the adaptive immune system of bacteria and archaea. Immunity is acquired by the integration of short fragments of the invading DNA known as spacers between two adjacent repeats at the proximal end of a CRISPR locus. Spacers are transcribed and processed into small interfering CRISPR RNAs (crRNAs) approximately 40 nt in length, which combine with the trans-activating CRISPR RNA (tracrRNA) to activate and guide the Cas nuclease to defend against invading nucleic acids such as viral RNA by cleaving the foreign DNA in a sequence-dependent manner.

A prerequisite for cleavage is the presence of a conserved protospacer-adjacent motif (PAM) downstream of the target DNA. The type of RNA-guided endonuclease typically informs the location of suitable PAM sites and design of crRNAs or sgRNAs. G-rich PAM sites, e.g., 5′-NGG-3′ are typically targeted for design of crRNAs or sgRNAs used with Cas9 proteins. T-rich PAM sites (e.g., 5′-TTTV-3′, where “V” is A, C, or G) are typically targeted for design of crRNAs or sgRNAs used with Cas12a proteins. Cpf1 endonuclease and corresponding guide RNAs and PAM sites are disclosed in US PG Pub. No. 2016/0208243. Specificity is provided by the so-called “seed sequence” approximately 12 bases upstream of the PAM, which must match between the RNA and target DNA.

CRISPR technology for editing the genes of eukaryotes is disclosed in US PG Pub Nos. 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US PG Pub. No. 2016/0208243 A1. Other CRISPR nucleases useful for editing genomes include Cas12b and Cas12c (see Shmakov et al. (2015) Mol. Cell, 60: 385-397) and CasX and CasY (see Burstein et al. (2016) Nature, doi:10.1038/nature21059).

Modifications of CRISPR technologies, such as prime editing (U.S. Pat. No. 11,447,770), are also known in the art and can be used be one of skill in the art.

Still other methods of homologous recombination are available to the skilled artisan, such as BuD-derived nucleases (BuDNs) with precise DNA-binding specificities (Stella et al. (2014) Acta Cryst. D70: 2042-2052).

### III. Methods, Polynucleotides, Vectors, and Kits

In other aspects, the present disclosure provides methods for generating a cell line (e.g., a CHO cell line) that expresses one or more heterologous polypeptide(s). In some embodiments, the methods comprise introducing a polynucleotide comprising an expression construct that comprises one or more open-reading frames (ORFs) encoding the one or more heterologous polypeptide(s) into a CHO cell of the present disclosure comprising a landing pad sequence for mediating targeted integration, such as RMCE, wherein the landing pad sequence is integrated in the CHO cell genome at an integration site of the present disclosure, under conditions suitable for RMCE between the landing pad sequence and the expression construct.

In some embodiments, the methods further comprise selecting for cell(s) that integrated the expression construct at an integration site of the present disclosure. In some embodiments, the expression construct further comprises a selectable marker. In some embodiments, successful integration of the expression construct results in loss of a marker that can be subject to selection. Suitable selection methods and markers are described herein and known in the art, e.g., DHFR, GS, antibiotics, auxotrophic markers, and visual markers (e.g., fluorescent or bioluminescent proteins, or enzymes that catalyze chemical reactions resulting in visible products).

Methods for introducing a polynucleotide comprising an expression construct into a CHO cell of the present disclosure, as well as conditions suitable for RMCE, are known in the art. Transfection is the process of introducing exogenous materials such as nucleic acid polynucleotides or molecules into target cells, typically by non-viral means. Transduction is typically used to describe the process of introducing foreign nucleic acid to a host cell by viral means. Exogenous nucleic acids are generally introduced to the host cell by methods involving opening transient pores or holes in the cell membrane in order to allow for uptake of the provided materials. Numerous methods of transfection are known in the art, including but not limited to electroporation, cell squeezing, DEAE-dextran mediated delivery, calcium phosphate precipitate method, cationic lipid-mediated delivery, liposome or nanoparticle mediated transfection, electroporation, microprojectile bombardment, receptor-mediated gene delivery, and delivery mediated by carriers such as polylysine, histones, chitosan, peptides.

In other aspects, the present disclosure provides methods for producing one or more heterologous polypeptide(s). In some embodiments, the methods comprise culturing a CHO cell comprising one or more open-reading frames (ORFs) encoding one or more heterologous polypeptide(s) as described herein, wherein the expression construct is integrated in the CHO cell genome at an integration site of the present disclosure, under conditions suitable for production of the heterologous polypeptide(s).

Methods for culturing CHO cells are known in the art. See. e.g., Sharker, S. and Rahman, A. (2021) Curr Drug Discov Technol 18(3):354-364. Media suitable for culturing CHO cells are known in the art; see. e.g., Gibco™ CD DG44 Medium (MermoFisher Scientific Cat. No. 12610010). Media can be chemically defined. Media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Media can include serum or be serum-free. Any other supplements may also be included at appropriate concentrations that would be known to those skilled in the art. The culture conditions, such as temperature, pH, and the like, are those previously used with the host cell selected for expression, and will be apparent to the ordinarily skilled artisan.

In some embodiments, the methods further comprise recovering the heterologous polypeptide(s) from the CHO cell. Expressed proteins may be secreted into the culture medium, depending on the nucleic acid sequence selected, but may be retained in the cell or deposited in the cell membrane. Various purification/separation methods for recovering heterologous polypeptide(s) from production CHO cells are known in the art and include, without limitation, affinity chromatography, ion exchange chromatography, ethanol precipitation, high-performance liquid chromatography (HPLC), ammonium sulfate precipitation, hydroxyapatite chromatography, dialysis, gel filtration, SDS-PAGE, and the like. For example, for recovery of antibodies, Protein A immobilized on a solid phase is used for immunoaffinity purification of antibodies. Protein A is a 41 kD cell wall protein from Staphylococcus aureus which binds with a high affinity to the Fc region of antibodies. The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants. As the first step of purification, a preparation derived from the cell culture as described above can be applied onto a Protein A immobilized solid phase to allow specific binding of the antibody of interest to Protein A. The solid phase would then be washed to remove contaminants non-specifically bound to the solid phase. Finally the antibody of interest is recovered from the solid phase by elution. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Methods 62:1-13 (1983)). Protein G can be used all mouse isotypes and for human γ3 (Guss et al., EMBO J. 5:15671575 (1986)). The matrix to which the affinity ligand is attached may be agarose, but other matrices are available. Mechanically stable matrices such as controlled pore glass or poly(styrenedivinyl)benzene allow for faster flow rates and shorter processing times than can be achieved with agarose.

As demonstrated herein, the integration sites of the present disclosure allow for stable, high levels of production, e.g., of a recombinant or heterologous polypeptide of interest. In some embodiments, the amount of heterologous polypeptide production by a CHO cell of the present disclosure is stable over time. For example, in some embodiments, the amount of heterologous polypeptide production by a CHO cell of the present disclosure on day 40 of culturing is at least about 85%, at least about 90%, or at least about 95% as compared to the amount of heterologous polypeptide production on day 1 of culturing.

In other aspects, the present disclosure provides polynucleotides and vectors comprising an expression construct of the present disclosure flanked by a first and a second homology arm, wherein the first and the second homology arms each independently comprise sequences with homology to an integration site of the present disclosure. For example, in some embodiments, the first and the second homology arms each independently comprise a sequence about 50 to about 1000 nucleotides in length from SEQ ID NO:1. In some embodiments, the first and the second homology arms each independently comprise a sequence from SEQ ID NO:1 that is less than about any of the following lengths (in nucleotides): 1000, 950, 900, 850, 800, 750, 700, 650, 600, 550, 500, 450, 400, 350, 300, 250, 200, 150, 100, 90, 80, 70, or 60. In some embodiments, the first and the second homology arms each independently comprise a sequence from SEQ ID NO:1 that is greater than about any of the following lengths (in nucleotides): 50, 60, 70, 80, 90, 100, 150, 200, 250, 300, 350, 400, 450, 500, 550, 600, 650, 700, 750, 800, 850, 900, or 950. That is, the length of the first and the second homology arms can each comprise a sequence from SEQ ID NO:1 having a range of sizes with an upper limit of 1000, 950, 900, 850, 800, 750, 700, 650, 600, 550, 500, 450, 400, 350, 300, 250, 200, 150, 100, 90, 80, 70, or 60 nucleotides and an independently selected lower limit of 50, 60, 70, 80, 90, 100, 150, 200, 250, 300, 350, 400, 450, 500, 550, 600, 650, 700, 750, 800, 850, 900, or 950 nucleotides, wherein the upper limit is greater than the lower limit. In some embodiments, the first and the second homology arms each independently comprise a sequence about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, or about 1000 nucleotides in length from SEQ ID NO:1. In some embodiments, the second sequence is 3′ relative to the first sequence within SEQ ID NO:1. In some embodiments, the first and the second homology arms each independently comprise a sequence about 50 to about 1000 nucleotides in length from SEQ ID NO:2. In some embodiments, the first and the second homology arms each independently comprise a sequence from SEQ ID NO:2 that is less than about any of the following lengths (in nucleotides): 1000, 950, 900, 850, 800, 750, 700, 650, 600, 550, 500, 450, 400, 350, 300, 250, 200, 150, 100, 90, 80, 70, or 60. In some embodiments, the first and the second homology arms each independently comprise a sequence from SEQ ID NO:2 that is greater than about any of the following lengths (in nucleotides): 50, 60, 70, 80, 90, 100, 150, 200, 250, 300, 350, 400, 450, 500, 550, 600, 650, 700, 750, 800, 850, 900, or 950. That is, the length of the first and the second homology arms can each comprise a sequence from SEQ ID NO:2 having a range of sizes with an upper limit of 1000, 950, 900, 850, 800, 750, 700, 650, 600, 550, 500, 450, 400, 350, 300, 250, 200, 150, 100, 90, 80, 70, or 60 nucleotides and an independently selected lower limit of 50, 60, 70, 80, 90, 100, 150, 200, 250, 300, 350, 400, 450, 500, 550, 600, 650, 700, 750, 800, 850, 900, or 950 nucleotides, wherein the upper limit is greater than the lower limit. In some embodiments, the first and the second homology arms each independently comprise a sequence about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, or about 1000 nucleotides in length from SEQ ID NO:2. In some embodiments, the second sequence is 3′ relative to the first sequence within SEQ ID NO:2. In some embodiments, the first and the second homology arms each independently comprise a sequence about 50 to about 1000 nucleotides in length from SEQ ID NO:3. In some embodiments, the first and the second homology arms each independently comprise a sequence from SEQ ID NO:3 that is less than about any of the following lengths (in nucleotides): 1000, 950, 900, 850, 800, 750, 700, 650, 600, 550, 500, 450, 400, 350, 300, 250, 200, 150, 100, 90, 80, 70, or 60. In some embodiments, the first and the second homology arms each independently comprise a sequence from SEQ ID NO:3 that is greater than about any of the following lengths (in nucleotides): 50, 60, 70, 80, 90, 100, 150, 200, 250, 300, 350, 400, 450, 500, 550, 600, 650, 700, 750, 800, 850, 900, or 950. That is, the length of the first and the second homology arms can each comprise a sequence from SEQ ID NO:3 having a range of sizes with an upper limit of 1000, 950, 900, 850, 800, 750, 700, 650, 600, 550, 500, 450, 400, 350, 300, 250, 200, 150, 100, 90, 80, 70, or 60 nucleotides and an independently selected lower limit of 50, 60, 70, 80, 90, 100, 150, 200, 250, 300, 350, 400, 450, 500, 550, 600, 650, 700, 750, 800, 850, 900, or 950 nucleotides, wherein the upper limit is greater than the lower limit. In some embodiments, the first and the second homology arms each independently comprise a sequence about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, or about 1000 nucleotides in length from SEQ ID NO:3. In some embodiments, the second sequence is 3′ relative to the first sequence within SEQ ID NO:3. In some embodiments, the first and the second homology arms comprise different sequences. In some embodiments, the first and the second homology arms have the same length. In some embodiments, the first and the second homology arms have different lengths.

In some embodiments, the vectors further comprise a sequence encoding a selectable marker of the present disclosure. In some embodiments, the selectable marker sequence is not flanked by the first and second homology arms.

In some embodiments, the vectors further comprise a sequence encoding a Cas nuclease, wherein the sequence encoding the Cas nuclease is not flanked by the first and second homology arms.

In other aspects, the present disclosure provides a single guide RNA (sgRNA) comprising a crispr RNA (crRNA) sequence and a tracr RNA sequence, wherein the tracr RNA sequence binds a Cas nuclease, and wherein the crRNA sequence comprises a sequence about 17 to about 23 nucleotides in length from one of SEQ ID Nos:1-3, or a sequence about 17 to about 23 nucleotides in length from a sequence having at least 97% or at least 99% identity to one of SEQ ID Nos:1-3.

In some embodiments, a vector of the present disclosure further comprises a sequencing encoding a sgRNA of the present disclosure, wherein the sequence encoding the sgRNA is not flanked by the first and second homology arms.

In other aspects, the present disclosure provides kits or articles of manufacture. In some embodiments, the kits or articles of manufacture comprise a polynucleotide or vector of the present disclosure and optionally further comprise instructions for using the polynucleotide or vector to integrate an expression construct of the polynucleotide or vector into a CHO cell genome at an integration site of the present disclosure. In some embodiments, the kits or articles of manufacture comprise a polynucleotide or vector of the present disclosure and a sgRNA or sequence encoding a sgRNA of the present disclosure. In some embodiments, the kits or articles of manufacture further comprise a polynucleotide encoding a Cas nuclease. In some embodiments, the kits or articles of manufacture further comprise instructions for using the polynucleotide or vector, sgRNA, and optionally Cas nuclease to integrate an expression construct of the polynucleotide or vector into a CHO cell genome at an integration site of the present disclosure.

In other aspects, the present disclosure provides methods for generating a cell line (e.g., a CHO cell line) that expresses one or more heterologous polypeptide(s). In some embodiments, the methods comprise introducing a sgRNA of the present disclosure, a polynucleotide encoding a Cas nuclease, and a polynucleotide or vector of the present disclosure into a CHO cell under conditions suitable for integration of the expression construct into the genome of the CHO cell via the sgRNA and the Cas nuclease. In some embodiments, the methods further comprise selecting for CHO cell(s) with integration of the expression construct, e.g., as described herein.

### EXAMPLES

The invention will be more fully understood by reference to the following examples. The examples should not, however, be construed as limiting the scope of the invention. It is understood that the examples and embodiments described herein are for illustrative purposes only and that various modifications or changes in light thereof will be suggested to persons skilled in the art, and are to be included within the spirit and purview of this application and the scope of the appended claims.

### Example 1: Identification of Successful Integration Sites

Production cell lines used for production of recombinant proteins (e.g., antibodies or other biologics) are typically generated by randomly integrating varying copies of construct(s) of interest into a cell line by stable transfection. However, the particular transgene integration site(s) in biologics-producing cell lines can influence productivity and stability. To identify features of desirable integration sites, a high-throughput, in-house integration site analysis pipeline was applied to clonally-derived production cell lines. Integration sites were identified for top clones from 7 different antibody-expression programs. From the 40 clones characterized, over 100 unique integration sites were identified, with individual clones having from 1 to 19 different genomic integration sites. To characterize these integration sites, multiple-omics technologies were utilized including Whole Genome Sequencing (WGS), RNA sequencing (RNAseq), and Assay for Transposase Accessible Sequencing (ATACseq). These -omics data were linked to phenotypic characteristics of the clones such as productivity and stability to identify features of desirable integration sites. These features were used to screen clones and select candidate integration sites for targeted integration.

### Targeted Genome Sequencing

Forty top performing clones of seven different production cell lines (PCLs) were identified. The seven PCLs are identified as PCL1-PCL7 (each PCL corresponding to a different recombinant antibody product).

Cell pellets were collected from different cell lines in passage during exponential growth. Genomic DNA (gDNA) was extracted from cell pellets consisting of 2×10⁶-5×10⁶ cells using a Qiagen® DNeasy® Blood & Tissue Kit. Targeting sequencing was used for identifying existing integration sites as in O'Brien et al. 2020 (O'Brien et al., 2020, Biotechnol Prog. 36(4): e2978). Briefly, the genome of the production line cell (PCL) was fragmented. Some fragments of DNA contained only sequence from the DG44 cell, others contained only sequence from the vector, and a final subset contained sequence overlapping the integration junction. Streptavidin-conjugated beads were used to bind to short biotinylated RNA probes. The RNA probes were designed to bind the vector sequence. Pull-down of the beads resulted in capture of DNA fragments with vector sequence. DNA fragments with vector sequence were subjected to paired-end sequencing. Sequencing reads were mapped to the genome and vector, and the reads were filtered. The integration site was identified as the junction between the genomic reads and the vector reads.

Library preparation was done using either the Agilent SureSelect XT HS2 Library Prep kit or the Twist Library Preparation EF Kit 2.0. Barcoded libraries were pooled in sets of 4 or 8 samples before hybridization to a custom probe library designed against a standard vector containing a human IgK light chain constant region and a human IgG1 heavy chain constant region. The probe library did not include the variable heavy and light chain sequences for cross-cell line compatibility. Hybridization and sequence capture was performed using the Agilent SureSelect XT HS2 Kit.

Targeted sequence capture libraries were sequenced 16 samples at a time on either an Illumina HiSeq 3000/4000 or an Illumina MiSeq using a 2×150 bp paired end sequencing configuration. More than one million read pairs were sequenced for each sample.

For samples library prepped using the Agilent SureSelect XT HS2 kit, Fastq sequencing files were pre-trimmed using Agilent AGeNT Trimmer v2.0.3. Pre-trimmed fastq files and raw fastq files from samples prepared using the Twist library prep kit were trimmed using Trimmomatic v0.39. Trimmed, properly paired reads were mapped to the CriGri-PICRH 1.0 RefSeq version of the Chinese Hamster Genome (Accession Number GCF_003668045.3) using bwa-mem v0.7.17. For each production cell line included in the data set, a specific genome contained the vector added as an additional scaffold was utilized. After mapping, mapped reads were de-duplicated using Picard Tools MarkDuplicates v2.24.2. For samples library prepped using the Agilent SureSelect XT HS2 kit, the Unique Molecular Identifier (UMI) tag was supplied to Picard to aid in de-duplication.

### Bioinformatic Analysis

For each program analyzed, a vector specific to the antibody in the production cell line was added to the Chinese Hamster genome and used for mapping. Since the vector was delivered to the cell line as a linearized fragment, the vector sequence was re-indexed to start at the linearization cut site, i.e. position 1 was defined as the PvuI cut site.

De-duplicated, mapped reads were used as input for a modified version of the SFP program (SAM Filtering Pipeline, doi.org/10.13020/9wgm-mj51). Several modifications were made to the program to improve its versatility and for compatibility with the expression vector design. First, the vector contained several regions with identical sequence, including the two sets of vector Promoters and Introns before heavy chain and light chain, and part of the 3′ Enhancer and the 3′ Flank having identical sequence. The original SFP program discards reads with low mapping quality (MAPQ scores), and so any reads which mapped fully or partially to these repeated regions would be automatically discarded by the program. Any integration sites in these regions would therefore also be missed. To address this issue, the program was modified to take a table of homologous sequence regions as input. Integration junctions found in these regions were recorded despite the low MAPQ score, and both possible vector positions were output in the list of integration sites.

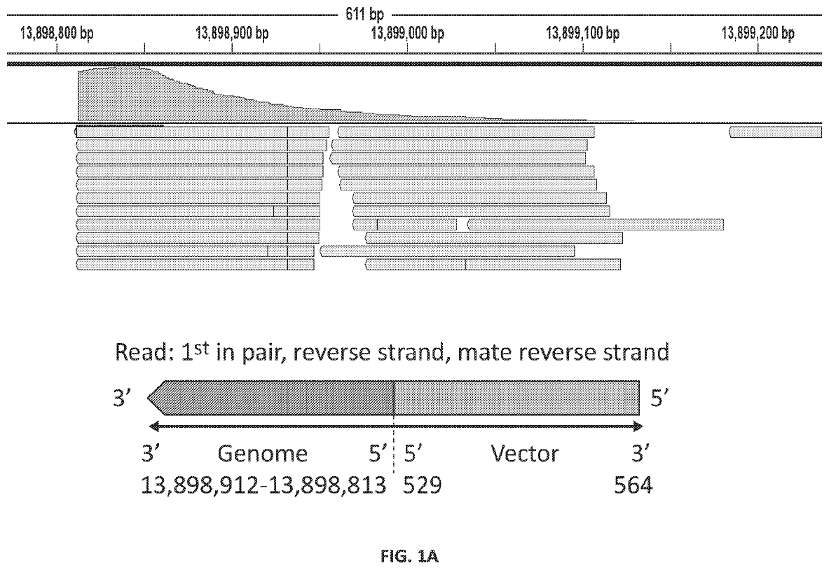

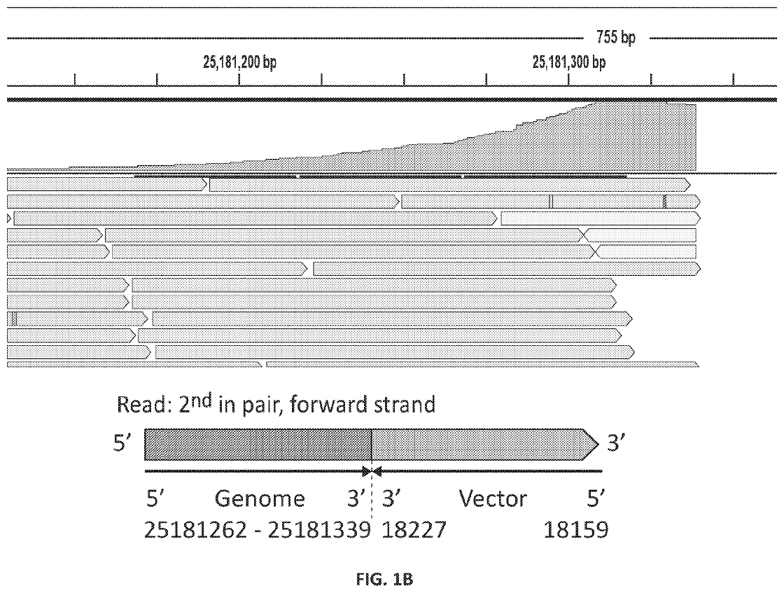

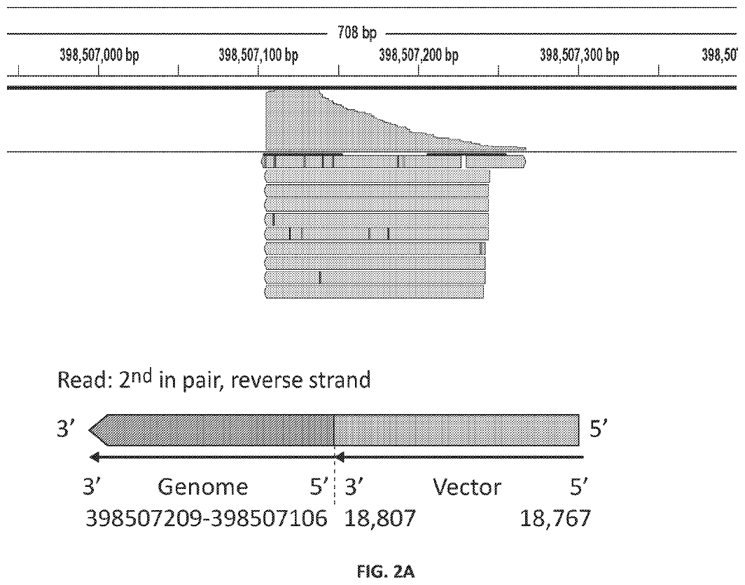

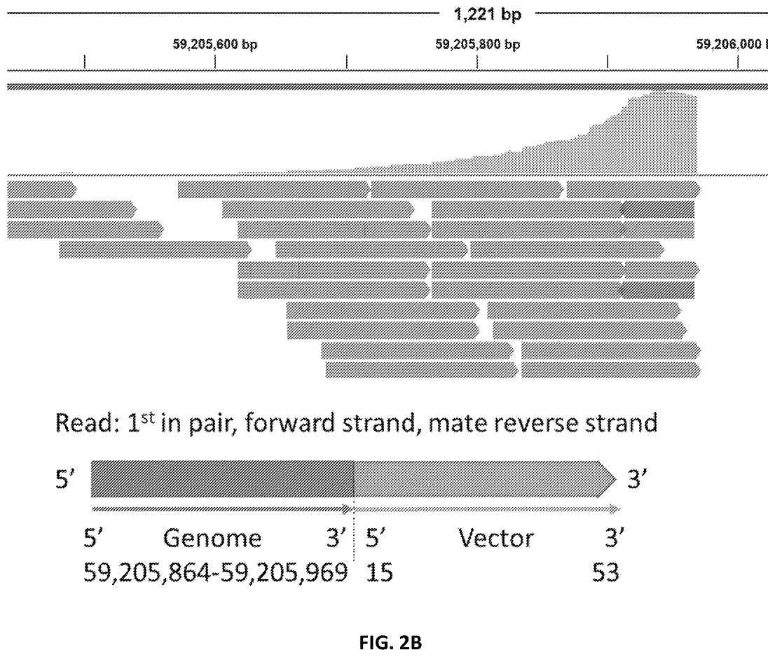

Second, to interpret the orientation of the vector and genome at the vector-genome junction, new columns were added to the integration site output which specified strand information (+ or − strand) for both the vector and the genome at the integration site. FIGS. 1A-2B show the interpretation of the strand information for each combination of genome/vector strands at four example integration site junctions.

Finally, during the typical read filtering process, reads with a low MAPQ score on either the vector or genome side of the junction were discarded, since these could be noise or could indicate that the fragment was too short for proper mapping. However, this may exclude integration sites that were within low complexity regions of the genome with poor mappability. The algorithm was modified such that reads which passed filtering for the vector mapping but had low mapping quality for the genome side of the junction were recorded separately. These “Ambiguous genome integration sites” were output in a separate list that was considered when evaluating specific clones.

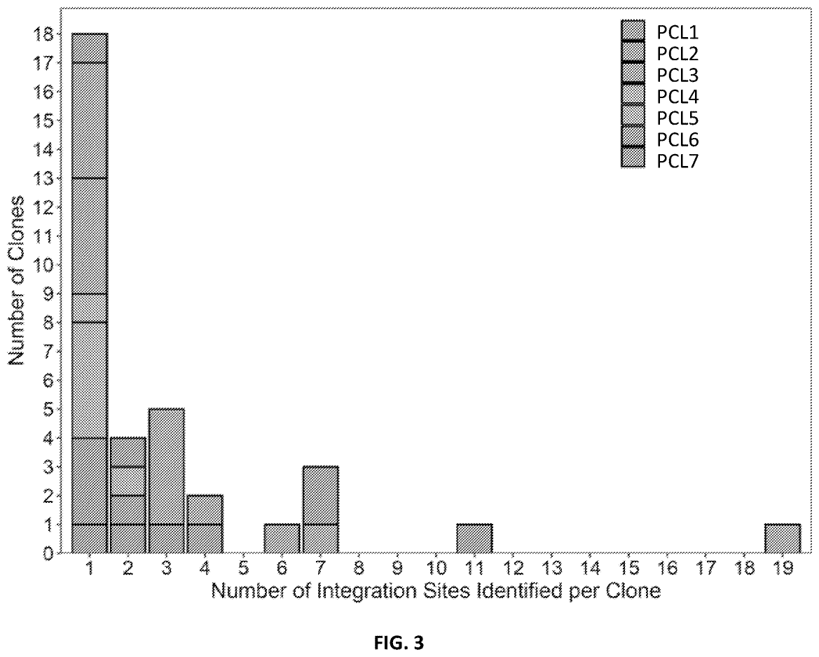

106 integration sites were identified, as well as an additional 23 integration sites with low mapping quality, as detailed in Table 3. Most clones had a single integration site identified, with the next most common number of integration sites being 3 (FIG. 3). Three of the PCL2 clones, C56, C69, and C76, were in a pool of cells which were electroporated twice. This may have caused the extremely large number of integration sites seen in PCL2-C56.

TABLE 3
Number of integration sites identified per clone, including
sites with low quality at the genome junction.

| | Number | Number of Sites | |
|---|---|---|---|
| | of Sites | with Low Quality | Total |
| PCL-Clone | Identified | Genome Junction | Sites |
| PCL2-C10 | 1 | 0 | 1 |
| PCL2-C31 | 7 | 1 | 8 |
| PCL2-C37 | 1 | 1 | 2 |
| PCL2-C56 | 19 | 2 | 21 |
| PCL2-C69 | 0* | 2 | 2 |
| PCL2-C76 | 4 | 1 | 5 |
| PCL2-C122 | 1 | 0 | 1 |
| PCL3-C2 | 1 | 1 | 2 |
| PCL3-C4 | 3 | 0 | 3 |
| PCL3-C11 | 1 | 0 | 1 |
| PCL3-C19/C22 | 3 | 2 | 5 |
| PCL3-C31 | 1 | 0 | 1 |
| PCL3-C32 | 3 | 3 | 6 |
| PCL3-C41 | 1 | 1 | 2 |
| PCL3-C46 | 6 | 0 | 6 |
| PCL3-C54 | 3 | 0 | 3 |
| PCL4-C54 | 1 | 0 | 1 |
| PCL4-C100/C126/ | 2 | 0 | 2 |
| C133/C186 | | | |
| PCL6-C3 | 4 | 2 | 6 |
| PCL6-C67 | 1 | 0 | 1 |
| PCL6-C76 | 3 | 1 | 4 |
| PCL6-C93 | 2 | 0 | 2 |
| PCL6-C126 | 1 | 0 | 1 |
| PCL6-C139 | 1 | 0 | 1 |
| PCL7-a1-a11/a2-b5 | 1 | 0 | 1 |
| PCL7-a2-e1 | 11 | 2 | 13 |
| PCL7-b6-h6 | 2 | 0 | 2 |
| PCL1-C12 | 7 | 1 | 8 |
| PCL1-C14 | 1 | 1 | 2 |
| PCL1-C27 | 2 | 0 | 2 |
| PCL1-C48 | 67 | 2 | 9 |
| PCL5-C35 | 1 | 0 | 1 |
| PCL5-C62 | 1 | 0 | 1 |
| PCL5-C74 | 1 | 0 | 1 |
| PCL5-C80 | 1 | 0 | 1 |

*A low quality site was used for the analysis in the absence of a clear integration site. This brings the total number of sites to 106.

### Identification of Sister Clones

Rows in Table 3 with multiple clones listed were identified as sister clones, as they had identical integration sites, and these were counted as only one site in following analyses.

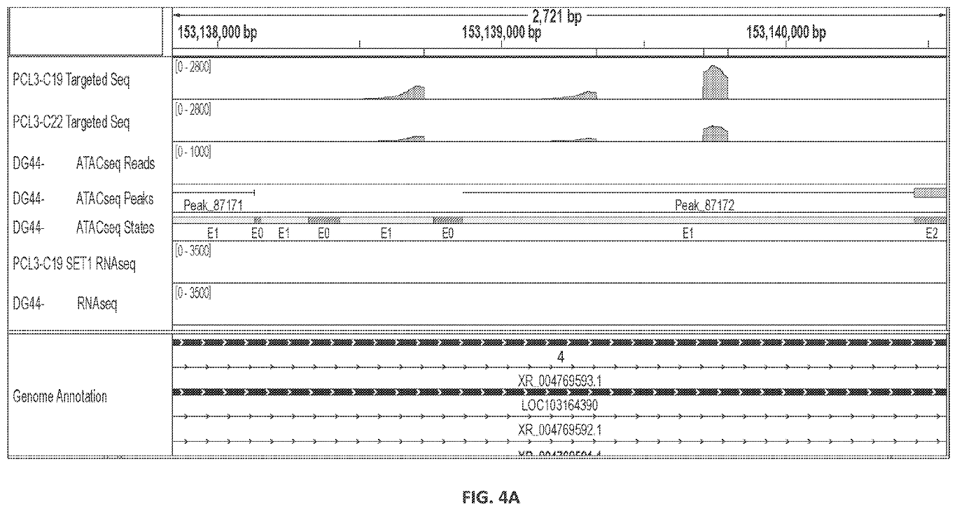

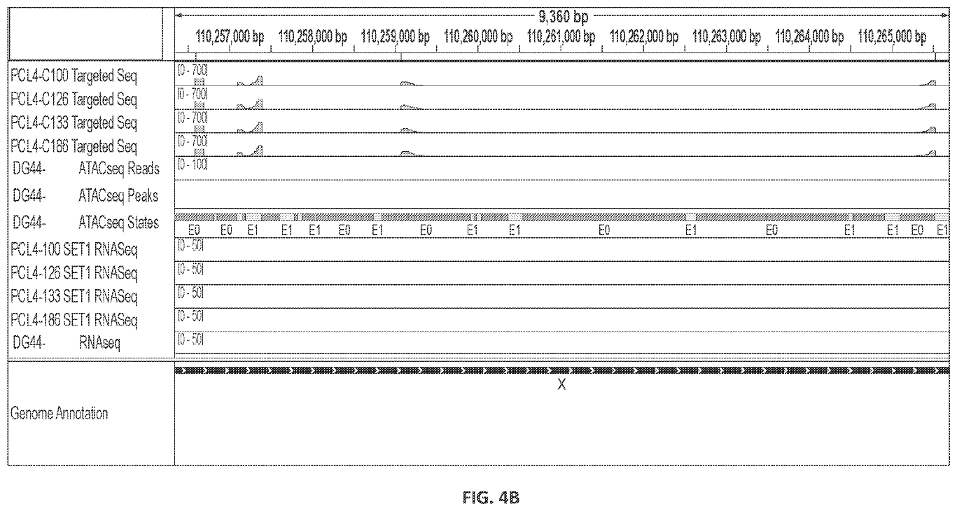

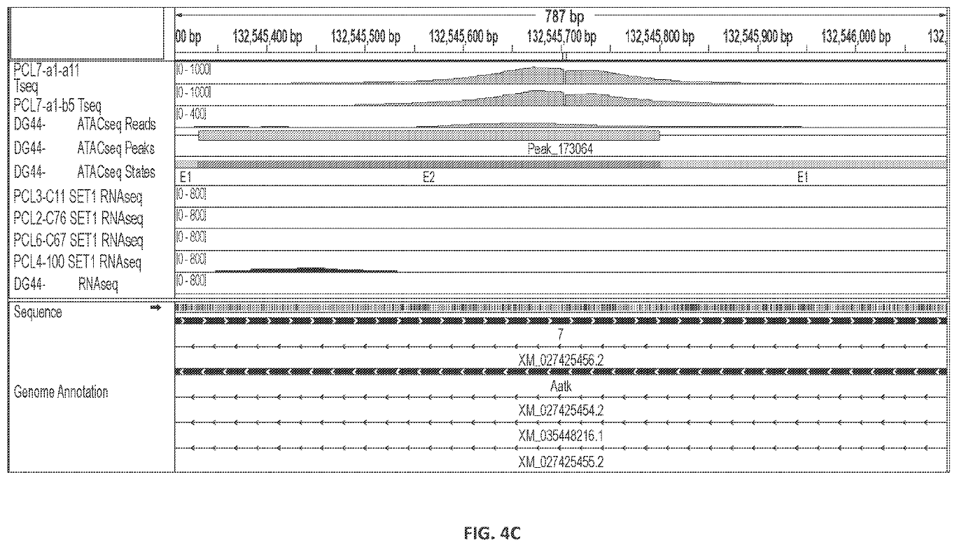

PCL3 clones C19 and C22 were determined to be sister clones, as these two clones have identical integration sites and were from the same pool of cells (FIG. 4A). The genome browser tracks show the targeted sequencing reads for both cell lines display identical breakpoint junctions.

PCL4 clones C100, C126, C133, and C186 were determined to be sister clones, as these four clones all had identical integration sites and were from the same pool of cells (FIG. 4B). The genome browser tracks show the targeted sequencing reads for all four cell lines display identical breakpoint junctions.

PCL7 clones a1-a11 and a2-b5 were determined to be sister clones, as they had identical integration sites (FIG. 4C). The genome browser tracks show the targeted sequencing reads for both cell lines at the integration sites display an identical junction.

### Example 2: Analysis of Integration by Chromosome

Normal Chinese Hamster cells have 22 chromosomes total, 11 pairs of chromosomes named 1-10 and X, as is evidenced in a karyotype from FIG. 2 of Biedler et al. (Biedler et al., 1988, Cancer Res. 48(11): 3179-87) showing a Chinese hamster cell line derived from bone marrow which is chromosomally “near normal.” DG44 and other CHO cell lines have abnormal karyotypes, and three studies were referenced to identify a base level DG44 karyotype for understanding which chromosomes were intact versus rearranged in DG44 compared to the chromosomally “near normal” CHO cell: Cao et al. (Cao et al., 2012, Biotechnol. Bioeng. 109(6): 1357-67; see, e.g., FIG. 3), Derouazi et al. (Derouazi et al., 2006, Biochem. Biophys. Res. Commun. 340(4): 1069-77; see, e.g., FIG. 4), and Bandyopadhyay et al. (Bandyopadhyay et al., 2019, Biotechnol. Bioeng. 116(1): 41-53; see, e.g., FIG. 2). Cao et al. compared their results to Derouazi et al., and determined that certain chromosomes (A, B, C, F, L, N, R, and S in Cao et al. corresponding to chromosomes 1, 2, 4, 5, 8, and 9 in Derouazi et al.) were intact in both studies and conserved between DG44 and CHO-K1, indicating that these may be stable.

Bandyopadhyay et al. showed karyotype images for high producing DG44-based subclones. These subclones have approximately 35 chromosomes per cell on average, compared to the 20 chromosomes shown in the DG44 cells from the previous two studies. From this last study the number of easily identifiable intact chromosomes for 7 different cells from each subclone was quantified. The numbering of chromosomes in this study followed the classical numbering based on size, with the numbering going from 1, 2, X, 4-11. So, chromosome 4 in FIG. 2 of Bandyopadhyay et al. corresponds to chromosome 3 in FIG. 3 of Cao et al. and FIG. 4 of Derouazi et al., chromosome 5 is chromosome 4, and so on. From this, intact copies of chromosomes 1, 2, 5, and 10 are present in all cells karyotyped in Bandyopadhyay et al., and chromosomes 6, 7, and 8 are present in some cells but lost in others (using the non-classical numbering method).

Combined with the chromosomes deemed stable from Cao et al. and Derouazi et al. (chromosomes 1, 2, 4, 5, 8, and 9), chromosomes 1, 2, and 5 are most consistently present across the CHO cells measured in these studies. Based on these results, the chromosomes of DG44 were characterized as shown in Table 4.

TABLE 4
State of normal Chinese hamster
chromosomes in DG44 based on literature review.

| | Number | | |
|---|---|---|---|
| | of Studies | | |
| | Shown as | | Corresponding |
| Chrom. | Conserved | Status | Scaffold(s) |
| 1 | 3 | 2 intact, some fused regions | NW_023276806.1, |
| | | | NW_023276807.1 |
| 2 | 3 | 1 intact, 1 rearranged | NC_048595.1 |
| 3 | 0 | 1 mostly intact, some fused segments | NC_048596.1 |
| 4 | 2 | 1 intact, some fused segments | NC_048597.1 |
| 5 | 3 | 1 intact, 1 truncated | NC_048598.1 |
| 6 | 0 | Fragmented/fused | NC_048599.1 |
| 7 | 0 | Fragmented/fused | NC_048600.1 |
| 8 | 2 | 1 intact, some fused segments | NC_048601.1 |
| 9 | 2 | 1 intact, some fused segments | NC_048602.1 |
| 10 | 2 | 1 mostly intact, some fused segments | NC_048603.1 |
| X | 0 | Fragmented/fused | NC_048604.1 |

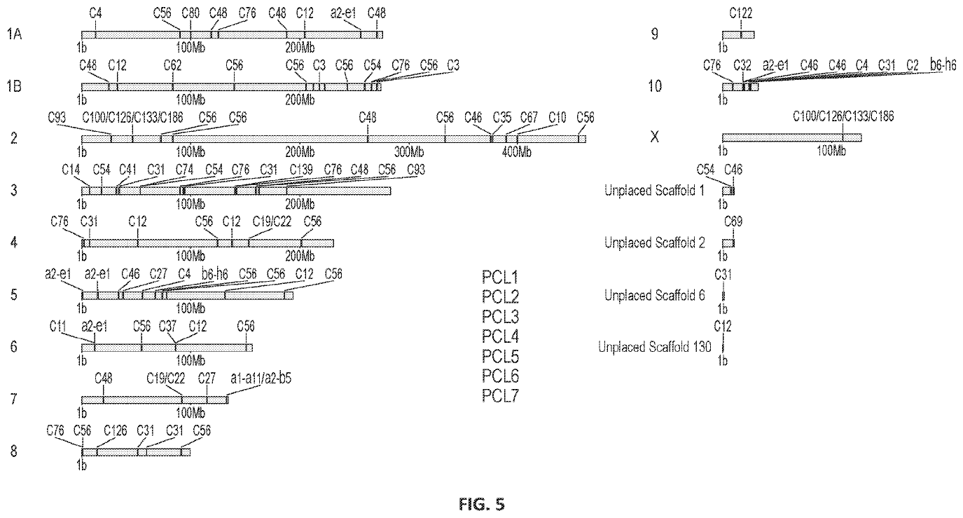

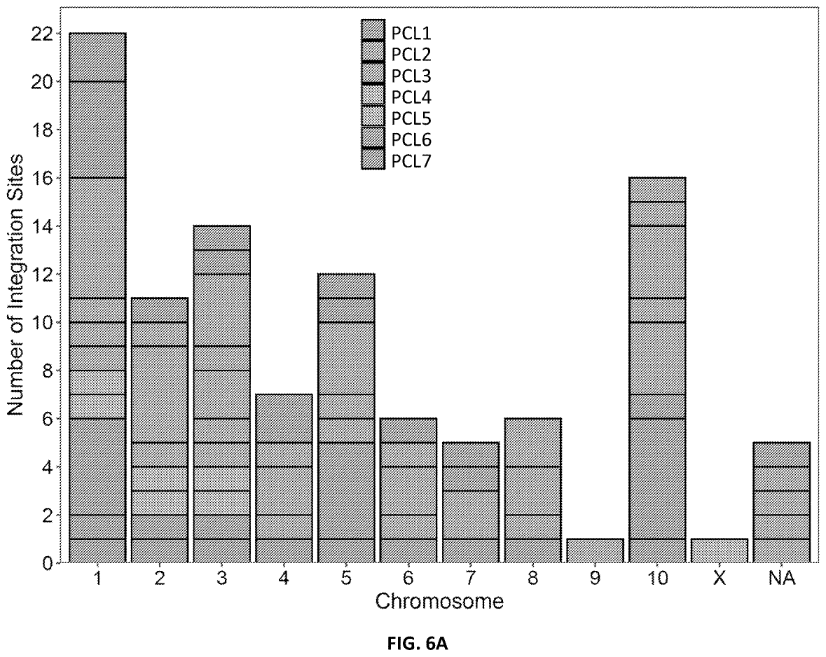

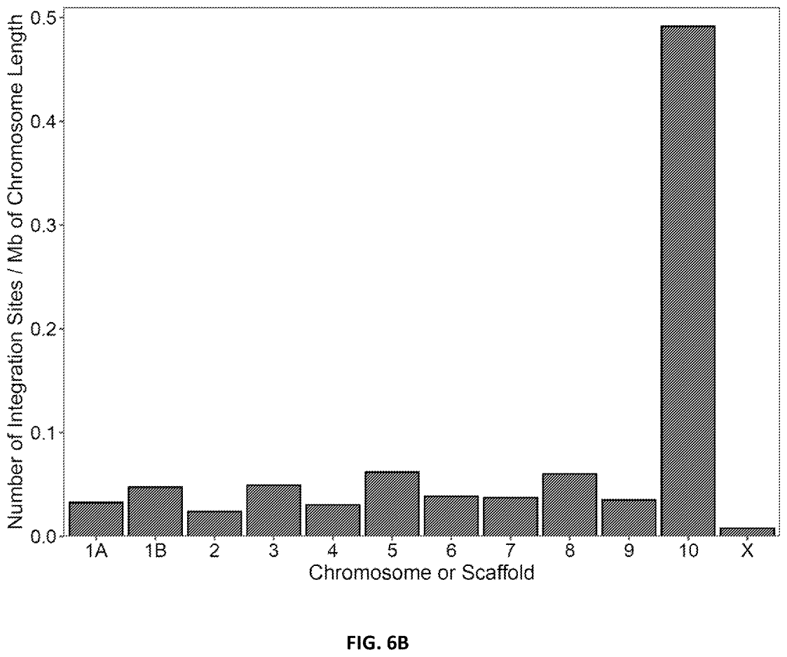

For the seven different PCLs, integration sites were found on most chromosomes, though there was some bias depending on the PCL (FIGS. 5-6A). PCL3 clones had many integration sites on chromosome 10, followed by chromosome 7. PCL6 integration sites were mostly on chromosome 1, with a few on chromosomes 2 and 3 as well. PCL4 clones in general had very few sites, and they were equally distributed across chromosomes 2, 3, and X. PCL2 clones had integration on many different chromosomes, though PCL2-C56 (and its 19 sites) represent most of them. For PCL2, chromosomes 3 and 8 had sites from 3 different clones each. PCL7 clones had integration sites primarily on chromosomes 5 and 10. PCL1 had integration sites on many different chromosomes, including 1, 2, 3, 4, 5, 6, 7, and some on unplaced scaffolds. PCL5 clones only had one integration site each, with two on chromosome 1, and one each on chromosomes 2 and 3.

Chromosome 1 is currently represented as two scaffolds in the most recent assembly of the Chinese Hamster genome (CriGri-PICRH) and is depicted as chromosomes 1A and 1B (FIG. 5). Although most of the CriGri-PICRH genome is assembled into chromosome length scaffolds, there are 635 unplaced scaffolds which are numbered in order of decreasing size. The overall length of all of these unplaced scaffolds is 69.4 Mb, which represents 2.9% of the total length of the genome. 4 unplaced scaffolds had integration sites on them, and so were included in the chromosome map (FIG. 5).

The number of integration sites per chromosome were plotted in FIG. 6A, showing chromosome 1 had the highest number, followed by chromosomes 10, 3 and 5. Chromosomes 9 and X had the fewest integrations (excluding unplaced scaffolds). The number of integration sites per chromosome per unit length was also plotted to see if certain chromosomes were more likely to have integration sites, with correction for overall size (FIG. 6B). Unplaced scaffolds were removed, as their small size in comparison to chromosomes resulted in very high enrichment per unit of size. Chromosome 10 was highly enriched for integration sites based on its size, followed by chromosomes 8, 5, 3, and 1B. Chromosome 10 mainly had integration sites from 2 programs (PCL3 and PCL7) though there was also one clone from PCL6. Myc is located at 26 Mb on chromosome 10 (out of 32.5 Mb total), which is near the large cluster of integration sites at the end of chromosome 10. Myc is amplified and highly expressed in CHO cells, so this may have made this location more accessible for integration.

### Example 3: Analysis of Integration Sites by Genome Features

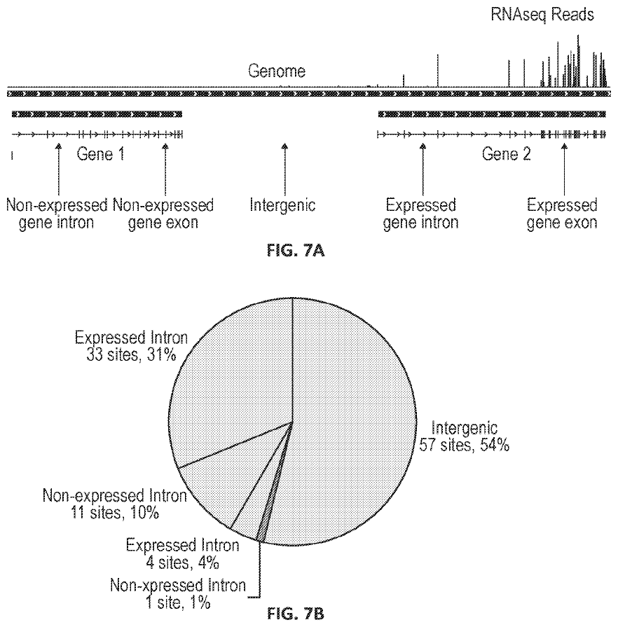

The 106 integration sites identified in Examples 1-2 were characterized based on the genome annotation at each site. Each genome location was categorized based on the diagram of FIG. 7A. The majority of the sites were in intergenic regions, followed by expressed introns, non-expressed introns, expressed exons, and a single site in a non-expressed exon (FIG. 7B). This roughly mirrors the distribution of these features in the genome, as shown in Table 5.

TABLE 5
Percent of integration sites and percent of the entire genome
within introns, exons, and intergenic regions.

| Gene Feature | Percent of integration sites | Percent of genome |
|---|---|---|
| Intergenic | 53.8% | 60.6% |
| Intron | 41.5% | 37.3% |
| Exon | 4.72% | 3% |

Note:
the percent of the genome that is represented by different features add up to slightly more than 100% due to some overlap between introns and exons based on transcript variants.

### Example 4: Analysis of Integration Sites by Expression Levels

For those integration sites within genes (exons or introns), the expression level of that gene was obtained from RNAseq data for the clone itself (if available) and from the average of 3 DG44 RNAseq runs. RNA sequencing data from Stability Evaluation Timepoint 1 (SET1)-aged clones was utilized in the analysis where available. 26 out of 40 clones that were analyzed had corresponding SET1 transcription data, as well as data from DG44 host cells. Specifically, the Transcripts per Million Transcripts (TPM) value for Heavy Chain and Light Chain was utilized, along with a calculated Reads per Kilobase per Million reads (RPKM) value for each identified integration region.

To generate the RNAseq data, cell pellets from either SET1 aged clones or from DG44 host cells were collected during Day 3 or 4 of a passage culture in CD OptiCHO™ media (for clones) or EX-CELL® 325 media (for DG44). RNA was extracted using a Qiagen® RNeasy® Plus Kit, and a directional RNAseq library was prepared using the NEBNext® Ultra™ II Directional RNA Library Prep Kit for Illumina. Prepared libraries were sequenced on Illumina HiSeq 4000, with 16 samples per lane in a 2×150 bp paired end sequencing configuration.

Sequenced reads were trimmed using Trimmomatic v0.39. Trimmed, properly paired reads were mapped to the CriGri-PICRH 1.0 RefSeq version of the Chinese Hamster Genome (Accession Number GCF_003668045.3). For each production cell line included in the data set, a specific genome containing the vector added as an additional scaffold was utilized. Mapping to the genome was performed using STAR v2.7.7a, while mapping to the transcriptome and quantification of gene expression was done using RSEM v1.3.3. Mapped genome reads from STAR were used to calculate RPKMs for each integration region and TPM values from RSEM were used for the IgG Heavy and Light Chain expression analysis.

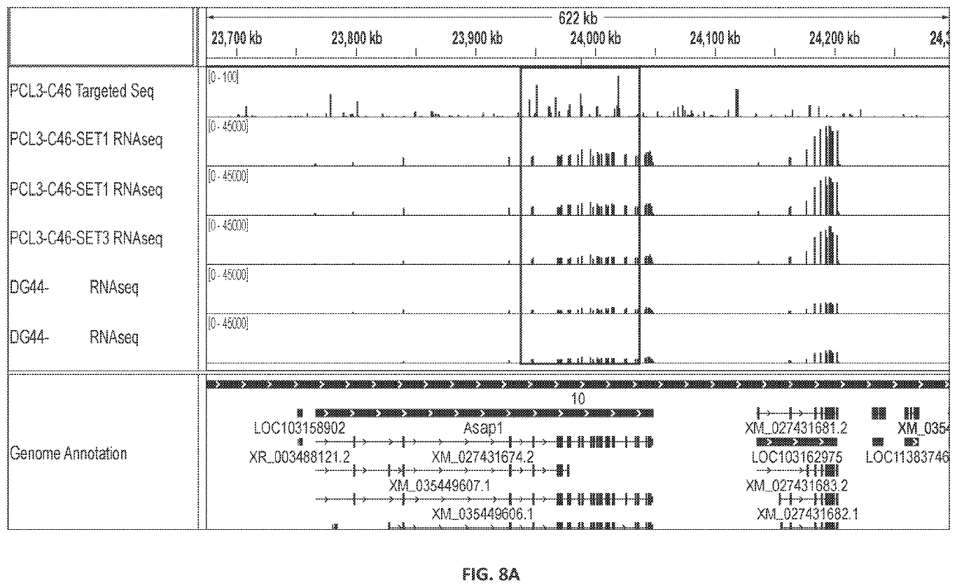

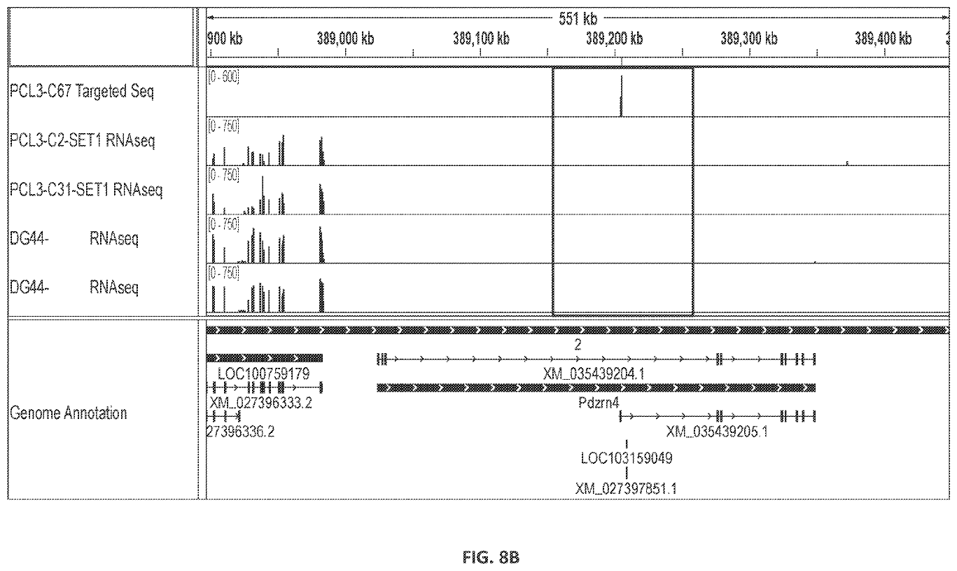

The average expression levels of regions around integration sites were analyzed. The region was defined as the integration site and 50 kb of sequence on either side of the integration site. All reads in this region for each integration site were totaled, divided by the size of the window in kilobases, and divided by the total million reads mapped in the sample, resulting in the reads per kilobase per million reads (RPKM) for each integration site. Some regions were determined to have high expression (FIG. 8A), while many had low or no expression (FIG. 8B).

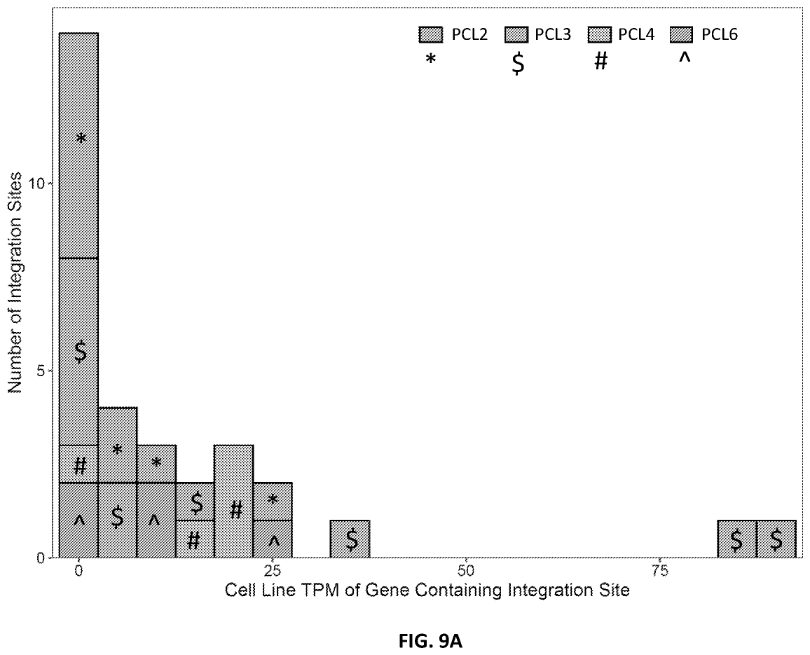

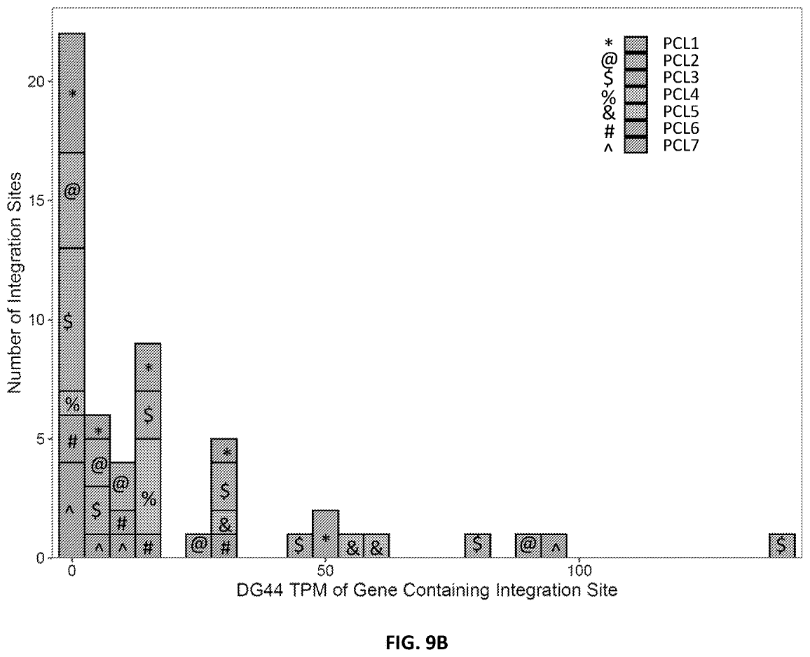

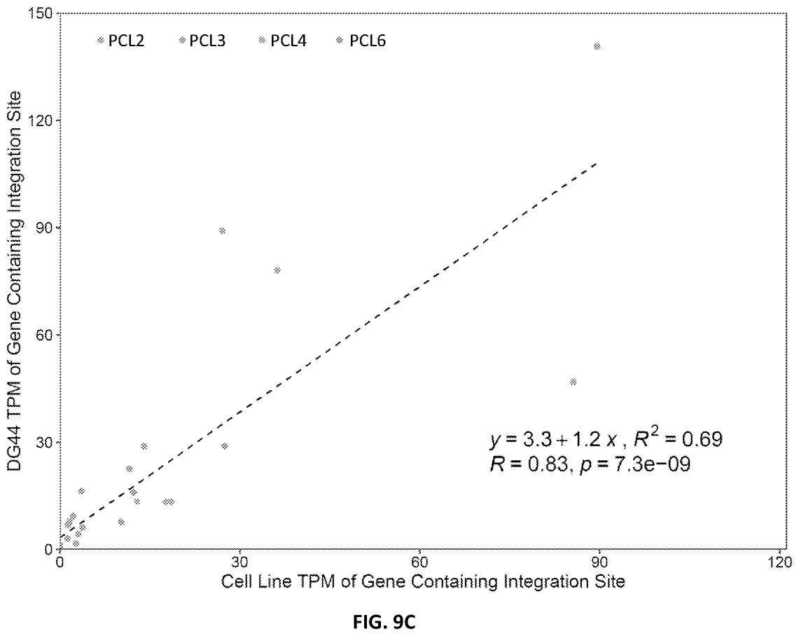

The majority of genes containing integration sites had zero or low expression, though there were many which had reasonably high expression (FIGS. 9A-9B). The average TPM for the SET1 RNAseq samples across all expressed genes (TPM>1) was 86.99, and 1/20 sites within genes which had SET1 data for their cell line had expression of the genes above the average (FIG. 9A). Similarly, for DG44 expression of genes containing identified integration sites, 3/37 sites within introns had DG44 expression above average (FIG. 9B). This only accounted for 5-8% of the integration sites within genes, so although most integration sites within genes were within expressed introns (FIG. 7B), most of those genes were lowly expressed.

The expression of genes containing integration sites in the clones containing the site was plotted against the expression of the same genes in DG44 cells (FIG. 9C). Expression values were significantly correlated, especially at lower expression levels. The slope of the linear regression trendline was 1.2, meaning that the TPM of a gene containing an integration site tended to decrease after integration.

### Example 5: Analysis of Integration Sites by Chromatin Accessibility

Assay for Transposase Accessible Sequencing (ATACseq) data from DG44 host cells was also analyzed. Cryopreserved DG44 cells were processed for nuclei extraction and transposase treatment. The ATACseq library was sequenced on Illumina HiSeq 4000 in a 2×150 bp configuration.

Sequenced reads were trimmed using Trimmomatic v0.39. Trimmed, properly paired reads were mapped to the CriGri-PICRH 1.0 RefSeq version of the Chinese Hamster Genome (Accession Number GCF_003668045.3) using bwa-mem v0.7.17. Mapped reads were filtered using Samtools v1.11 to remove mitochondrial reads, low quality reads (MAPQ <20), and unproperly paired or unmapped reads. Duplicate reads were removed using Picard Tools MarkDuplicates v2.24.2. Read positions were then shifted +4 on the positive strand and −5 on the negative strand to account for the 9 bp duplication introduced during DNA repair of Tn5 nicked DNA using ATACseqQC v1.14.4. BAM files with shifted positions were passed to HMMRATAC v1.2.10 for peak calling and annotation of genome chromatin states.

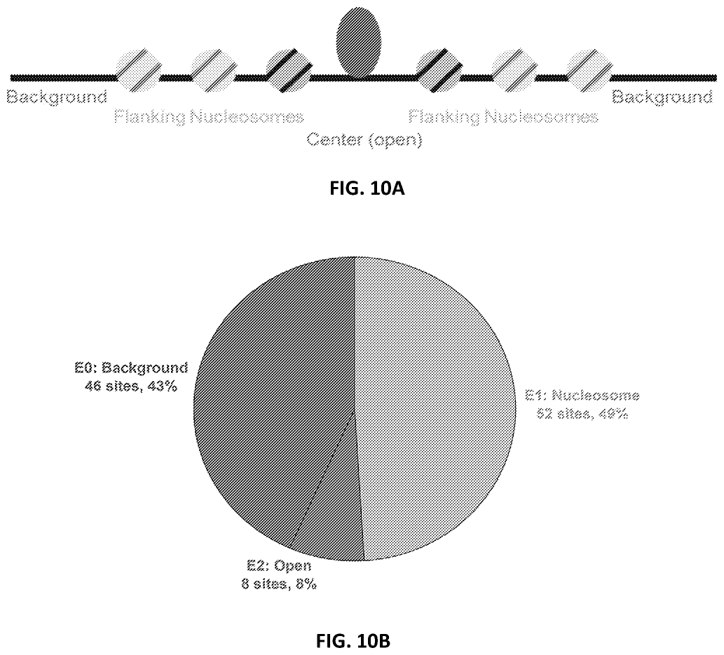

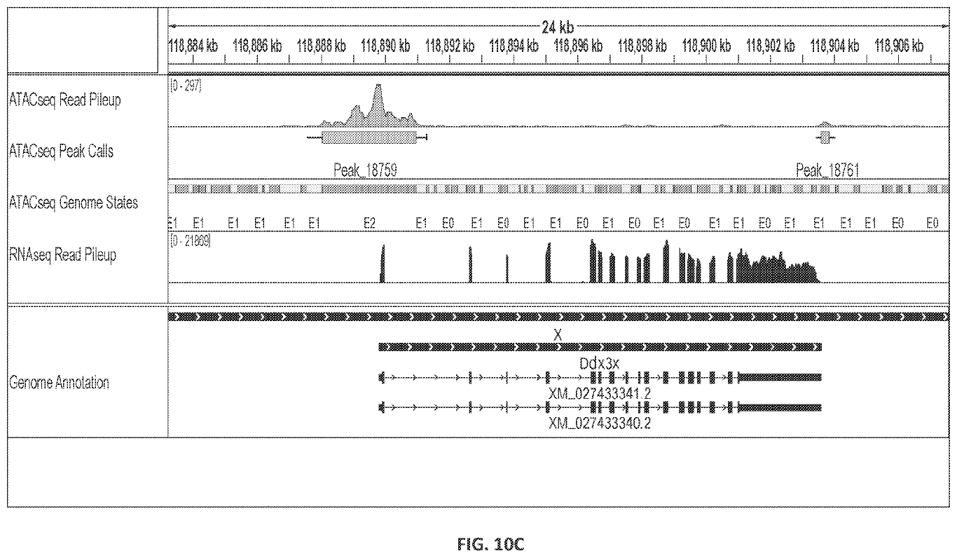

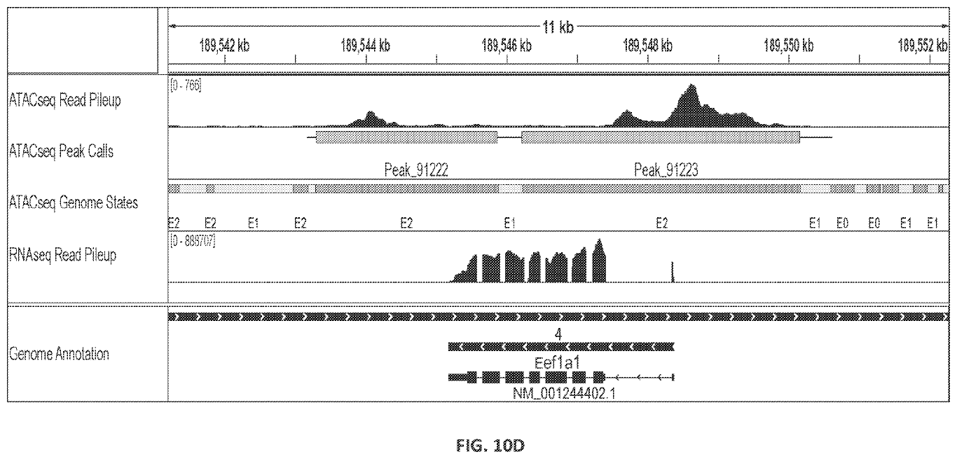

As a part of the ATACseq analysis, the ATACseq model predicted the genomic state at each location in the genome, categorizing it into one of three states: an open, accessible state (E2), a nucleosome containing state (E1), and a background closed state (E0) (FIG. 10A). Peaks were called where there was an open region flanked by nucleosome regions, commonly in promoter regions (FIGS. 10C-10D).

Most of the identified integration sites were in nucleosome regions, followed by background regions, with the smallest number of integration sites being located in open regions (FIG. 10B). This distribution was slightly different from the distribution of these states throughout the entire genome, as shown in Table 6.

TABLE 6
Percent of integration sites and percent of the entire genome
in the different chromatin accessibility states.

| State | Percent of integration sites | Percent of genome |
|---|---|---|
| E0: Background | 43.4% | 57.1% |
| E1: Nucleosome | 49.1% | 39.3% |
| E2: Open | 7.55% | 3.6% |

A Chi-squared test was performed to test for differences in the distribution of these chromatin accessibility states between the integration sites and the genome, resulting in a p-value of p=0.044. This test showed that integration sites were significantly different from the genome as a whole in their distribution among the different chromatin accessibility states. Integration sites were enriched in nucleosome and open regions compared to the genome overall.

### Example 6: Analysis of Integration Sites by Estimated Genomic Copy Number

For each integration site, the estimated copy number of the genomic region was identified based on WGS data of DG44. WGS data from DG44 host cells was used to estimate the genome copy number at each integration site. Genomic DNA was extracted from a DG44 cell pellet from cells in passage using a Qiagen® DNeasy® Blood and Tissue Kit, and libraries were prepared using the NEBNext® Ultra™ II DNA Library Prep Kit. The prepared library was sequenced on 2 lanes of Illumina HiSeq 4000 in a 2×150 bp paired-end sequencing configuration.

Sequenced reads were trimmed using Trimmomatic v0.39. Trimmed, properly paired reads were mapped to the CriGri-PICRH 1.0 RefSeq version of the Chinese Hamster Genome (Accession Number GCF_003668045.3) using bwa-mem v0.7.17. Mapped reads were pre-processed using the GATK v4 pipeline. Briefly, MarkDuplicates was used to mark duplicate reads before running several cycles of mutation detection and base quality recalibration due to the absence of a list of known mutations in the CHO cells used. Mutect2 was used for SNP detection. Copy number estimation was performed using Control-FREEC v11.6.

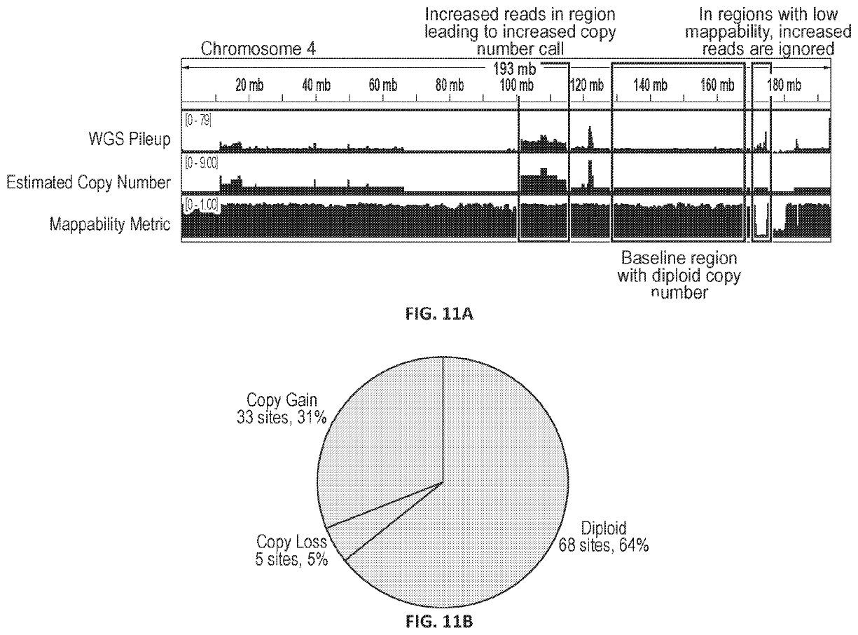

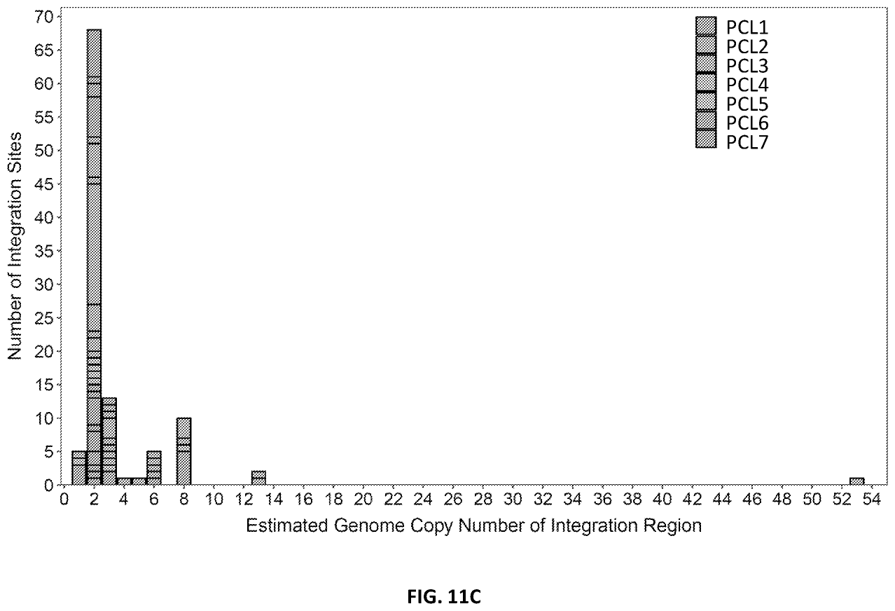

Regions with baseline levels of whole genome sequencing reads were determined to have a diploid copy number. Regions with increased reads and high mappability scores were determined to have increased copy number. Regions that had low mappability scores were ignored, even if they had increased read counts (FIG. 11A). Most of the integration sites were in regions that were called as diploid, around a third of the integration sites were in genomic regions with copy gain, and 11% were in genomic regions with copy loss (FIG. 11B). Most integration sites in regions of copy gain gained 1-5 copies, though there were a few integration sites in regions with 13 or 53 copies (FIG. 11C).

### Example 7: Analysis of Integration Site Regions

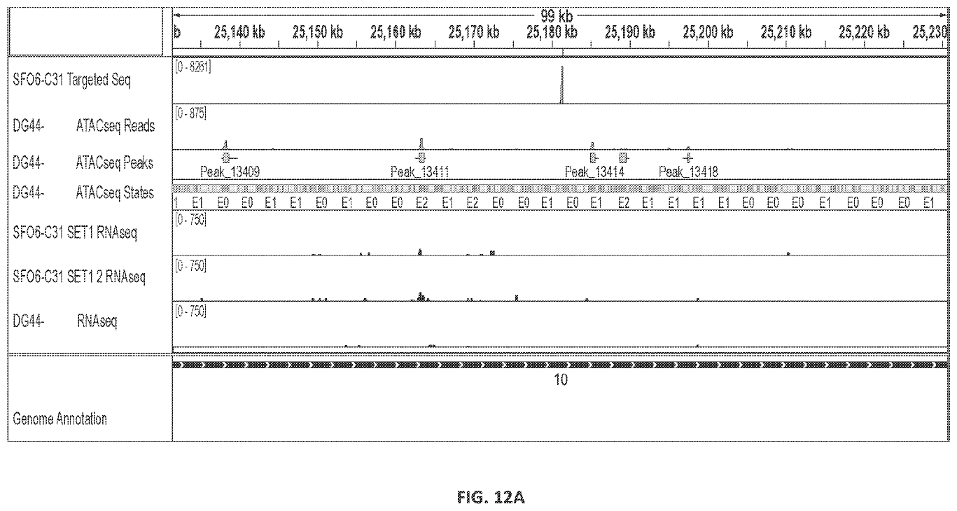

For each integration site, an integration region was defined as the region −50 kb from the left side of the integration site to +50 kb from the right side of the integration site (the left side refers to the junction with the smallest genome coordinate, and the right side refers to the largest genome coordinate). For some integration sites with only one end, the size of the integration region was 100 kb (FIG. 12A). For other more complex integration sites with multiple ends and a potentially large number of bases between the ends, the region could be much larger, such as in the example shown in FIG. 12B for the PCL6-C126 site 1 region. For this region, the two ends of the integration site were ~18 kb apart, and so the integration region was 118 kb. To reduce the bias from differences in window size, all metrics calculated based on the integration region are normalized to the size of the integration region.

### RNAseq Reads Per Kilobase Per Million Reads (RPKA)

For each integration region and each RNAseq sample, the total number of RNAseq reads was summed, divided by the size of the integration region in Kb, and divided by the total number of reads in that sample (in millions). This gave an RPKM metric that was normalized to integration region size and sequencing depth for each sample. Based on this data for all RNAseq samples, the following metrics were calculated for each integration region: RPKM of the integration region for the clone containing that integration site (when available), average RPKM of the integration region for all other clones in the same PCL, and average RPKM of the integration region for the three DG44 RNAseq samples with no integration sites.

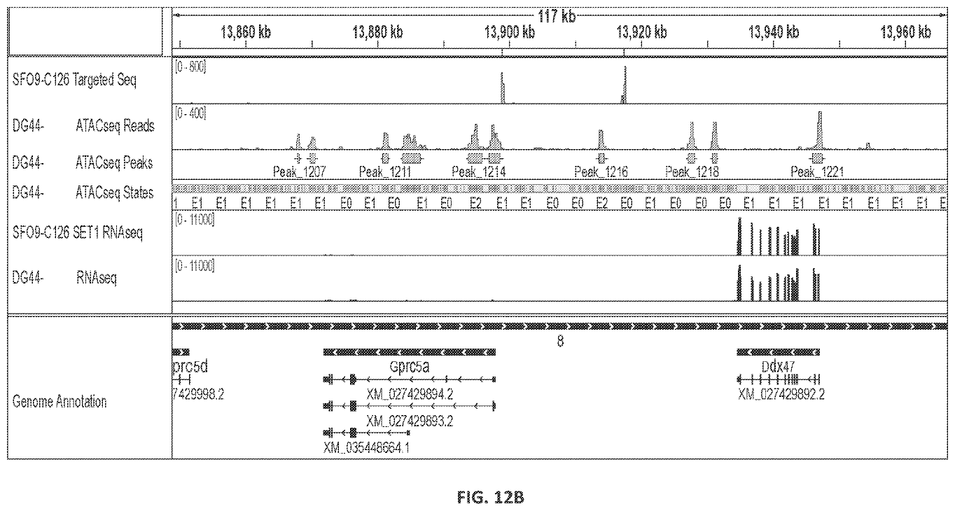

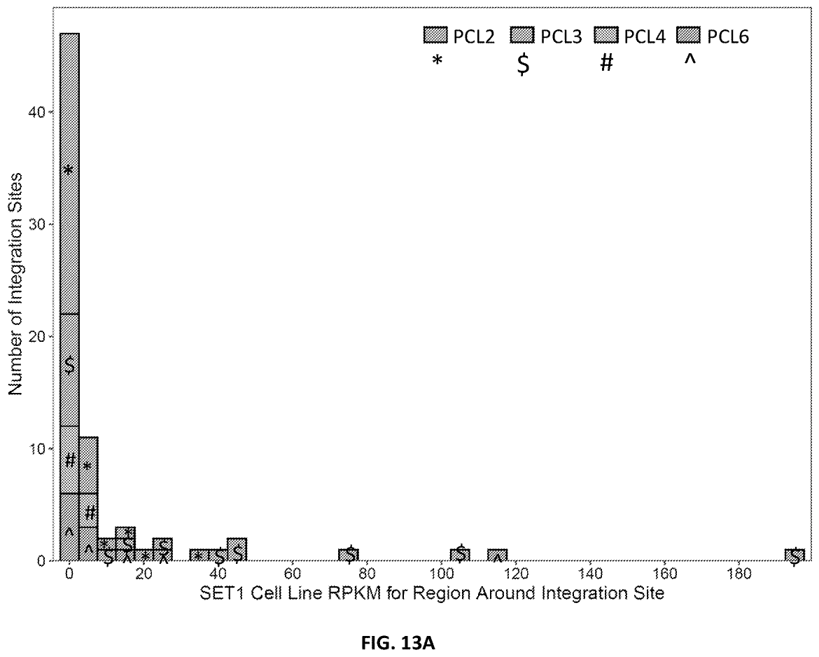

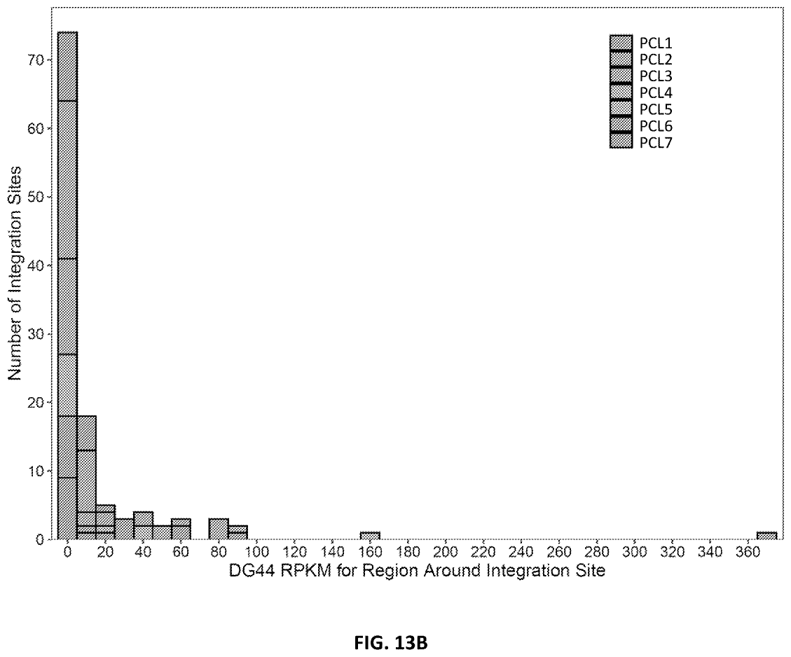

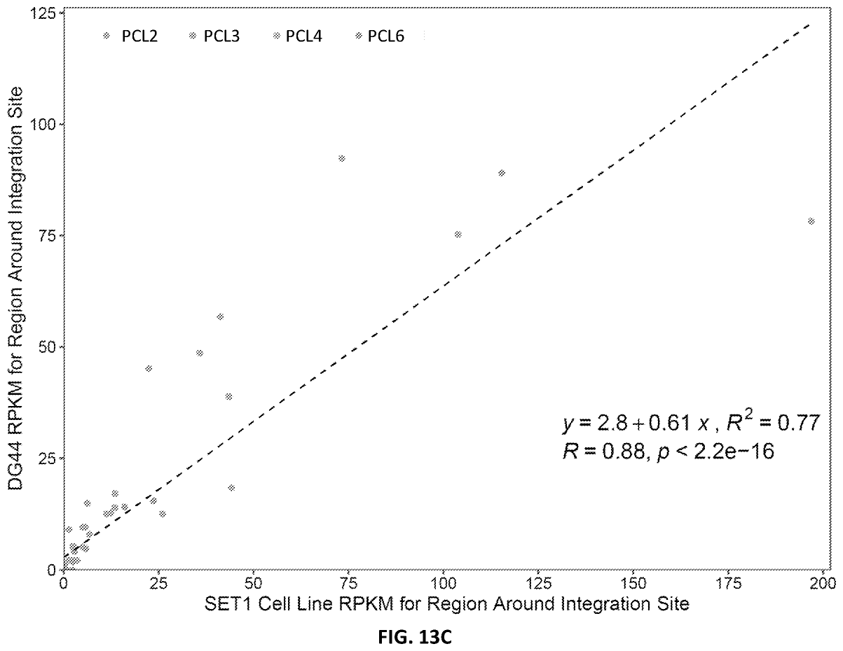

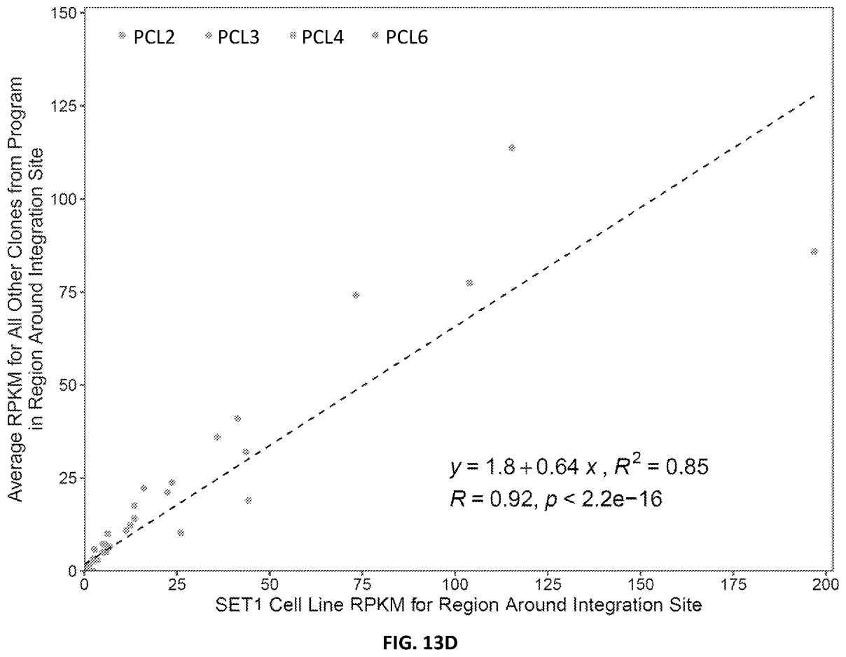

Most integration sites had very low RPKM in the integration region, though outliers exist, primarily in PCL3 clones (FIG. 13A). 70/116 (60.3%) of integration sites had a DG44 region RPKM of less than 2.5 (FIG. 13B). The trends were very similar for the SET1 (FIG. 13A) and DG44 (FIG. 13B) data. RPKM of integration regions in clones containing the sites were significantly correlated with RPKM of the same regions in DG44, especially at lower expression levels (FIG. 13C). The slope of the linear regression trendline was 0.61, meaning that the expression in the region around the integration site tended to increase after vector integration.

To make sure that the data was not biased by differences between the host (DG44) and PCLs in general, the expression in the integration region from the clone with that integration site was also compared to the average expression of all other clones from the same PCL (FIG. 13D). This comparison showed the same trend with a very similar slope, indicating that the RPKM in the region around the integration site increases approximately 1.5-fold after integration, without bias for a particular PCL.

### ATACseq RPKM

Similar calculations to those used for the RNAseq data were performed for the ATACseq data. The total number of ATACseq reads in an integration region were summed, divided by the size of the integration region in Kb, and divided by the total number of reads in that sample (in millions). This gave an RPKM metric that was normalized to integration region size and sequencing depth for each sample.

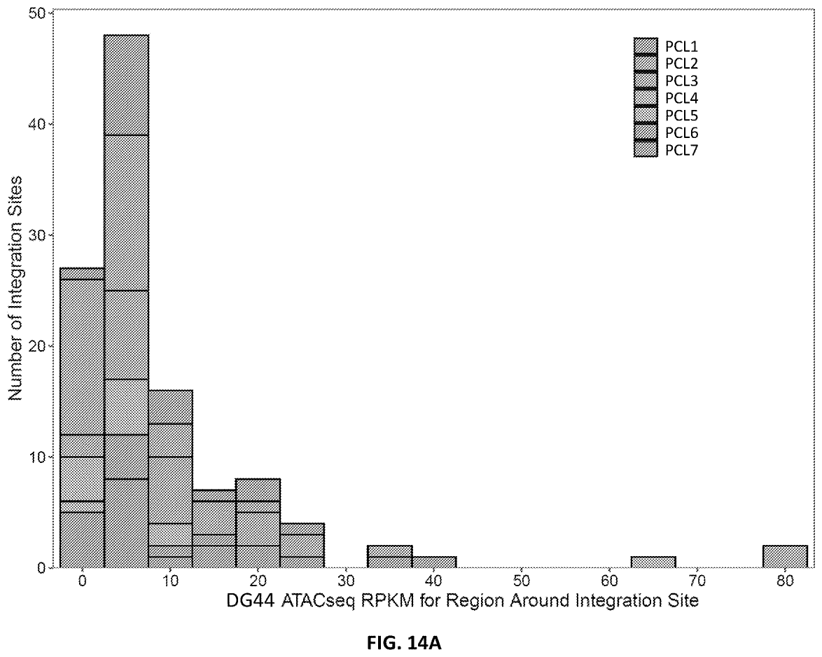

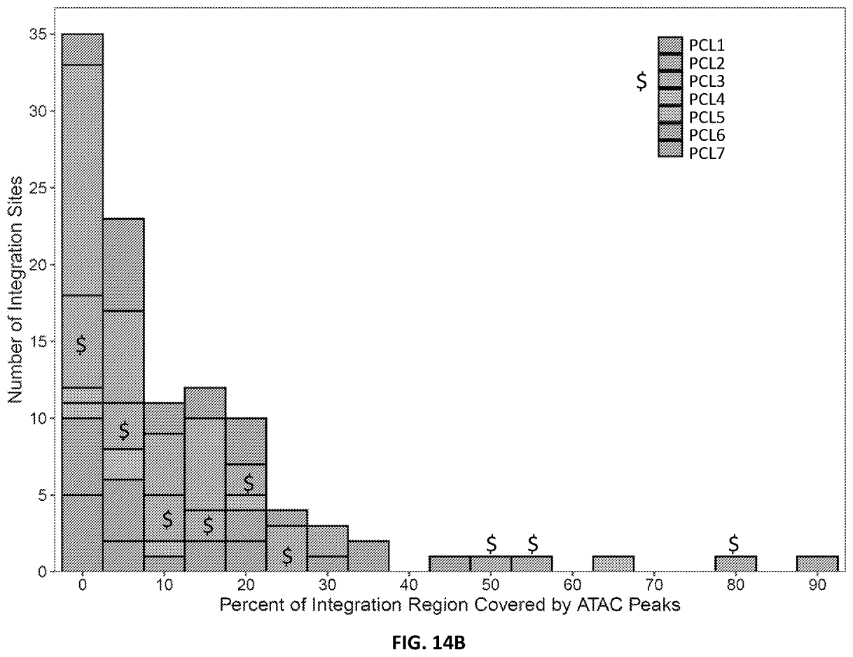

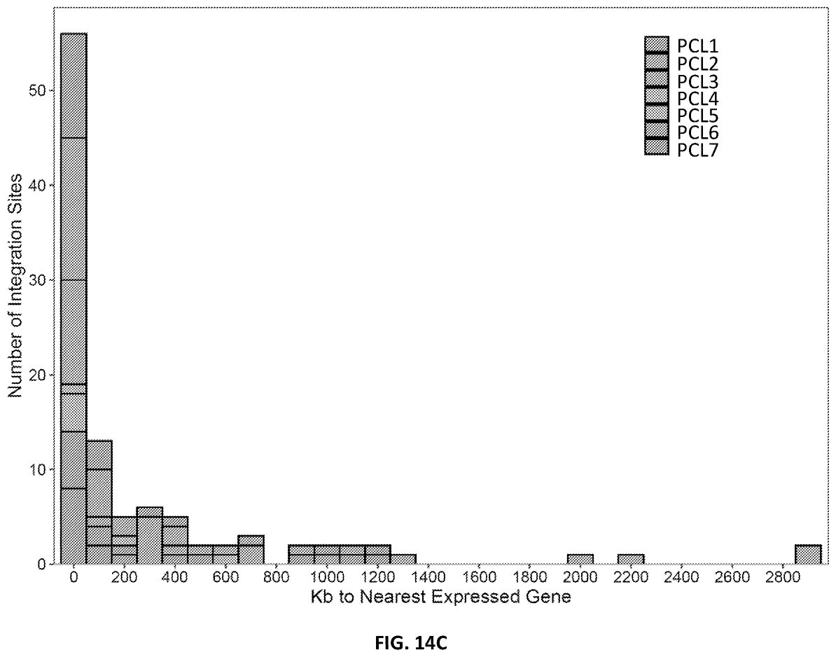

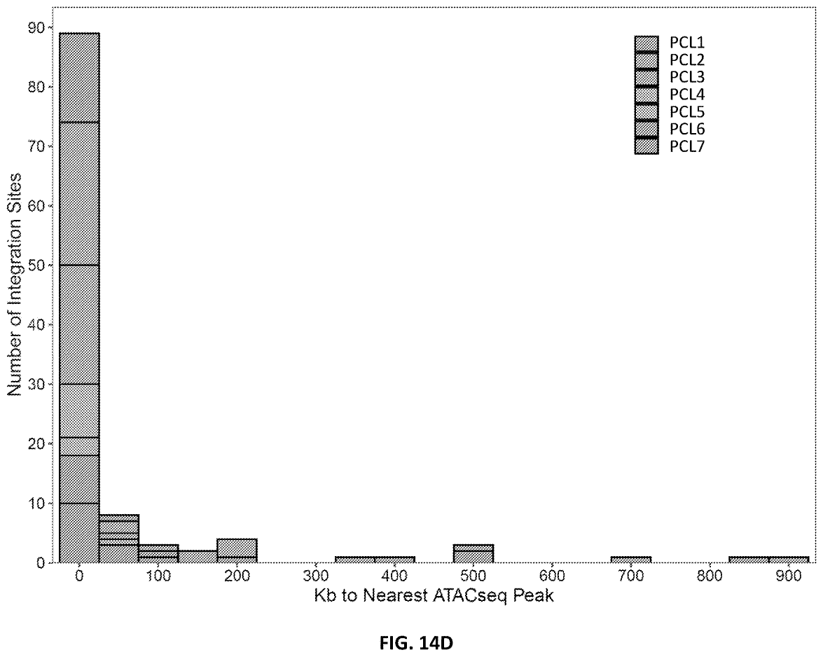

Most integration sites had low levels of ATACseq reads (FIG. 14A), similar to the RNAseq RPKM (FIG. 13A). The percent of the integration region covered by statistically enriched ATACseq peaks was also examined. Most integration regions were in the 0-10% range, but there were a few above 50%, primarily in PCL3 integration sites (FIG. 14B). One important consideration for this data is that each integration site identified was treated independently in this data set—however, within a cell line, not all integration sites were likely to be contributing equally to the expression of the transgene. Some integration sites may not have been contributing any expression at all, while others may have been the primary site in a cell line with many integration sites.

Four of the five integrations sites with the highest percent of the integration region covered by ATACseq peaks had only a single primary integration site (PCL3-C2, PCL3-C11, PCL5-C74, and PCL2-C122 had single sites; PCL3-C46 had multiple). Additionally, PCL3-C11, PCL5-C74, and PCL2-C122 did not have any ambiguous genome integration sites, potentially indicating that these sites could be helpful for supporting high transgene expression from a single site.

To determine the distance from the integration site to the nearest expressed gene, the closest genes to each integration site were identified and their expression level determined from the DG44 RNAseq data. If the TPM was less than 1 for the gene, the next closest gene was examined, until a gene with a TPM greater than 1 was found. For each identified gene, the distance to the integration site was calculated. Integration sites within genes had a distance of 0 kb. 53% of the identified sites were within 50 kb of an expressed gene (FIG. 14C).

The distance of integration sites to the nearest ATACseq peak was also plotted. Integration sites within peaks had a distance of 0 kb. 18% of identified sites were within an ATACseq peak (19 sites), and 83% of identified sites were within 50 kb of an ATACseq peak (FIG. 14D).

### Example 8: Analysis of Single Integration Sites

Since an individual clone could have had multiple integration sites that together generated the resulting phenotype for the clone (for example, IgG expression), one way to avoid confounding the data was to focus only on clones with a single integration site. This included integration sites in genome regions with poor mapping quality, since that indicated an additional integration site even if it was not well defined. The following 14 clones meet the criteria of having a single integration site: PCL2-C10, PCL2-C122, PCL3-C11, PCL3-C31, PCL4-C54, PCL5-C35, PCL5-C62, PCL5-C74, PCL5-C80, PCL6-C67, PCL6-C126, PCL6-C139, PCL7-a1-a11, and PCL7-a2-b5. Six out of the seven PCLs were represented in this dataset, although there was only one PCL4 clone and the two PCL7 clones were sister clones with the same integration site. None of the PCL1 top clones had single integration sites. Out of these 14 clones, three had been designated top clones that were moved forward for the given PCL program (PCL2-C122, PCL5-C35, and PCL7-a2-b5), and three were designated backup clones for their program (PCL5-C74, PCL6-67, and PCL7-a1-a11). For further analyses examining features around integration sites, the PCL7 clones were only counted once as they shared the same site.

### Integration Sites by Chromosome

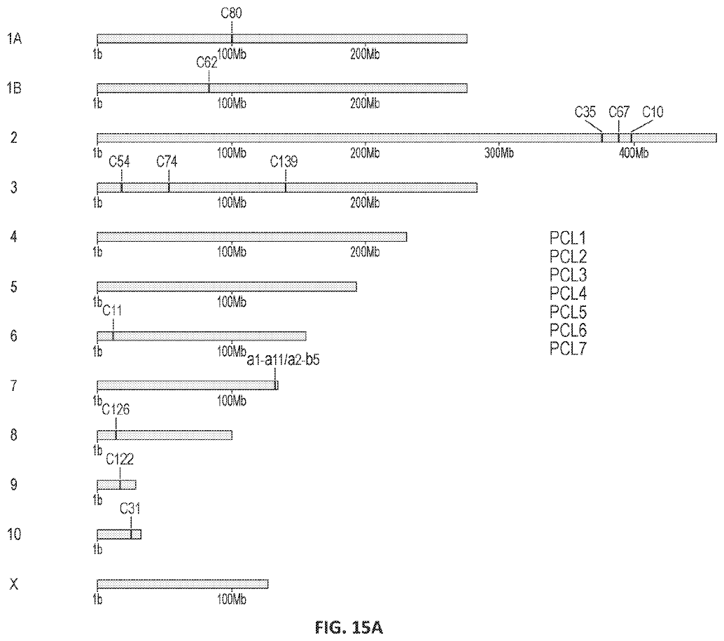

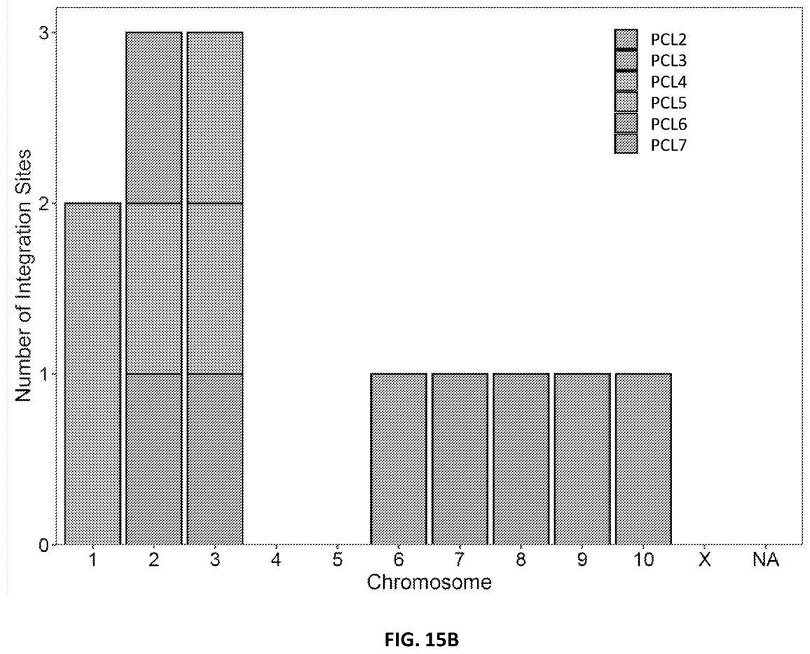

There was no clear trend for a preferential chromosome for the thirteen single integration site clones, as many chromosomes had 1 or 2 sites on them (FIGS. 15A-15B). Chromosomes 2 and 3 had three sites each, chromosome 1 had two sites, and chromosomes 6, 7, 8, 9, and 10 had one site each. Based on comparison of this list of chromosomes to Table 4, chromosomes 1, 2, 8, 9, and 10 would be the best choices based on chromosome integrity in DG44.

### Genomic Features

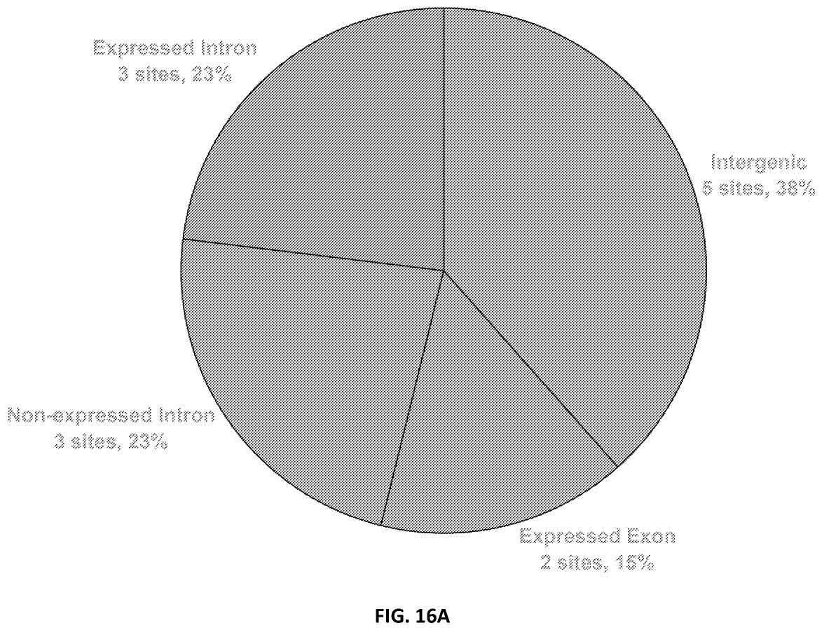

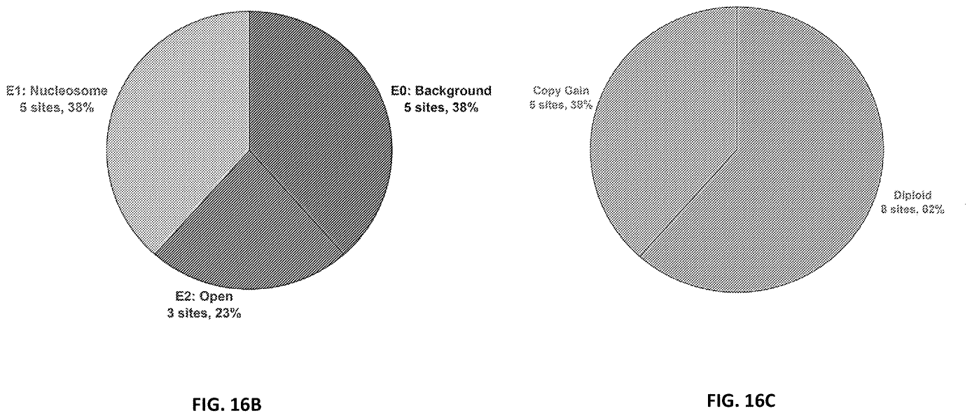

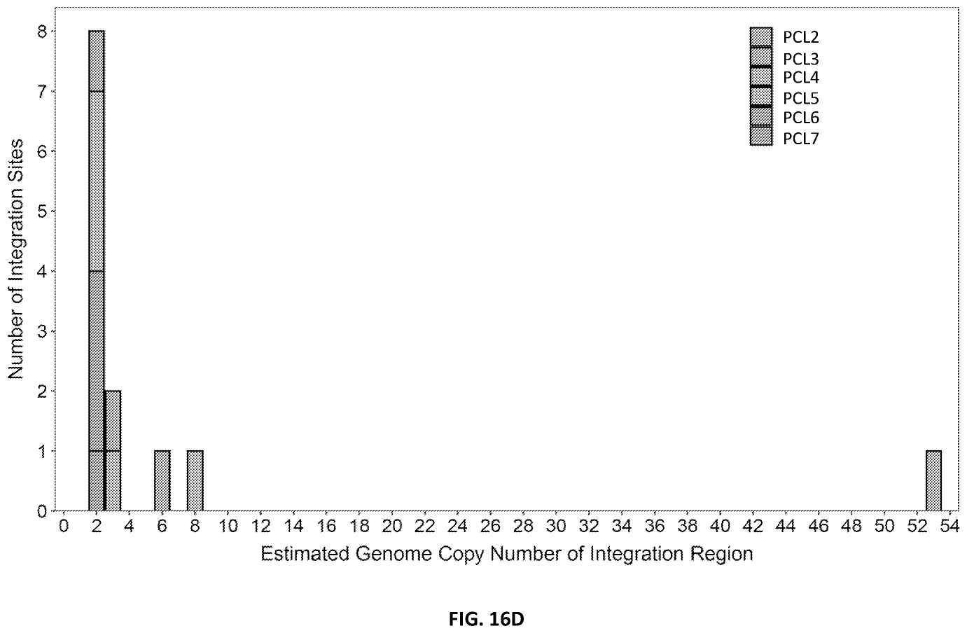

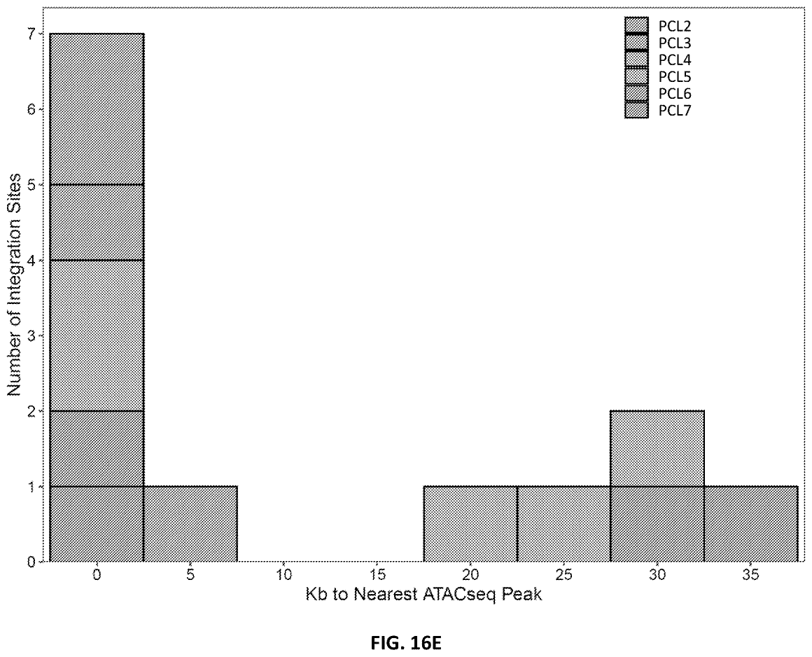

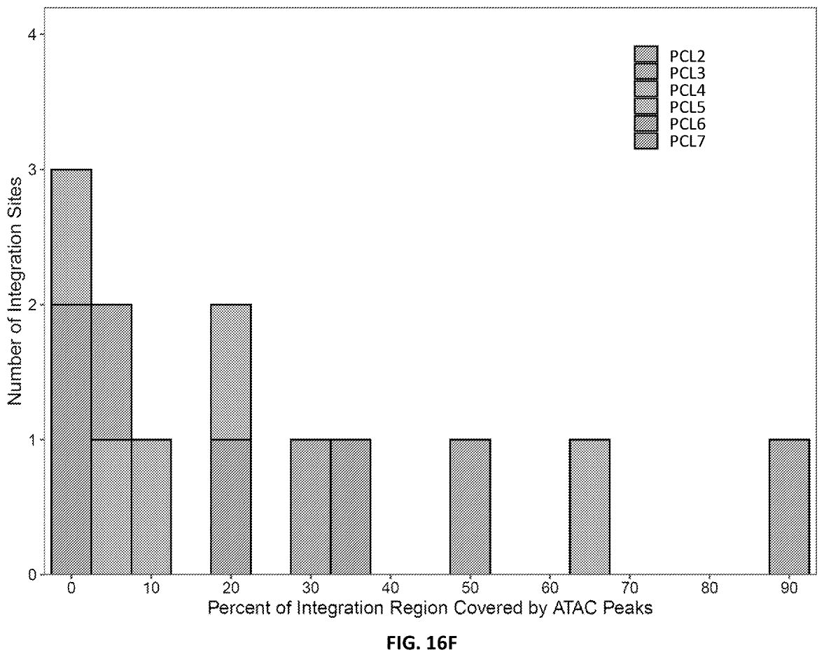

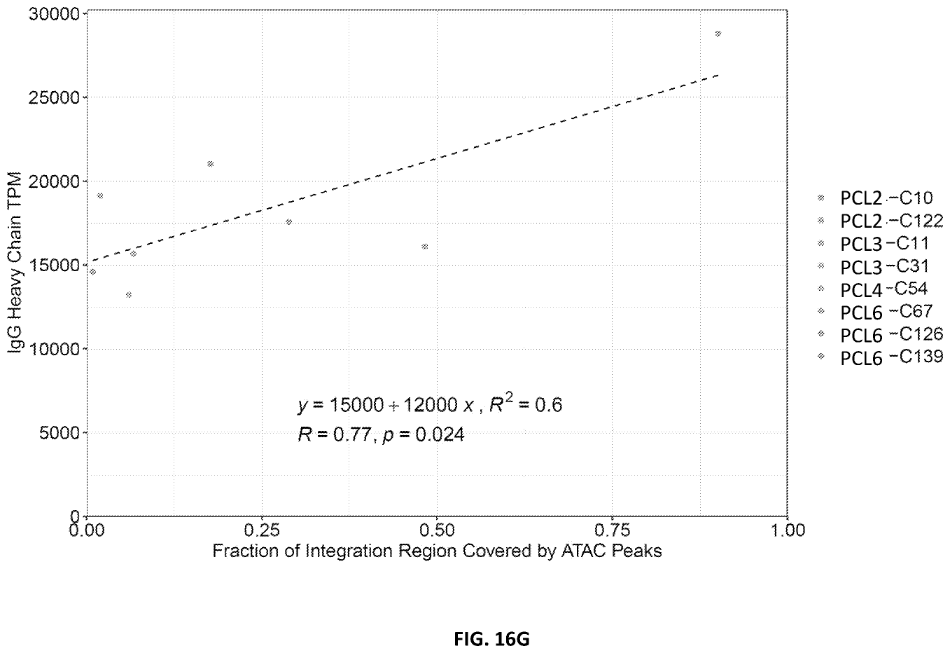

The thirteen integration sites identified for the fourteen single integration site clones were characterized based on the genome annotation at each site, as in Example 3. The thirteen sites were somewhat evenly distributed among the different features (FIG. 16A). This differed slightly from the distribution of these features in the full dataset and in the genome, which were more biased towards intergenic regions, as shown in Table 7.

TABLE 7
Percent of single integration sites, percent of all integration sites,
and percent of the entire genome contained within introns,
exons, and intergenic regions. Note: the percent of the genome
that is represented by different features adds up to slightly more
than 100% due to some overlap between introns and
exons based on transcript variants.

| | Integration sites | | |
|---|---|---|---|
| Gene | (single integration | | |
| Feature | clones) | Integration sites (all) | Genome |
| Intergenic | 38.5% | 53.8% | 60.6% |
| Intron | 46.2% | 41.5% | 37.3% |
| Exon | 15.4% | 4.72% | 3% |

### Chromatin Accessibility

The single integration sites were categorized based on the genomic state predicted from ATACseq data, as in Example 5. The distribution of sites was roughly similar between the different chromatin accessibility states (FIG. 16B). The percent of sites in E2 was much higher than when looking at all integration sites (FIG. 10B). This distribution varied from the distribution of the chromatin states in the full dataset and in the entire genome, as shown in Table 8.

TABLE 8
Percent of single integration sites, percent of all integration
sites, and percent of the entire genome contained
within different chromatin accessibility states.

| | Integration sites (single | Integration | |
|---|---|---|---|
| State | integration clones) | sites (all) | Genome |
| E0: Background | 38.5% | 43.4% | 57.1% |
| E1: Nucleosome | 38.5% | 49.1% | 39.3% |
| E2: Open | 23.1% | 7.55% | 3.6% |

To test for differences in the distribution of these genome states between the integration sites and the genome, a Chi-squared test was performed, resulting in a p-value of p=0.016. This test showed that the single integration sites have a significant difference in their distribution among the different chromatin accessibility states compared to the genome as a whole. Single integration sites were enriched in nucleosome and open regions, and depleted for background sites compared to the genome overall. While this difference could have been due to small sample size, E2 regions may be preferred to support high productivity for a single insertion site.

### Estimated Genomic Copy Number

Analysis of the genomic copy number for just the single integration site clones was performed as in Example 6 (FIG. 16C). A similar percentage of single integration sites were in diploid regions compared to the entire set of integration sites. The remainder of the integration sites were all in copy gain regions. One integration site was found in a region with a very high estimated copy number (53 copies), despite being a single integration site clone (FIG. 16D). Aside from this clone, the distribution of copy numbers, as shown in Table 9, was similar to the distribution for the entire set of integration sites.

TABLE 9
Estimated genomic copy number at each integration
site for the single integration clones.

| | Genome Copy Number at |
|---|---|
| Clone | Integration Site |
| PCL2-C10 | 2 |
| PCL2-C122 | 53 |
| PCL3-C11 | 3 |
| PCL3-C31 | 6 |
| PCL4-C54 | 3 |
| PCL5-C35 | 2 |
| PCL5-C62 | 2 |
| PCL5-C74 | 8 |
| PCL5-C80 | 2 |
| PCL6-C67 | 2 |
| PCL6-C126 | 2 |
| PCL6-C139 | 2 |
| PCL7-a1-a11/a2-b5 | 2 |

### Integration Region

The distance of integration sites to the nearest ATACseq peak was plotted for the single integration site clones, as in Example 7. Integration sites within peaks had a distance of 0 kb. In contrast to the full dataset, all of these clones are within 50 kb of an ATACseq peak and 46.2% are within a peak (FIG. 16E, compared to 83% and 18.3% respectively for full data set in FIG. 14D).

The percent of the integration region covered by statistically enriched ATACseq peaks was also examined for the single integration site clones, as in Example 7. Whereas most integration sites in the full data set had very low ATACseq peak coverage in the integration region, the single integration site clones had a higher percentage of the integration region covered by ATACseq peaks (FIG. 16F compared to FIG. 14B, Table 10). Almost half of the integration sites in clones with a single integration site were in regions with at least 30% ATACseq peak coverage, indicating that this may be an important consideration for selecting a good integration site. The percentage of integration region covered by an ATACseq peak was also correlated with IgG heavy chain expression for cell lines within single integration sites (FIG. 16G). A positive correlation was found.

TABLE 10
Percent of integration regions for all integration
sites and single insertion sites with different
ranges of percent ATACseq peak coverage.

| Range of Percent | All integration | Single integration |
|---|---|---|
| ATACseq Peak Coverage | sites | site clones |
| 0-7.5% | 58/106 = 54.7% | 5/13 = 38.5% |
| 0-22.5% | 91/106 = 85.8% | 8/13 = 61.5% |
| 22.5-100% | 15/106 = 14.2% | 5/13 = 38.5% |

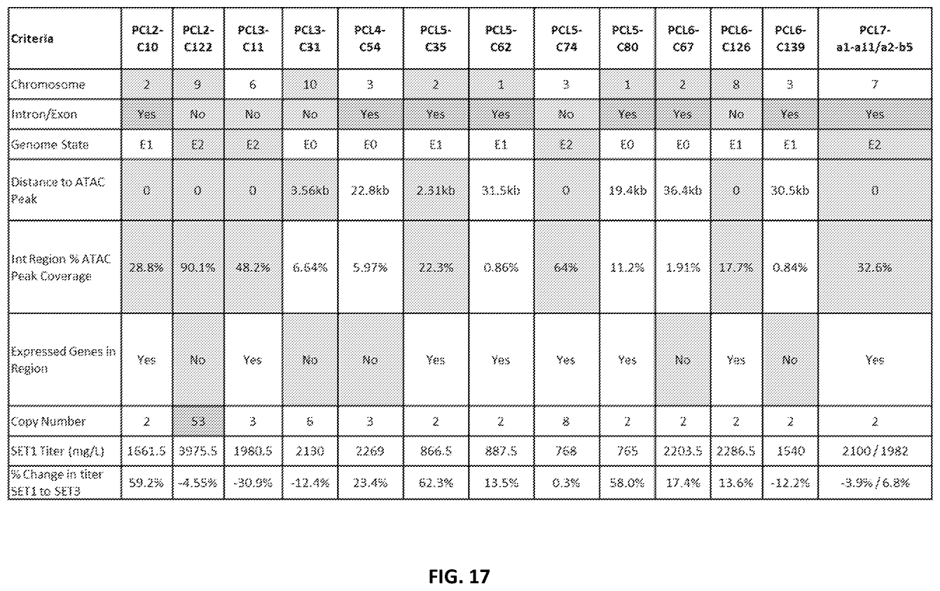

Based on all these parameters, thirteen integration sites from single integration site clones were evaluated (FIG. 17). The thirteen unique integrations sites were selected from a total of forty top-performing production cell lines because they were single site integrations. Characterization of the thirteen sites identified the five best integration sites based on their intergenic localization (Table 11).

PCL2-C122 met all criteria, but had a high copy number at the insertion region. The sequence of the insertion region is provided in SEQ ID NO:1.

PCL3-C31 met most criteria with the exception of having a low percentage of the integration region covered by ATACseq peaks, and an overall chromatin state of E0. Five ATACseq peaks were within the region however, and were located close to the integration site. The sequence of the insertion region is provided in SEQ ID NO:2.

PCL6-C126 met most criteria except genome state and having expressed genes in the integration region. This is likely still an accessible region however, as one end of the integration site is in the nucleosome part of a peak. The expressed genes, however, Gpcr5a and Ddx47, could be investigated further, especially as expression levels were found to decrease after transgene integration. The sequence of the insertion region is provided in SEQ ID NO:3.

Among the additional 10 clones, PCL3-C11 met most criteria except being on chromosome 6 and having expressed genes in the integration region. Chromosome 6 was not intact in any of the DG44 chromosome studies examined in Example 2. This may imply that this chromosome is at increased risk for genomic rearrangements since the DNA exists as a fusion with other chromosomes. This is not ideal for selecting an integration site. The expressed genes, plasmanylethanolamine desaturase (LOC100760484) and Ube2v1, should be investigated further before considering this site, especially as expression levels were found to decrease after transgene integration.

PCL5-C74 met most criteria except being on chromosome 3 and having expressed genes in the integration region. Chromosome 3 was not intact in any of the DG44 chromosome studies examined in Example 2. This may imply that this chromosome is at increased risk for genomic rearrangements since the DNA exists as a fusion with other chromosomes. This is not ideal for selecting an integration site. The expressed genes, Scy11, Ltbp3, and Znrd2 should be investigated further before considering this site.

The top three clones were determined to have the most favorable genotypic and phenotypic characteristics. Genotypically, the three sites are single integrations in a preferred chromosome, localized in an intergenic region with close proximity to an ATAC peak. Phenotypically, the three clones produced higher than average titers with stable production over time. Genotypic information from each clone is shown in Table 11 below. Antibody titer info from each clone is shown in Table 12. These phenotypic and genotypic criteria were used to select these top three genomic integration sites.

TABLE 11
Genotypic information for top 3 clones.

| | PCL2- | PCL3- | PCL6- |
|---|---|---|---|
| Genomic Integration Site | C122 | C31 | C126 |
| Single genomic integration (Chromosome) | Yes (9) | Yes (10) | Yes (8) |
| Intergenic (Not within an Intron/Exon) | Yes | Yes | Yes |
| Distance to ATAC Peak (kb = kilobase) | 0 | 3.56 kb | 0 |

TABLE 12
Phenotypic information for top 3 clones.

| | PCL2- | PCL3- | PCL6- |
|---|---|---|---|
| Cell Line | C122 | C31 | C126 |
| SET1 Antibody Titer (mg/L) | 3975.5 | 2130 | 2286.5 |
| Average Antibody Titers (mg/L; | 1276 | 1794 | 1752 |
| n = number of cell lines) | (n = 40) | (n = 40) | (n = 36) |
| % Change in Titer Over Time (~40 days) | −4.55% | −12.4% | 13.6% |

### Example 9: Vector Design

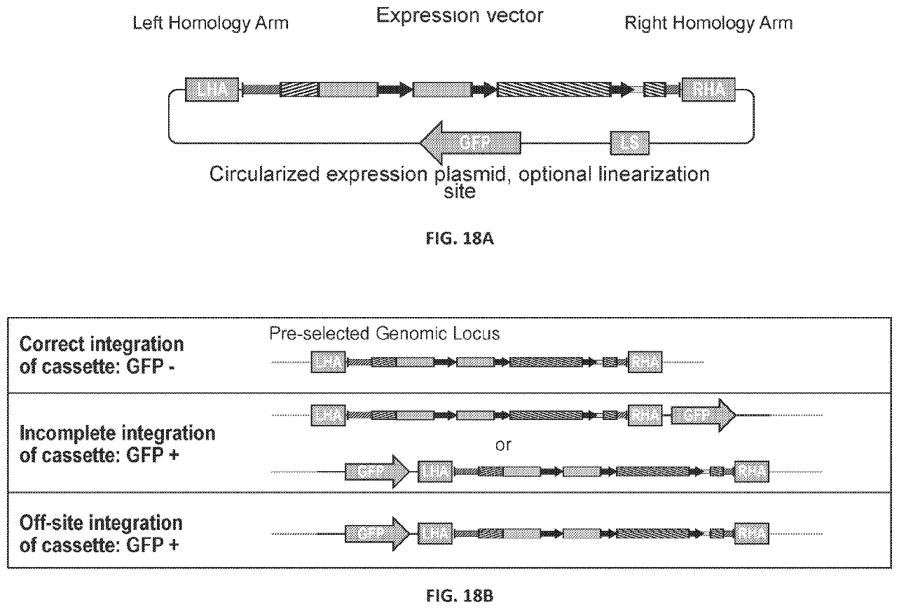

A targeted integration vector was designed to include a standard expression vector with interchangeable homology arms that could be specific to each target location (FIG. 18A). The presence of GFP outside of the homology arms can be used for screening purposes (FIG. 18B). Correct integration of the cassette results in GFP-negative cells. Incomplete integration or off-site integration of the cassette results in GFP-positive cells.

Additional vectors are generated that can serve as landing pads, such that a multitude of target antibodies can be inserted in different cells at the specific insertion site through use of the landing pad.

### Example 10: CHO Cells with Insertions at Selected Integration Sites

CHO cells are generated in which expression cassettes for expressing a transgene of interest are integrated into the selected integration sites. Expression of the inserted transgene is assayed.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

SEQUENCES
All nucleic acid sequences are presented 5′ to 3′ unless otherwise noted.
Targeted integration site 1 (PCL2-C122) genomic sequence
(SEQ ID NO: 1)
TTTTGTTTATTGGGGCAAAACTCTGTATAGTTTTGGAGCCTGATGTGGAAGTTTCTCTGTAGAGCAGGCTAGCCCCAA
ACTATAGAGGTCTTCCTGCCTCTGACTTCTGAGTGCTGGGATTAAAGGTGTGGGCCACTAAAGACTAAAGAAATAGAA
GCAAACCACAACTAAGTTGTGAAGTAGAAGAGTATAATTGCTGATATAAAGGATTTGCCAATTCTTAGGTTTTGTGAG
TCCCCCTAAATAGAATTGAGCTGAGGGAGTACTGCCTATAAAATAACCCAAAGATAAGTAGTATGCTAACTAGGCCTC
TTACAGTTTAGCATGCTAAGAAACACATTTGTTCTCCAGGGTTGTCCACACATTTGGCTCCAAACCATGACAAAGGAG
AAACCCCTGGTTTTAAAACAGCCTCCAGGCCAGAACATGGCTTTTACAAGCTGCTAAGGGAGCATTACTGGATCACAA
TGAGCTTTAGTGCATGGTGTGCTATAAAGGTCCAGTTTCCTTCTAGGGAAAATCATAGTCATGCCAAATGGGCAAGAT
GAGACCAGGTGACTAATATTTGAAAAGGGAAACACTGCTGTTGGACTTAGCAAGCAAAACTTTCTGTTTAGGTCCTCA
ATCATGGAGAAGTAACATAGCTCCTTCCCAAATAAAGGAACATGCTTGGAAGAGCCAGTAACAGCCTGGGGCCTAGCC
TTGGGAGAATCCCATCAGTCCCTGGGGTATGGAGAATCAAGATGTTTTGGAGAGAATGTTTGTTCTGGAGCCAGGGCC
TTTGTGGAACAGTTGCTTCAGATCCTGAGAACACAACTCTTCCCACCACATGGAGCTGTGATTATTCTGTCCCAGGGC
CACTATGGGATTAGACAGTAATGACAGTAATGTTGTTGGGATACCCTGTGTTCCCCTTGTGTTGCATTGTTGGAGTAA
TCTTTTCCTGAGTTTGTCCCATTGTATGATAATTTCTGCTGACTTCCTAGAAGTCTCCTATTTCCCTCCATTTCCCCA
TGAAATTGGTATAACAGAAGCTATTCCTCTTAAGAAGCTTAGTTAGAACAAGACATAGCTCAAGCAAGACCTCCCTAC
CCCAGTTCTATCTCTTTTTAAAATGCCTTACCTGTATTTCTTATCTTCTGGCAACTCCGCCTCTGGACCTGTCAGTAA
ATTATGGTGTGCTGTTTCGTGCTACTGTGTACTATGAAATACTTCTGGTAATATCTTTTCAGTCGAAGTCTTTCATTT
GTCTACCTAGCCTAACTTGCATTTTCCTGTCACTATTTAAATTCTTTAAAGTGTAAGCTGGAGTCGGACCCTAAAGGA
ACTGAAATATGATAACATATGTGGAAAGCAAAATCTGTTTTAGGAGCCAGGTGGTGGTGGTGCATAACTTTAATCCCA
GCAATGCAGAGTCAAAGGTAGGGTGATCTATGTGATTTTGAGGCCAGCGTGATCTACACTGCTAGTTCCAGGACAAGT
AGTGCTCATACACAGAGAAACCAATATCTAAGAAAATAAACAAAAAATCTGTTTTAGCCTCTCTCCTCTTTGAGAATT
AGCCAGAGATGGATGCATGTTATCATTCAAGAGATGTTTCTTTATTAGTCTCTTAAGTCAGTGGAATTATTTTTCACT
GGAAAGGCATATTCTAGAACAGCTGCAATTCCAGCCTTTTAGAACAAGGTTTCAGAGACAAGCTATCTCATGTTCTCC
AGGGCAAAGACCTTGCTGTCTTCTGTACAAAACCTTCAAAAATCTTAAATGAATGTACAACCTCAGAGATGTAAATTT
GTCCATGAGAGTTACATAGCCAACTATCCAATGAATTCACAGAAAATTATTCCAATGGAGGAGCATGGCTTCTACCTT
CAGGCCAAAATAGGAGGAATAAATTAAAATGCAGGAGGATTCAGGGAAGGAACCAGGTGCTGGAGTCATCTTTATGAA
AAGGGACCTGATTGTGGTAGTGGGAATTATTTTGTAATTCCCCAACATGTTTTAATTCATAACATGTAAATAAACACC
GACCAGATTGTGTGGGTGTACAGTACGGAAGGTTATCTTGAACCAATGAATATTGAATTTTCAATTAGTCAACTATCC
TGTGCAGATATGTTACGTCAGCTCAGAGGTTTGTGAATCAGGCCATTGACTGTAACTTTGTGTCAGATTACAAATCTC
AGTAGCTAACATATGCCTATGGATGACATCAATCTATCTCTTGTTAATGACTATAGTAAAATTTTGTTTGTAATCTAA
CAAATAAGGCTTCCCTGAATATCAGTGTGCAGAGCTAAGTCACTAGTTAGCTACACATCTGGTTAAAACACACTAAGT
CACTAGTTAAGCACAAACCTAATCTACTAGTTAGCCATAGAGGCAGAGAGGTCCTGCTTAGGATATGTCTTATTATAA
GTAAGCATCTTAAGAGGAATAGCTTCTTTTTTACTATTTCCCGGGGGAAATGGAGGGAAACCAGAGACTTCTAGGAAG
TAAGCAGAAATTGTCATAAAATGGAACAAACTGAGGAGAAGGTTACTCAAACAATGCAGCACACAGGGAATACAGGGT
ATCCCAAGAACATTACTGATGAAGCTCACAGAGGCCTTCGGTCCAATAACCACAGCTCCATGTGGTGAGGAGAGTTGT
GTTTTAAGAATGTGAACCAATGACCCCACAAAGGTGCTGGATCCATACAATTATATTCTCTCTAAAACATCTGCCTAG
TTTTAGTGGATGAGCTCTATTTTATTCTCCATACCTCAGGGCCTAATGTAAAATTCCTAGGGCTAGGCACCACTGGCT
CTGCCAAGCATGTTCCTGGGTTTGAGAAGGAGCTATGTTCCTTCTCCATGAGAGAGGACGCAAACAGAAAGTCTTCCT
TTCTCAGACCAACCCCAAACACAACAGTGTTTTCCTTTTCAAATACCTGTCACCTGGTTTCATCTTGCCCATCTGGCA
GAACTGTGATTTTCTCTAGAAGGAAACTGCAACTTTATGGCACACCATAACCTAATGATCATTGTAAATATTATTCTT
TAGTAATCTCTTAAGTCTGTGGAGATATTTCTCACTGGAAAGGAATATTCTAGAACAGCTGCACTTCCATCCATTGAG
AAAAAGTTATCTGAGACAAACTCTTTCATGTTGACACAATTGTCTTGATCCCAGTCTGTTGCTTTCCAGGGAAATGAC
CTCACTGTCCTCATGATTACACCTCCTTCAATTAAGTTACTATGTGACCACAGAGTTTTGAAATCATGCATTCAAGTC
ATGCAGATGACTGTCAGTTGCAGTCACAAAAAAAGGATTTGCGTGTGGAGCATGGTTTCAGCCTTCATATCAGAGTAG
AAAGAATAGTTTATTGAGTTCACTGCAAGGGGGAAACAGGGTAGGAAATCAACTACTTAAGTCACCATAGTGAAACAG
TACCTGATTGTGGTAATGTGATAGTTTGAATGTAATTTCATAGGGAGGGGCACTATTAGAAGGTGTGACTTTGTTGCA
GCAAGTTTAGAATTTTTGGAAGAAGTGTGTCATCACTGGTGATAGGCTTTGGGGTTTTCCATCCTTCACATCACACTC
CCCACTATGGTGACAATTGACTGAACCTCTGAATCTGTAAGTCAGCCAGTCCAATTAAGTGTTTTATTTATAAGAGTT
ATGGTAGTCATGAAGGTTATTCATAGCAATAAGAATCCTATATAAGACAGGCAGATTCAGTGATTTTTTTCTTTAAAT
TCTCAGGATTTATTAATTACTGTAATGTGAATAAAGATAGACCTGATTTGTGGGTTTACATTTTGTGAAGGTTATATT
GATCTCAGACCTTTCCACAGGATATGTAGTTGAGAGTTGCTTTGAACAAAGCTAATCCTGAATCTGCTAATCATTACT
GGGAAGACACTGCTGCAATTCTTTCAAGAGTGGGATTTGACTGGTGATTGCGAAGGAAATAGAGACCTTTTAATTAGT
GTTTTCCAATCCCTTTGGCTTGTATCAGGGACTGCAAATAAAAGAGATATATGAAAGAATTGAGTTCATTTACCAATT
TTAAAGCTGGTATAGGCACATTGCAAAAAGGGGTGGGAGAAGGAAGGAACATGGGCAAATACCTGCTGGTAGGCAGTG
GAAATTAGCAGTATGGAGTCTTTTCATCAATTTTAGAAGATCAATAAAATGCCCGAGTGGCCATTTCTTGGGGTCCAC
AGACAAGGGTAATCAATAATCTTGAAAAAAAGGCCTGATGTTATCCTGATACCAAGTTGTTGTAGTTATGCTAGAAAA
TATGTAGATGATATATAGAAAGTCATCATAAATTGCTGTGACCCTGCCTAACGAGCCTGTTATTTTACTGGGTTAAGA
TGTATAGCCTGAGAAAAGATGTGAGTTATACTTGGTGTTCTCTCATCCAGATACCTAGTCTCTGGAGCAGATAGCCAT
AAAGTCTGTTTCAACTTTTCAAAAGAATTGTCAAGATTATAACTGCATCATATCTATAGTATCCATAGTCTCTTTCAT
CCCTCTAATCCATACCATGATGCCTCACTATCAGTTACACTGTAACAATACTGTGTGACGTATGAATGTTTGAATGTG
ACCTGTGTTTGGCTGTGGGACTTCCCAGAATTAATCAAGGCATTGGCTAGCCTGGATAGATACTGAGTGTGTAATAGT
ATGTGCACAAAACTTACCCTGTGTCTTGGAAGAGAACAGGAAGTGCTCTGGATGACTGGGAGAAAGTCTCCTAAAACA
CAAACTTTCCAGACTAGGGCACTGTCTGTGGGGATGGAAGGGCAAAGCTAGGAATGCCTATGCTGTCTGAGTTATTTG
GTCTTTCCATACCGTGGAGGGGAGCTGCTGATGCTCTATGGTCCAACTGTGTGTCCCTAGCTATTTAGGCACTCTGTG
AAGGTTTAGCACTCAGGAGGACCAGTTGAAGGTAGCTAGCATGTTATCTCCAAGACTCTCTGCTGGCTCTCTTGTAGG
AGCTATAACGGAGACGACAGCCACAACATGGTGAGTGTGGAGGCTTCAGAACAGGAACAGCAGGTCTGCCTGGCTGTC
ATTGCTGTTGGAAATGAGAGGGTCTGTGAGCCATGTACTGACCTGGGCAGTGGACCTTATCCTTTGATCCTCCATGAA
TGTAATTTCCCTGGGGAAAGAGAGGGAGGTGTTTTAGCCCTGGCTGTGGGAAGTTGATATGCTTGTAATTTCAGTGCC
CAGTGTGCATCACGGGATTGTTTTGGGTGTGTGCTGGAACATATATTGAAAAACTGAAAATGAGAAAAGATCCCTTTT
TTCTGACTGGCGGGTCTCCTAGAGGTTCTGATTGGCTAGTGAGGCCTGGTGACAGGACCAGGTAAGCAGAAGAGACAT
AGAACAATGGCTTAATCTTGAGGGTGTGGCCCCAGGAGCACAGAAATCCAGAAACCAGGAGGTGAGTCTTAAGGTGGG
GGCTTTTAATGCTTCTGAGGGTGGGCCTGCCAATGTCGGGCTTACTAAGGGCAGAGAGAACTGAGGTGATTTCAGGGT
AGGGCCCACCATGGTACCTTGAGGATGGGGTTTGGAATGGGGCTACCAAGTATTCTAACTACTTTGCATAGATATAGA
AGCCTGTAAGGCTTATACCCAAAAGACCCAGACTCCCTCGGTAGAGGGGTGTCCATTTAAGTCTGGCTTCTTCCTCCC
TTTGACAAGTAACAATGTGGGGAGGGTGTAAGTCAGGACTTGGTGGGTTCATACACAGCAGGAGGTATGTGTGGAGAA
GTATTCCTTTAGCTGCCATTCTGTGTACGTTAGGCAGCTGTTCCTTGAGGAGGTGGAGAAGGCAAGTTGGGTGAAGCT
TCTGCCTTATCAGGGAGTTTCAGGTAGAGAAAGGCTGATGCCAGTCCAGATGTTGATTAGCAGCACCATTCCAAACCC
CACCCTCCAGGTGCCAAGGGCAGGCAAACCCCTGGATTCTTGTCAGTTCACACCCAGCCTTTAAAAAGTCCACCATTT
TCCACCAGAGAAGGAGAAAGTGTCAACATCTCCTCAATAAATTGGGAGAATGGGGAGGGGAGAGTGAGGTAGGAAAAA
GAGAAGAGGAGGAGAGGAGAGGAGAGGAGAGGAGGACATGAGGGAGCAGGAAGATTGAGTTGGGTGAAGAAAAGGGGG
GAGCAAGAAAAGATATACCACAATAGAGGGAGCCATTATAGGTTTATAGAGAAATCTGGCAGTATGGAACTGTCCAGA
TATCCACAAGGATGACCCCAACTAACAATCTAGGCAATAATGGAGAGCCCTTCAAGGCCCTTCCCCTATAATGAGACT
GATGACTACCTTAAATGCCACCCTAGAGCCTTCATCTTGTAGCTCATAGAAGCAGAAGCAGACACCCACAGCTAAGCA
CAGAGCTCTACTCCTGGAATCCAGTTGTAGTGAGGGAAGATTGATGAGCAAAGTGGTCAAGAACAGTCTGGAGAAACC
CACAGATACAGCTGACCTAAACAAGTGAGAGCGAATGGACCTAGGCTGATATCTGGTTAGCCAGCATAGGACTGATCC
AGAACCCCTGAATGTGGGTGAGCATTAGGAGGCCACAGCAATCTATGGGGCCTCTGGTAGTGCAACAATATTTATCCC
TAATATACCAATGGATTTTTTGAAGCCCATTCCCCATATAGGGATACTATCTCAGGCTAGAAGCATGGGGGCAGGCCT
GAGCCCTACCCAAAATAAAATCACAGATTTGGTGATCCCTCTAGGAAGGCCTCACCCTCACTGTGGAGTAGATGAGGG
ATGGTATTTGGGGGGTTTGTGGGGAGCATGGCAGAATGAGAAGATGAGGGAACTGGGATTGATATATAAAATAACAAA
CATTGTTTATAATTTAAATACAATTTATAAAATACAAAAAGAATAGGAAAGACAGGGTGGCAAATACCTTAGCTAACT
ATTGCTGGTGCCATAGCCATGAAATACTGGAGCTGCAGCGAAGTTTATGTACATTACCCAGAATCAGTCACAGTGTCC
CTTTCTTCTGTGGTCCTCCCACCATACACCATTGACCATTTTCATCTTGGATGAATTTGAAAGCCTGAAGTTCCTGTC
TCCACATTCTAAGTACCATGATTACAGGCGTCTGCCAACACGCCTGGGTTAGGTGGTTTCAGCTTCACTTAGTGAGGC
ATTAGAAGGAGTAACATGAGGAGTTTCACTTGACTATATTCAAACTGTGTTTACCTCTGTGAGCCTGGGTTTTAACCA
TCTGCAGAATAAGGCAGAACTCTTCCCACGTCCTTTGATGAGTTATAACAGTCTCAGTGATAAAGAAAAAAAGTATTT
ATAATAATTGGAAGACAGTGGGCTTTGAGTCAGGAAGGCTGCATTTGATGGGTGCCTTCTTCTGTTACTGAAGTTAGG
ATCAGCACCTGTATCCTGGTTTCCCTAACCAGCCAGGCTCAGTCACTCATTCTCAGGAAGCAAAGTAGATGAAGCAAC
TTATCTAGTGGCATGCCCAACTATGATGCAGGTACTCTGGAGTCAAGTTTGACAGATCTCTCTGATGTCCTAGGATAC
CCTAGTCTATACTGTGAGTTCTAGGCCAGACAGGGAGCCACAGTAATACTAGTCTTGAAAAGAAGAGAAAAATAATAC
AAATGGAAAAATAACAGAAATAATAACAATTTAAAACTTATTTATTATGTTTGACTAATTTGGGACAATAAAGAAAAT
GTATTGCAAATAATACATCCCTAGGGGTCCTGACAGACTGAGATAGAAAGAAAATATATGCAAAACTAAATAATCTAA
TGAAGCCAAATAATGAATTTTCCCCATCTCTGAAACACTCCCACATTCTCAGTTTCTGTAATTACATCACACACACAC
ACACACACACACACACACACACACACACACACACACACCAGAATTTTGTTTTGTTTTTAATGAATCTTATTGGCATGA
TCTATAAAGCATCTCTGGATTATCTATCCCCTTGGAAGGCAGGAGTACTTCAAGTTGAGCTGTCCCGTGGGAAAATAA
TGGGGACCTGAAAATTCATGGAAATTGAAATAATAATTACTTATATGTATATCTATAGCATAGGAAGCTAGGTTGTAT
GATAATGTTATGGACCATGTGGTGAACAATGCTGCTTGACAAAGTTCCAGCATTTTAACACTGCAGCCTGCCAAACTT
CTCCAAAACATGCTGAGGCAGGTAGAGCTCTATGAGCTCCTGGTCAGACTGGCAGAATGACTTCCAGGCCATCCAAAG
CCACAGAGAGAAGCACTGTATCAAAAAAGTTGATGACATCCACCTTCATCTGCTTGGTAGCAATGCACATGTTTAACC
CCAGCACTCAGGAGGCAGAGACAGTCATATTTTCTGAGTTTGAGGCCAGCCAGGTTCACAGAATGAGCTCCAGGGCTA
CACAGATAAACTCTGTCTTGACAACACAAAAATGAGGCACAACAAAACAAAACACATCAAAAAAGAATAAAATCCGCC
TTTAGTATGGATCACACCATCTTATGGCAGCCTGCATATATGAAGGGTATTTAAGGAGCAAGCTCTCACTCTTTGGAT
GCTTACCCTAGCTCTTGCTACCAAGTTCAGTTCTTTACTAGCATTTCAGCCTATGCATTTGGAATTTTGGTGTGTAAT
GAAACCAGTTGAGACATCCAGGCTGATGAACTAAACAACTACTGGACTTGAATTTTCATTGGTCAACAGCCATTGTTG
GACAAGTTGGGCCTGAGCCTGTCAGACATTCTAATGAATCTACTTTCTACATACATGGAGAGATCATTCTACCAGTCC
TGTTCATGTGGAGAACCTTGAATAATGAAGTTAGTTATTTTTAATTTTTGTTTTATTTAAACTGTTCACTGAAGAAGT
TGCACTGGCAACAATTGTGTAAGGAGCCTTGTAACCAGGCAGGCAAAGCCTGGCTGGAGAGAGATTAAGACAAAGGTT
ACAACCCAGCAGGCAAAGGCTGGCTGAAAAAAGATTGAAAGAAAGGGAGTATAGGCCCACTACCCAACCATTGTAGAT
AAGAATGAGAATGTGTGACAAAGTGTGATTAGCAAGATTTGGCAAATTATTTAAGGACCAGCTCAGAACAGGTAACTT
TAGAGTGTACCATGACTCTATGTCCTGGAGCTCTCCTACGGTAACCTGAAATAACCAGTCCATCAAGCTGAGTGCCAC
CCGTTCTCAACCCGATTCCATGACACTGGGTCCTCAGCAGACTAGACAGCCTGTATCAAGAGGAACACAAGTCTGGAG
CCCTAATCTGCAGGGCTGCTGCAGAAGAGGTACCCCCTGCTGTGAGCCTGTTGTCTGTGGTTCTTAGGATTGTTTGCG
TATGCTTGTGTGTTACTTTGACATAAGTGCGAAGTAAAACTTATATACTCAAAACCAACTCACTGAGTTTGTCATTCT
TCCCAGGTCAAAACCTGGTAAGGGATCGAGACAAATAGTGTTTATAAACACAATCTTCCTAGCTCAAACACTCTAAAT
ATTCAACAATCTATAAAGACTACTCTTTTATTATTATGCATTTGCCAAGAGTTTGCTCTGAATTCCAGGAATAAAACT
CCTCTAGCAATGGAGCAATTACCCATCTTTCTAATGGGAGAAAGACCTATTATCTTGTTTGTGATCTGGTTTCTACTT
TTCTTTCCTAATTGTCTCAAGTTCCTGTGACACCCAGGTAAAAAGTACCTTCAATTCACCTGACCACAGTGTAAAGCC
ACATAACAAACATCTCCTTTTGTATTCTTAAATCTCTCTTTAGTAGTAAATATTCTGGGACCCTACCAGTTTTTTGAG
AAAGAATCATTCCATACACATAGTAAGTGTAGTGGATTTTACAAGATAGCTAGCCACATTGTGTCCAGGAGGAAGAAG
GGGCAATGCCTACGGAGTGTAAAATGATAGCAGTGTCTGCTTTTGAGCCATGGGCAATTGCTGTAGAAATGCATATCT
CCGCTGATTAGTACAGGTCTTTTTCTGGGGTGGAAAGTAGGGTCAGGAACCATTGTACTGTGTCTTTAAGCGAACTGT
AACCTTATGGGAGGTACTCTTGTCACTCAAACATTACTAGCCAATCAGGCCCCTAGGGGAACTGCCAGATCCAAGGGC
AGGCCCCTTTTGGCCACTGGCTTCAGGCTTGGCTTTGAAGAGAAACTGGAACCAGTGGTTTTGTGTCCTATGCTTTGA
GATGGGTCAAACAAGAAAACTACAACATGGTGAGTGTGGGGCTGCTGTCCTCAGATGGGGAGTGGCAGAGGATGAGGT
GCTCTAGGCAGTGTGGTGGGAACTCCCTCGGTCTGGGTGGGAAGCTGCAACCACATGCCCTGTCTTGTGGGGTCCCAT
GCAGTCCTTGAAATGCCCTTGACCCAGAAAGGCAGGCTTCTGAATAACTTTGCTTTGTCGGATGAGTCTGTTCTGAGC
ATTGGGAACACGGAGCAGTGTGTAGAATCATCTTGGTAGTCCACTTCAACAACCTTTTGGTGCACCCCTGGAAAGCAG
GTTGCCAAACCTGAAGACACCTAGTTTCTTTGTTAACTTCTTGCATTTAATCTTTAGGTGTAATATCTGTTTATTGCT
AATTTTGTGGAAGCTATAAAAGGCATCTCATTTGTTGGGTAGCACACCAATAATAAGCTGTGATGTTGAAGAGCATAA
TTTGTAATACAAAGCATTTACCAAGGCTTAGGAGATAGAAAGTCTCCCAAAGCAGAAATTACCTGAGGGAGGACTGCC
TCTAAAACAACCAAAGATAAGTATATGCTAACAAGGCCTGTTAAAAATTAGGATGCTAGGATATAAATTTCTTCACCA
GGATTTGCTAAACATTTGGCTCCCAATCATTACAAATGAGCTTCCCCTGATTTACAACCAGCCACTAGGCCCATCTCC
ATATCAAAGGACAGGCATGTGACAAGCTGCTCAAGGTGGCATTACAGGGTCACAGCAACCTTATGGCCTGTTGTGTCA
AAAAGTTCCAGTTTTCTTCTTGAGAAAAGCAGAGTTCTGCCAGGTGGGCTATAGAACACCAGGAGACAACTATTTGAA
ATGTAAAATAATGCCTTAAGGACTTCCCTGATCAATTAAGGCTTTCATTTAGGCCCTCTGTCAGGAAGGGGGAAGAGT
CTTGTGATAGCTAGTAACAGCCTGGGGCCTAGGATTTGGCAGCTTTCCCTCAGGGCGTGATATATTAACAATAAAACA
GAAAGCCTCCTCTAAAACCAGGAATTTGCTTGCAAGAGCTGATAGTGGTCTGGAAGAAAGCCTTTGTGGAGAGGTTGC
TTCAGATCTGTACACAGGGGTGAGTAATATCGAAACTTACATTTCAGCACTTTTTATTGAAAGACTCCCCCCCCATCT
CAGACCAGGACCGCAGCCACCCCAATCACCATGAGGAGGCAGAAACTTGATGCAAACAGCAAGAGGCTTTAATGAAAG
GTGAATGTTTGTACAAAGGGGCCTCCTCAGCACGCAGGCTAGAGGGCATCCCCCGTCCCCCAGGCACAGGTGTTTTTA
AAGGCAAAACCCACAAGTTTAGGGAGGGGCCTCGACTCTGTCCATTGAGTACCTGATCAGAGTCATGGGTTCCTAGGA
GGCCCTGTGTCTTGAGTGGAGGGCTGGGAATCAGATGGCCTCTGGACCCACCAGTTGTAAAACCCACAGACCTCAGGA
TGTGCTAACTAATCTTACCTGAGCAAATGGTAACTATCTCTTTGTTTCATAGGGACCCTTAATTGTTAATTATTGACA
CATTGGCCAGTGGGGCAATCTGTTTCCTTCATGAGCCCCTTGGGTGAGGGTGGCCATCTGGGAATTCTTGGTACCCAG
TTATCTTATGGTCTTGTAAGGGAGTAATTGCTGGCGTCCGGTAAATTCCAGGCACTAACTCATAGTAGTTGCCTTGGT
TAGTTTCTGGGCTATATTTCTTTGCTAATTTCTAAGTTTTTCATTATAAAAATTCCAGGTTTGGAGTAGAAGCCCGGG
CCAGTGACACCCATGCTGTGTGTTTAAGCACACCCTAACCCTTAAGGAAGTGCTCTTGTTGCTCAAGCCTCACTAGCC
AATCAGGGACACCAGGCCAACATGTCAGTCAAAAGTCTTGCAGGATGCTTCCTGGAGCCATGCTGCAAGCTTGGTTTT
ATAGCAGAAAAGGTGCCAGTGGATTTACTTCTGTGCTGTGAGTAATACTGTAGAGGATTGTGGGGGTAGCATGGATGA
GTGCAGAAGCCACAGCATGGTGATTGAGGGGATGCTATCCCAAGATGGGGAGTACAGAATGGGGAGGCATGGGAGATT
GTGTGGCAGGCCATCCAGGAAGCTGGGTGGGAACCTGCTGCCACATTGTGTTCTGTGAGGTCCCATTTGATCCGCAAA
ACTTCCCAGACTCATGTGCTGGCCTATGAATACTTCACTCTGTGGGCTGCATCCGTGGAAAGCATTTTCATCAGGGAT
CTTTGTGTAGAAACATCTTTGTAGTCAATACCAGTGTGCTTTGGGACATATGGAAGGCAACTTGTCAAACTTGGAGAG
GCTTTGTTTCTCCCATATTTTCTCAAATTTCTGCACTTCATATTTGACATTTAGGTTTATAATGCATTTGGAGTTAAT
TTTGTGTATGGTATAAATGGCATTCTTTTTCTGCATTTTATATTTACTTATTTGTGGATTCTGAGACAAGATTTCACT
GTATAGCTTTGGAGCCTGACCTGAAACTTGCTCTGTAGAGAAGGTTAGCCACAAACATAGAGACCAGCCTGACTCTGC
CTCTCAAGTGCTGGGATTAAACGTGTGTGCCACTAAATACTTCTGGAGGGAAGGGTGCTATAATACACATTTATTCTC
CAAGATTGTCCAGACTCATGGCTCCAAACCATGAAAAAGGAGAAACCCCTGATTTAAAAATGGCCCCTAGGTCCATTC
ACATATCAAAGAACAGGGCATTTGCAAGCTTCACAAGGGGGCATTACTGGCTCACTATGAACACTAGGACATGGTGTA
CCAAAAACGTCTAGTTTTCTTATAAGGAAAATCATAGTTCTGCCAGATGGGCAAGATGAGATGAGGTGATGACTGTTT
AAAAATGAAAACAGTGCTGTGTTGTGGGTTGAGCTGAGCAAGCAAAACTTACTCTTTAGGTCCTCACTCATGGAGAAG
GAACATGTCTCTTTCTCAAACCCAGGAACATGCTTGGCAGAGGTAGTAATAGCCTGGGGCCTAGACCTAGGAGTTTTT
CATCAGGCCCTGAATCATCAAGAATAAAATATATGTCCACCATTAAAACTAGGAAGATGTTTTGAAGAGAATGTAATG
GTCTGGATCCAGGGCCTTTGTGGAGACATTCTGAGAATATAACTCTTCCCACCACATGGAGCTGTGGTTATTGGTTCC
AAGGCCACTGTGAGCTTCATCAGTAATGTTCTTGGGATATCCTGTGTTCCCCTTGTACTGCATTGTTTGAGTAGCCTT
CTCCTGAGTTTGTCACATGGGATGATAATTTCTGCTGACTTCCTAGAAGTCTCTGCTTCCCCTCCATTTTCCCTGGAA
GTAGTATAAAAGAAGCTATTCCTCTTAAGCTTACTTAGAATCAGACGTAGATTTAGCAAGACTTCCTTGCCCCAGCTT
TTATCTTTTCTTTGTTTTGCTCTTCCTGTCTTTCTCATCTTCTGTCACCTCTACCTCAGGACTTGTCGGTAAAGTATG
ATGTTCTGTCTCATTTTACCCATGTACGGTGAAATAATTTTGGTAATATCTTTTCTGTTGAAGTCTTTCATTTGCCTA
ACTGGCCCAACTTGCAGTTTCCTGTCATTATTTTTCAAAGTGTAAATTTTTCAAATAAATCTGACCCTAAATAAATTA
AAGATGACAGCATATATTTAATGCAAAGTCTGTTTTAGGAGCCAGACTGAACTGGTGCACACCTTCAATCCCTGCCTT
TGGGATTGCTGTGAGGTTGAGGCCAGCCTGGTCTCCATAGCAAGTTCCAGGACAACTTCTGCTGTTGCATAGATAAAC
CCTGTCTTAAAAAAACAAAAAAAAATCTGTTTTAGCTTCTCTTCTCTTTGAGAATTAGCTAGAGGTGGAAGTATGGTA
TCATTACAGAGAATTTCCAACTTTTCATCTCTTAAATATGTAGAGTTTCTTCTCATTGGAAAGAGATATTAATTCTAG
AACACCTGCTCTTCCAGTCTTTTCAAAAAAGGTTTCTGAGGCAAACTCTCTCTTGTTGACACATGTGGTCTTGATCCC
ACTCTGTTGATTTCCAGGGAAATGACCTCGATGTTCTCTTGATTATGCCTTCTCAAATTAGGTGACTTTCATGTATTT
TACTGTCCATTGCAGTCACAAGAATCGGATTTTCATGTGGATCATGGCTTCTGCCTTCAGACAAGAGTATCAAGAAGA
GTTAATTTAGTTCAATGCAAGGGGAAAACAGTAGGAAATCAATTGCTTGATTCACAATCATGAAAAAGGAACTGATTG
TGGCAATGTGATAGTTTGCATGTAATTGGCACAATAATATAGGGAGTATCACTAATAGAAATCATGGAACTTAGCTGA
GGAAGGACTGCTTCTAAAACACCCAAAGCTAAATTTATGCTAACCAATGTTAGAAATTAGTATGTTTCTGGGTGTTGG
TGATGCACACCTTTAATCCCAGCACTCAGGAGGTGGATCTGTGAAACTGAGGCCAGCATCCTCTACAGAGCAAGTGTA
GGTATAGGCTCCAAAGCTGCAGAGAGATGTTCTGAAAAAAAAAAGAAATTAGGGTAATAGGATACACATGTGTTCACT
AGGATGGTCTAGATTGTAGCTCTAAACAATTACAAATGAACCATCCATAATTTTTCTTTTTTTCCCTTTTTTTCCTTC
CCCGTGTGACCCACTTCAGTGCCAACAGCCTGCAGGCAGAGGCCAGGCAACGGTTAGGAGCCAGTTTGCTCTAGGATG
GGACACTATGAGGAACCATCCATAATTTAAAACCAGCCTTTGGCCCATCCCTATATCAAAGCACAGGCCTGTAACAAG
CTGCTCAAGGAGGCATTACAGGGTCAAAAGCAACCTTAAGGCATTCTGTGCCATAAAGCTCCAGTTTTCTTCTTGAGA
AAAGCAGAGTTCTGCCAGGTGGGCAATAGGACACCAGGCTACAACTGTTTGAAAATTAAAGCACTGCTGTGTTAAGGA
TTTGCCTGGTCATTCAAGGCTGTCATTTAGGCCTCAAGCTGGAGGGGGAACATTCTTGGGATAGCTAGTGACATCCTG
GGGCCTACGCCTTGGGAGCTTCCCCTAAGCACCTGGTGTATTGACAACGAAACATAGAGCCTTGTCTAAAATCAGTAA
ATACCTAGAGGAGATGGTTATGTTCTGGAACAAAGCCTTTCTGGAGAGTAGGCTTCATATTTGGAGAACAAACTTCTT
CCCACTACCTGGAGCTATTGTTATTGGTCACAAGGCCACTATGGGCTTGGTCAGTAATGTTTGTGGGAAACCGTCTGT
TCCCCTTGTGCTGCATTGATTGAGTAGTCTTCTGAGTTTGTCTCATTGTATGAAAATTTCTGTTCACTTCCTAGAAGT
CTGCTATTCCCCTGCGTTTCCACCTGGATGTAGTAAAAAAGAAGCTATTCCTCTTAAAAAGCTTACTTAGAATAAGAC
ATAGATTTAGCAAGGCTTTCCTTCCCCAGCTTTTTTTTCTTTATTCTATACTGCCTTTCCTGTTTTTCTCATCTTCTG
GCACCTCTACCTCAGGAACTGTCAGTAAAGTATGACCTGCTGTTTCATGTTACTCTTGTACTATAAAATAATTATGGT
AATATCTTATGTATTGAAGTTTTCATTTGCTTAACTGGCCCAACTTGTAGTGTTCTGTTACTATTTAAAATTTTCAAA
GTGCAAGCTGTAGTCTGAACCTAAAAGAACTTAAAGATGACAGCACATGTGGAATGCAAATCCTGTTTTAGGAGCTAG
GCAGAGGTGGTGCATGCCTTTAATCCCAGCACTCAGGAGTCAAACGCTGGCAGATTGCTGTTAGTTTTTGGACAGCCT
GATCTACAGAGATAGTTCCAGTAAAACCAGGACTTTGCACAGAGAAACACTATCTCAAAAAAACAAAAAATATATTTT
ACGCTCTCTCCTCTTTGAGCATTAGCCAGAGATGGATGCATGCTATCAATACACAGATTATTCTTCATTAATCTCTTA
AGTCTGTGGAGTTATTTCTCATGGGAAAAGGCATATTCTACAACAGCTGCCCTTCTATCCTTTTAGAAAATGATTTCT
AAGAAAAGTCATGCTGACACAGGTGGCCTTGATCCCAGGTTGTTGCTTTCCAGGGCAATGACCTCGCCTTCCTCTTGA
TTACCCCTTCTTAAATTAAGTGACTGTGTGACCTCAGAGTTTTAAAATCATTCATTTGGGTCATGTAGTTGACTGTCC
TTTGCAGTCACAAGAAAAGTATTTGCATATGGAGAAAGGTTTCTGGCTTCCGACCAGAGTAGAAAGAATAGATTATTG
AGTGTGAATGTAACATATGTATGGCTGAAGGACTTCCAAGAACTTAGCAAAGGGATGGCTTCCCTAGATAATGTGTGT
GTAGTGGTATGGGCACCAAAATACCCTGTTCCTTGGAAGAACAGGAAGTGCTCTGGGAGACTGGGAGGATGTCTACTA
AGACACAAACTTTCCAGACTTGGGCCCTGTATATTGGGATGGAAGGACAGAGCAAGGAATGCCTATGGCGTCTGATTT
ATTTGATCTCTACATACTCAAGAGGGGAGCTGCTGATGTTCTGTGGTTCAACTGTGTGGACCCATTTAAGGAGGCACT
GTGGGAAGGCTTAGGACTCAGGAGGACAAGTTAGAGTTATCTAACCTTTTTCCTTCCAGGATTCTCTGCTGGATATTT
CGTAGGAGATGTAATGAAGACCACAGTCACAGCAAGGTAAGTGTGGAGGCTTCAGAGCAGGAGCAGCAGTTCTGTCTA
GCTGTCATTGCTGCTGGGATTGGGAGGGACTATGAGCCATGCACTGACCTGGGTAGTGATCCCTGTCATCTGAGCCTC
CCTGAGTTTAGGTTTCCTGGGAAGAGAGAGAGAGGTATGCAACCCTGGGTGTGATACTTATAATATCAGTGTACAGAG
TGTATTACAGGTTGCTTTGAATGTGGGGTGGAACCATACATTATAAAACTGAAATTGAGGCTTCTGGAATTTATGAAA
ACTGCATTCCACTTTCAGGTTTGTCTTACATGTGTTACACAGTTTAGAAACTGGTAGAAGGAAGTTATGGGGTTCATC
CTTCAGCAATACATAAGAATCCTTTTAACTTTCTGCTACCACCAGCTACAATGATGGGATTTAGGAAAGTTCAGTTCT
AGAGAACAAGAGGAATTTCTGATCTGCAGAAGTTTCTCAGTTGTTCATGGTGTTGGCTGTGTGGATAGGGCAGATTCC
ACATTTTGTGTCATGATACATGTGGCAAGAAGAAGATGTTGAATTATTCTTCTACATTATTTTCTTTGTGGGCAGAAA
TGGAGCTTGGTTTGCCACAAACCACAAGGGGACATGAGAGTTTCAGAGAGATAGCAAGAGTTTACCCACATTTGTAGA
CAGAGCTCTAGATCATAGGGAATCTCTCTTTGGAAAAATATTGAGGACTGGAGGGTTAGAAGCAGAAGAGTTTAGGGG
TCCTTTTGAGTTAATTTTGTGGTAGTTGTGAAGTCTGTTTATTTAGTTTCTAAACACATACAATTAAGGGTAACTTGG
TAACATTGGACATGATCTCAGTTTACCTGGTTTTCCTTTGGTCTTTTGATGAAAAGTTGATTGTATTTTCCTGCCATT
GTTCCTGTCTCTTTCTCTCCATCAACTTTATTCTTTCCCAAATATTGTACACCTTGATTACTGTAGATTTGTTTATGT
ATAGAAGTCACTTTGTGTTAATGTGTATACTATTTTTGTAATCCTCTTGAGAATTCTGTTTCCAATTTTCATATAGCT
TTGGAATCAGATATTCTGTCATAAGTTGTTGAGAATTTTGTTTGGATTCAGTTCATGTGGCAAATTTTGCTTACATGG
TATCTTAATATTGGGGTATCAAGTTCCTAAAATTAATCATTTACTTCTTCATTAATGTAGACTCTTTTCTTCTTGTTG
TTTTTAATATGTGAAAGACTTTTACTGTTTCAACTAAAAAGGACTATGGGTGTCTCAGGACACATTCTTCTTGGGGAT
GACCTCAGTTCAGATCCTGCCACCCAAATCTGGAGGCTCATACCTACCTGCTTCTCAAGATCTGGTATATCTGACACC
CTCTTCTCTGGTATGTCATTTGTACAAACATGGAAAACCTCTCTCCCAAACCAACTCTAGATACCAAAAAAAGTGGGA
TAAACTTCAAAATTTTCCATAGACTACTCAGGGAGAAAACCACACTGGGAGAGAATCAGGGTCTTGGTACTCATGTAG
GGATGGATTCGTCATGACAGACAGACACAAACACAGGAGAAGTTTGATGCTGCTACAAAAATCTTTTTCCTTAGTGAC
AGTAGTAGGAGGTGTGGCTTTGCTGTGATGGACTATCCTTTGTTAGAGGAAGTATGTCCCTGTGGGTTCAGGCATTGA
GGTTTCTTATACTGGTGATGCTGCCCAGTGTGTCAATCAAATTCCTGCTGCCTCTGAGTCAAGATTTCAGGACTCTAA
ACTTCATCACCACATCTGCCTGCATAGTGCCATGACCCGTGACAAGATGATGATTAACTGAAACTCTGAAATTTTAAG
GGAGATGTCTAAATGAAATTTTTGTAAGAGCTGCCTTAGTTCATAGTGTCTCTTCACGGGGGAAAAAAAAACTAACAA
AGACAGACATTGGTACCAGGATCTGGGGTATTTTTTGATAGGCTGGCCCTATCATACAATTTCTCTGATGTGTTCTCA
GTAGTGTTGCTCTGACAGCTTGTCTCTCCCTTTATGTCTATTCATTGGGATTTTTTTTTAGTAGCTCTTTTTTTAAAT
TTATTTATTAGTTCAAGTTAGGGAACAAGCTTTTTTCACATGTAAATCCCTTCTCCCTTCTCCCTCTCCCACCCCTCC
CCCCATCACTCCTCCCCCATCATTGAGATTTCTTTCTCAAGGTTGTATGCTATTTAAAGGGCCAAGAATTGGTGCATG
TGAACCTCCTATTGAGTTTCCTTCTCAAGTGACTGGGTGAATACGTTATAATGTGCATGTCATGAAATAAGTGTGGCA
AACACCAGCATTTTATGACTATATTGTCAAAGAAGCATACACTTGCAACGTATCACACTTCCTGTCTCACACATGTCT
GGGTCATTTTAGGATGCAGTGACCTATGATGATGTGCACATCAACTTCACTCAGGAAGAGTGGGATTTGCTGGATCCT
TCTCAGAAAAGTCTCCACAAAGATGTGATGCTGGAGACCTACAGGAACCTCATTACTATAGGTAAGAGTGAATTTCCC
TTCATGATTTAAAATAAGGGAAAGACTTTTTCTTGGTTATTGAGATTCTTCTGTAATTTCAATTGAGAAAATGGATAA
TTATGTGAACAAGTCATATTTCTAAAGTTCATTGAAGATATTACATAATTTTATACCTTTCCATTAATATATCATATA
TTTCCTGGTATTGTATTTTAGGCTACAGGTGGGAAGACCATAATATTGAAGAACATTCTCAAAGTTCTAGAACAAATA
GAAGGCAATTTTCATTTAAATGTTCATACAAATTTGCCTCTGAAGAAATTTTAATGTGTCCTGGAAATTTTAAAGAAA
AACAACAGTTTAAATAAGCACAATTAAAGTGTATTGATGGTTCTTGGCTGTGTAGTGGTGATGCACCACTTTAATTCC
ATCACTTGGAAGGCAGAGGCAGCCAGATCTCTGTGAGTTCAAGACCAGCCTGTTGTACAGAGCATGTTCCAGGACAGC
CTCTAAAGCCAAAGAGAAACCCTGTCTCAACCCTCCCTAAAAAGTATATTGATGAGTCTTATTTTCTCAAAAACCCAT
GTATTCCAATATCATGTAGCTATTCAGTGTTTGCAAGGCATGTCTTCTGAAAAAGAAGACAAGGAAACAATACCTTAA
CAGATACCTCCATTTGCAATATAACCCTATGAGAGCTATGCTGTAGAAATGTCAATCCACTTAGTTTCATTCCATTCA
GATATTTTAAGTGGTATACATGTGGATTAGATGATACATATATGTCCAAGACCTTGTAATAAAAGTATCTATAGTGAA
GCTGTCATTCCTCATTTATTTGTGACTTTGCTAAGTATATTGAAAGCATCAGTTTATGAGAGTCAAGAATATTGTCGT
GGAGAAACTTTAATCCCTATACCACACATGTGTGAGGAAAAGAAAGTAAAACTGTGTGAATGCAATATGGAAAACTTT
TATTAGTTATTCTTCCTTTAATACAAATATCATCAACAACTGCATGGTGGTGGTGCACACCTTTATCTCAGCACTTGA
TAGGTAGAGGCAGGCAGATCTTGTAAGTAGGCTAGCCTGGTCTAAAGGTTCCAGGATAGGATCCATAACTACACAGAG
AAACCTATCTCAAAAAAACAAGAAAAAAAAGGAATATAATATGTCACTGTAAATACAAGCCGTGTGAGCATAGAATAT
GGAAAGAGGCAATGTACATCTCTGACACTCTCACTACAATTAGAAGATATAAACAATATTTAGCAGTCTCCACTTTGA
GTAGATTTGCTGAATGTGATACAAGTTTACAATTAATTGCTTTTCCAACTTCATTGAAAAGAAGTAAAAAAAAAACTC
ACACTGCAGAAAAGCCCTATAAGTTTTAGAATGTAGGACTACTTTCAGTTGTCCTGATACATTCTGAGACTGTAGTAT
GACTCATAATATGGAAAACCTTTTGAATGCAGTAAGTATAGCTAAGCTCTGAGTTCCTCCAGTTCTCTTCAAATATGT
GAGAAATCTCACATTGAAAACATGACATGTGGCCCTTTCCACCGACTCATCACAGACCAGATCCCTCCAGGATTGTCC
CTGCTTGAAACAGACACACCCAGGCAAGGAGGAGCTCCCTGAATCTGGGTACAGGCCACTCTTCCTTCCGTTCCCGGC
GACCGATCCAACAGGTGAGTGAGGAGACTACGAGGAGAGGCAAGACCATCCAGAGCTTTGGACATTGAATTTCTGGCC
CACAGACACTACAGCGTAAAAAGAGGAGATAGAGAGACCCCACCTGCACTCACTGGAAAAAGATATGGGAAGAAGACA
GAATAAGAACTCGCTCAACAACAGAAAG
Targeted integration site 2 (PCL3-C31) genomic sequence
(SEQ ID NO: 2)
GAAGGGAGTGTTTCAAGGCTGTGGAATATTGAACAATTAATACGAGGATAGTTAATAAAAGATATTTAATGATGCGGC
TGTAATTATTACAAAGCCATCTGCCTGGTGGTGATATTATAAGGCCTCGTTGAGCATTTATATTAACATAAAACTGAA
TGTTAAGTTGGTTCTGTTAAACAATCTATGTAAATGACTGAGAAAGAACCATGGTCTCCCTAGTCTCGCAGATGGTAT
AAAGGACTTCTAATTCATTGTAGAAGGAGAGGCGTGGGCTTCAGGGTAACAGTTAATTATCCCTGAGCTGGATGGGAA
GATTACAGAGATATTACCAATGATGGTAGGATTTCCGTTTTACTGACAACACTCTAGCTCTAAAAACAATAAAGTGGG
AATTAAACATCACAACACACTAAGCATTTTTATTTTTTAGTGCTTCTTTGTATACTTGGCGAGCTTACATAAAGCTCC
TCAAACTTGGGTCATATTTTAACGGCATTGTTTGCAAAAAGCAAAAGTTATATAAGCTCTGATGAGAACCATAAGCAT
CAATTCGGAGCTAACACACAGTATTAGCTGGTGGCGCGGAGGACCTTTGGAGTTAAAGAAACAGTGGAAGCTATGTGG
GAAGGCTGTGTTCTAAACAAACAACCCACAACCACATAGTACAGGGGACAGCAGAAAATCAAGCTGGTGGCCCCAGTC
TGGACGGCAACCCTCTGAAAGTGCCTTTTGGTCTTCATAGATCACCAGCTCTCCATCCACTCTTTTCTCTGACTTGCC
TAGGTTTGTAGGATGTTGTTTTTTTTTTTTTTTTCTGTGAAGTGAGTTATGAGTCATTCAGAAGAGCACAGGCCTCTG
TACATTTTTTACCATGTGTCATCAAATGTCACACAGCTCAACAGCAGGAATTTTTTTTCTCATAATGATAAATGCAAA
GTCTGGGTTAGTTTGGGGGTTTCAGATTTTTGTTTCTGCTAATCCTGAGAAAACATTAGTGAACAGAGAGAGAGAGAG
AGAGAGAGAGAGAGAGAGAGAGAGAGAGAGCATCTTCCATGCACGGAGACCATCTTGCTGCTAAACTATTTCCCATCT
TCCAGATTAATAGACTTTTCAGTATTATTTGAAAATAATTTTACAATGCATATTTCCAAAATAGAAGTATCTGAATAA
AATAAATGAAGAGAATTATGAATGTCGTCTTTTTTTTTTTTTTTACTTTCTACAAAATGAAAACACAGTCTTATGTCT
GGGCAATAACTACATTCACTTTACTCTCACACCTCTTGACTTACCAAGTAATAGTAACAGTAATAGTAATAGTAAGCC
TAACAATAATTTAAAATCAAGACACTTACTTAGTGCTAACTGTGTATCAAAAACTACTCTAAATATTTCCCCAACTTA
ACTCATTTAGCACCCAGCTCTGTAAGCTAACCTTACTATTTTTTCTTGGAAAAGATAAGGGAGATAGAAGATGGAGAG
GGAGACAAAGACAGAGACACAGAGAATCTCCTCAGCTGTAGGGACCAGGATACTAACCTAGCAGTTTTCTTACAGAAC
ACAATTGATCAAAGACAACAGGTGTCTTTTATTATCAGCTTTAAATTCTAAAGAAGAACACGTCCAATCTTCTTAAGC
AAAGGTCAGATGGTGGCTCCCTTTTCAGTCCCAGCTCTGGGAGTTCGAGACAGCGGAATCCTTGTAGATTACTAGCTA
GGTGGTCTAGCCAAGATGGCAGATGCCTGGTTTCATGCAAGATCTTTCCTCAAAAAAACAGGAAGAAGAGCAATTGAA
GAAATTATGCTGATGTAAACCTCTGCCTCCAACACACAAATGATTCCCTATACAAGTACACATGTGAAAACATCCCCG
ACACACTAGTTTAAAAAAATAGTGGGAGGCCCCTGGCAGTGGGAGGAGAACTTAACCCAGTTGCATGAACAGGTGGGA
TACCTAGCTCAGCCTTGATCCTGCCTCAACTTGGTCTTCCAGACTTTGTTGATTCCCTAAGGGAGGCCTTACCCCCCT
CTGAGGAGTGGATGGAGAGTGTGGGAAGGGGGTGAGGTGGGGGCGGGAGAAGGGGAGGGAGAGGGAACTGGGGCTGCT
ATGTAAAATGAATTTTTTTTAAATAAAAAAACAGAAACTCATCTAACATGGATGTCTTACCAGTTGCCCTCTTCTATC
CACTCCTTTGGCTACTAGATACTTGGTTACTAGATCCAGGTTAGTCAGCATGAGATGAAGCTGGGATCTTTAGCCACT
GTGAAAGGATGTGGTCTCTCAGGGAAACTGATGGCACAAGTGTGCAAAACTTCTGTGGTTTCAGGACATGGCTATTAA
GGTGTGATTTAAAAGATGGCTTTTCACTCACATTTGGCATGATATCTTTATCACCAATGTGATATGTGTGTGTTGGTT
GGTTGGTTTGTTCCCAGACAGGGTCTCCCCATGTATCTCTGACTGTCCTGGAACTCACTATGTAAATGAGGCTGGCCT
CAAACTCACAAATCCACTTGCCTCTGCCTCCCAAGTGGTAGTATTACCACGCCTAGCTAACTTTATCACTGATATCAA
TGGTGAGCAAAGTATCCAGTAATTCTAATTCTGCATGAAGAGGAAGACAGTTTTTTGTTTTTGTTTTTTTTTTTTTTT
GCAAATGTACTAGTTTACCTATTGAACAAACACTTATGGAGCCATGGTCCTGCTATAACTAGAGTTAAAATATGAATA
AAGCAGACAGCCCCATCTTAAAGGAATTCATCAACTATTGGAGGCATGACTATTTAGGCCTTTAGTCATACAACATAT
ATTTCAAAAAGATAATTCCACTACCAAAATTTGAAGTTTTCTCAGAAAAGTAACTACTAATACTAATTTAAAAGCCAA
ATGGTGTTAAACTACAAAATGTAGGAATATGAGAAATGCATGTAAATTAGTCAATAAATGAGCAATACCTTGAGTCTT
ATCATTGTTCTTCTGGCCCCAATAACAAACAGGTGGAGATAAAAATAATAGAAAAGCACAGGAGAGGGTGTTAACTCT
GCCTGGGGAAGACACTTTTAGAAAGAATAAGAAGCTAAGTATTTGGCAGGAAGACAAATTGACAAAGAGTAATTTCAA
GCAAGTGACACATATTCAGCTCGTGTTACCTATTATCACTATAATTAAAGGAAGTGTCCTTATAGTAGGTTTTGAAAG
ATAAAATGAGACTAGGGCAAAAGAAGGGTCTTGGAAGCAGAGAAAATTGTTGTGCAAAAGAGGGGTACCGTGCTTATT
GTGCCACCACAAAATGAGCAATGGTCCAGGACCACAGCAATGTTGTGTGTGAGAAGCTGATGCAGTTATACCCAGTCC
AAGAGGGCTTTCATATCAGACTTACAAGTGTATATTGTGCCCTACTGCCTGCAAGAGCTCAATAACATGGTTTACTTG
TTTTTGTTGTTGTTGTTGTTGTTGTTGTTTGAGATACCATTGATCACCTTTTAAACTGGTCACCAGTGTGGCAACAGA
CAGAACGTTTTAATGGACAGCGAGGGGCAGAAAAATGTTTGAAATACACAAGAACATGATAAAGTTTTTCATGCGGAG
GGCTTTCAATGTTTCCCAGGCTGATCTCATATCCCTGGGTTAAAGGGACTCTCTTGCCTCAGACTCCCTTTTAGCTAG
GAAGTTTGTACCTTAGAGAAAAATCAAACACAATAGAACTTTTCTCTTTCCTTAAAACATCACTTATAATAGCAAAAA
GGAGAAAGGCATTATCCAAACATCCAACTTCCAGGGTAGAATTATATGAACAGCACTATTTGATGAAGGCATGAATAG
AATATTATGCACCAAAGCTAATGAGTATGAGTGTGATTATGTAAGCGGGATTGAAACTATAAATTGGCATTATTAAAT
ATAATCCATCTAATGTTGTGACTCGTTAGGGAACATTCCTGCTTGTGGATGCACACAACACAAATATAGCTCTACCCT
TATCTGTTCCAGCAACTTCGACTTAAGCCCAAGAATTGGGTAGGGGTAGTCATTGCATACTCCTAGCTACCAACTCTC
ACAAATAAGACTAAGGTTCTTCACAGTTTTCAAATGAAGGAAGGAAATGAGACCAAGTACTGGCCATGAAGAGGAGTC
TTCAGGGGAGATAGAAGTGGGGTGGGTGTGATAGCCCAAGCTTTGCAGCAGGAATGAGTCAGTCTTGGGGTTTCAGGG
AAATGGGACAGTACCTTTGCAGCTCCTGTTGAAACATTTTACTTCCCCTCCTACCTCCTTTCACAAATAGCTTTAAAT
TGTTTTTAAGGGAGGGATGGAAAATATTATCTTCCATCATACCTTTTTGCTGTGACTGAAAGTTGCAATGAAAGACCT
TACTGAAGAGAGACATGATACTGTAACACAGACCCAAAATGGGTCAAAGAGAAGAGAAGGAACTGCAGAGTTCTTGGT
GCTAACAGTGACATATGTATCACACACTCTTTTCCCAAGGCTCAGGGATCATTATGGAAATGGGGGAGCAGATTATGA
TAGCCAGAGGAGGTGAGGGACTACACAGAAACAGTGATTTCAAGACAAAGCAGGGTGACCACACAGGGTGATTCACAG
CAGTTGAGACAATATGTTCACTGGAGCTAAACAAAAGCTCACAGTTGAGACAATATGCACAAAGTCTCAGCATGAAGA
TGGGAGGTGGGCAGGGAGTCAGCATCCTAGGTGAGGGTCTATTAGCAGCTCATAGCTGCTAGGAGAGGGAGAGTCTCT
TTTCTCTAAGGGTGGCCCCTAGTGAGTAGACTGTGCTTCCTTCCTGGAAGTCTATGTATCTAAGAGTATGTGGGCATC
ATCAAAGGTGCTTGATAGTTTAAAAGGGACACAGGGTTTGAGGGGGTGGATAAGAGTGGAGTTTGGAGGGCATGGATA
TCCTCAAAATATATGTTATAACATTCTGAAAGAACTAATAAAAAATGAGGGAAAATAATATAAATGAAATACCCTCTT
CAATATTTGCAGCATTCCATTCAGTTCAGGTTCAGCAATCCCCTTGTGCTTGCTTTCCGCAGTATGTTCTGAAATTTG
TTACACGTTGACTACAGAGGGTATAAACTGTAAGGAAATACTTTTTGCAATCATGAATATTTACAGTCTAAATTGCAA
GAAGGAAAGCCCCTTGAGTGGCTAGCTGTAAACTGGTCATCTGCATCACCTTTCCCAAGCCTCAGGTAACCTTACAAG
GAAGAAGGGGGAGAGATTGGAAGAGCCAGGGGGTGGGGGTCTTTGTTTCAAAAAGGCACCTGAATAGGACAGGGCCAC
TGTATTTGTGAACTCACTGAAGCTGAGATTGTATATGTCCTGCATGAGATTGGACCTCTCGGTATTTCATCATAGGAA
GGGAAAGGACTCATGGGGCTCCACCTATCCCTTGAGAATTATTGACAATTAATAGCATCTGTGGAGGAGTATTGTTTC
ACTTTCTTCAGTGACTGACAGCCACTGACAAATTGCATATGCTTCTGTAAATGACCTCTGACCCATGCACATTCGAGT
CACTATAGTTAAACTCAGTGGATAACACATACACACAAACATACATACAGGATGTCGGAAAGGTTCTCACTGGGGAAC
AGAAGGATTTAAGTGGGAGGAGAAGAGGGGATAAGTGAGGGTAATGAGGGTGAAATGTTTAAAAATCATTTTAAACAA
ATATGAAATAGCGAAAGAATACAAATGAATTTAAAATACCGTGTTTACACTCAAAAGTGCTGTTCTTAAATGATATAC
AATAAGATATGTGTTTCATGCATGCCATGATTATCATCTCAAAGCCGGAAAACCAAACAAATATTGCATAACAAGAGG
GATAATGGGTACAAGTATTTGCATTTTCACTGTGTCTTATAAAGATAACAGATTTTGGGGAAGAAAAGATCAAAGAAA
AGAATGGTTGGACATGGAATGAGGAAATCCATCAGGAAGGAGTTACAGCTATTAGGAAGTCTGCTGATAGTGCAGTCT
AGGTACCTGCAAGGTTTAAGAACTCATAGAGTATAGATCTTTGGAAATATGCCCATCTTCAACAGGCAATAAAAAGGA
GTTCAGAAAAACGTATGTAGGTGTAATACTTTGGAGCATTCCTTTAGTTGCTTCGATCCTGAACCATAGACAGCTTCC
TGTTTCACACACTTGGAAATCCCTACTGGGAGACCAAAACCAGGGGTAACCAAAACCCCTCTTCCAAAGATATTAAAG
ATACCATGAGCAATGCCCAGGAATTTCAACGCAACCATCCCAGACCATCAAATCACCAGGTGCCTTAACTACTACTGA
AGGTGCCTGAAAAAGTACAGTAGGGACACCCAATTATATCACTTTCCCATGGAACCATGACCCAGTAATTGGTTGTTC
TGTTAACAATCACATAACAATGTGATATGTCAGGGCGTTTCACTACCTAGTAATAGAAATACATATAGTCTTTATCAT
TCAGGCATGAATTAAAATGGAGGCATGGCTGGGAGGTGGTGGCACATACCTTTAATCCCAGCACTCAGGTCGCAGAGG
CAGGCAGATCTCTGTGAGTTTGACATCAATCTGGTCTACAGAGCGAGTGCCAGGATAGGCTTCAAAGCTACACAGAGA
AACCCTGTCTTGAAATAACCAAGAAAGGAAGAAAGAAAGAAAGAAAGAAAGAAAGAAGGAAGGAAGGAAGGAAGGAAG
GAAGGAAGGAAGGAAGGAAAGAAAGAAAGAAAGAAAGGAAGGAAGGTAGGAAGGAAGGAAGAAAGGAAGGAAGAAAGG
AAGAAAGGAGGCATGTGTTTACATTATTCAGTATTGAGTTATATTTATTAACTACTGCAAGTCTTAGATGTGATAGAA
CTCCATAGAACTCCACCCTTCCTAATGTTTTACTAGCTTTACCTGTCTACTCTCATGCCTCTTCTCTGTGCGTTTGAG
GAAACATAACCCTTAGTATTTCCTTCTATTCCTTTCAGGCCAAACAGAGTCAGAGTCAAGATTAAGGCACCTTCTCTT
CCACCCCTGCCCATCTCTCTTCGTCCTCCTCCCATTGTACCAGGATGCTCACACTTAGGGCGTGTAAATGAAGGATAT
GAAGAAAGACAAGCCAGGCTGCAGTTTTTGTCCTTTCTAATGACACCAAGAAGAAAAGCCCTAGCTACTTCTTCACTT
ACAATTCTTACCATTTCTCTCCATTTAAACATATATTTTCCTGTCCTATTGTGTTTGATTTCTGTCCATGTATTTATC
ACATTTACTAACTATAAAGCCTCACGTCTCACACTCAACCTAAGCTTGCTGAGTAGAGAATATGACTACAATTTACAT
AGAGCCTGGTGGTGCATAGTAGACAGCCAAGAAGTAGCTAGAGAAAGTGGCCACAGACTCTTTTCATGTCAGGGAATT
CTTCTTGCCATTCTGGGCATGGAATACATGGTTCCATTTTACATTCAGTTAGAATGTTAACTTTGGCATAAATCAAAG
AAACAAAAATCAGATCTCCACAGATTTCCTGAGTGTTAAAGACAACTGTGGAAGAATGGCTGAGCAGATTTATCCATG
TTTCTTAATGTCTCACCAGATCCACCTTTTTAACTATAAAGGGATAGTTTAATCCCACTGACGGGACCAAAATATTGA
AGACTCAATGAAAACTTATTTCAACTGTTATGACTAGTGCCTCCTTGCTATGGTTTCAGTAAGTGCTTACTGACTTGG
CTCCCAGATCTTCTTGTAAACAACTACCACATATCACTTGATGCGTTTATCAGTCATCTACTTGGGAGTATAAACCAC
TTCAAACATTGGCATTTAGCAAGCAGTTAAGTGGTTCATGATTCTTCATACCAAGAAATTGGGGTATTCTCAGCTTAG
AGGTACTTTTGCTGGTCTTGCTTGAAGTCATTTTTGTGCCTAGAAGCTCCACAAGGGGCTGATGGTCCAAGCTTGCCT
CTCCTGCTCATGTTTCAGACAAACAATTCCGCTGTTGATGAGCTACTCTTGGACCTTCAGTGCGGATGTGCTGTCTCT
TCTCCATCTTACTTCTACTTTTCCATGAAACCAAGCTTCTTTATGGGAGGGTAGAACCATTACCAGAGAAAAACCAAG
ACGAGCCTAAAATCTCCCCCGCCTGAGTTTTGCAAGTTGTAAGATGTAACTGCTGATACATTTAGTTGTTCACAGAAA
TCTTAGGATCTGCCTAAAGTACAATGAGGGAAAATAGTCATAACTGGATATTAAAAAAATAAAACAGTCAGATAAAGG
GCTGTGTGATGGTTCAGTGGGTAAAAGTACAACACAAGTGTGTGAACTTGACTTAGGATATTCAGAACCTACCGAAAA
GCCAGGCATGAACAGCTATAATCCCTTTGGTCCTTCCAGGTGAGAGGAAGTCGTGAGGGGCAAATCCTCGAAGCTTCA
ATGCCAGCCAGCCTACAGTTCACAGCAGAAAAACAACAGAGGCACTGTCTCAAGCAATGTGAGAAGATGATGGCATGA
ATATGTGCTTGTTCTCTGGCCCCTACATGCATGTGTGGCATACATATACTAGGGTTAACATATGTACTACACACTTGT
ACAATGTGCACACATACAGAAACCTACAGACACATAAGTGAAATGATCGCATTGCAAATATGTGTGCCAGGATAGGAG
AAACTCTTGAGGCCATCCTTACAAACTACATGCTAAAGTTGGTTTGTTGTTCTCTGACAAAGGACACTAGATGCCTCA
GACTGGTTTGTCTAGCCAGTCAATGGTGAACAAAAGACGATCTCGGTCCCTGTTTGAATTTCACAGAATCCAGTCTTC
ATACAGACTTATTCTGTGACTGCATAAAGATTTCTCTGACAGTGTCTATTTTAACTATTCATTTCTTTCTTCCCAGCA
ATATATAGAGCATAGACTTCAGAGCCAATAAAAAAAGGTGCATTTTAATTATTTCCTAAAGCATTATCTCTCCAAAGA
GAGCCCCAGTGTGAAGGCATTTTTCTATACTCCATTTATTTTTGTTAGGATGTTGAAACTTCCAGTTAAATTTTACAG
TATAGCATCCTTCAATGTGACCACATCTACAGGACTGACGTGAGAGTTAAATTTGGCAAGGCAGCGCCGTAAGAATTG
TGGTTAGCTCCAGGGAGCTTTCAGCCTGAAGAGCGGATTGATTTACACATGGCGATAAGTACCACAGAAAGAATGTGC
AACTTCTTTGCTTCACCTTGCTCCGTGATCTATGAGTCCCTACTGACTCCAATGCCTTTTTTTAATTTTTTAAGGAAC
TATGTGTATTTGTGTTTGTGTGCGGAGTAGGTGTGCATAGAGTATACATGCCAGGAATTGACACTGGGTATCTTTTCC
AGCTACTCACCATCTTAGTTTCTTTTAGTATGATATTCTTTGAGAATTTTGCACATGCATACAATATATTTTGATCAA
ATGCACTCTCCACTCCTTCCCTTAACTCCTCAGCATCTATGTCTTCTCTCATCTTCCCAACTCTGTGCTTTGTGTCTT
TTTTTTTGTTTGTTTTTTGACACCCACCAAATTCAGTTTTTGCTGCCTTAATGTTTTTGCTACCCACAGCACGGTCAA
CATACCAAGGACTAGACACCTAAAGAAAACTCCACGTTAGTCTTTTGAAGCAGGGTCTCTCCCTTAACCTGGATTAGC
TAGACTGGCTGGCTAGTGAGCTCTAAGGGTCTGATGGAACTCAAGCCACTCTACCTGCCTTTGACAGTGTACTAGGGA
TCGGGCCTTAGGCCCCCATACTTGGGAGGTGGGATTTTCCAACTGAGCCATCTGCTCAAGCCTCACTCCATTGTCCTA
AAGAGGCAGCTTATAGTTTCAACCTGAATTGCTGCAAAGCCAGTCTTTAAACATATTTTCTGCTATTACTACTTAGAG
GGCCATCTATGCACACATAAATATATGAGTAGCATAAGAGAGAAATGAACAATGGACACATGGGGAAGGGCCTAGGCC
TGGTCCAGGATGATGTGGTAGACTTTGGGGAGCCCCTTTTGAGGGCCTTACCCTGCCTGGGGAGTGGAGGGGGGATGG
CTAGAGGCAGGTGGGATGTTGGGGGAGGGGAGGGAGAAGGAGAAGGGATTGACATGTGAAGCAAGCTTGTTCCTAATT
TGAACTAATAAAATAAAAAAATAAATAAACATAGAAAAAAAGAGAGAGAAATGGACAAAGTCCATGGGAAACTAGATT
GTGTGTGTGTGTGTGTGTGTGTGTGTGTGTGTGTGTGTGTGTGTGTGTGTGTGTTAAGCCTTATCTCCAAGTGTCTAC
CTTTTACTTACATAACAGGCTAATGAGAGACAGTGGACCTCAAATGTAGCAATCCCAAAGCACAGAATTTTGTACTTT
TAAACAAAGGGAACTCTTCCTGCTTGTACAGAGCTGCCCCTACAGTAAGCATATCACCACTTGTCAGTTTCATTGCTC
TGCTATTCAAATAGCACAAAGAGGAAGCAGAAATATTTGTTGCCAGCTAGCGAGCTCCAATGCTCATCTCTCCATCTG
TTGGACAGCTGGACAAGGCAACAGCTGCCCATTAAAGCTTCTTTGCCTGGCATGATTAGGTCTATTGGCACCCAAAGC
CTGTGCCAGCTGGCTCCTGCTCTTAGTGAATAGACCATATTTGAACAGAGTAAGTCTCCAGTTGCTACAGAACACAGT
CAGACTCCTCACCTCCCTTCAAGAGGCAAGACTTAACCTCATAACAAGAAAAATGAACCAGACTCATTGCTGAGGCAA
GTTTACTTTGGAGGGCTAATTTTCAAACAGGACAGGACACAAGTCCTGCATTTTCCATCTCTCAGCTGTGAAATGAAA
GTTCCTTCATCTACAGTGGAAACAGCACCCGCCTTGGTGATGTTTCTTCCAATTGGATCAATGTCAGTGAAAGGGCCC
TGTGTGTTTTGGACTTTCCTTCTGTGTGAACCAATTCTAAAGCCACAGAAGCCCTAACTCGGGAGCAATCTCAAGAAA
GGAAGTCTCTGGGGTCATTTGTCGTCAATCCTTAATTACCCGAAAGGGCTTGAATGTGCTACTTTGCATGACTCTCGT
GAGAGTACATGCAATGGAAGTTTTAAACATTGCCCCCAAAATAAAATTAGATCAAAATTCATAGCAACTTTCCATCAC
CCTAAGAGTTGAGCAGTAGGAATAGGCCATCAAGTTATATACACTCACCTGACAGCCCCCTAGGAAGACTGTAAGAAC
CAACCTCTATATAATCCACAGAGAGTGCCTTCTCTTTCCATTCTTTCTTGCATTCACCAATCGGGCCACTCCCCATTC
CAAATGGATTGTGCAGTGATGCAAGTCACTAATAAATATCCTTACCTTGAAGAAACAACACACATCATGAGGCAGTAC
GTGAAAAGAAACGGAGACAACGTGTTGTAGAAATTAGCAGGACTGAGAGGCAATGAAAATCTGCATGTATTCATCGTG
TGATAAGTAAGTGTTAAGTCACCGGGGAAGCGTCCTGTGTGTTTTCATCACCAAGCTAAACCCCTTTGAAGCCAAGGA
AATAACTTCACCACATCCATCTCATGAGCAATTCTTTTGCAACGCAGTACGTTTGGACTGAATTAGTCTCCTAGGGCT
GCTACACAGCCGATGGTTTAAGAATAGACTTGCTTTCTCTCATAGTATGTGTGTTCAGCATCTAAGATGTAGGGGTCA
GCAGCGTAGACTTTTCCTGGGACTGTGTAGCACCATCAGTTCCACACTTCTCCCTGAAATTCTTCTGTTTTGGCTAGC
ATTAAGCCCCCTTGCCTGTGAAAGTTTGATCTCTGCCTCCATCTCCCTTTGAGCATTCAAATATGCTCATTTCTTCAC
CTGAATTTCCTTGTGTTCCATTATGGCCTGTCCTAATGACCTCATCCTAAACAACATCATGGGTCATAACTATTTCCA
ATAAGTCACACTCTGAGATACTGAAAGTGAGAACTTTAATATATGAGTTTTATGTGAACATAGTTTAACTCATAATGC
GGCCTTGACTGGCTAATCGGACTCCCTGTGTTTCTCCATAAGCATACCTGAAAATATCATCTTCAAACCTGTGAGTTA
CAAGTAAATGGATGAAGCATGCTCTGTACCTCCTATACTTAAATCCTATTTGCAATTTCAGCATTCCTTCCAACCCCA
CGATTACAGTAATCAGGTACAAGACTTTGGATTCACAAAGGTAGGATAAATATTTCAGCATTCTCTCTGAATTTGTCA
AATGCAAATGGACTAGACATTGTTGATGTACTTATTGCCTGTATATGTTGCATTATACATATTCTATATAGTTTTTTC
ATATATTAGTTATAGCCTTCTTTTTGTATTAGACAAGAAGGGGAATTGTGGTGATGTATTATTTATGATTTATTAAAG
CTTTATGGCTTAATTAAAGGGAAAACAAATGAACACGGAGGACACTAGATTAATAAACGATTGCTTAAAGAATTTGAC
TCTCGCCAGGTGTTGGTGGCACATGCCTTTAATCCCAGCACTTGGGAGGCAGAGGCAGGCAGATCTCTGTGAGTTTGA
GGCCAGTCTGGTCTCCAGAGCGAGTGCCAGGGTAGGTTCCAAAGATACACAGAGAAACCCGGTCAAAAAAAGCCAAAA
AAAAAAAAAAGAAAAGAATTTGACTCTCCTTGTTTGTTAGTAAACTGTTAATGCCTTCTGTGTAACTACCAAAAGCAA
AATATTGAAAACAGACACTTAAAAATTCTATGAATCACTTATGATGTGCCAGAGACTATACAAGGTTATTTCTCTGAG
TGTGTGAGAAACATGATACAGCAGCAAAAGTATAGGCATTCACCCTCGGCGCACAGAGGAGATAGTTAGACTTCAGGT
AATGTGTGGACTATCTTACAGGTTGAAAAGTATCTAGAGGTTTGGGGGAAAATGGGGACAAAAAAACCTCAGCTTATG
CCAAAGTCTCATGTAGCAATCCTGGTCTGTATTTACTCTATGTTTTCTTCATGATGCAATTAAAATAAAGCCTCACTG
GCCCAGGGTGAAAGTGGAGTGTGGTAAGGATGTTTACATAAGCAGCCATGAATAAAACCAAAGACTTTTAAAGAGAAA
AGGAAGTGGGGAGGAAGAATAAGAGACTCAGTTCAAAGAAGGGAAAATACTACTTTATGTTCCTCCCAAGGAGGTGGT
AAGAAGGTGGGTGGTTTACAACTTGCTCTCTTCAAGTTAAATATGGCTGTTGGCACCACCAAGCAATACATGCCATTC
AGAAAATCTTGTTTCCTAGAAAGGCACTGGGCCCTCTCTTCTGTGGGGGTGTGAGATAGAGCATCAGATGTGAGGGTT
TGTCTACCTAGCATTCATTCAATGGTCTTCACCACTGTCTCTAGAAAAGAAATTATGGTAGGTGAGTTCATTGAATTA
AAGAGGAAATGGAGTTGTACTTGGTCTGTGACACCACAATAGTGCTGAATAGTCTTTGCTATTCTGTGGCTTGATGTC
AGAAAACTATCTGTGAACCTGTTGACCAAAGCAAGAAGGCATAAGATGTTCAATATGCTGCCTTTCCTGGCACATACA
CTGACATTAACTCTTCAAATATCTTCCCATTTATTCCCAACTTGATGCTGAAGACGTCTACTCTTTCACCAAAGTTCC
ATTGTGGAGGATTCCAGAATATCCTGACCATGTTTCTCTATATCCGTCATCCACACATTTGTCTGACAGTCTCCAAAT
GTGTGTCACTATGGATGCGTGTGGTATATTTACTCAGTATGGTAAAGTTATTTGATATGAATTATCTAACCATCAGAA
ATATAGAGTTGCGAAGTACCTAGGGAAAATGATGTTTCTTATTATATAAGTTTAAAGCATTTAAACAGCAGTTTTATG
TACAACTCATTTGTTCAATAAAAATTTATATTGTGCTAATGGGGATAGAAAATAAATAGTGAGATATTATATAAGTGC
TAGACACTTATAGCTCCTATAAATATACATTAAAGAAAGTGTGTATAAATGAAAGTCCCCATCTGGCCTGTTAGAAGT
CCTTTGAGGAAACATAACTTAAGTCAAGACATGACAGATAAGCAAATGTTAGCCTGCAAGGGAGATGGCTGATGAGAG
TCATTATCCAGCAATACATAGGAGAACAAAGACCCAGATGAATGAGAAGGCACAGCGTGTAATATGGTTGGCGTTATA
GAATGAAATAGAATCAGAAAACTTGACTGAATCCATTTCAAAGCCGGTATATATCCTTAGAATGAGAGAACACAAAGT
AAAGGAGAGTCACAGGTCAGAAAGATCACTTTTGCTAGAATAGGGAGAACAGGGGCAGGAACAGATGGTTGAAATCTT
GTTATAATTCTCTGGAGATTCCACAGAATCATGACACAAAATTATGACTGCAAAGCTTTAAATATGTAACCAGAGTTA
GAAAATAAATGATGTCACGATGATATTTGATTCTTCAGTTGGTATGCAATTGAGGATGAAGGCGATATCCAAGATGAC
TGTTTGGGTAGGCAGAGATGCCAGTTGTTGAGATAATCATTGGAGATGTGGCTGATAACTGGGGGAAGAGGGAATGTT
TGGCAACATTAGTTTTGAGCTACTTATGAAACAACCAGACACCAGTGACTGGATATCAGGCAGGGATCTAGAATCTGT
AGACCTGATTTAATTTGGAAATATCAGCATGAAATTCATGGTCTTTGAGGTGTTAATGAATCCCCCAATACATAAAAA
ATTATCAAATTAGAAGCCTACAGACAAGATAACCAAGAAATCCCTGAGGAAACTTGAAGTGTAGAGGTCTAGATGTCA
AGTAGGGTGGTTGGGAAAGGCTGTGTCCAAAGGCACAGTTAGCAAAAGGAAATTATTGCATTAGGAAATTCAAAGGGT
TGCCTCAAGGAGGCAGAGACCAAGAGCAAGAGTGGTAGAAAGTTGAATGATGTTGCTAAGTCCACAGATTGTATGGAA
TTGATGGATTGGGTGTCACTGAAGCCCTTCTGGCCAGCGACTGCTAAGAATTAGCATGCAGAGAAAACCAGAACAGAG
TGGGAGGTGAGCAAGTGGGTCATACCCAGAAATCTCTGAAAGCGTGGTAAGAAAAGGGGAAAAGCAGAAAGCAGCCAG
AGTGGAATAACATGCTCCAGACATCACAGAAGAAACTTTTCTCCACTGTCCTCTGATCCCAATTACCACCCCCACCAA
AAGCCCACCAGAGTATCATAGTAGACAGTCTGATAACTTCAAACGTTCTGAAATTGTGACAGAAAGCTTAAAGACTAA
AGTTAGTATGGTACAGTCTCTGCTAAGCTCAAGAAATACCCACAGGAAATGGAAGAACTTTTTGACTGGAGGCCTAAT
CAGAGTAAAGGCCAAGGTCTTGAAAAACCAAATATGTGATATCCTTGTGAGCAGAATGACTAAGTGAGCAAGGGGGGC
CACAGAGCTCCTGCTAAGGGGCTGTACTCTTACTGGTCAATGAAACCACCAATGCAGACACATCTCATTAGAAATGAT
TAATTTAGAAAGCAGAATGGTCCAACAGTTAGAAGCCCCTAGAAGGTCTACTGTAACAGTCAGTATTGAAATGACCTT
TCTCACCATGTACTGCTATGAAATTAGTGTTAGACCTCAAGGAAATGATTTAAATCCAAACAGAGTAAAGACGAAAGT
GCCTTGTACCTTCAGTTTCTCAAAAAAAATGTATTAGCTTGATTGCTTATCAATAATCATTGGGTTACTGGTTTTCAA
CCCTGACACTGGGAAAGTTTGAGAATTTTTCTAGGTTGATTATTTTCTCATGGGCCTAATATTAAGTGATACCCCAAA
CCCATACCAATAACCCAGTTTGTGCCACACCACCCTTGGTAACAGAATATCTACACCCTCCACTAAAATTAATAGGGC
CTGCCCATTTGCTTTTAAATTTGTTGGTTGGGCTTGCTTAATGTTTAAAATTAATAATGATAGTCACAACTGAGACTG
ATTTAGTGGTTAATATTTGTCAAACACTAGATAAGACTAAGCTCAAAATAATCTCCATTCCATAGAAAAAATTAAGAT
GGGCAATGTGTTGATTGAGATTGTGTAACTGTAAGTGCTTCATGAGCATGCTAATCAAAATTAATGATGTTTACTGGT
ATTTTATATGGAATGCAATCCAAGCAAATACTGGGTATTGGGTAAGAAGGTTCCATTCCTAACAAAGATGCAGTGACA
GGAGGGCAGATCAAAGATCCCATCAGAAGGTAGAGGAAGCTCTCTGATACTCAACAGTGTCCCTGTTTGAAAGAAGCA
AAAGGAGTGAGGAAAGTGAAATGTAGATGGTTTACTCCAAAAATGTTCTTTTCAAGGAATTCTGTAGGAATCCTAAGC
ATCGAGAGGAGTGTCATGGGCCAGAAAGGTGCTTCTTACGTAGAAGATCCCGGACAAATTGTTAAATACGTAAAGGCC
ATCTCTGTCCCCAGGAATTGTCTAACATATGAAAAATTAACACATTGCTGCAACAGAAATACATGATCATTTTGAAGA
ACACATTCTAATATTATTTTTATTAAAATCTCATGTATTACTGTGCATGGAAACAATGTATATGCTGATGAATGCATA
GAATCATGAGTTCTCTGGAGCAGGGAAGGACAGAAGGAAGAAGGAAAAAAATAAAACAACAGCATGTAATCCCTGGGC
TGTTCTGAAGAGCCCTTTATGTTTGAATTGAGATTTTTATTTGATCTGGGAATTCACTGCTTCACAAAGTACAAAAAT
GACAAATCTAACTGACTCTTTAATTTTCAAAGTTAGGAAATTGGAGATCAAGAAGTTAAGTCTCTTGACAGGGTTCCA
GGGCTGGTTAGAAGCCAAAGAGACGTGAGGGGAAGCAGTCTGCTTCCTGTGTGATAGCCCTTCCACCAGGCTTCCTCT
CTTCCTTCTTCACTTGAATCAATAGTCCCTTGATGGAATGTGAAAACCACTGACCCTTGTTCCCGCACGTGAGGGTGG
CATTTCTGTATGTCTTATAGAATAATTTGGCTCTTGAACCCTTAACAGAACTGGACTGGCTTTCACTAAGAGATCTGT
GGGACAGGACAGTTTTCCTATTCCCCGGAAGAATACTCTGTATTTCATCAAAGCTGCCATCATAATAAAGAGAGCAGA
TGTGTTTCAAATCACCCATTAAAGTTGGCACAGAAGTACATATGGATAGCATTATGCAGAATGAGCAGGTTGTATTTA
TGGATATATATGTATATCTACACACACACACACACACACACACACACACACACACACACACACACACACATATGCATG
GGAAGGAGGCCATGGATTTGAAAGAGAGAAAGGAAGGGGTATCAGGGAGGGTTTGGAGGGGTGAAAGTGAAGGGGGAA
ATGATGTGATTATATTGTAACCCTAAAAATATAAGAATGATTGGGAAAAATTATTACTTCTCTAAGTACAATAATCCA
TGATAACAACAATCAAATAAAATAAGAGTCATGTGTGTAGTCACAATCAAAATGAAAACCCAGCACACATATGCAAAT
ATACTTAGGAAGGAGTCTATGAAAAGGAACATACAACTTCAGGCGTCACAGTCAACATGAGAGAATAATATTATCTAC
CTAAAAAACAGAACGGTATGAATTTTTTTTTATTTTATCCTGTCATTTTGTCCGTAGATGATCATCAGTGAACATGGA
CGAGTTTTACACCTAATTATATTATTTGATACTCATATTATTATATGTAAATACAAACTAAACTATTGTGATTGGCAT
GTAAAATGTTCTCAGAAGCCCATGCTTTTAACCAATTCATCTTCAGCTATAAGTAAGACCTTTTCAGGAGCTGGAGAG
ATGGCTCACAGGTTAAAAGCCCTGGCTGCTCTTCCAGAGGACTTGAGTTCAATTCCCAGAAGGTACGATCTGGGGGAA
GAAGCAGGGAAGGCATTGGGTTTATAACTTATTTCACCTTCTGGATCTGTCTCTTACTTGCTGATCCAATTTAATGTA
AACAATGTTCTGTAGCCACCGTTATGAGCTGCTCCTACTATAATGTCTATGCATCATGGACAGTATCTCCTCAAACTA
AATAATATATAATGTGTATATATTATATAATATTGACAAATATAATATAATACATTATATGTATTATAAAGACTATCT
TTCCTTCCTTCAGTTGCATTTTTGGGTATTTTATTATTGCAATGAGTAAATTGACTTATATATGAGTCAATATATGAA
TGATTATGAATAATTACTTATGAACATGAGTTCCAGTTAGAGTTCTTTTTACAAGACCAACTAAAAGCCAAAGCATAT
TGTGAGATTTTTGTCGGCTGTCTTTTGTAAACTATCTTGAAAGCTAGACTCAAAGGCGTTAGCACCCATCTGCTCTGA
ATTCTCCATTACACTGACCTGGCATTCTTTCCTATAAAAGGTCAAATAGTAACTGTATTATATTGATTCTCTGACACA
TATCATTTCTATCAAGAGTCAACTCTTGATACATGACGTGTCAGCAAACAGGCATGGCTGTCTTTCAAAAAATCTAAA
TGTTTTTATATAGTCTCTGAGAATATCATATATTTTGGTCATATCCATTGCCCTCTTGCAACTCATTGTATATTATGC
TTTGTGTTACTATGCAATAAACATTGTGTTCTTTCTCGGTCTTAATGAATAAGCAAGCTATGAAATCCATAGTAGACT
ATGACTGCTACTGCATAAGGGGCCTGCCTGGAAGTGTGGTGGTTATACTCAATATCACTCCATTGAAGAAAACTCACT
CTCCTTCTTCCAGGTTCTGTCAATTGTCAATAGCTAGAACCTGAGGCTAGAAAGGGCCTGGGCCTGAGGGACAACCAA
CTACTATTATCAAGCAACCTTTTGTAAGCCAATCATTTTAATTTCATCATAATTTTAACAACACTATTTCTTCCGCAA
AGCAGGATGCATTTGACTTTTATTCCATAATATAAAGGCCCTTTCCTACAGGGTCACAGACTCTTTCATATGCAGTCA
AGAAGGTTTCTTACCCATAACAACAGAGATAGCTCTCTGGAGGATAGAATAGCATGCTCTTCTGATCATGAATCCAAT
GGGTTGTAATGGGAAGTACTAAGTGCCATAAATTTGAGTTGATCAAATATTATGGATACTTGGCTCTCAAAAACTCAG
AGAGGTTTACAAACACAGCAGGGAAATGATAACCTTTTGAAAATGTATCCCCAAGCTCCACAGTTTCACATTTACGGC
TCAATATTTGATAATTTGATATAGTTTTTAAAGGTTTTTTTCCTCTTTCTTTTAATTTATGAAGTATCTTTCATGTAG
ACACTTATGGAGTGTGTCTTCCCAAAACTCATATGTATTTCAAATTTATTTAAATTCAGAATGCTTTAAGATTATTTA
TAGCATAGCATTGTATGCTCAGCCTCTGAATAGATAAGATTGATGGCTTCCCATCAAATCACTCAGCAAATACCTATT
AAGCAACAATGAATGCTGGGCACATTTCTAAGAATAGGTCTACACAACACTGAACAAAGGTGACGTTCTTGCTCTCCA
AAAGCCTACATTCTAGGTTCCTCATTATATTGTTAAACATAGATAAAGACATCGATGAATCAGCTTATTGTATATATT
GCAAGTGACGGAGGTGCAAAGCAATGGTGGCTTGATAGCCTGGAATATGAATGACATTAGGAGCCAGTCATGGAATAT
TCTGAAGAAACCTCATCCAAGTCCAGAGGTATGTCTAATGCAAAGGCCAGACGTAGGAAGGAAATGGAAATGTTAAAG
AAAAAACAATGAAAGTTACTCATGGCCAAGGTACAGGTACAGCACTAAGGTGAATTAGAGAACAGAGCCTTATACACC
ATGGTTGATGGCTTTGATGCTGCTCATATTCTATGGACAGGATAAATATACATACACACACACACACACACACACACA
CACACACACACACACACACACACATATATATATATATATATATCAGGCAAAGTCAACTTTATATTTGTACAAATGTTA
CCTCTTCATAAAAAATAGAAAATAAACAGTAGGGAGTCTAGTCACATTTTGTTAAGGAAATCCAAATAAAGATGAAGA
TGATACAGAAAATCATGAAACTTGGGGTCAATGTGAATTCAGCTACATCCATCTGGAAGATGCAGTTGCTAATGTATC
CATTTTACTGCATGAAGGGAATAGGGGCTTCTGTGAGGAACTCCTGAATGCTACCATGAGGAATTGTGTAGCTATTGT
CATTTACAAAAGAAATTATCGCCGGGCATTGGTGGTGCACGCCTTTAATGCCAGCACTCGGGAGGCAGAGGCAGAGGC
AGGCAGATCTCTGTGAGTTCGAGGCCAGCCTGGTCTCCAGAGCGAGTGCCAGGATAGGCTCCAAAGCTACACAGAGAA
ACGCTGTCTCGAAAAACAGAAGAAGAAGGAGACGAAGAAGAAGACAAAGAAGAAGACGAAGAAGACGAAGAAGACGAA
GAAGAAGAAGAAGAAGAAGAAGAAGAAGAAGAAGAAGAAGAAGAAGAAGAAGAAGAAGAAGAAGAAGAAGAAGAAGAA
GAAGAAGAAGAAATCTTCTGTAACGGCTGTTCACAGTACCACCACACCAATCAGACTACTTCTTTCATTGTTTCCTGT
AATCTTACCACACCGTGCCTCTCATTCTTCCCTATGAAACAAGGACATAAGACCCGTCTTAGTCCACCGACCAATGAG
ATATTCTATCTGTGCTGTTTCATCTTCATTGTTAATCTGATAGGATTACAAATCACCAAGGAAACACACTCTTGTGTG
TATCTATGAAATGTTTTAATAAAGGGTTACTTGAATAAAAGATAAATCCTTAGTGTGGTGGCACCGTTCTGCATACTG
GGACCCTACAGTGGATAAGGAAATAGGCCTAAGATCCAGCCAGGCAATGTCTCAGCAGAACTTGTGAACAGTGTTCAC
ATGGGACTGGGTTTGTAATGCAAGATTCAGGATCCAGAAATGCTTGTGCCTGGTTACTGAGAGCTACTAAGACTGTGT
TAGGGATAGACTGTCTGCAAGGAAGATCCCCAATGCCATTGTGAATTGCTGTAAGAGTGAATCTTAAATTATAAAATA
TAACACAAGATATTAGAGATATCAAAACGATGGGTTATCTGCGTATTAAAGCTGCAAACAAGGAATAGACTGGTCTAA
AAGAGATGCTGTATCTGCAGCAGATAGTAGAGCTGAAGATAAGGAGGCTACTACTACTCTATCCCCTCAAATCCCAAA
TAATTCCATCACAAATCCCATATGACAGAATTTGGTATTTGCCCTGCTAGATTTCAGTCTTGTTTGGTTCAGTATAGC
CTTGTTATTCTCCCGTGACTACCTTTGGGATGGAAAGTTCACTCTATGACACTGTGTATTTTGTTATTTTATGGGGTC
TCACAATTTAGAGACTCTTTTCATCTCAGAAGAAACTTTTGACTTTTGCATAGTGGTAGCCCTGTTAAAGACCATGAG
GACTATTTGAGACACAGTTAGTGAATTGTTCATAATGAAATAAAAAGACATTATGTGACTGAAAAGTGATGTCTTGTG
TCGGGTTAACAGGGAAAGAATTTGTGATCCTTCCTCTTGATTGTCAACTTGAGAGGACTAAGAGTCTCCCTGGAAGCA
TCTATCTGTCTCTGAGGAGATTTGTAGAAATATTTATTTGGAGATGGAAGGCATGCCTTGACTGTGGCACCACTGCTC
CTTGGGAAGGGGTCCAAACTGAAGAATACAGAAGCAGCCATCTCTAGGCCTCCTGGTACTACGGGCACACTGTGACTA
GCCACCATGGCTTTCCCAGAACAATGGACTACACCCTCTCAAGCTATAAACTTCTTAAGCTCTTTCTTGTCAGCTATT
GTTCACAGCAATGAGGAAAAAAATACTTAAAACAGAGTATTACCCATCTGCATAGTGCCAGAATTTGTTACAAATTAC
TATGTGGTCCTTGGAACTATCTGAACTTGATTTTTAATACAAGTCATTAAACACAAATTATGAATAAATTAGGCATGC
TATAATTCTTTATCTTCATATTTCAAATGCCCATGTAAGGTATATATTTATAGTTCTACATCATATGTCATACAAGTA
GGCCACCTGAATTAATAATTACTTGGTTATTTCTCCTCATGAAAACTCACATTTTATCATTTATACATGCACTTACTT
TTCCTCAAAAATGGTCTTTTTTTTTTCAAGAAAGAAATATCTGTTGCAAATAAAGGTAATTACTTTTAAAAAAGAAAC
CATCCTGACCACCTTTGATCATCATGAAGCCTGTTGGAAAGATAACCACTTACATTATTCTCTTTCTTATGGATATAT
GTTAAACCAAGCCCAGGTAGAAGATTCTGGCTTCAAAGATTCCACTACATCTTTGGCTTTAAAACAGAGGACATAAAG
TTGCAAAGGGTGGAAAATGAGAAGACGGTCTAGGAGAGGTCGGTGAATATAATAGAAATCCACTGTGTGCATGTATCA
AATTCTCAAAAACAATGAAATAATACATACATCTGTGTATCTGTTATTCGAGGACCTCAAATTACTTCCTTTTGCAGG
AGCTGAGCAGGAAATGACAGCCAAAGCATCCATGAGGGGAGTTCCAGTCAGTGGACTCAATGAGTCAGTGCTGTAGTT
AGGAATTTAGTCCTGTGGTAGAGTGTTGCCCAGTCTAGTATGCATGCAAGCAGGGCCCCTGAGAGGGACAAAGGAAGG
GAGAAAATCAGGGAAGAAGGGAGGGAGGGAGGAAGGAGTGATGGATAAAATAGAGAGGGCAAGAAGAGGAAGAGTAGG
AAAGGAAAAAAGAGAGTTATTATTGGTGAGTATAGAATTTGGAGAAACTCTAACCTCATTAATCAGTTATGGGGGGGT
TGTATAATTAGAAAAAATTCTCTCTTACACATGAGGTTCAAAAAGAAAAACAGGATAAAGTTTCACATTTGGGTTGAC
ACAAATTAGAAAGAGGTACAATATATTGAGGATTTGTTTTATTGTATTTTAAATACTACGTATGGAGGGAGGGAGGCT
TATGAATTCATGTCTCTAAAGAATCCAGGAGACGGCATCTGATCCCCTGGAACTAGAGTTACAGGCAGTTGTGAGGCA
CCCAATATGGTGCTGGGGCCTTCTGCTAGAATAATACATACTCTTAATCGTCGAGCTACGTGTCTTGCCCTAGCCACA
ATTTCTGGGTGCAAGAATCTAGGAAAGAATCTTAGGGGAAAGCAAGTATGGGCAGAATAGGTGCCCTTGTCAGGCAGA
GATCTGAGCAAATAAGAGGGTTGCTGTGGCTGTAATCTGTGGTGTATACAGAGGCAGTGCGGCCCCTGGATCTCAGTG
TTAGGCAGTGGTGGCTGAGGACTATCAGCTGGAGGCCCTACGATATCTGGCTACCGGCTCACAGAGAGAACTTTGTAG
TG
Targeted integration site 3 (PCL6-C126) genomic sequence
(SEQ ID NO: 3)
TATCCCAGGCTCAATCTCCTTATATAGCTGAAGATGGCCTTGAACCCCTGATCCTGCTGCTTCAGCCTCCCAAGTACT
AGGATGACAGGCATGGGCTGCCAGTACCCAACTTCAGCCAGCCCTTGCTCACAAGTCTGTCTCTGTTCATGCTATTGA
AACCCTGTCACCAGACCATAAGGCAATCTATCGGAATGAATTTGAACTTTGTGTCAGAAAGAACTGGCCTGGCTGGGT
GGTGGTGGCACATACCTTTAATACCACACTTGGGAGGCAAAGGCAGGCAGATTTCTGTGATCTCTGTGAGTTCAAGGC
CAGCCTGGTCTACAAAGTGAGGTCCAGGACAGGCTCCAAAGCTACACAGAGAAACCCTGTATAAAAAAAAAGAAGAAA
GAAAGAAAGAAAGAAAGAAAGAAAGAAAGAAAGAAAGAAAGAAAAAACTGGCCTGGAAAATGAAGAGATGGCTCCACA
GTTAAGAGCACATACTGCTCTTTCAGAGGACCTGTGTTCAGTTCCCAGCACCCAAGTGGTAGCTGACAACCATCCACA
ACTTCAGTTTCAGGAGATGTGGTGCCCTCTTCTGAGCTCTGCGGGCACCCGGCACACATATGGTGCACAGACATACCA
CACAGACAAAACACTCACACACATAAAATAAAATTCAAAAATCCAAAAACAAGGAGGGAAAGGAAGGGCTATCCCGAA
TCCTGCTACTAATTCCACCAAGTTTCTTCATCAGAATAAAGAGCATCCCTAGCCCCAGTTCACAGCCTCTTGGTGTGC
TTTAGATGCTCCATTCAGCTCAGGAGGCTGAGTGCTTGGATTACAGTCAAGAACCACTGCACTAGCCAGGTTTTTGGT
TTGTTTTTAATAGGGATATGGAAAACTTTCAAACACTGGACATGCATAATGGCACATGCCTGCAGTCCCAGGACTTAG
AGGGCAGAGGCAGGAGGATTATCATAAATTAGAGGCTAACTGGTCTACACAGCGAGTTTCAGGTTAGCTGAGGCTGTA
TATTAAGATCTGGATTCAAAAAGACTCAAACCAGGGGGCCATGGCTCCGTTGGTGGAATGCTTGTCTAGCATGCTTGA
TCTTCAGCGCTGCACAAACCAAGTGTGGAAGCACACACCTGTAATCCCAAATCTTGGCTAGGAGGCTAGAGGGTTGGG
AGTTCAAGACCAACCCTTGCTACATGAGACCCTTCCTCAGCTGAAAACAAACTGAAAGTTATCCAGGGTTGAGAATTC
CTTTGAGGGCCTTAAGGACTTTTTCAGGGAGACACTGTGGTAGCTATTGTACAACCGTATCCATTTCTCAAACTCAAC
AAATTGGGTTGGAGATAAGGTTCTGTGGTTAAGAGCACTGGCTGAACTTCCAGAGGGCCCGGGTTCTATTCCCAGTAC
TCACATGACAACTTATGAGAATTGATTCCCTCCTTCCATTAAGTTGGCTCCAGGGATTGAACTCAGGTTGTCCGGCTT
GGCTGCAAGCACCTTGAACACGTAAGCCATCTCGCCAGCCCTTGGGGTTTTCTCTGTTGTTTCCCAGTGAAGACAGTG
GCACCAGCTTGAGCTGGACAGACGGAACACTTTCATGATGAAACAGGATAAAGCCTGGTGGTACAGGCTTTAAGGTAC
AAAAATTGAAGCTGGGCCTGGTGTCTCATGTCTGGATGTCAGCACCTAGAACACTGAAGCACAATGGTCTTGAGTTGG
AGACCAGCCTAGACTAGGTGACAAGGCCCTACTTAAGAAGAGAAGGGGTAGGGCTCGAGAGATGGCTCAGAGGTTAAG
AGCATTGACTGCTCTTCCAGAGGTCCTGAGTTCAATCCCAGCAACCACATGGTGGCTCACAACCATCCCTTATGAGAT
CTGGTGCCCTCTTCTGATGTACAGATATACATGGAGGCAGAATGTTGTATACATAATAAATAAATAAAATTTAAAAAA
AAAGAAGAGAGGGGGTAAATGGGAGGGACAGAAACAAATTAACATGGATTTTCCAACCCCTAGAGAGGTTTGGAATGA
ATCAATTGTCATTTCTCCGAAGAAGGGAAGTAGAGAGAACCAGATGTTGAGTGAAGGCCTACTGTGCACCAGATCCCT
TTTCAGAGCTGCAGAACTAAGGAGAGCTGATTGTGCGTTCTCTCTGCACGCTTGTATGACTCCTACTATGTGCCGCAC
ACAGGCGGTGTGTCCTATGAAACACGCAGCAAATAAAACGCTGACTCCCGTCGGTGCACTGTTTGCATCACAACGCCC
TTCTCTGGGAAACGCATCATGTAGCCACTTTTTGACATTTCTTCCTTTGGAGGCTTAGAATATGTGTTGGTGGGAATC
CCAGAAACGGCCAAGAGGGCTGGGTTGGCATCCCAGAGGGGTAACAGCAGAGGGCCCCTCACCTGTTCTCCAACCAAC
TCCTGAGCAGTACCTGAGTCTGGTCCTGTCTGCTTCCCGGTGGCACCAGGGACACGGGATCTTGTCTGATTAACTGCC
TAATTCCAAGAACAACACGGACCCAAGGCACAGTGAAACTTCTGTGGGGACTCTGGGCCGGGAAAATACCTCAGGCCT
CTTGGGAGACACCCTGTGGTGGCACTGCCCAAGCCACCCTGTCCCTGGCAGGCAGCAGAACAAGGATACCCTTGGGTT
CTGAACTCACCTGCCAGCCTTTCCTTATTTCTCTTGCGGTTCCCCAAACCACTGAAATATCTTTTTGAGAAGCCGATG
GGTGTGGCCTTGGAAGGAGGCAGTGTAAACAGAGGCCCCACAAAGGAACTCCGAAAATTCAGAGGAGGTTTCTGAATT
TCGAACAGCCTGAGAAGGCCGAGCTAAACAGGAGACCCCAGGTACTTCCACTATGGTTTTCCACAATGAGCGGCCGCC
TGAGAAAGCATAGGAACTCAGATCTGTGGGAATCTCATTTCCATGACAGACAAGGAGAGATGTCTTCCCACAAAACTG
CAGAGCTGGGCGGGTGTGCGCGCCATAAGCATATACGCATGCGCGTGCAGCTGCGGAAAAAGTCATGCGTTACAGTTG
TCAGAAGCAAGCTGGAGCGTTTGGGTTGGCATAGCTATCTCTGTCTGTCCGTCTGTCTTTTGAGATGAGGTCTTACCA
AATAGCATTGACTGTCCTAGAATGTGATATGTACATCAGACTGGTCTTGAACTTTTGGAAATCCTCCTGCCCCAGCCT
TCCCAGTATTGGAATTATAGTCTTGTGCTTTTTTTCCCCTCTGTTTTGTTTTTTTAATTATATTTACTTACTTTTCTT
GTTTTGTTTTTTTTTTATTTTGTTTGTTTCATTTTGTTTTGTTTAGAGATGGGGTCTCTCTCTCTCTCTACACAGCCC
TGGCTGTCCTGGAACTCAGTAGATCAGGCTTGAACTCAAAGAGATCCACCTGCCTCTGCCTCTCAAGTGCTGTGATTA
AAGGTATGTGACACCATGCCCAGCTTTTTTTCTACATATTTATTTGTTTGTTTACTTATGTATTATGTGTATGTATGT
CATGCTTGCATGTATGTCTGTGCACCTTGTGAATGCCTGGTGTCCTCAGAGGCTAGAAGAGGGAGACAGATCCCCTGA
TCCCCTGTAACTGGAGTTATAGACTATCGTGAGCTGCTGTGTGTGTGTGTGTGTGTGTGTGTGTGTGTAGCATGCCAG
GACACAGAGAGAGCAACCGAAATCAAGGAATACCTTCAGGAGTTGCTTCTGTCTTGTGGGTCCTGTGCTCAAACTCAG
GTCATCAGGCTGGGCAGCAAGAAGTTTCAAACCCAGAGCCATCTTGACAGCCCATTGTCTTTGAAATATCAATAAGTT
AATTTAGTGGGTGTTTGTGTGAGTGAATGGGAACCCAGGTATCACAGGTTCTCAAACCAGCGCATGAACACAGAGGCC
AGAAAGGGCCCTGACATCCTCCTCTGTCATTTCCCATTCATTCATTTGAGGCAAGGACTCTTCCTGACCCTGGAGCCC
ATGTTTTCTCAGGTAAAGCCAGAAGCCAGGGAGCCCCTGTGTTGCTCCTGTGTTCATCCTCCTGGGAGCTGAGGTCGC
AGATGTGGGATGCTCGGCTTATTCTTGTTCTATGGCCGTGCGTGTCAGAACACTGGTGCTCACTCTCAGTGGCTGAGC
CATCTCCCCGGATCCTTTCCTTGTTAGTTGATTTTTGTTTTCATTTGTTTATTGTTTGTTTGTTTTTAAAGACAGAGT
CTCATGTTATAGTCCTGAACTCCCTATTTAGTCCAGGGCAGCTGATTAGCAGCTGCCCTCCTGCCTCAGTGTCTTGAG
TTCTAGGATCGCAGGCACACGACAGTATTCAAAAGACTCTCCCACACTTTCAAATTCTCACCACACAACCGTGATGCC
AGAGGAACTGTGTTCGGCAAAAGCCCAAAGTTGACTAAGCATGAAAGCTGGCCGTAGTTCGTTTTCTAACCAGCAGTG
CTACCTCACGCGGCTATTTTATCTCCCCATGACTCCACTTCCTCATCTGTAAGTGGGAACAATAATTCTTCCCAGTAA
TCGTATGGGAAGAAAGACACAGAACAGTGTCTGGCCTGTGGTGTTGCATCAGTACACAGTAGCATCCGGTGTGGCTAA
GCTTGAACAATGAGACAATGGAAGTGCTGAGTTAGCCCCACCCCCACCCTCGAGTCTGCATGATCTGGGCCAGGACTA
GAACCCTGGTCTCTAGACTCCTGCTCCATAAGGTCTTCTACAGCGTTACCCATCTAAGAATAGTTTTTTTTTGTTGTT
TGTTTGTCTTTTAAAAAAACTAGGTCTCAGTCTACAGCCAGGGCTGGCCTGGGATTCCCTATGTACCCTAGGCTGGCT
TCCAACTGTGTGTTGTCTTCCTGCCTCAGCTTCCCAGGTGCTGGAATTACAGGCAGGCATCAGCACATTCTCCATGTA
CCCAGGCTGACCTCGAACTCAGAGACCTACCTGCTTCTGCCTCCCCAATGCTGGGATTAAAGGCGTGTGCCACCACTG
ACTGAGATTTACTTATTTTTAATTTATGCATATGAGTGCTTTGCCTGAATGTATGACTATGCACCACCCAACCACCTG
GTGCCCTCAGAGACCAGAAGAGGTAACAAATCCGCTGGACCTGTAGTTACAGATGGTTTTGAGCCACCATGTGCATGC
TGGAAACCAAACCCAGGTCCTCTGCAAAAGCAGCCAGTGCTCTTAGCTTCTGAACCAATCTCTCCAGCCCAGAATAGT
AGTTTTAAATAATAAACTCTGCCATTCTGCCTTTCCAGACAGGGTTTGGATATGGGGTCCAGGGTAGCCTTAAACTTT
CAGAGATCTTGTCTCTTTTTTTATTTTGCACTTTTCTTCTCTTTTTCTTTAAAGTAAAATTTATTTATCTTGTTTTCA
AAGCAGTATAAAGAAGAAGAAGAGAGGGAGGGAGGGAGGGGAAGAAATCTGTCTGTCGACTCCAGTGTTGAGCAGTGC
AAGGCTCACGGCCTGCCACTGTGGATTCATTTGTGCCCTGCCCCCAACAACACACACAAATTCACATGCCAAAGTCTC
AACCCTCAGTGCATCACAAAACCAAGTCCTCTGTCCACAGCTTTGCAGAAAAACTGCGAAACAGGCCCTAGTAAAAAG
AAATGGGTGTGTGGGGTGTGTGTGTGAGAGAGAGAGACAGAGAGGGAGGAAGGGAGGGAGGGAAGGAGGGAGGAAAGG
AGGGCTTCTGTCTGCCTGGCCAGTAGCAAGCCTACAATTTACAGAGAAGACTGAACAAGGGCCAGGAGCGTGCATATG
TAGATTAGCAGGGAGATAATCTTGCCCCTCAGGGCATTTAGCTTCTGCCTTGGCAGGTCTCACCCTTAACCCCACCCA
GCTATGGGATTTTCTACAAACAATTTACTTCCTTAGGGTTCCCACTACCTTTACTACAGTGTGATGATATCTGGAGGC
AAGACCATTAGGAGTCACTGGCTTTAGGTGGGGTCCAGGTTAGGGTTTCATGATGGCACCCGTGTCTTCTTAAGAGTG
GACGCCAAACGCTGGGATGCAGCTCAGCTGGGAGACTGCTTGCCTAGCAGACACAAAGCCCTGGGTCTGTTCTCCAGC
ACTGCGTGCATGGGCTGTGGTGGCCCACGGTGCTTCTGTGGACAGAGCTGTGCACCACACCCTCCACAAATTCATATA
TTGAAGCCCAGCACTCAAGACGTGCAGGCAGGAGGATGAGGAGTTCCAGGCCAGGCTGGACTACTTGAGACCCTGTCT
CAAAAACAAGTAACAACAACAAAGGAATATGAAAGGAAAATGCACTACGTGTATGTTTGAAAAATGGCACAATGAAAC
ACATTAGTATGCATAACATTTCCCAGTAGGTTGCAAAAAAAACCCAAAAAACCAAAAAAACAAGGAAGAGATGTGTAT
GGCTTTTTATCGATTTATTTTTAACTATATGTGTGTCTCTTGGGGGTGGGGGGAGGTACACTTGTGAGTGCAGATGGC
TTCAGAGTCCATCCAGAAGGCATTGGGTCCCCTGGAGCTGGAGTTCTAAGTGTTGCGAGCCACCAACATGGGTGGGAA
CTGAACTTAGTGATGTGTGATCTTCACGGCTAGGCTGTCTCTCCAGTCCCAACATATAGCCTTTAATTTTTCTTTTTA
TTATTTTTATTTAGTGCGTACGTGTGTGTGTGTGTGTGTGTGTGTGTGTGTGTACATGAGTGCAGGTGTCTGTGAAGG
AGATGGCCATCAGCTGCCCAGGAGCAGAAGTTACATGATGCTGGGAACTGAACTCAGACCCTCAGAAAAACAGCTTGT
GCTATTAAGCAGTTGGCCATCATTCTAGCTCCAAGAATAGTTTTATTTTGTTTTGTCTAAACCAGTATAGTTTCTTTT
ACATGATAATGACAACTGATAGCCAGAATTTTCTGAAAGACTTAAAGTTGTGTCATTTTTTAACAGGCCAGAAACTCT
ACACTGACAGTACTGGTTCATTGTCTTCACTTTTGTTTTGTTTTGGTCGCATTTTGTTTGTTTGTTTGGTTGGTTGGT
TGGCTGGTTGGTTGGTTTGGTTTTTGAGATAACATCATGTAGCCTAGGCTGGCCGTAAACTTGCTATATAGTCTAGGC
TGCCATGCTTGCCTCAAACATGTGACAATCCTCTTGCCTCCCAGGTGCTGGGATTCCAGGCATGAACTACACATAACC
ACATCACCTTCCTCTCCATCCCTCTGTCCCTTGTGAGGTCATCTGGTCCACCTGTGGCTGACTCTATAGGGACTTAGG
TGACTATGCCACTTTCTCCATGGAGAACTCAGCAGCCTCCCTTGGGGCCCCACCCTGCTGAGTTCCTCTGTAGCTGGT
GATGCCTTAGGCTCACACAGTCCCTTGGTCTATTTCCTAGACTGTTAGGTTTGGCAGCGTCACAGTTCCTCCTTTAAC
TGTTCTTTCCCAAGTGAAAATAGCTGGCACAGTGACCTCACCGGAGCCAGGCTGGCATCTCTCGGCCAGCGCCTCCAC
CCACGGTTTGTTTTCCATCATTCGTGTTTGCAGTGTTACTCAATTTCCCTGCACTCTACCACACAGGGCGAAGCTCTC
CAAAGTTGTCAAGATTTTGTGTTAACCATGGACAAGGCCTCATTTGGGGGGTCTTGAGGGCATTTCAGGCGAACTGTC
CATAACAAACTGGGAAGGGCATGATGCTCTTAAGAACTATGGCTCCAGCCAGTCCCTGTGTCCCTCCCATGTACTCTC
TGTCCTCTAAACACCTGCCATCTCCAAGTTGCCTCCTTATCTTTATCCCAGAAATCCATTACTTAGTTTATATATTGT
CACCTCCTTCCCTTCCTCCCCCTCCTCCAGGATAAGCAATAATTTATTCTGGAGCCAAATGTGAGCAACCGTGGACCA
AGAACAAACACATTTTCAGGATACACTAAATGCCCTCTTTCTTTCTCTCCCTGGTAAGGTTTCATGTGGCTCAGACTG
GGCTTCAACTTGCTATACAGTGAGGATGACCTTGAACTCATCATCATCCTGCACCCCCCCCCCCAGTGCTGGATCACA
GGCATGCACCACTACATCCAGTCTGTGCAGTGCTGAGGACCAAACTCAAGCTCCCCGTTTGTCAAGCAAGCACTCCAC
CGACTGAGATACATCTCCAGGTATTTGTTTGTTTATGTATGTGCATTGGTGTTTTGCCTGCATGTATGTCTGTATGAA
GGTGTCAGATTCCCCAGAACTGGAGATACAATCAATTGTAAGTGGCCATGTGGGTGCTGGGAATTGAACCTGGGTCCT
CTGGAAGAGTAGGAGGTGTTCTGAATTGCTGAGCCATCTCTCTGGCCCATATTTTTTAAATTATTGTTGTTTGTTAGG
TTGATCCAGGTTCTGTAACCCAGGCTGGCTGGAGCCAATGGCAATCCTCCTACCCCAACCTTCCAAGGTCTAGGATTA
CAGCATGCACCTGCAGGCTGCTTATCTAGATTCACTATCTGAGTCAACTTTCCCAGGCACAGGAAACGGAAGCCAGGT
GTGGTGATCTCAGCACTCAGGAGACTGAGGCGGGAGAGCTCCTCAAGCTCAACGCTTGGCTACATAGTACATTTTAGG
CCAGCCTTGGTTACAGAGCAAAATCCTGCCTCAAAAAGAAGAGGAGGAGGAGGAACAGGAAGTAGTCAAGAATAAGAA
ATGGCTGGAGGATGAAGACAGGATATAGCCAGGGTTTGTTTCGACAAGGACTTGTTTGGGTTTTGATTTATTAACCTA
TATCTTTGCCGAATGGAAATTAAACCTAGGACTTCGCACTGGCAGTAGCCAAATTACCCTATCACTGAGATCCACCCA
TATAGCCTCAGGGCAGGATTCATGAGACAGTCTGTTAAGTGGCTTAACTCCTGAACTCTGAAGGGGAAGAGTTGAGAC
AGATGTCTCCCTCCCCTCCCCTCTCCTCCCCTTTCCCTTTCCTTCTGGCCATACAAAGATTGCTGTGCCCAAAGCTTC
CCAAACTCCAGAGATCATGGAAAGTCTGTGTACCTGCCACTCTTCAGAGGGAAAGTTTCAGATTTACCAGCGATTCAG
AAAGACGGCTTCACAAGTGTGTACTGCAAGATAAGCCTGCGTATATCAAGGCACTTTTCAAAAGTATATAAACAGAGC
TAATTTATTAGCATAGGAGAGAGAGAGAGAGAGAGAGAGAGAGAGAGAGAGAGAGAGAGAGAGAGAGAGAGGCCTCTT
CTTCTGTTCCATCTGTTCCCAGAGCATGCTGAACTGGCGTGGAAAGGAAAGATCTTTTGACTACTGGCTTTTATCCTC
TGGCCCCCTCCAGTCACTACCAGGGAGTGATGTGATGTTTACCCTCAGGCATTTGGCCAATCAGAAAGGCCCAGTAAG
TGATCAGCTTTTGCTTAATGGGGCCACTGATATTCCCCGTCACTCCTGTATGTCAGCTGTTTGACTTGTTCCCTCAGC
TATATGGCTCTCAAGATGAATGGAGTTTTGTTGATGTTTGTTCATTTGGGGAGAGGGGGTTGGGGTTTTGGGGGGTTT
GATTTTGTGTTTGTTTTGAGACAAGGTCTCACTTTGCAGCCCTGGCTGACCTGGCACTCACTGTGTATACCAGGCGGG
TCTTGAACTCACAGACATCCACCTGCTGCTTCTGCCTTCCAAATGCTAGGATTACAAGTGTGGGACACCATGCCCAGC
TGTCTTTATATTTACCCTACCTACTCACTTACTTTGAGACAGGTTCTCACATAGTCTAAGCTGACTTTCAACTCTCAA
CAGAGCCAAAGATGGCTTTGAACTTCTGATTCTCCTGTTTGTACCTCAGCAGTGCTGGAATTGTGGGCATGTCCCACC
ATGGTGGCTCATCACCATGCTCAGTTTATACAGTGTTGGGGACGGAACTCAGGGTATAGTGCATGCTGGCAAGCAAGC
ACTCTTCTTAGCTGCAGCTCCAGGCCCAAGGGAAATAGTTTTAATTACACACCCACCCACATACGATTTAGGCAACAC
TCAGCTTTGCCAACTGGTTCATTCATTTAGTGAACAGCCACCTTCTTCCCAGAGGCCAGGCTCTGGAGTGGAGGGTGT
AAGCATATCCCCTGTGAAAGGTTCAGGCTTTAATGCTGATCGGCTCTACCTCTTTAAGAGTCAGACCCCACCCCCTGA
CAACAGTCAGGGCATGCACAGGAAAATGTGCTACGTCATCACCACCTCACCTGACATGCATTATGGCCAGCGTGGGGA
CTCTGGGCGTGCGTGGAACCTCAGTGCCCATGCGCAGTCTTCCCTTTAAAAGTTCCAACTTCCCTGCTTCGCTCTTTC
TGCTTCTTCTACCCTCTCTCTCTACTCACTCCTTCTCTGCTCTCACCTACGTGCTCTCTATGGTGGTCAGCCATTACC
CCTGTTCCTCTTCTCTCTTGGTCTCCCTACTCTGCCCGGCCTATAATAAACTAGTCAATATGTCTCATCTTGCGCCCT
CCTCTTTTTCTACAAAAACATAACACCCTGTCCATCAGCCCTCACCATCTACTGGAGGAGAAAGATGTCAAAGCACAT
GATTAGAACACAGTCAGTCAACGAAGAGGAGCGGATAGCCTGGGAATTCAAAAGCAAGATCATGTGTGCCGCCCTCCT
CATAGCGCAGGGTAGGAGCAGACTCGGCCGGACAACGGACATCATGGGATGGCTTACCGATCCCCGCCTCACATGCTG
TGTCTCAAGGCAGCACTGGCAACAAGCTGGGTTAATTTTCTTTTCCATTGTCCTCCCTGGTTGAGGTGCCCTTCCCTC
CTTCACACTCGTTTTCTGGGCTTCCAATTAGCTGATAACCTGATGACTATGTAGCCCAGGCTTGCCTTAAACTCTGGG
CTGACCATCCTGCTGCCTCGGCTTCCCAGGTGCTAGGATTATATGCATGAGCTACCACACCAGGCTCAGCATGAGCAC
CTTAAAACAGGACCCTTAGGCATGTTTAGGTCAGGCATCTGGGGACATCGAGGAGCTGGAGAAACATCGGTAATCAAA
TTCCTCCAGAGGCAGGTTATCATTTGACTAGCTTTTCTCGTAAACTTCCCAGGCTCACAAATCCCTTGCTGTTCCTCA
AGTGTGTAGCCAGCCTTTGCAATGCAAAGTAACCTCATTCTGCAGGAGAAGCAGAGACCACGCTTGCACGCAATTTGA
CTATGGTGACAGCTATGAAGTGGTGGCAGACAGAGAGAAGTTAGTGTTTAGTTTGAGAAGGGTGTCAGGTTAGGGTTT
GTTGAGTAAATGCATAAAGGAAGGAAAGGCTACTTAGGTAGAGGGAGTGTTGTCATTTCTACCTTAAATCCCACCCCT
CACCCCACCCCCACCCCCCACCATCTTCACCTCCCACACTGCACTATAAGGAGGGCTGTACACACATCTCACACCACC
AGACGGAGGTCTGAGGATATAGCTCAGTGGTCCCCCACGTTCCTGGTATGCACAAGGCCCTGGGTTCTGCCCCAGCAA
CATACATAAAGCAATAATTATAACAAACATCCAGAAGGAGTGGGCCAGCAATGGGCTTAATAAGGTAAATAAAATGGC
CCAAGGTAAAAACCTAGCCAGAGAGCCGGCCAGGATAACCCAAGTCTCACTGACCAGAGACTGGGTCTATCTTCATAC
CCAGGGTGAAGGCTGATGCGTGCACCTCTCTAAGACCTTCTAGGAATTCAGTCTAGGAATTCAGTCAGATCTAGACAG
AAGAAAGTCAGAAGCATAACCAGGACTCGGTGGCTCACATATGTAATCCTAACATAGAGGAGGTAGCAGCAGAAGGAT
CAGGAGTTCAAGACCATCCTTGGCTACATATGAAGCTAGCCTGAGTTACATGTCTCAAAAAAACTCAAGCACATTAAG
GAGGAGACTCATTCCATAAAGTCCTTGCCTCACAAGCTTGATGACCTGAGTTGGGATTTTCAGAGTCCATAGGTGTAG
TGGGAACATTTAACTTTTTTTTGTTTTTAAATTTCTACAAGTTAACACAATGTATTAGATATGTAACAGCTACACTGG
TTTGACTACAGAAAATAATAAAAGTGTTCTCTTAACAATGAAGGAAAATATCTGTAACCTGGATATATATGGTGGCAC
ACATCTGTCATCCCAGCACTCAGGAGACAGAGGCAGGAAGATGTCAAGTTCAAGGTCCGCCTGGGCTGCTTAATGATT
TTTGAGGCCAGCCTAGACTATGCAGCAAGGCTTAGTCTCAAAAATAAAATAGAGCTGAGTATACAGTGCCTACCATCC
TTCAGGGGTTGTTTAGAGAGGACAGAAGTGGTACAAAGATGACCTCTAAGACCAGTATAGTAAAGGGGGCCTCTCTCC
AGGGAACCTGGTCCCTATCTCTCAGTCAAATCCCACATAGTATCAGATGTTCCCAGACAAAGGTCTGTGGGATCCCAA
AGGCATCATTGGCCATCCTGGCCACTTTACTCAGTGCCTGAGGTCTCCACCAGGCTCTTGCTGTGCCTCAGTTCCAGC
AGCCACCTTTGTTGTCTGTGATGGTTTGAATGAGAATGGTCCCCATAGGCTCTTACGGTTGAATACTTGATCCCCAGT
TGTTGGACTGTTTGGGAAGGATTAGAAGGTATGGTATACCAAGGTTTTTTCTTTGGGGCCTCCAACCACCTCCCAAAT
CATGACACGGAGATTTCTCAGTAGACCAGGCTAGCCTCAAACTCACAGAGACCCACCTGCCTCTGCCTCCCCAGTGCT
GGGATTAAAGGCAAGTGCCACCACCGCCCGGCTACAAGGCGTATTAGTTATGAATGCTCAGTCTAGCTTAGGCTCGTT
TCTGGCTAGCTCTTTCAACTTAGATTAAACTGTTTCTCCTTAACTACCTTTTGTCTCAGGGCTTTTTACATTTCTTTC
CTTCTGTATATCTTCCTTTCTCTGCTTCTGGGGTCTGACTGGCTGGTGGCTGGCTGGCTTCTTGTTTCAGGCTTGCCC
CTCTCTTTCTCTCTCATTCTCTCCTCCTCCTCCTCCTCCTCGCTCTTCTCTCCTATTTATTCTCTCTGTCTGGCAGCC
CCACCTATCCCTCTCCTGCCTAGCTATTGGCCGTTCAGTTCTTTATTAGACCAATCAGGTGCCTTAGGCACACAAGGC
GAAACAAATGCATCACATCATTAAATAATTAAACAAAGCATTAAACAAATGTAACACATCTTTGCCCAGTTAAACTAA
TATTCCACAATAGTATGGCCCTGTTGAAAAAGGTGCCTTACTAGAAGTGGGTGTTGAGGTCTCAAAAGGCTCCTGTCA
ACCCCAGTATGCTCTCTCTTTCCTGTTTGAGATGTGAACTCTAAGGCATTCAGGCAGCCAGGCCTTCACCCCACCGTA
ATGGACTCTAACCCTCTGAAAGGGTAAGCCCAAGTAAACATATATGTGAGGCTGTGCTGGCTGGTCTTGACGCACTAA
CTGGAGTTATCTGGAAGGAAAGGAGTGAATTTCAATAGAGATAATGCCTCAATGGTTATGTAGATCCAGCTATATATA
ACCACATGGTGGCTCTCGGCCATCTGTAATGGAATCAGACTCCTGGTATGCATGAAGACAGAGCACTCACATACATAA
AATACATGAATAAATACATCTTAAAAAAACATTTATACTAGATAATTTTTAAGGCTGGAGAGATGGCTCAGCCTTTAA
GAGCACTGTCTGCTCTTACAGAGGACCAGGGTTCAATTTCCAGCACTCACATGGCAGCTCACAACTGTCTGTAACGCC
AGTTCTAGGGTATCTGACATCCTCACTCAAGACACACATACAAGTAAGACACCAATGCACATAAAATAAAAATAAATT
TTTGTTTGTTTTGTTTTTCAAGACAGGGTTTCTCTGGGTAACAGAAAAATAAAATAAAATAAAATAAATGTAAGATCC
AACTGTAAAGCATTTTCTTAATGATTCATGGGGGGAGGGTTGAGCCCATTGAGGGTGGTTCCATCCCTGGGCTGGTGG
CCCTGGGTTCTATCAGAAACACACTGAGGCCAGGCAGTGGTGGGCATGCTTTTAATCTCAGCAGAGGCAGGTGGCAGG
AGGCAGAGACAGGTGGATCTCTGTGAGTTTGAGGCCAGCCTGGTCTACAAAGCTGCACAGAGAAACCCTGTCTCGAAA
AGCGGGGGAAAAAAAGAAGGAAGGAAGGAAGGAAGGAAGGAAGGAAGGAAGGAAGGCAGACTGAGCAAGCCAGGGGGA
GCAAGCCAGAAAGCATGCAGGACCCTCCATGGCCTCTGCATCAACTCCTGCCCTGATTGAGTTCCTGCCCTGACTTCC
TTCAATGATGAACAGTGCTGTGGAAGTGTAAACTGAAAAAATCCTATCCTTCTTAACTTGCTTTTTGTTTGGTCGTGG
TGTTTCATCCCAGCAATAGAAACCTTAAGAAGTGCCTTGATCATGATGTTTTAGCACAACAATCAAAATGTAACTAAA
ACCCCTTCCTCAGAACCCCTCCCCCACACGGCAACCTCCAGAAGCCTTTGTCTATATAACGGGGTCTTCAGCACAAAC
TTGTGACCACTGAGGATGTTTAGGAAACACATCCTGGAAAGCTAGCGACATCCCCAACACAGCGGCCAGTCACGTGTA
ACTGTTATTTTACATGGCAATTAAATGAGAAAATGCAAATAAACGCTCCCAGGCTGACCCAAAGGAGAACAAATAAAG
CGGCCATGAGAAGGTGGGGTGTGACCCCACAAACAACCTGGAGGTTTGGAGTGAGGGAAAAAATCACCTCAAAGACAC
CGGTGGACTTTGTGTGGTTTCCCCAGGTGCACTGGCCACTTGGCTGAGCCATTATCAATCACACAGATCCATTATCAG
CCCTGCAAGAGTGGGCAGGAGAAAGGCAACCTGTTCTTCCCTCCTAAGCCACCTGTGCTGGTCACTCCTCCTATATAT
ATCTGTCTCTATCAAACAACACATATTAAACTCAGGCCTAAACAAACAAGTAAAAAAGGTTAATTTCCTGGCAGGAAA
TTCCTGTGTAATCAGTCAGCAGCTCCGGTCCCAGCACAGTCGCTACCCAGCACTGTGACCTTTGGCAGGCCCACCCCT
CTCCAGACCCATTAGTCACCCCAACCCTGCAGGCTGCGGCCTCTCTCCTGGTCTCTGCATCTCTGGTTTCCCTTCCCT
AGTAGAGACCCGCAGAGTGGAGATCCTTCTAAAAGACCTGCTCTGTAGCTCAGAGTTAGGGCATGCGCAAAGCCCCAG
GATCAGTCTCCAGTGCTGTCGGTACTTGAAGGAGGGATCTGGTCGGTACTGCCTGGTTTAAATCTATAATCTCTGGGT
TAATGGGGTACCAAGAGAACACACACTTGTCATGCACGAAACCCCGGGCTCAATCTCCAGTGCCACCAAAACAAACAA
ACAAACAAACAAAAGCCTTATATTTAAAATCTGTAACCTCTAGAAAAACAATAATAAAATCTGTATCCTTTACTCCTG
GATGGAATTTGTAATGATTAATATTGCCAACGTGGCAGAATCTTGAATCACCTGGAAGGCAAGCCTCTGAGTTCCCCT
GAAACAGATTATGTAGATCAAGTTAATTGAGTGGGAAGTAGCATTGCTGGGCTACATAAAAGGAAGAAAGCAAACTGA
ACAGGAGATCCCGCACTGCTTCCGGCTGTGACCAGGAGCCTGCCTCCAGGACCTCCCCCACCATGATGGACTGACGGG
ATTCTTGAACTGTCGGCCCATATAACCCTTCTCCCTTCAGTGGCTTTATTTATTTGTTTGGAAAGTAGGTTTTCAAGC
TGGGCATGGTGGTACAAACCTTTAATCCCAGTACTCAGGAGGCAGAAGCAAGTGGATCTCTGTGAGTTCAAAGCCAGC
CTGGTCTACAGATCGAGTTCCTAAGACAAGCAGCTCTGCTAAGAGACCCTTTGTCTTCATCGCTGTTCCACTCACTGC
TGTAAAGAGACACCATGACCAAGGCAACATTTTAAAGAGAAATCATTTAATTGGGGCTTGCTTACAGTTTCACAGGTT
TAGAGGCAGGGAAGGGGGGAGGAAGGGCTAGGGAGAGGAGAAACACTGACTGGCTGGTGGGGAAGCCCCCCCCCCACC
ATCCAGTGATGCACTTTGTCCAACAAGGCTACACCTCCGAATCCTTCTAATCACGTCAATCTCCACACCCTGGAGACT
AAGCATTCAAACATATCAGCCTATGGGGGCCACTCTTATTCAAGCCACCATAACCTGTCTCAGATAAAAAGAAGTTTT
TAGTTGAAATAGAATTACATCACTTTCCTCCCTACAGCCCCTCTCCTTACTCACTCCGTAGCTGGGGATGACTTTGAA
CCGACCCTGCTTCCTCTACCTTCTCCGTACTGAGATTGGGGGCATGCACCACTATGATCTAAGTCTCTCTACAGGTTC
CCTCTCTTGTTTCATGTCCCTGTCTAGTTTCATGTCACCGAGCCCCATTTACCCAGGTTATTCTGCATTGTGCCAAGC
TCAACAATCAAAACTAAACATCACAACTATACAGTAAGATTCCAAACAGAACAAATAAACAAATACGATAAAAACAAA
TAGAGCCAACTCTAGAAAAAAAAGTCAGGTCTGATGATAACTCAGGCTTGAGAAAGTGCTTATCAGATGAACACAACG
GGAGACGTGGCTTCTAAGCAGGGAGCTTACCTCCATGCTGTAAAAGTCATGACTACTTTGTTAATCACCTCCCACATG
AAAGGCCTTATCTTTGGAGGAAATAAAAAGGAGTTCTTTCTGTTTGTCTTTCTTTGAGGCAGGATCTTACTATATACC
ATGGCTGTCCTGGAGCTCGTTCTGTAGAACAGGCTGGCCTCAAACTCACAGGATCTGCCTGCCTCTGCCTCCCAAGTG
TAATAGGGGATGCCCTTCTGTATATATGTTTCTCTTGTTGGTTGATGAATAAAACACTGTTTGGCCAATGAGGCAGGA
AGATTCTGGGAAAGGTAGGCAGAGGAAGACAGCAAAACAGTCGCCATGTGGAGACCAGGAGGATAGGATGCACCAGGA
GTTCTCCAGTAAGATAAGGCCATGTAGAAATACATAGACTTATAGAAATGGATTAATAATTAAGACAGAGCTAGCCAA
TAAGAAGTCCTAGCCATCAGCCAACAGTTTTATAATTAATACAGCATCAGTGTTTTTATTTGGGGCTCAAGGACAGCA
GGACCTGGCAAGACAAGAAAACCGCCGGCAACACAAGTGCTAGCATTAAAGGCAGGCTCCACCATGCCTGGTTTGCCT
CAGCCTCTGGAGTGTTAGGATAAAAGGATGAGCACTTTGCCCAACTTGGGATGCTTTTGTTTATTGGTTTGTTTGGAG
TCAGGGTCTCATAGAACCAGGCTGACCTTTAACTTGCTATGTGCTGAAGAAGCCTTTAAGCTTGTGCTCTTTCTATCT
CAACTACCCAGAGTGGTACAATTACAGTTGTGAACCATGCAGCCAGTTTCATGTGTGCTGGGAAATAACCTCGGAGCT
TCCTACAAGCTAGGTAGGCACTCTACCAACCGAGAGCTACATACCCAGCCCCCAGCATTCTTTCTTATTCTTAGCCTT
ATACTGAATGGGCTGGCCTCCTTAGATCCTGACATTGATGATAGTACCTGGAAGGTGGTAGCTGCTGCATGAATGAAT
GAATGAATGAATGAATGAATGAATGAATGAATGAATAGCTCACAGTAACTAAGAGAATCCAGTGCCAGCAGTGAGCAG
AGGACTTTACTTAGAGTGTCTCATTGACTGCTCTAATTGCCCTACTAGATATGGCATTCTTTCTAGTTGAAGCCATTC
TCCAGTTCATGCAGAACAGTGTGATGTTTCTATGTCGGCTTAGCGGCTGCACATGAATCTACTGATATTTTAATTATC
GTCTGTGGCCCCAAGGCTCTGCAGTCAGCACCCTGCCAGCCACTCAGTCCTCAATCTTACGGGGCTTCAGGTCCCACA
GCCCCAATTCTGCTCTGGGGCAGAGATACGTAGGTTTAAATTAAGTCTTTATCCTTCTATTTACCAACAAAGACTCAG
AAGTCAGATGTGGGGTTGAAAACCTGCTAGATCAGAGAAGTCGAGAAGCAATCAGCTTTCATCCCCCAAAAGAGACAT
GATGGGGGAGGTCCTTCTGCATATATGATTGTCTTATTGGTTGATAAATAAAGCACTATTGGCCAATAGGGGAGCAAG
TTAGGCAGGACTAAGAGAGAAGGAGAATTCTGGGAAATGTAGTAAGAGAAGTCTTGTGATGCAGGCAGGAAGTGACAT
AGCAGGCAGACTCAGAATATAAGCAGGGACAAGCAGGAAATCGCTGTCTTCCTCTTTCTCCTGCTCTCTTCTCTGTGG
AGCCACCATGTGATCC

What is claimed is:

1. An isolated Chinese hamster ovary (CHO) cell, comprising an expression construct that comprises one or more open-reading frames (ORFs) encoding one or more heterologous polypeptide(s), wherein the expression construct is integrated in the CHO cell genome at an integration site within about 20 kilobases (kb) of a sequence having at least 97% sequence identity to a sequence selected from the group consisting of SEQ ID Nos:1-3.

2. The cell of claim 1, wherein the integration site is within about 10 kb of a sequence having at least 97% sequence identity to a sequence selected from the group consisting of SEQ ID Nos:1-3.

3. The cell of claim 2, wherein the integration site is within about 5 kb of a sequence having at least 97% sequence identity to a sequence selected from the group consisting of SEQ ID Nos:1-3.

4. The cell of claim 3, wherein the integration site is within a sequence having at least 97% or at least 99% sequence identity to SEQ ID NO:2.

5. The cell of claim 4, wherein the integration site is within the sequence of SEQ ID NO:2.

6. The cell of claim 3, wherein the integration site is within a sequence having at least 97% or at least 99% sequence identity to SEQ ID NO:3.

7. The cell of claim 6, wherein the integration site is within the sequence of SEQ ID NO:3.

8. The cell of claim 3, wherein the integration site is within a sequence having at least 97% or at least 99% sequence identity to SEQ ID NO:1.

9. The cell of claim 8, wherein the integration site is within the sequence of SEQ ID NO:1.

10. The cell of any one of claims 1-9, wherein the integration site is the only genomic site at which the expression construct is integrated in the CHO cell genome.

11. The cell of any one of claims 1-10, wherein the cell lacks dihydrofolate reductase (DHFR) activity.

12. The cell of claim 11, wherein the cell comprises loss-of-function mutations or deletions in both copies of a DHFR gene.

13. The cell of any one of claims 1-12, wherein the CHO cell is a DG44 CHO cell.

14. The cell of any one of claims 1-13, wherein the integration site is an intergenic site not within an intron or exon.

15. The cell of any one of claims 1-14, wherein the integration site is located in open chromatin in the CHO cell genome.

16. The cell of claim 15, wherein the integration site is within about 5 kb or less of a peak based on Assay for Transposase Accessible Sequencing (ATACseq) analysis.

17. The cell of any one of claims 1-16, wherein the expression construct comprises one or more ORFs encoding an antibody, enzyme, or fusion protein.

18. The cell of any one of claims 1-16, wherein the expression construct comprises:
(a) a first ORF encoding an antibody light chain and a second ORF encoding an antibody heavy chain or antigen-binding fragment thereof; or
(b) an ORF encoding a single chain antibody or antigen-binding fragment thereof.

19. The cell of any one of claims 1-18, wherein the expression construct further comprises a promoter operably linked to the one or more ORFs.

20. The cell of any one of claims 1-19, wherein the expression construct further comprises a sequence encoding a selectable marker.

21. A method of producing one or more heterologous polypeptide(s), the method comprising culturing the CHO cell according to any one of claims 1-20 under conditions suitable for production of the heterologous polypeptide(s).

22. The method of claim 21, further comprising recovering the heterologous polypeptide(s) from the CHO cell.

23. The method of claim 21 or claim 22, wherein the CHO cell is cultured for at least about 40 days, and wherein an amount of the heterologous polypeptide(s) produced by the CHO cell on Day 40 of the about 40 days is at least about 85% of an amount of the heterologous polypeptide(s) produced by the CHO cell on Day 1 of the about 40 days.

24. An isolated Chinese hamster ovary (CHO) cell, comprising a landing pad sequence for mediating recombinase-mediated cassette exchange (RMCE), wherein the landing pad sequence is integrated in the CHO cell genome at an integration site within about 20 kilobases (kb) of a sequence having at least 97% sequence identity to a sequence selected from the group consisting of SEQ ID Nos:1-3.

25. The cell of claim 24, wherein the integration site is within about 10 kb of a sequence having at least 97% sequence identity to a sequence selected from the group consisting of SEQ ID Nos:1-3.

26. The cell of claim 25, wherein the integration site is within about 5 kb of a sequence having at least 97% sequence identity to a sequence selected from the group consisting of SEQ ID Nos:1-3.

27. The cell of claim 26, wherein the integration site is within a sequence having at least 97% or at least 99% sequence identity to SEQ ID NO:2.

28. The cell of claim 27, wherein the integration site is within the sequence of SEQ ID NO:2.

29. The cell of claim 26, wherein the integration site is within a sequence having at least 97% or at least 99% sequence identity to SEQ ID NO:3.

30. The cell of claim 29, wherein the integration site is within the sequence of SEQ ID NO:3.

31. The cell of claim 26, wherein the integration site is within a sequence having at least 97% or at least 99% sequence identity to SEQ ID NO:1.

32. The cell of claim 31, wherein the integration site is within the sequence of SEQ ID NO:1.

33. The cell of any one of claims 24-32, wherein the landing pad sequence is heterologous to a Chinese hamster genome.

34. The cell of any one of claims 24-33, wherein the landing pad sequence comprises a first and a second target sequence recognized by a site-specific DNA recombinase, wherein the first and second target sequences are heterologous to a Chinese hamster genome.

35. The cell of any one of claims 24-34, wherein the landing pad sequence further comprises a sequence encoding a selectable marker.

36. The cell of any one of claims 24-35, wherein the integration site is the only genomic site at which the landing pad sequence is integrated in the CHO cell genome.

37. The cell of any one of claims 24-36, wherein the cell lacks dihydrofolate reductase (DHFR) activity.

38. The cell of claim 37, wherein the cell further comprises loss-of-function mutations or deletions in both copies of a DHFR gene.

39. The cell of any one of claims 24-38, wherein the CHO cell is a DG44 CHO cell.

40. The cell of any one of claims 24-39, wherein the integration site is an intergenic site not within an intron or exon.

41. The cell of any one of claims 24-40, wherein the integration site is located in open chromatin in the CHO cell genome.

42. The cell of claim 41, wherein the integration site is within about 5 kb or less of a peak based on Assay for Transposase Accessible Sequencing (ATACseq) analysis.

43. A method for generating a cell line that expresses one or more heterologous polypeptide(s), the method comprising introducing a polynucleotide comprising an expression construct that comprises one or more open-reading frames (ORFs) encoding the one or more heterologous polypeptide(s) into the cell according to any one of claims 24-42 under conditions suitable for RMCE between the landing pad sequence of the CHO cell and the expression construct.

44. The method of claim 43, further comprising selecting for cell(s) that integrated the expression construct at the integration site.

45. A polynucleotide, comprising:
(a) an expression construct that comprises one or more open-reading frames (ORFs) encoding one or more polypeptide(s); and
(b) a first homology arm and a second homology arm, wherein the first and second homology arms flank the expression construct, wherein the first homology arm comprises a first sequence of about 50 to about 1000 nucleotides in length from SEQ ID NO:2, and wherein the second homology arm comprises a second sequence of about 50 to about 1000 nucleotides in length from SEQ ID NO:2, wherein the second sequence is 3′ relative to the first sequence within SEQ ID NO:2.

46. A polynucleotide, comprising:
(a) an expression construct that comprises one or more open-reading frames (ORFs) encoding one or more polypeptide(s); and
(b) a first homology arm and a second homology arm, wherein the first and second homology arms flank the expression construct, wherein the first homology arm comprises a first sequence of about 50 to about 1000 nucleotides in length from SEQ ID NO:3, and wherein the second homology arm comprises a second sequence of about 50 to about 1000 nucleotides in length from SEQ ID NO:3, wherein the second sequence is 3′ relative to the first sequence within SEQ ID NO:3.

47. A polynucleotide, comprising:
(a) an expression construct that comprises one or more open-reading frames (ORFs) encoding one or more polypeptide(s); and
(b) a first homology arm and a second homology arm, wherein the first and second homology arms flank the expression construct, wherein the first homology arm comprises a first sequence of about 50 to about 1000 nucleotides in length from SEQ ID NO:1, and wherein the second homology arm comprises a second sequence of about 50 to about 1000 nucleotides in length from SEQ ID NO:1, wherein the second sequence is 3′ relative to the first sequence within SEQ ID NO:1.

48. A vector comprising the polynucleotide of any one of claims 45-47.

49. The vector of claim 48, further comprising a sequence encoding a selectable marker, wherein the selectable marker sequence is not flanked by the first and second homology arms.
